(19) United States
(12) Patent Application Publication
(10) Pub. No.: US 2018/0213800 A1
(43) Pub. Date: Aug. 2, 2018

(54) PENICILLIUM ENDOPHYTE COMPOSITIONS AND METHODS FOR IMPROVED AGRONOMIC TRAITS IN PLANTS

(71) Applicant: INDIGO AG, INC., Boston, MA (US)

(72) Inventors: Slavica DJONOVIC, Malden, MA (US); Elizabeth Alexa MCKENZIE, Milton, MA (US); Gerardo V. TOLEDO, Belmont, MA (US); Craig SADOWSKI, Somerville, MA (US); Geoffrey VON MALTZAHN, Boston, MA (US); Karen V. AMBROSE, Cambridge, MA (US); Xuecheng ZHANG, Newton, MA (US); David Morris JOHNSTON, Cambridge, MA (US); Trudi A. GULICK, Topsfield, MA (US)

(73) Assignee: INDIGO AG, INC., Boston, MA (US)

(21) Appl. No.: 15/739,063

(22) PCT Filed: Jun. 24, 2016
(86) PCT No.: PCT/US2016/039191

§ 371 Date: Dec. 21, 2017

Related U.S. Application Data
(60) Provisional application No. 62/185,471, filed on Jun. 26, 2015.
(60) Provisional application No. 62/185,429, filed on Jun. 26, 2015.

(51) Int. Cl. A01N 63/04; C12N 15/82

(52) U.S. Cl. CPC A01N 63/04; C12N 15/8226; C12N 15/8227; C12N 15/8225

(57) ABSTRACT

This invention relates to methods and compositions for providing a benefit to a plant by associating the plant with a beneficial endophyte of the genus Penicillium, including benefits to a plant derived from a seed or other plant element treated with said endophyte. For example, this invention provides purified endophytes, synthetic combinations comprising endophytes, and methods of making and using the same. In particular, this invention relates to compositions and methods of improving soybean and maize plants.

### CROSS REFERENCE TO RELATED APPLICATIONS

This application claims the benefit of U.S. Provisional Application No. 62/185,471 filed 26 Jun. 2015, and of U.S. Provisional Application No. 62/185,429 filed on 26 Jun. 2015, all of which are hereby incorporated by reference in their entireties.

### SEQUENCE LISTING

The instant application includes a Sequence Listing which has been submitted via EFS-Web and is hereby incorporated by reference in its entirety. Said ASCII copy, created on Jun. 24, 2016, is named 33898PCT_sequencelisting.txt, and is 2.20 MB in size.

### FIELD OF THE INVENTION

This invention relates to compositions and methods for improving the cultivation of plants, particularly agricultural plants, such as soybeans and maize. For example, this invention describes bacteria, such as strains of the genus Penicillium, that are capable of living in a plant, which may be used to impart improved agronomic traits to plants. The disclosed invention also describes methods of improving plant characteristics by introducing bacteria to those plants. Further, this invention also provides methods of treating seeds and other plant elements with bacteria that are capable of living within a plant, to impart improved agronomic characteristics to plants, particularly agricultural plants, for example soybeans and maize.

### BACKGROUND OF THE INVENTION

According the United Nations Food and Agricultural Organization (UN FAO), the world's population will exceed 9.6 billion people by the year 2050, which will require significant improvements in agricultural to meet growing food demands. At the same time, conservation of resources (such as water, land), reduction of inputs (such as fertilizer, pesticides, herbicides), environmental sustainability, and climate change are increasingly important factors in how food is grown. There is a need for improved agricultural plants and farming practices that will enable the need for a nearly doubled food production with fewer resources, more environmentally sustainable inputs, and with plants with improved responses to various biotic and abiotic stresses (such as pests, drought, disease).

Today, crop performance is optimized primarily via technologies directed towards the interplay between crop genotype (e.g., plant breeding, genetically-modified (GM) crops) and its surrounding environment (e.g., fertilizer, synthetic herbicides, pesticides). While these paradigms have assisted in doubling global food production in the past fifty years, yield growth rates have stalled in many major crops and shifts in the climate have been linked to production instability and declines in important crops, driving an urgent need for novel solutions to crop yield improvement. In addition to their long development and regulatory timelines, public fears of GM-crops and synthetic chemicals have challenged their use in many key crops and countries, resulting in a lack of acceptance for many GM traits and the exclusion of GM crops and many synthetic chemistries from some global markets. Thus, there is a significant need for innovative, effective, environmentally-sustainable, and publically-acceptable approaches to improving the yield and resilience of crops to stresses.

Improvement of crop resilience to biotic and abiotic stresses has proven challenging for conventional genetic and chemical paradigms for crop improvement. This challenge is in part due to the complex, network-level changes that arise during exposure to these stresses.

Like humans, who utilize a complement of beneficial microbial symbionts, plants have been purported to derive a benefit from the vast array of bacteria and fungi that live both within and around their tissues in order to support the plant's health and growth. Endophytes are symbiotic organisms (typically bacteria or fungi) that live within plants, and inhabit various plant tissues, often colonizing the intercellular spaces of host leaves, stems, flowers, fruits, seeds, or roots. To date, a small number of symbiotic endophyte-host relationships have been analyzed in limited studies to provide fitness benefits to model host plants within controlled laboratory settings, such as enhancement of biomass production (i.e., yield) and nutrition, increased tolerance to stress such as drought and pests. There is still a need to develop better plant-endophyte systems to confer benefits to a variety of agriculturally-important plants such as soybean and maize, for example to provide improved yield and tolerance to the environmental stresses present in many agricultural situations for such agricultural plants.

Thus, there is a need for compositions and methods of providing agricultural plants with improved yield and tolerance to various biotic and abiotic stresses. Provided herein are novel compositions including bacteria that are capable of living within a plant, formulations comprising these compositions for treatment of plants and plant elements, and methods of use for the same, created based on the analysis of the key properties that enhance the utility and commercialization of an endophyte composition.

### SUMMARY OF THE INVENTION

In an aspect, the invention provides a method for preparing a plant reproductive element composition, comprising contacting the surface of a plant reproductive element of a plant with a formulation comprising a purified microbial population that comprises a Penicillium endophyte that is heterologous to the plant reproductive element, and comprises at least 500 nucleotides at least 95% identical to a nucleic acid sequence selected from the group consisting of: SEQ ID NO:1, SEQ ID NO: 2, SEQ ID NO: 4, SEQ ID NO:5, wherein the endophyte is present in the formulation in an amount capable of modulating at least one of: trait of agronomic importance, transcription of a gene, level of a transcript, the expression of a protein, level of a hormone, level of a metabolite, and population of endogenous microbes; in plants grown from said plant reproductive elements, as compared to isoline plants grown from plant reproductive elements not contacted with said formulation.

In an aspect, the invention provides a method for preparing a plant reproductive element composition, comprising contacting the surface of a plant reproductive element of a plant with a formulation comprising a purified microbial population that comprises a Penicillium endophyte that is heterologous to the plant reproductive element, and comprises at least 100 nucleotides at least 95% identical to SEQ ID NO: 3, wherein the endophyte is present in the formulation in an amount capable of modulating at least one of: trait of agronomic importance, transcription of a gene, level of a transcript, the expression of a protein, level of a hormone, level of a metabolite, and population of endogenous microbes; in plants grown from said plant reproductive elements, as compared to isoline plants grown from plant reproductive elements not contacted with said formulation.

In an aspect, the invention provides a method for preparing a plant reproductive element composition, comprising contacting the surface of a plant reproductive element of a plant with a formulation comprising a purified microbial population that comprises a Penicillium endophyte that is heterologous to the plant reproductive element, and comprises a Deposit selected from the group consisting of: ______ Deposit ID ______, ______ Deposit ID ______, ______ Deposit ID ______, ______ Deposit ID ______, or IDAC Deposit ID 081111-01, wherein the endophyte is present in the formulation in an amount capable of modulating at least one of: trait of agronomic importance, transcription of a gene, level of a transcript, the expression of a protein, level of a hormone, level of a metabolite, and population of endogenous microbes; in plants grown from said plant reproductive elements, as compared to isoline plants grown from plant reproductive elements not contacted with said formulation.

In an aspect, the invention provides a method for preparing a plant reproductive element composition, comprising contacting the surface of a plant reproductive element of a plant with a formulation comprising a purified microbial population that comprises a Penicillium endophyte that is heterologous to the plant reproductive element, and comprises a Penicillium species selected from the group consisting of: SMCD2206, chrysogenum, olsonii, griseofulvum, or janthinellum, wherein the endophyte is present in the formulation in an amount capable of modulating at least one of: trait of agronomic importance, transcription of a gene, level of a transcript, the expression of a protein, level of a hormone, level of a metabolite, and population of endogenous microbes; in plants grown from said plant reproductive elements, as compared to isoline plants grown from plant reproductive elements not contacted with said formulation. In certain embodiments of any of the preceding methods, the plant optionally comprises in at least one of its plant elements the Penicillium endophyte. In certain embodiments, the progeny of a plant of any of the preceding methods optionally comprises in at least one of its plant elements the Penicillium endophyte. In certain embodiments, the Penicillium endophyte is optionally present in the plant reproductive element in an amount capable of providing a benefit to a plant derived from the plant reproductive element, as compared to a plant derived from a plant reproductive element not treated with the Penicillium endophyte.

In an aspect, the invention provides a method of modulating a trait of agronomic importance in a plant derived from a plant reproductive element, comprising treating the plant reproductive element with a formulation comprising a Penicillium endophyte that comprises at least 500 nucleotides at least 95% identical to a nucleic acid sequence selected from the group consisting of: SEQ ID NO:1, SEQ ID NO: 2, SEQ ID NO: 4, SEQ ID NO:5.

In an aspect, the invention provides a method of modulating a trait of agronomic importance in a plant derived from a plant reproductive element, comprising treating the plant reproductive element with a formulation comprising a Penicillium endophyte that comprises at least 100 nucleotides at least 95% identical to SEQ ID NO: 3.

In an aspect, the invention provides a method of modulating a trait of agronomic importance in a plant derived from a plant reproductive element, comprising treating the plant reproductive element with a formulation comprising a Penicillium endophyte that comprises modulated production of auxin.

In an aspect, the invention provides a method of modulating a trait of agronomic importance in a plant derived from a plant reproductive element, comprising treating the plant reproductive element with a formulation comprising a Penicillium endophyte that comprises modulated production of acetoin.

In an aspect, the invention provides a method of modulating a trait of agronomic importance in a plant derived from a plant reproductive element, comprising treating the plant reproductive element with a formulation comprising a Penicillium endophyte that comprises modulated production of a siderophore.

In an aspect, the invention provides a method of modulating a trait of agronomic importance in a plant derived from a plant reproductive element, comprising treating the plant reproductive element with a formulation comprising a Penicillium endophyte that comprises utilization of a primary carbon source selected from the group consisting of: L-Arabinose, L-Proline, D-Xylose, L-Glutamic acid, D-Ribose, L-Asparagine, Sucrose, Tween 80, Adonitol, L-Alanine, L-Alanyl-Glycine, L-Galactonic-acid-γ-lactone, β-Methyl-D-glucoside, m-Inositol, D-Galactose, D-Trehalose, D-Glucuronic acid, D-Gluconic acid, D-Mannitol, D-L-Malic acid, α-D-Glucose, Maltose, D-Melibiose, Maltotriose, Pyruvic acid, D-Galacturonic acid, D-Mannose, L-Threonine, Inosine, L-Lyxose, D-Alanine, L-Lactic acid, D-Galactonic acid-γ-lactone, Uridine, α-Hydroxy Glutaric acid-γ-lactone, D-L-α-Glycerol phosphate.

In an aspect, the invention provides a method of modulating a trait of agronomic importance in a plant derived from a plant reproductive element, comprising treating the plant reproductive element with a formulation comprising a Penicillium endophyte that comprises secretion at least one protein listed in Table 4C with at least a 2× higher rate, as compared to the strain represented by SEQ ID NO:6.

In an aspect, the invention provides a method of modulating a trait of agronomic importance in a plant derived from a plant reproductive element, comprising treating the plant reproductive element with a formulation comprising a Penicillium endophyte that comprises secretion of at least one protein selected listed in Table 4D with at least a 0.8× lower rate, as compared to the strain represented by SEQ ID NO: 6.

In an aspect, the invention provides a method of modulating a trait of agronomic importance in a plant derived from a plant reproductive element, comprising treating the plant reproductive element with a formulation comprising a Penicillium endophyte that comprises secretion of at least one protein selected from Table 4A.

In an aspect, the invention provides a method of modulating a trait of agronomic importance in a plant derived from a plant reproductive element, comprising treating the plant reproductive element with a formulation comprising a Penicillium endophyte that comprises no secretion of a protein selected from the proteins listed in Table 4B.

In an aspect, the invention provides a method of using a beneficial Penicillium endophyte that confers a trait of agronomic importance to a plant, said endophyte comprising at least 500 nucleotides at least 95% identical to a nucleic acid sequence selected from the group consisting of: SEQ ID NO:1, SEQ ID NO: 2, SEQ ID NO: 4, SEQ ID NO:5.

In an aspect, the invention provides a method of using a beneficial Penicillium endophyte that confers a trait of agronomic importance to a plant, said endophyte comprising at least 100 nucleotides at least 95% identical to SEQ ID NO: 3.

In an aspect, the invention provides a method of using a beneficial Penicillium endophyte that confers a trait of agronomic importance to a plant, said endophyte comprising modulated production of auxin.

In an aspect, the invention provides a method of using a beneficial Penicillium endophyte that confers a trait of agronomic importance to a plant, said endophyte comprising modulated production of acetoin.

In an aspect, the invention provides a method of using a beneficial Penicillium endophyte that confers a trait of agronomic importance to a plant, said endophyte comprising modulated production of a siderophore.

In an aspect, the invention provides a method of using a beneficial Penicillium endophyte that confers a trait of agronomic importance to a plant, said endophyte comprising utilization of a primary carbon source selected from the group consisting of: L-Arabinose, L-Proline, D-Xylose, L-Glutamic acid, D-Ribose, L-Asparagine, Sucrose, Tween 80, Adonitol, L-Alanine, L-Alanyl-Glycine, L-Galactonic-acid-γ-lactone, β-Methyl-D-glucoside, m-Inositol, D-Galactose, D-Trehalose, D-Glucuronic acid, D-Gluconic acid, D-Mannitol, D-L-Malic acid, α-D-Glucose, Maltose, D-Melibiose, Maltotriose, Pyruvic acid, D-Galacturonic acid, D-Mannose, L-Threonine, Inosine, L-Lyxose, D-Alanine, L-Lactic acid, D-Galactonic acid-γ-lactone, Uridine, α-Hydroxy Glutaric acid-γ-lactone, D-L-α-Glycerol phosphate.

In an aspect, the invention provides a method of using a beneficial Penicillium endophyte that confers a trait of agronomic importance to a plant, said endophyte comprising secretes at least one protein listed in Table 4C with at least a 2× higher rate, as compared to the strain represented by SEQ ID NO:6.

In an aspect, the invention provides a method of using a beneficial Penicillium endophyte that confers a trait of agronomic importance to a plant, said endophyte comprising secretes at least one protein selected listed in Table 4D with at least a 0.8× lower rate, as compared to the strain represented by SEQ ID NO: 6.

In an aspect, the invention provides a method of using a beneficial Penicillium endophyte that confers a trait of agronomic importance to a plant, said endophyte comprising secretes at least one protein selected from Table 4A.

In an aspect, the invention provides a method of using a beneficial Penicillium endophyte that confers a trait of agronomic importance to a plant, said endophyte comprising does not secrete a protein selected from the proteins listed in Table 4B.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising modulation of expression of at least one gene involved in a pathway selected from the group consisting of: symbiosis enhancement, resistance to biotic stress, resistance to abiotic stress, growth promotion, cell wall composition, and developmental regulation.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising modulation of at least one hormone involved in a pathway selected from the group consisting of: developmental regulation, seed maturation, dormancy, response to environmental stresses, stomatal closure, expression of stress-related genes, drought tolerance, defense responses, infection response, pathogen response, disease resistance, systemic acquired resistance, transcriptional reprogramming, mechanical support, protection against biotic stress, protection against abiotic stress, signaling, nodulation inhibition, endophyte colonization, fatty acid deoxygenation, wound healing, antimicrobial substance production, metabolite catabolism, cell proliferation, and abscission.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising modulation of the level of expression of least one protein.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising modulation of least one metabolite in at least one of the following plant metabolic pathways: alkaloid metabolism, phenylpropanoid metabolism, flavonoid biosynthesis, isoflavonoid biosynthesis, lipid metabolism, nitrogen metabolism, and carbohydrate metabolism.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising modulation of at least one transcript involved in at least one of the following pathways: symbiosis enhancement, resistance to biotic stress, resistance to abiotic stress, growth promotion, cell wall composition, and developmental regulation.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising upregulation of at least one gene in root tissue, selected from the upregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising upregulation of at least one gene in leaf tissue, selected from the upregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising upregulation of at least one gene in stem tissue, selected from the upregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising downregulation of at least one gene in root tissue, selected from the downregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising downregulation of at least one gene in leaf tissue, selected from the downregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising downregulation of at least one gene in stem tissue, selected from the downregulated genes listed in Table 7A.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in hormone level in root tissue, selected from the group consisting of: SA, CA, OPEA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in hormone level in root tissue, selected from the group consisting of: ABA, JA, JA-ILE, OPDA, TA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in hormone level in stem tissue, selected from the group consisting of: ABA, CA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in hormone level in stem tissue, selected from the group consisting of: SA, JA, JA-ILE, OPDA, OPEA, TA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in hormone level in leaf tissue, selected from the group consisting of: JA, JA-ILE, OPEA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in hormone level in leaf tissue, selected from the group consisting of: ABA, SA, CA, OPDA, TA.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in metabolite level in root tissue, selected from an increased metabolite in root tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in metabolite level in root tissue, selected from a decreased metabolite in root tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in metabolite level in stem tissue, selected from an increased metabolite in stem tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in metabolite level in stem tissue, selected from a decreased metabolite in stem tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in metabolite level in leaf tissue, selected from an increased metabolite in leaf tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising a decrease in metabolite level in leaf tissue, selected from a decreased metabolite in leaf tissue listed in Table 10.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising an increase in the level of at least one protein, selected from the increased expressed proteins listed in Table 8.

In an aspect, the invention provides a method of modulating a trait of agronomic importance in a plant derived from a plant reproductive element under normal watering conditions, comprising treating said plant reproductive element with a formulation comprising a Penicillium endophyte that is heterologous to the plant reproductive element and comprises an ITS nucleic acid sequence that is at least 95% identical over at least 500 nucleotides to a nucleic acid sequence selected from SEQ ID NO:1 through SEQ ID NO:5, and modulating at least one characteristic of said plant as compared to an isoline plant not grown from reproductive elements treated with said Penicillium endophyte, said characteristic comprising decrease in the level of at least one protein, selected from the decreased expression proteins listed in Table 8.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:2 through SEQ ID NO:18, and modulating at least one characteristic of said plant as compared to an isoline plant not grown from a reproductive element treated with said Penicillium endophyte, said characteristic comprising modulation of transcription of at least one gene involved in at least one of the following pathways: symbiosis enhancement, resistance to biotic stress, resistance to abiotic stress, growth promotion, cell wall composition, and developmental regulation.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:2 through SEQ ID NO:18, and modulating at least one characteristic of said plant as compared to an isoline plant not grown from a reproductive element treated with said Penicillium endophyte, said characteristic comprising modulation of transcription of at least one transcript involved in at least one of the following pathways: symbiosis enhancement, resistance to biotic stress, resistance to abiotic stress, growth promotion, cell wall composition, and developmental regulation.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:2 through SEQ ID NO:18, and modulating at least one characteristic of said plant as compared to an isoline plant not grown from a reproductive element treated with said Penicillium endophyte, said characteristic comprising modulation of levels of at least one hormone involved in a pathway selected from the group consisting of: developmental regulation, seed maturation, dormancy, response to environmental stresses, stomatal closure, expression of stress-related genes, drought tolerance, defense responses, infection response, pathogen response, disease resistance, systemic acquired resistance, transcriptional reprogramming, mechanical support, protection against biotic stress, protection against abiotic stress, signaling, nodulation inhibition, endophyte colonization, fatty acid deoxygenation, wound healing, antimicrobial substance production, metabolite catabolism, cell proliferation, and abscission.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:2 through SEQ ID NO:18, and modulating at least one characteristic of said plant as compared to an isoline plant not grown from a reproductive element treated with said Penicillium endophyte, said characteristic comprising modulation of at least one metabolite in at least one of the following plant metabolic pathways: alkaloid metabolism, phenylpropanoid metabolism, flavonoid biosynthesis, isoflavonoid biosynthesis, lipid metabolism, nitrogen metabolism, and carbohydrate metabolism.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:2 through SEQ ID NO:18, and modulating at least one characteristic of said plant as compared to an isoline plant not grown from a reproductive element treated with said Penicillium endophyte, said characteristic comprising modulation of microbiome community profile.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one gene in root tissue, selected from the upregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one gene in leaf tissue, selected from the upregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one gene in stem tissue, selected from the upregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one gene in root tissue, selected from the downregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one gene in leaf tissue, selected from the downregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one gene in stem tissue, selected from the downregulated genes listed in Tables 7A, 7B, 7C, 7D, and 7E.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one transcript in root tissue, selected from the upregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one transcript in leaf tissue, selected from the upregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising upregulation of at least one transcript in stem tissue, selected from the upregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one transcript in root tissue, selected from the downregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one transcript in leaf tissue, selected from the downregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising downregulation of at least one transcript in stem tissue, selected from the downregulated transcripts listed in Table 7F.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in hormone level in root tissue, selected from the group consisting of: JA, JAOILE, OPDA, OPEA, TA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in hormone level in root tissue, selected from the group consisting of: ABA, SA, CA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in hormone level in stem tissue, selected from the group consisting of: SA, CA, JA-ILE, OPDA, OPEA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in hormone level in stem tissue, selected from the group consisting of: ABA, JA, TA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in hormone level in leaf tissue, selected from the group consisting of: SA, CA, OPDA, TA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in hormone level in leaf tissue, selected from the group consisting of: ABA, JA, JA-ILE, OPEA.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in metabolite level in root tissue, selected from an increased metabolite in root tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in metabolite level in root tissue, selected from a decreased metabolite in root tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in metabolite level in stem tissue, selected from an increased metabolite in stem tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in metabolite level in stem tissue, selected from a decreased metabolite in stem tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in metabolite level in leaf tissue, selected from an increased metabolite in leaf tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in metabolite level in leaf tissue, selected from a decreased metabolite in leaf tissue listed in Table 10.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising increase in abundance of microorganisms of the family Glomeraceae.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising increase in abundance of microorganisms of the genus Rhizophagus.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in abundance of microorganisms of the genus Glomus.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in abundance of microorganisms of the family Enterobacteriaceae.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising a decrease in abundance of microorganisms of the genus Escherhia-Shigella.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising presence of at least one OTU described in Table 11A or Table 11B.

In an aspect, the invention provides a method of modulating a trait of agronomic importance in a plant derived from a plant reproductive element under water-limited conditions, comprising associating said plant reproductive element with a formulation comprising a Penicillium endophyte that is heterologous to the plant reproductive element and comprises an ITS nucleic acid sequence that is at least 95% identical to a nucleic acid sequence selected from SEQ ID NO:1 through SEQ ID NO:5, and modulating at least one characteristic of said plant as compared to an isoline plant not grown from a reproductive element treated with said Penicillium endophyte, said characteristic comprising an increase in presence of at least one OTU selected from Table 11C.

Certain embodiments of the invention are any of the preceding methods; wherein said trait of agronomic importance is selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, improved plant visual appearance.

In an aspect, the invention provides a method of altering the native microbiome community of a plant, comprising deriving said plant from a plant reproductive element treated with a formulation comprising a beneficial Penicillium endophyte. In an embodiment, the microbiome community alteration optionally comprises an increase in abundance of microorganisms of the family Glomeraceae. In an embodiment, the microbiome community alteration optionally comprises an increase in abundance of microorganisms of the genus Rhizophagus. In an embodiment, the microbiome community alteration optionally comprises an increase in abundance of microorganisms of the genus Glomus. In an embodiment, the microbiome community alteration optionally comprises a decrease in abundance of microorganisms of the family Enterobacteriaceae. In an embodiment, the microbiome community alteration optionally comprises a decrease in abundance of microorganisms of the genus Escherhia-Shigella. In an embodiment, the microbiome community alteration optionally comprises a presence of at least one OTU described in Table 11A or Table 11B. In an embodiment, the microbiome community alteration optionally comprises an increase in presence of at least one OTU selected from Table 11C.

Certain embodiments of the invention are any of the preceding methods, wherein the said plant is optionally soybean or maize. Certain embodiments of the invention are any of the preceding methods, wherein the formulation optionally comprises a purified population of the Penicillium endophyte at a concentration of at least about 10̂2 CFU/ml in a liquid formulation or about 10̂2 CFU/gm in a non-liquid formulation. Certain embodiments of the invention are any of the preceding methods, wherein the Penicillium endophyte is optionally capable auxin production, nitrogen fixation, production of an antimicrobial compound, mineral phosphate solubilization, siderophore production, cellulase production, chitinase production, xylanase production, or acetoin production. Certain embodiments of the invention are any of the preceding methods, wherein the Penicillium endophyte is optionally capable of localizing in a plant element of the plant, the plant element selected from the group consisting of: whole plant, seedling, meristematic tissue, ground tissue, vascular tissue, dermal tissue, seed, leaf, root, shoot, stem, flower, fruit, stolon, bulb, tuber, corm, keikis, and bud. In an embodiment, the plant element is optionally a seed, and wherein the Penicillium endophyte is present in at least two compartments of the seed, selected from the group consisting of: embryo, seed coat, endosperm, cotyledon, hypocotyl, and radicle.

Certain embodiments of the invention are any of the preceding methods, wherein said plant reproductive element is optionally a seed or optionally, a transgenic seed. Certain embodiments of the invention are any of the preceding methods, wherein the plant reproductive element is optionally placed into a substrate that promotes plant growth or wherein the substrate is optionally soil. In an embodiment, a plurality of the plant reproductive elements are optionally placed in the soil in rows, with substantially equal spacing between each within each row. Certain embodiments of the invention are any of the preceding methods, wherein the formulation optionally further comprises one or more of the following: a stabilizer, or a preservative, or a carrier, or a surfactant, or an anticomplex agent, or any combination thereof. Certain embodiments of the invention are any of the preceding methods, wherein the formulation optionally further comprises one or more of the following: fungicide, nematicide, bactericide, insecticide, and herbicide. Certain embodiments of the invention are any of the preceding methods, wherein the formulation optionally further comprises at least one additional endophyte.

In an aspect, the invention provides for a plurality of plant reproductive element compositions prepared according to any of the preceding methods, wherein compositions are confined within an object selected from the group consisting of: bottle, jar, ampule, package, vessel, bag, box, bin, envelope, carton, container, silo, shipping container, truck bed, and case.

In an aspect, the invention provides for a synthetic combination comprising a plant reproductive element treated with a formulation comprising a purified Penicillium endophyte population, wherein said Penicillium endophyte is heterologous to the plant reproductive element, and comprises at least 500 nucleotides at least 95% identical to a nucleic acid sequence selected from the group consisting of: SEQ ID NO:1, SEQ ID NO: 2, SEQ ID NO: 4, SEQ ID NO:5 wherein the endophyte is present in the synthetic combination in an amount capable of modulating at least one of: a trait of agronomic importance, the expression of a gene, the level of a transcript, the expression of a protein, the level of a hormone, the level of a metabolite, the population of endogenous microbes in plants grown from said plant reproductive element, as compared to an isoline plant grown from a plant reproductive element not contacted with said endophyte.

In an aspect, the invention provides for a synthetic combination comprising a plant reproductive element treated with a formulation comprising a purified Penicillium endophyte population, wherein said Penicillium endophyte is heterologous to the plant reproductive element, and comprises at least 100 nucleotides at least 95% identical to SEQ ID NO: 35 wherein the endophyte is present in the synthetic combination in an amount capable of modulating at least one of: a trait of agronomic importance, the expression of a gene, the level of a transcript, the expression of a protein, the level of a hormone, the level of a metabolite, the population of endogenous microbes in plants grown from said plant reproductive element, as compared to an isoline plant grown from a plant reproductive element not contacted with said endophyte.

In an aspect, the invention provides for a synthetic combination comprising a plant reproductive element treated with a formulation comprising a purified Penicillium endophyte population, wherein said Penicillium endophyte is heterologous to the plant reproductive element, and comprises a Deposit selected from the group consisting of: ______ Deposit ID ______, ______ Deposit ID ______, ______ Deposit ID ______, ______ Deposit ID ______, or IDAC Deposit ID 081111-01 wherein the endophyte is present in the synthetic combination in an amount capable of modulating at least one of: a trait of agronomic importance, the expression of a gene, the level of a transcript, the expression of a protein, the level of a hormone, the level of a metabolite, the population of endogenous microbes in plants grown from said plant reproductive element, as compared to an isoline plant grown from a plant reproductive element not contacted with said endophyte.

In an aspect, the invention provides for a synthetic combination comprising a plant reproductive element treated with a formulation comprising a purified Penicillium endophyte population, wherein said Penicillium endophyte is heterologous to the plant reproductive element, and comprises a Penicillium species selected from the group consisting of: SMCD2206, chrysogenum, olsonii, griseofulvum, or janthinellum wherein the endophyte is present in the synthetic combination in an amount capable of modulating at least one of: a trait of agronomic importance, the expression of a gene, the level of a transcript, the expression of a protein, the level of a hormone, the level of a metabolite, the population of endogenous microbes in plants grown from said plant reproductive element, as compared to an isoline plant grown from a plant reproductive element not contacted with said endophyte.

In an embodiment, the invention provides for any of the preceding synthetic combinations wherein the plant is optionally soybean or maize. In an embodiment, the invention provides for any of the preceding synthetic combinations wherein the formulation optionally comprises a purified population of the Penicillium endophyte at a concentration of at least about 10̂2 CFU/ml in a liquid formulation or about 10̂2 CFU/gm in a non-liquid formulation. In an embodiment, the invention provides for any of the preceding synthetic combinations wherein the Penicillium endophyte is optionally capable of auxin production, nitrogen fixation, production of an antimicrobial compound, mineral phosphate solubilization, siderophore production, cellulase production, chitinase production, xylanase production, or acetoin production. In an embodiment, the invention provides for any of the preceding synthetic combinations wherein the trait of agronomic importance is optionally selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, improved plant visual appearance. In an embodiment, the invention provides for any of the preceding synthetic combinations wherein the Penicillium endophyte is optionally capable of localizing in a plant element of a plant grown from said seed, said plant element selected from the group consisting of: whole plant, seedling, meristematic tissue, ground tissue, vascular tissue, dermal tissue, seed, leaf, root, shoot, stem, flower, fruit, stolon, bulb, tuber, corm, keikis, and bud. In an embodiment, the invention provides for any of the preceding synthetic combinations wherein the plant reproductive element is optionally a seed or a transgenic seed. In an embodiment, the invention provides for any of the preceding synthetic combinations wherein the plant reproductive element is optionally placed into a substrate that promotes plant growth and wherein the substrate is optionally soil. In an embodiment, the invention provides for any of the preceding synthetic combinations wherein the plant reproductive elements are optionally placed in the soil in rows, with substantially equal spacing between each seed within each row. In an embodiment, the invention provides for any of the preceding synthetic combinations wherein the formulation optionally further comprises one or more of the following: a stabilizer, or a preservative, or a carrier, or a surfactant, or an anticomplex agent, or any combination thereof. In an embodiment, the invention provides for any of the preceding synthetic combinations wherein the formulation optionally further comprises one or more of the following: fungicide, nematicide, bactericide, insecticide, and herbicide. In an embodiment, the invention provides for a plant derived from any of the preceding synthetic combinations wherein the formulation optionally further comprises at least one additional endophyte. In an embodiment, the invention provides for a plant derived from any of the preceding synthetic combinations wherein the seed is optionally a transgenic seed.

In an aspect, the invention provides a plant derived from any of the preceding synthetic combinations wherein the plant comprises in at least one of its plant elements said endophyte. In an embodiment, the progeny of the plant optionally comprises in at least one of its plant elements said endophyte.

In an aspect, the invention provides a plurality of any of the preceding synthetic combinations, wherein the seed compositions are optionally confined within an object selected from the group consisting of: bottle, jar, ampule, package, vessel, bag, box, bin, envelope, carton, container, silo, shipping container, truck bed, and case.

In an embodiment, the Penicillium endophyte of any of the preceding synthetic combinations is optionally present in the colonized seed in an amount capable of providing a benefit to the seed or to agriculture. In an embodiment, the endophyte of any of the preceding synthetic combinations is optionally present in at least two compartments of the seed, selected from the group consisting of: embryo, seed coat, endosperm, cotyledon, hypocotyl, and radicle.

In an aspect, the invention provides a plurality of any of the preceding synthetic combinations, wherein the synthetic combinations are shelf-stable.

In an aspect, the invention provides a plant grown from any of the preceding synthetic combinations wherein the plant comprises modulation of the transcription of at least one gene involved in at least one of the following pathways: symbiosis enhancement, resistance to biotic stress, resistance to abiotic stress, growth promotion, cell wall composition, and developmental regulation.

In an aspect, the invention provides a plant grown from any of the preceding synthetic combinations wherein the plant comprises modulation of the transcription of at least one transcript involved in at least one of the following pathways: symbiosis enhancement, resistance to biotic stress, resistance to abiotic stress, growth promotion, cell wall composition, and developmental regulation.

In an aspect, the invention provides a plant grown from any of the preceding synthetic combinations wherein the plant comprises modulating the level of at least one hormone involved in a pathway selected from the group consisting of: developmental regulation, seed maturation, dormancy, response to environmental stresses, stomatal closure, expression of stress-related genes, drought tolerance, defense responses, infection response, pathogen response, disease resistance, systemic acquired resistance, transcriptional reprogramming, mechanical support, protection against biotic stress, protection against abiotic stress, signaling, nodulation inhibition, endophyte colonization, fatty acid deoxygenation, wound healing, antimicrobial substance production, metabolite catabolism, cell proliferation, and abscission.

In an aspect, the invention provides a plant grown from any of the preceding synthetic combinations wherein the plant comprises modulating at least one metabolite in at least one of the following plant metabolic pathways: alkaloid metabolism, phenylpropanoid metabolism, flavonoid biosynthesis, isoflavonoid biosynthesis, lipid metabolism, nitrogen metabolism, and carbohydrate metabolism.

In an aspect, the invention provides a plant grown from any of the preceding synthetic combinations wherein the plant comprises upregulation of at least one gene in root tissue, selected from the upregulated genes listed in Tables 7A, 7B, 7C, 7D, and 7E.

In an aspect, the invention provides a plant grown from any of the preceding synthetic combinations wherein the plant comprises upregulation of at least one gene in leaf tissue, selected from the upregulated genes listed in Tables 7A, 7B, 7C, 7D, and 7E.

In an aspect, the invention provides a plant grown from any of the preceding synthetic combinations wherein the plant comprises upregulation of at least one gene in stem tissue, selected from the upregulated genes listed in Tables 7A, 7B, 7C, 7D, and 7E.

In an aspect, the invention provides a plant grown from any of the preceding synthetic combinations wherein the plant comprises downregulation of at least one gene in root tissue, selected from the downregulated genes listed in Tables 7A, 7B, 7C, 7D, and 7E.

In an aspect, the invention provides a plant grown from any of the preceding synthetic combinations wherein the plant comprises downregulation of at least one gene in leaf tissue, selected from the downregulated genes listed in Tables 7A, 7B, 7C, 7D, and 7E.

In an aspect, the invention provides a plant grown from any of the preceding synthetic combinations wherein the plant comprises downregulation of at least one gene in stem tissue, selected from the downregulated genes listed in Tables 7A, 7B, 7C, 7D, and 7E.

In an aspect, the invention provides a plant grown from any of the preceding synthetic combinations wherein the plant comprises upregulation of at least one transcript in root tissue, selected from the upregulated transcripts listed in Table 7F.

In an aspect, the invention provides a plant grown from any of the preceding synthetic combinations wherein the plant comprises upregulation of at least one transcript in leaf tissue, selected from the upregulated transcripts listed in Table 7F.

In an aspect, the invention provides a plant grown from any of the preceding synthetic combinations wherein the plant comprises upregulation of at least one transcript in stem tissue, selected from the upregulated transcripts listed in Table 7F.

In an aspect, the invention provides a plant grown from any of the preceding synthetic combinations wherein the plant comprises downregulation of at least one transcript in root tissue, selected from the downregulated transcripts listed in Table 7F.

In an aspect, the invention provides a plant grown from any of the preceding synthetic combinations wherein the plant comprises downregulation of at least one transcript in leaf tissue, selected from the downregulated transcripts listed in Table 7F.

In an aspect, the invention provides a plant grown from any of the preceding synthetic combinations wherein the plant comprises downregulation of at least one transcript in stem tissue, selected from the downregulated transcripts listed in Table 7F.

In an aspect, the invention provides a plant grown from any of the preceding synthetic combinations wherein the plant comprises an increase in hormone level in root tissue, selected from the group consisting of: JA, JAOILE, OPDA, OPEA, TA.

In an aspect, the invention provides a plant grown from any of the preceding synthetic combinations wherein the plant comprises a decrease in hormone level in root tissue, selected from the group consisting of: ABA, SA, CA.

In an aspect, the invention provides a plant grown from any of the preceding synthetic combinations wherein the plant comprises an increase in hormone level in stem tissue, selected from the group consisting of: SA, CA, JA-ILE, OPDA, OPEA.

In an aspect, the invention provides a plant grown from any of the preceding synthetic combinations wherein the plant comprises a decrease in hormone level in stem tissue, selected from the group consisting of: ABA, JA, TA.

In an aspect, the invention provides a plant grown from any of the preceding synthetic combinations wherein the plant comprises an increase in hormone level in leaf tissue, selected from the group consisting of: SA, CA, OPDA, TA.

In an aspect, the invention provides a plant grown from any of the preceding synthetic combinations wherein the plant comprises a decrease in hormone level in leaf tissue, selected from the group consisting of: ABA, JA, JA-ILE, OPEA.

In an aspect, the invention provides a plant grown from any of the preceding synthetic combinations wherein the plant comprises an increase in metabolite level in root tissue, selected from an increased metabolite in root tissue listed in Table 10.

In an aspect, the invention provides a plant grown from any of the preceding synthetic combinations wherein the plant comprises a decrease in metabolite level in root tissue, selected from a decreased metabolite in root tissue listed in Table 10.

In an aspect, the invention provides a plant grown from any of the preceding synthetic combinations wherein the plant comprises an increase in metabolite level in stem tissue, selected from an increased metabolite in stem tissue listed in Table 10.

In an aspect, the invention provides a plant grown from any of the preceding synthetic combinations wherein the plant comprises a decrease in metabolite level in stem tissue, selected from a decreased metabolite in stem tissue listed in Table 10.

In an aspect, the invention provides a plant grown from any of the preceding synthetic combinations wherein the plant comprises an increase in metabolite level in leaf tissue, selected from an increased metabolite in leaf tissue listed in Table 10.

In an aspect, the invention provides a plant grown from any of the preceding synthetic combinations wherein the plant comprises a decrease in metabolite level in leaf tissue, selected from a decreased metabolite in leaf tissue listed in Table 10.

In an aspect, the invention provides a plant grown from any of the preceding synthetic combinations wherein the plant comprises an increase in abundance of microorganisms of the family Glomeraceae.

In an aspect, the invention provides a plant grown from any of the preceding synthetic combinations wherein the plant comprises an increase in abundance of microorganisms of the genus Rhizophagus.

In an aspect, the invention provides a plant grown from any of the preceding synthetic combinations wherein the plant comprises an increase in abundance of microorganisms of the genus Glomus.

In an aspect, the invention provides a plant grown from any of the preceding synthetic combinations wherein the plant comprises a decrease in abundance of microorganisms of the family Enterobacteriaceae.

In an aspect, the invention provides a plant grown from any of the preceding synthetic combinations wherein the plant comprises a decrease in abundance of microorganisms of the genus Escherhia-Shigella

In an aspect, the invention provides a plant grown from any of the preceding synthetic combinations wherein the plant comprises a presence of at least one OTU described in Table 11A or Table 11B.

In an aspect, the invention provides a plant grown from any of the preceding synthetic combinations wherein the plant comprises an increase in presence of at least one OTU selected from Table 11C.

1. In an aspect, the invention provides a plant grown from the synthetic combination of claim 28, said plant exhibiting a trait of agronomic interest, selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, improved plant visual appearance. In an embodiment, the plant is optionally soybean or maize. In an embodiment, the plant or progeny of the plant comprises at least one of its plant elements said Penicillium endophyte.

### BRIEF DESCRIPTION OF THE FIGURES

These and other features, aspects, and advantages of the present invention will become better understood with regard to the following description, and accompanying figure, where:

FIG. 1: Culture plate of Strain A

FIG. 2: Culture plate of Strain B

FIG. 3: Culture plate of Strain D

FIG. 4: Culture plate of Strain E

FIG. 5: Culture plate of Strain F

FIG. 6: Culture plate of Strain G

FIG. 7: Greenhouse phenotypes of plants grown from seeds treated with

Penicillium Strain A as compared to plants grown from seeds treated with formulation control, under water-limited conditions.

FIG. 8: Greenhouse phenotypes of plants grown from seeds treated with Penicillium Strain B as compared to plants grown from seeds treated with formulation control, under water-limited conditions.

FIG. 9: Greenhouse phenotypes of plants grown from seeds treated with Penicillium Strain D as compared to plants grown from seeds treated with formulation control, under water-limited conditions.

FIG. 10: Greenhouse phenotypes of plants grown from seeds treated with Penicillium Strain E as compared to plants grown from seeds treated with formulation control, under water-limited conditions.

FIG. 11: Greenhouse phenotypes of plants grown from seeds treated with Penicillium Strain F as compared to plants grown from seeds treated with formulation control, under water-limited conditions.

FIG. 12: Greenhouse phenotypes of plants grown from seeds treated with Penicillium Strain G as compared to plants grown from seeds treated with formulation control, under water-limited conditions.

FIG. 13: Greenhouse phenotypes of plants grown from seeds treated with Strain B as compared to plants grown from seeds treated with formulation control as well as untreated seeds, under normal watering conditions

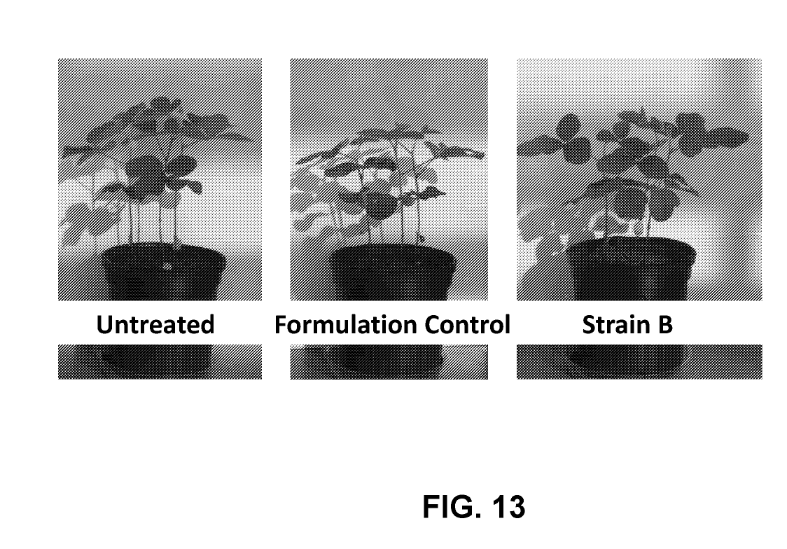

FIG. 14: Greenhouse phenotypes of plants grown from seeds treated with Strain B as compared to plants grown from seeds treated with formulation control as well as untreated seeds, under water-limited conditions

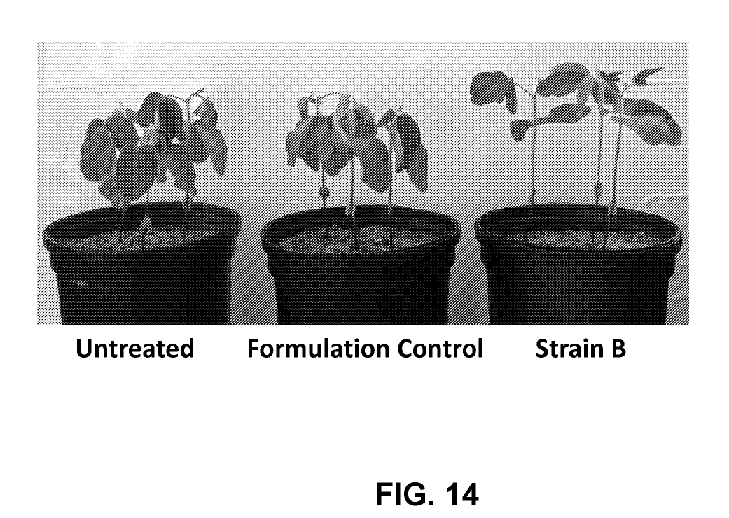

### DEFINITIONS

Terms used in the claims and specification are defined as set forth below unless otherwise specified.

It must be noted that, as used in the specification and the appended claims, the singular forms “a,” “an” and “the” include plural referents unless the context clearly dictates otherwise.

An “endophyte” is an organism capable of living within a plant or otherwise associated therewith, and does not cause disease or harm the plant otherwise. Endophytes can occupy the intracellular or extracellular spaces of plant tissue, including the leaves, stems, flowers, fruits, seeds, or roots. An endophyte can be for example a bacterial or fungal organism, and can confer a beneficial property to the host plant such as an increase in yield, biomass, resistance, or fitness. An endophyte can be a fungus or a bacterium.

A “plurality of endophytes” means two or more types of endophyte entities, e.g., of simple bacteria or simple fungi, complex fungi, or combinations thereof. In some embodiments, the two or more types of endophyte entities are two or more strains of endophytes. In other embodiments, the two or more types of endophyte entities are two or more species of endophytes. In yet other embodiments, the two or more types of endophyte entities are two or more genera of endophytes. In yet other embodiments, the two or more types of endophyte entities are two or more families of endophytes. In yet other embodiments, the two or more types of endophyte entities are two or more orders of endophytes.

As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte. In some embodiments, a microbe is an endophyte, for example a bacterial or fungal endophyte, which is capable of living within a plant. In some embodiments, a microbe or microorganism encompasses individual cells (e.g., unicellular microorganisms) or more than one cell (e.g., multi-cellular microorganism). A “population of microorganisms” may thus refer to a multiple cells of a single microorganism, in which the cells share common genetic derivation.

As used herein, the term “bacterium” or “bacteria” refers in general to any prokaryotic organism, and may reference an organism from either Kingdom Eubacteria (Bacteria), Kingdom Archaebacteria (Archae), or both. In some cases, bacterial genera or other taxonomic classifications have been reassigned due to various reasons (such as but not limited to the evolving field of whole genome sequencing), and it is understood that such nomenclature reassignments are within the scope of any claimed taxonomy. For example, certain species of the genus Erwinia have been described in the literature as belonging to genus Pantoea (Zhang and Qiu, 2015).

The term 16S refers to the DNA sequence of the 16S ribosomal RNA (rRNA) sequence of a bacterium. 16S rRNA gene sequencing is a well-established method for studying phylogeny and taxonomy of bacteria.

As used herein, the term “fungus” or “fungi” refers in general to any organism from Kingdom Fungi. Historical taxonomic classification of fungi has been according to morphological presentation. Beginning in the mid-1800's, it was became recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, 2011). With the development of genomic sequencing, it became evident that taxonomic classification based on molecular phylogenetics did not align with morphological-based nomenclature (Shenoy, 2007). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, 2012). However, systematics experts have not aligned on common nomenclature for all fungi, nor are all existing databases and information resources inclusive of updated taxonomies. As such, many fungi referenced herein may be described by their anamorph form but it is understood that based on identical genomic sequencing, any pleomorphic state of that fungus may be considered to be the same organism. For example, the genus Alternaria is the anamorph form of the teleomorph genus Lewia (Kwasna 2003), ergo both would be understood to be the same organism with the same DNA sequence. For example, it is understood that the genus Acremonium is also reported in the literature as genus Sarocladium as well as genus Tilachilidium (Summerbell, 2011). For example, the genus Cladosporium is an anamorph of the teleomorph genus Davidiella (Bensch, 2012), and is understood to describe the same organism. In some cases, fungal genera have been reassigned due to various reasons, and it is understood that such nomenclature reassignments are within the scope of any claimed genus. For example, certain species of the genus Microdiplodia have been described in the literature as belonging to genus Paraconiothyrium (Crous and Groenveld, 2006).

“Internal Transcribed Spacer” (ITS) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (LSU) sequence is used to identify fungi. LSU gene sequencing is a well-established method for studying phylogeny and taxonomy of fungi. Some fungal endophytes of the present invention may be described by an ITS sequence and some may be described by an LSU sequence. Both are understood to be equally descriptive and accurate for determining taxonomy.

As used herein with respect to fungi and bacteria, the term “marker gene” refers to an organism's 16S (for bacteria) or ITS (for fungi) polynucleotide sequence, by which a microbe may be specifically identified and assigned taxonomic nomenclature.

The terms “pathogen” and “pathogenic” in reference to a bacterium or fungus includes any such organism that is capable of causing or affecting a disease, disorder or condition of a host comprising the organism.

A “spore” or a population of “spores” refers to bacteria or fungi that are generally viable, more resistant to environmental influences such as heat and bactericidal or fungicidal agents than other forms of the same bacteria or fungi, and typically capable of germination and out-growth. Bacteria and fungi that are “capable of forming spores” are those bacteria and fungi comprising the genes and other necessary abilities to produce spores under suitable environmental conditions.

“Biomass” means the total mass or weight (fresh or dry), at a given time, of a plant tissue, plant tissues, an entire plant, or population of plants. Biomass is usually given as weight per unit area. The term may also refer to all the plants or species in the community (community biomass).

The term “isolated” is intended to specifically reference an organism, cell, tissue, polynucleotide, or polypeptide that is removed from its original source and purified from additional components with which it was originally associated. For example, an endophyte may be considered isolated from a seed if it is removed from that seed source and purified so that it is isolated from any additional components with which it was originally associated. Similarly, an endophyte may be removed and purified from a plant or plant element so that it is isolated and no longer associated with its source plant or plant element.

As used herein, an isolated strain of a microbe is a strain that has been removed from its natural milieu. “Pure cultures” or “isolated cultures” are cultures in which the organisms present are only of one strain of a particular genus and species. This is in contrast to “mixed cultures,” which are cultures in which more than one genus and/or species of microorganism are present. As such, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants. Further, as used herein, a “biologically pure” strain is intended to mean the strain separated from materials with which it is normally associated in nature. A strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more that 50%, 60%, 70%, 80%, 90%, or 95% of the isolated strain.

A “host plant” includes any plant, particularly a plant of agronomic importance, which an endophytic entity such as an endophyte can colonize. As used herein, an endophyte is said to “colonize” a plant or plant element when it can be stably detected within the plant or plant element over a period time, such as one or more days, weeks, months or years, in other words, a colonizing entity is not transiently associated with the plant or plant element. Such host plants are preferably plants of agronomic importance.

A “non-host target” means an organism or chemical compound that is altered in some way after contacting a host plant that comprises an endophyte, as a result of a property conferred to the host plant by the endophyte.

As used herein, a nucleic acid has “homology” or is “homologous” to a second nucleic acid if the nucleic acid sequence has a similar sequence to the second nucleic acid sequence. The terms “identity,” “percent sequence identity” or “identical” in the context of nucleic acid sequences refer to the residues in the two sequences that are the same when aligned for maximum correspondence. There are a number of different algorithms known in the art that can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). In some embodiments, sequences can be compared using Geneious (Biomatters, Ltd., Auckland, New Zealand). In other embodiments, polynucleotide sequences can be compared using the multiple sequence alignment algorithm MUSCLE (Edgar R C, 2004).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, or at least about 90%, or at least about 95%, 96%, 97%, 98% 99%, 99.5% or 100% of the nucleotide bases, as measured by any well-known algorithm of sequence identity, such as FASTA, BLAST, Gap, MUSCLE, or any other method known in the art.

As used herein, the terms “operational taxonomic unit,” “OTU,” “taxon,” “hierarchical cluster,” and “cluster” are used interchangeably. An operational taxon unit (OTU) refers to a group of one or more organisms that comprises a node in a clustering tree. The level of a cluster is determined by its hierarchical order. In one embodiment, an OTU is a group tentatively assumed to be a valid taxon for purposes of phylogenetic analysis. In another embodiment, an OTU is any of the extant taxonomic units under study. In yet another embodiment, an OTU is given a name and a rank. For example, an OTU can represent a domain, a sub-domain, a kingdom, a sub-kingdom, a phylum, a sub-phylum, a class, a sub-class, an order, a sub-order, a family, a subfamily, a genus, a subgenus, or a species. In some embodiments, OTUs can represent one or more organisms from the kingdoms eubacteria, protista, or fungi at any level of a hierarchal order. In some embodiments, an OTU represents a prokaryotic or fungal order.

In some embodiments, the present invention contemplates the synthetic combinations comprising the combination of a plant element, seedling, or whole plants and an endophyte population, in which the endophyte population is “heterologously disposed.” In some embodiments, “heterologously disposed” means that the native plant element, seedling, or plant does not contain detectable levels of the microbe in that same plant element, seedling, or plant. For example if said plant element or seedling or plant does not naturally have the endophyte associated with it and the endophyte is applied, the endophyte would be considered to be heterologously disposed. In some embodiments, “heterologously disposed” means that the endophyte is being applied to a different plant element than that with which the endophyte is naturally associated. For example, if said plant element or seedling or plant has the endophyte normally found in the root tissue but not in the leaf tissue, and the endophyte is applied to the leaf, the endophyte would be considered to be heterologously disposed. In some embodiments, “heterologously disposed” means that the endophyte being applied to a different tissue or cell layer of the plant element than that in which the microbe is naturally found. For example, if endophyte is naturally found in the mesophyll layer of leaf tissue but is being applied to the epithelial layer, the endophyte would be considered to be heterologously disposed. In some embodiments, “heterologously disposed” means that the endophyte being applied is at a greater concentration, number, or amount of the plant element, seedling, or plant, than that which is naturally found in said plant element, seedling, or plant. For example, an endophyte concentration that is being applied is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than that which is naturally present, the endophyte would be considered to be heterologously disposed. In some embodiments, “heterologously disposed” means that the endophyte is applied to a developmental stage of the plant element, seedling, or plant in which said endophyte is not naturally associated, but may be associated at other stages. For example, if an endophyte is normally found at the flowering stage of a plant and no other stage, an endophyte applied at the seedling stage may be considered to be heterologously disposed. For example, an endophyte that is normally associated with leaf tissue of a cupressaceous tree sample would be considered heterologous to leaf tissue of a maize plant. In another example, an endophyte that is normally associated with leaf tissue of a maize plant is considered heterologous to a leaf tissue of another maize plant that naturally lacks said endophyte. In another example, an endophyte that is normally associated at low levels in a plant is considered heterologous to that plant if a higher concentration of that endophyte is introduced into the plant. In yet another example, an endophyte that is associated with a tropical grass species would be considered heterologous to a wheat plant.

The term “isoline” is a comparative term, and references organisms that are genetically identical, but may differ in treatment. In one example, two genetically identical maize plant embryos may be separated into two different groups, one receiving a treatment (such as transformation with a heterologous polynucleotide, to create a genetically modified plant) and one control that does not receive such treatment. Any phenotypic differences between the two groups may thus be attributed solely to the treatment and not to any inherency of the plant's genetic makeup. In another example, two genetically identical soybean seeds may be treated with a formulation that introduces an endophyte composition. Any phenotypic differences between the plants derived from (e.g., grown from or obtained from) those seeds may be attributed to the treatment, thus forming an isoline comparison.

Similarly, by the term “reference agricultural plant,” it is meant an agricultural plant of the same species, strain, or cultivar to which a treatment, formulation, composition or endophyte preparation as described herein is not administered/contacted. A reference agricultural plant, therefore, is identical to the treated plant with the exception of the presence of the endophyte and can serve as a control for detecting the effects of the endophyte that is conferred to the plant.

A “reference environment” refers to the environment, treatment or condition of the plant in which a measurement is made. For example, production of a compound in a plant associated with an endophyte can be measured in a reference environment of drought stress, and compared with the levels of the compound in a reference agricultural plant under the same conditions of drought stress. Alternatively, the levels of a compound in plant associated with an endophyte and reference agricultural plant can be measured under identical conditions of no stress.

A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, kelkis, shoot, bud. As used herein, a “plant element” is synonymous to a “portion” of a plant, and refers to any part of the plant, and can include distinct tissues and/or organs, and may be used interchangeably with the term “tissue” throughout.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keikis, or bud.

A “population” of plants refers to more than one plant, that are of the same taxonomic categeory, typically be of the same species, and will also typically share a common genetic derivation.

As used herein, an “agricultural seed” is a seed used to grow a plant typically used in agriculture (an “agricultural plant”). The seed may be of a monocot or dicot plant, and may be planted for the production of an agricultural product, for example feed, food, fiber, fuel, industrial uses, etc. As used herein, an agricultural seed is a seed that is prepared for planting, for example, in farms for growing.

“Agricultural plants,” or “plants of agronomic importance,” include plants that are cultivated by humans for food, feed, fiber, fuel, and/or industrial purposes. Agricultural plants include monocotyledonous species such as: maize (Zea mays), common wheat (Triticum aestivum), spelt (Triticum spelta), einkorn wheat (Triticum monococcum), emmer wheat (Triticum dicoccum), durum wheat (Triticum durum), Asian rice (Oryza sativa), African rice (Oryza glabaerreima), wild rice (Zizania aquatica, Zizania latifolia, Zizania palustris, Zizania texana), barley (Hordeum vulgare), Sorghum (Sorghum bicolor), Finger millet (Eleusine coracana), Proso millet (Panicum miliaceum), Pearl millet (Pennisetum glaucum), Foxtail millet (Setaria italica), Oat (Avena sativa), Triticale (Triticosecale), rye (Secale cereal), Russian wild rye (Psathyrostachys juncea), bamboo (Bambuseae), or sugarcane (e.g., Saccharum arundinaceum, Saccharum barberi, Saccharum bengalense, Saccharum edule, Saccharum munja, Saccharum officinarum, Saccharum procerum, Saccharum ravennae, Saccharum robustum, Saccharum sinense, or Saccharum spontaneum); as well as dicotyledonous species such as: soybean (Glycine max), canola and rapeseed cultivars (Brassica napus), cotton (genus Gossypium), alfalfa (Medicago sativa), cassava (genus Manihot), potato (Solanum tuberosum), tomato (Solanum lycopersicum), pea (Pisum sativum), chick pea (Cicer arietinum), lentil (Lens culinaris), flax (Linum usitatissimum) and many varieties of vegetables.

The term “synthetic combination” means one or more plant elements associated by human endeavor with an isolated, purified endophyte composition, said association which is not found in nature. In some embodiments of the present invention, “synthetic combination” is used to refer to a treatment formulation comprising an isolated, purified population of endophytes associated with a plant element. In some embodiments of the present invention, “synthetic combination” refers to a purified population of endophytes in a treatment formulation comprising additional compositions with which said endophytes are not found associated in nature.

A “treatment formulation” refers to a mixture of chemicals that facilitate the stability, storage, and/or application of the endophyte composition(s). Treatment formulations may comprise any one or more agents such as: surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt.

In some embodiments, an “agriculturally compatible carrier” can be used to formulate an agricultural formulation or other composition that includes a purified endophyte preparation. As used herein an “agriculturally compatible carrier” refers to any material, other than water, that can be added to a plant element without causing or having an adverse effect on the plant element (e.g., reducing seed germination) or the plant that grows from the plant element, or the like.

The compositions and methods herein may provide for an improved “agronomic trait” or “trait of agronomic importance” to a host plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, compared to an isoline plant derived from a seed without said seed treatment formulation.

As used herein, the terms “water-limited condition” and “drought condition,” or “water-limited” and “drought,” may be used interchangeably. For example, a method or composition for improving a plant's ability to grow under drought conditions means the same as the ability to grow under water-limited conditions. In such cases, the plant can be further said to display improved tolerance to drought stress.

As used herein, the terms “normal watering” and “well-watered” are used interchangeably, to describe a plant grown under typical growth conditions with no water restriction.

Additionally, “altered metabolic function” or “altered enzymatic function” may include, but not be limited to, the following: altered production of an auxin, altered nitrogen fixation, altered production of an antimicrobial compound, altered production of a siderophore, altered mineral phosphate solubilization, altered production of a cellulase, altered production of a chitinase, altered production of a xylanase, altered production of acetoin, altered utilization of a carbon source.

An “increased yield” can refer to any increase in biomass or seed or fruit weight, seed size, seed number per plant, seed number per unit area, bushels per acre, tons per acre, kilo per hectare, or carbohydrate yield. Typically, the particular characteristic is designated when referring to increased yield, e.g., increased grain yield or increased seed size.

“Nutrient” or “seed nutrient” refers to any composition of the associated plant element, most particularly compositions providing benefit to other organisms that consume or utilize said plant element.

“Agronomic trait potential” is intended to mean a capability of a plant element for exhibiting a phenotype, preferably an improved agronomic trait, at some point during its life cycle, or conveying said phenotype to another plant element with which it is associated in the same plant. For example, a plant element may comprise an endophyte that will provide benefit to leaf tissue of a plant from which the plant element is grown; in such case, the plant element comprising such endophyte has the agronomic trait potential for a particular phenotype (for example, increased biomass in the plant) even if the plant element itself does not display said phenotype.

In some cases, the present invention contemplates the use of compositions that are “compatible” with agricultural chemicals, including but not limited to, a fungicide, an anticomplex compound, a bactericide, a virucide, an herbicide, a nematicide, a parasiticide, a pesticide, or any other agent widely used in agricultural which has the effect of killing or otherwise interfering with optimal growth of another organism. As used herein, a composition is “compatible” with an agricultural chemical when the organism is modified, such as by genetic modification, e.g., contains a transgene that confers resistance to an herbicide, or is adapted to grow in, or otherwise survive, the concentration of the agricultural chemical used in agriculture. For example, an endophyte disposed on the surface of a plant element is compatible with the fungicide metalaxyl if it is able to survive the concentrations that are applied on the plant element surface.

As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

As used herein, the terms “contacting” and “associating” (and their derivatives) can refer to the method of introducting an endophyte to a non-endophyte, for example to a plant reproductive element, e.g., a seed. The result can include the endophyte being present in a stable relationship with the plant reproductive element, for example on the surface of a seed, in the interior of a seed, or in a formulation that itself is associated with a seed.

The terms “decreased,” “fewer,” “slower” and “increased” “faster” “enhanced” “greater” as used herein refers to a decrease or increase in a characteristic of the endophyte treated plant element or resulting plant compared to an untreated plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

### DETAILED DESCRIPTION OF THE INVENTION

As demonstrated herein, agricultural plants may be associated with symbiotic microorganisms, termed endophytes, particularly bacteria and fungi, which may contribute to plant survival, performance, and characteristics. However, modern agricultural processes may have perturbed this relationship, resulting in increased crop losses, diminished stress resilience, biodiversity losses, and increasing dependence on external chemicals, fertilizers, and other unsustainable agricultural practices. There is a need for novel compositions and methods for generating plants with novel microbiome properties that can sustainably increase yield, improve stress resilience, and decrease fertilizer and chemical use.

Currently, the generally accepted view of plant endophytic communities focuses on their homologous derivation, predominantly from the soil communities in which the plants are grown (Hallman, et al., (1997) Canadian Journal of Microbiology. 43(10): 895-914). Upon observing taxonomic overlap between the endophytic and soil microbiota in A. thaliana, it was stated, “Our rigorous definition of an endophytic compartment microbiome should facilitate controlled dissection of plant-microbe interactions derived from complex soil communities” (Lundberg et al., (2012) Nature. 488, 86-90). There is strong support in the art for soil representing the repository from which plant endophytes are derived. New Phytologist (2010) 185: 554-567. Notable plant-microbe interactions such as mycorrhyzal fungi and complex rhizobia fit the paradigm of soil-based colonization of plant hosts and appear to primarily establish themselves independently of seed. As a result of focusing attention on the derivation of endophytes from the soil in which the target agricultural plant is currently growing, there has been an inability to achieve commercially significant improvements in plant yields and other plant characteristics such as increased root biomass, increased root length, increased height, increased shoot length, increased leaf number, increased water use efficiency, increased overall biomass, increase grain yield, increased photosynthesis rate, increased tolerance to drought, increased heat tolerance, increased salt tolerance, increased resistance to insect and nematode stresses, increased resistance to a fungal pathogen, increased resistance to a complex pathogen, increased resistance to a viral pathogen, a detectable modulation in the level of a metabolite, a detectable modulation in the level of a transcript, or a detectable modulation in the proteome relative to a reference plant.

The inventors herein have conceived of using endophytes that are capable of living within or otherwise associated with plants to improve plant characteristics, as well as methods of using endophytes that are capable of being associated with plants, to impart novel characteristics to a host plant, as well as to distinct plant elements of the host plant. In an embodiment of this invention, endophyte compositions are isolated and purified from plant or fungal sources, and synthetically combined with a plant element, to impart improved agronomic potential and/or improved agronomic traits to the host plant. In another embodiment of the invention, endophytes that are capable of living within plants are isolated and purified from their native source(s) and synthetically combined with a plant element, to impart improved agronomic potential and/or improved agronomic traits to the host plant or the host plant's elements. Such endophytes that are capable of living within plants may be further manipulated or combined with additional elements prior to combining with the plant element(s).

As described herein, beneficial organisms can be robustly obtained from heterologous, homologous, or engineered sources, optionally cultured, administered heterologously to plant elements, and, as a result of the administration, confer multiple beneficial properties. This is surprising given the variability observed in the art in endophytic microbe isolation and the previous observations of inefficient plant element pathogen colonization of plant host's tissues.

In part, the present invention provides preparations of endophytes that are capable of living within plants, and the creation of synthetic combinations of plant elements and/or seedlings with heterologous endophytes, and formulations comprising the synthetic combinations, as well as the recognition that such synthetic combinations display a diversity of beneficial properties present in the agricultural plants and the associated endophyte populations newly created by the present inventors. Such beneficial properties include metabolism, transcript expression, proteome alterations, morphology, and the resilience to a variety of environmental stresses, and any combination of such properties. The present invention also provides methods of using such endophytes to benefit the host plant with which it is associated.

### Endophyte Compositions and Methods of Isolation

The endophytes of the present invention provide several unexpected and significant advantages over other plant-associated microbes. Different environments can comprise significantly different populations of endophytes and thus may provide reservoirs for desired endophytes. Once a choice environment is selected, plant elements of choice plants to be sampled can be identified by their healthy and/or robust growth, or other desired phenotypic characteristics.

In some embodiments of the present invention, endophytes may be fungi identified from a plant source. In some embodiments of the present invention, endophytes are fungi identified from a non-plant source, yet be capable of living within a plant, to create a new endophyte entity.

### Endophyte Selection: Sourcing

In some embodiments of the present invention, endophytes may be isolated from plants or plant elements. In an embodiment of the present invention, endophytes described herein can also be isolated from plants, plant elements, or endophytic fungi of plants or plant elements adapted to a particular environment, including, but not limited to, an environment with water deficiency, salinity, acute and/or chronic heat stress, acute and/or chronic cold stress, nutrient deprived soils including, but not limited to, micronutrient deprived soils, macronutrient (e.g., potassium, phosphate, nitrogen) deprived soils, pathogen stress, including fungal, nematode, insect, viral, and complex pathogen stress.

In one embodiment, a plant is harvested from a soil type different than that in which the plant is normally grown. In another embodiment, the plant is harvested from an ecosystem where the agricultural plant is not normally found. In another embodiment, the plant is harvested from a soil with an average pH range that is different from the optimal soil pH range of the agricultural plant. In one embodiment, the plant is harvested from an environment with average air temperatures lower than the normal growing temperature of the agricultural plant. In one embodiment, the plant is harvested from an environment with average air temperatures higher than the normal growing temperature of the agricultural plant. In another embodiment, the plant is harvested from an environment with average rainfall lower than the optimal average rainfall received by the agricultural plant. In one embodiment, the plant is harvested from an environment with average rainfall higher than the optimal average rainfall of the agricultural plant. In another embodiment, the plant is harvested from a soil type with different soil moisture classification than the normal soil type that the agricultural plant is grown on. In one embodiment, the plant is harvested from an environment with average rainfall lower than the optimal average rainfall of the agricultural plant. In one embodiment, the plant is harvested from an environment with average rainfall higher than the optimal average rainfall of the agricultural plant. In another embodiment, the plant is harvested from an agricultural environment with a yield lower than the average yield expected from the agricultural plant grown under average cultivation practices on normal agricultural land. In another embodiment, the plant is harvested from an agricultural environment with a yield lower than the average yield expected from the agricultural plant grown under average cultivation practices on normal agricultural land. In another embodiment, the plant is harvested from an environment with average yield higher than the optimal average yield of the agricultural plant. In another embodiment, the plant is harvested from an environment with average yield higher than the optimal average yield of the agricultural plant. In another embodiment, the plant is harvested from an environment where soil contains lower total nitrogen than the optimum levels recommended in order to achieve average yields for a plant grown under average cultivation practices on normal agricultural land. In another embodiment, the plant is harvested from an environment where soil contains higher total nitrogen than the optimum levels recommended in order to achieve average yields for a plant grown under average cultivation practices on normal agricultural land. In another embodiment, the plant is harvested from an environment where soil contains lower total phosphorus than the optimum levels recommended in order to achieve average yields for a plant grown under average cultivation practices on normal agricultural land. In another embodiment, the plant is harvested from an environment where soil contains higher total phosphorus than the optimum levels recommended in order to achieve average yields for a plant grown under average cultivation practices on normal agricultural land. In another embodiment, the plant is harvested from an environment where soil contains lower total potassium than the optimum levels recommended in order to achieve average yields for a plant grown under average cultivation practices on normal agricultural land. In another embodiment, the plant is harvested from an environment where soil contains higher total potassium than the optimum levels recommended in order to achieve average yields for a plant grown under average cultivation practices on normal agricultural land. In another embodiment, the plant is harvested from an environment where soil contains lower total sulfur than the optimum levels recommended in order to achieve average yields for a plant grown under average cultivation practices on normal agricultural land. In another embodiment, the plant is harvested from an environment where soil contains higher total sulfur than the optimum levels recommended in order to achieve average yields for a plant grown under average cultivation practices on normal agricultural land. In another embodiment, the plant is harvested from an environment where soil contains lower total calcium than the optimum levels recommended in order to achieve average yields for a plant grown under average cultivation practices on normal agricultural land. In another embodiment, the plant is harvested from an environment where soil contains lower total magnesium than the optimum levels recommended in order to achieve average yields for a plant grown under average cultivation practices on normal agricultural land. In another embodiment, the plant is harvested from an environment where soil contains higher total sodium chloride (salt) than the optimum levels recommended in order to achieve average yields for a plant grown under average cultivation practices on normal agricultural land.

Endophytes can be obtained from a host plant or a plant element of many distinct plants. In an embodiment, the endophyte can be obtained a plant element of the same or different crop, and can be from the same or different cultivar or variety as the plant element to which the composition is heterologously associated.

In another embodiment, endophytes used in a composition or used to make a synthetic combination can be obtained from the same cultivar or species of agricultural plant to which the composition is intended for heterologous association, or can be obtained from a different cultivar or species of agricultural plant. For example, endophytes from a particular corn variety can be isolated and coated onto the surface of a corn plant element of the same variety.

In another embodiment, endophytes used in a composition or used to make a synthetic combination can be obtained from a plant element of a plant that is related to the plant element to which the composition is intended to be association. For example, an endophyte isolated from Triticum monococcum (einkorn wheat) can be coated onto the surface of a T. aestivum (common wheat) plant element; or, an endophyte from Hordeum vulgare (barley) can be isolated and coated onto the plant element of a member of the Triticeae family, for example, plant elements of the rye plant, Secale cereale).

In still another embodiment, endophytes used in a composition or used to make a synthetic combination can be obtained from a plant element of a plant that is distantly related to the plant element onto which the endophyte is to be coated. For example, a tomato-derived endophyte can be isolated and coated onto a soybean plant element.

In some embodiments, a purified endophytes population is used that includes two or more (e.g., 3, 4, 5, 6, 7, 8, 9, 10, 15, 20, 25 or greater than 25) different endophytes, e.g., obtained from different families of plant or fungus, or different genera of plant or fungus, or from the same genera but different species of plant or fungus.

In yet another embodiment, endophytes used in a composition or used to make a synthetic combination can be obtained from different individual plants of the same variety, each of which has been subjected to different growth conditions. For example, an endophyte obtained from a drought-affected plant of one variety can be isolated and coated onto the plant element that was derived from a plant of the same variety not subjected to drought. In such cases, the endophyte is considered to be heterologously associated with the plant element onto which it is applied.

The heterologous relationship between the endophyte and the host plant element may result from an endophyte obtained from any different plant or plant element than that which with it becomes associated. In some cases, the endophyte is obtained from a different cultivar of the same species. In some cases, the endophyte is obtained from a different plant species. In some cases, the endophyte is obtained from the same plant species but from two different plants, each exposed to some different environmental condition (for example, differences in heat units or water stress). In some cases, the endophyte is obtained from the same plant individual but from different plant elements or tissues (for example, a root endophyte applied to a leaf).

In some embodiments, compositions described herein comprise a purified endophyte population is used that includes at least two or more, at least 3, at least 4, at least 5, at least 6, at least 7, at least 8, at least 9, at least 10, at least 15, at least 20, at least 25, or more (e.g., 3, 4, 5, 6, 7, 8, 9, 10, 15, 20, 25 or greater than 25) different endophytes, e.g., obtained from different families of plants, or different genera of plant or fungus, or from the same genera but different species of plants.

The different endophytes can be obtained from the same cultivar of agricultural plant (e.g., the same maize, wheat, rice, or barley plant), different cultivars of the same agricultural plant (e.g., two or more cultivars of maize, two or more cultivars of wheat, two or more cultivars of rice, or two or more cultivars of barley), or different species of the same type of agricultural plant (e.g., two or more different species of maize, two or more different species of wheat, two or more different species of rice, or two or more different species of barley). In embodiments in which two or more endophytes are used, each of the endophytes can have different properties or activities, e.g., produce different metabolites, produce different enzymes such as different hydrolytic enzymes, confer different beneficial traits, or colonize different elements of a plant (e.g., leaves, stems, flowers, fruits, seeds, or roots). For example, one endophyte can colonize a first and a second endophyte can colonize a tissue that differs from the first tissue. Combinations of endophytes are disclosed in detail below.

In an embodiment, the endophyte is an endophytic microbe isolated from a different plant than the inoculated plant. For example, in an embodiment, the endophyte is an endophyte isolated from a different plant of the same species as the inoculated plant. In some cases, the endophyte is isolated from a species related to the inoculated plant.

Endophyte Selection: Compatibility with Agrichemicals

In certain embodiments, the endophyte is selected on the basis of its compatibility with commonly used agrichemicals. As mentioned earlier, plants, particularly agricultural plants, can be treated with a vast array of agrichemicals, including fungicides, biocides (anticomplex agents), herbicides, insecticides, nematicides, rodenticides, bactericides, virucides, fertilizers, and other agents.

In some embodiments, the endophytes of the present invention display tolerance to an agrichemical selected from the group consisting of: Aeris®, Avicta® DuoCot 202, Cruiser®, Syntenta CCB® (A), Clariva®, Albaugh, Dynasty®, Apron®, Maxim®, Gaucho®, Provoke® ST, Syngenta CCB®, Trilex®, WG Purple, WG Silver, Azoxystrobin, Carboxin, Difenoconazole, Fludioxonil, fluxapyroxad, Ipconazole, Mefenoxam, Metalaxyl, Myclobutanil, Penflufen, pyraclostrobin, Sedaxane, TCMTB, Tebuconazole, Thiram, Triadimenol (Baytan®), Trifloxystrobin, Triticonazole, Tolclofos-methyl, PCNB, Abamectin, Chlorpyrifos, Clothianidin, Imidacloprid, Thiamethoxam, Thiodicarb.

In some cases, it can be important for the endophyte to be compatible with agrichemicals, particularly those with anticomplex properties, in order to persist in the plant although, as mentioned earlier, there are many such anticomplex agents that do not penetrate the plant, at least at a concentration sufficient to interfere with the endophyte. Therefore, where a systemic anticomplex agent is used in the plant, compatibility of the endophyte to be inoculated with such agents will be an important criterion.

In an embodiment, natural isolates of endophytes that are compatible with agrichemicals can be used to inoculate the plants according to the methods described herein. For example, endophytes that are compatible with agriculturally employed anticomplex agents can be isolated by plating a culture of endophytes on a petri dish comprising an effective concentration of the anticomplex agent, and isolating colonies of endophytes that are compatible with the anticomplex agent. In another embodiment, an endophyte that is compatible with an anticomplex agent is used for the methods described herein.

Bactericide-compatible endophyte can also be isolated by selection on liquid medium. The culture of endophytes can be plated on petri dishes without any forms of mutagenesis; alternatively, endophytes can be mutagenized using any means known in the art. For example, endophyte cultures can be exposed to UV light, gamma-irradiation, or chemical mutagens such as ethylmethanesulfonate (EMS), ethidium bromide (EtBr) dichlovos (DDVP, methyl methane sulphonale (MMS), triethylphosphate (TEP), trimethylphosphate (TMP), nitrous acid, or DNA base analogs, prior to selection on fungicide comprising media. Finally, where the mechanism of action of a particular bactericide is known, the target gene can be specifically mutated (either by gene deletion, gene replacement, site-directed mutagenesis, etc.) to generate an endophyte that is resilient against that particular chemical. It is noted that the above-described methods can be used to isolate endophytes that are compatible with both bacteriostatic and bactericidal compounds.

It will also be appreciated by one skilled in the art that a plant may be exposed to multiple types of anticomplex compounds, either simultaneously or in succession, for example at different stages of plant growth. Where the target plant is likely to be exposed to multiple anticomplex agents, an endophyte that is compatible with many or all of these agrichemicals can be used to inoculate the plant. An endophyte that is compatible with several agents can be isolated, for example, by serial selection. An endophyte that is compatible with the first agent can be isolated as described above (with or without prior mutagenesis). A culture of the resulting endophyte can then be selected for the ability to grow on liquid or solid media comprising the second agent (again, with or without prior mutagenesis). Colonies isolated from the second selection are then tested to confirm its compatibility to both agents.

Likewise, endophytes that are compatible to biocides (including herbicides such as glyphosate or anticomplex compounds, whether bacteriostatic or bactericidal) that are agriculturally employed can be isolated using methods similar to those described for isolating compatible endophytes. In one embodiment, mutagenesis of the endophyte population can be performed prior to selection with an anticomplex agent. In another embodiment, selection is performed on the endophyte population without prior mutagenesis. In still another embodiment, serial selection is performed on an endophyte: the endophyte is first selected for compatibility to a first anticomplex agent. The isolated compatible endophyte is then cultured and selected for compatibility to the second anticomplex agent. Any colony thus isolated is tested for compatibility to each, or both anticomplex agents to confirm compatibility with these two agents.

Compatibility with an antimicrobial agent can be determined by a number of means known in the art, including the comparison of the minimal inhibitory concentration (MIC) of the unmodified and modified endophytes. Therefore, in one embodiment, the present invention discloses an isolated modified endophyte, wherein the endophyte is modified such that it exhibits at least 3 fold greater, for example, at least 5 fold greater, between 5 and 10 fold greater, at least 10 fold greater, between 10 and 20 fold greater, at least 20 fold greater, between 20 and 30 fold greater, at least 30 fold greater or more MIC to an antimicrobial agent when compared with the unmodified endophyte.

In one embodiment, disclosed herein are endophytes with enhanced compatibility to the herbicide glyphosate. In one embodiment, the endophyte has a doubling time in growth medium comprising at least 1 mM glyphosate, for example, between 1 mM and 2 mM glyphosate, at least 2 mM glyphosate, between 2 mM and 5 mM glyphosate, at least 5 mM glyphosate, between 5 mM and 10 mM glyphosate, at least 10 mM glyphosate, between 10 mM and 15 mM glyphosate, at least 15 mM glyphosate or more, that is no more than 250%, between 250% and 100%, for example, no more than 200%, between 200% and 175%, no more than 175%, between 175% and 150%, no more than 150%, between 150% and 125%, or no more than 125%, of the doubling time of the endophyte in the same growth medium comprising no glyphosate. In one particular embodiment, the endophyte has a doubling time in growth medium comprising 5 mM glyphosate that is no more than 150% the doubling time of the endophyte in the same growth medium comprising no glyphosate.

In another embodiment, the endophyte has a doubling time in a plant tissue comprising at least 10 ppm glyphosate, between 10 and 15 ppm, for example, at least 15 ppm glyphosate, between 15 and 10 ppm, at least 20 ppm glyphosate, between 20 and 30 ppm, at least 30 ppm glyphosate, between 30 and 40 ppm, at least 40 ppm glyphosate or more, that is no more than 250%, between 250% and 200%, for example, no more than 200%, between 200% and 175%, no more than 175%, between 175% and 150%, no more than 150%, between 150% and 125%, or no more than 125%, of the doubling time of the endophyte in a reference plant tissue comprising no glyphosate. In one particular embodiment, the endophyte has a doubling time in a plant tissue comprising 40 ppm glyphosate that is no more than 150% the doubling time of the endophyte in a reference plant tissue comprising no glyphosate.

The selection process described above can be repeated to identify isolates of endophytes that are compatible with a multitude of agents.

Candidate isolates can be tested to ensure that the selection for agrichemical compatibility did not result in loss of a desired bioactivity. Isolates of endophytes that are compatible with commonly employed agents can be selected as described above. The resulting compatible endophyte can be compared with the parental endophyte on plants in its ability to promote germination.

The agrichemical compatible endophytes generated as described above can be detected in samples. For example, where a transgene was introduced to render the endophyte compatible with the agrichemical(s), the transgene can be used as a target gene for amplification and detection by PCR. In addition, where point mutations or deletions to a portion of a specific gene or a number of genes results in compatibility with the agrichemical(s), the unique point mutations can likewise be detected by PCR or other means known in the art. Such methods allow the detection of the endophyte even if it is no longer viable. Thus, commodity plant products produced using the agrichemical compatible endophytes described herein can readily be identified by employing these and related methods of nucleic acid detection.

### Endophyte Selection: Combinations

Combinations of endophytes can be selected by any one or more of several criteria. In one embodiment, compatible endophytes are selected. As used herein, “compatibility” refers to endophyte populations that do not significantly interfere with the growth, propagation, and/or production of beneficial substances of the other. Incompatible endophyte populations can arise, for example, where one of the populations produces or secrets a compound that is toxic or deleterious to the growth of the other population(s). Incompatibility arising from production of deleterious compounds/agents can be detected using methods known in the art, and as described herein elsewhere. Similarly, the distinct populations can compete for limited resources in a way that makes co-existence difficult.

In another embodiment, combinations are selected on the basis of compounds produced by each population of endophytes. For example, the first population is capable of producing siderophores, and another population is capable of producing anti-fungal compounds. In an embodiment, the first population of endophytes or endophytic components is capable of a function selected from the group consisting of auxin production, nitrogen fixation, and production of an antimicrobial compound, siderophore production, mineral phosphate solubilization, cellulase production, chitinase production, xylanase production, and acetoin production, carbon source utilization, and combinations thereof. In another embodiment, the second population of endophytes or endophytic component is capable of a function selected from the group consisting of auxin production, nitrogen fixation, production of an antimicrobial compound, siderophore production, mineral phosphate solubilization, cellulase production, chitinase production, xylanase production, and acetoin production, and combinations thereof. In still another embodiment, the first and second populations are capable of at least one different function.

In still another embodiment, the combinations of endophytes are selected for their distinct localization in the plant after colonization. For example, the first population of endophytes or endophytic components can colonize, and in some cases preferentially colonize, the root tissue, while a second population can be selected on the basis of its preferential colonization of the aerial parts of the agricultural plant. Therefore, in an embodiment, the first population is capable of colonizing one or more of the tissues selected from the group consisting of a root, shoot, leaf, flower, and seed. In another embodiment, the second population is capable of colonizing one or more tissues selected from the group consisting of root, shoot, leaf, flower, and seed. In still another embodiment, the first and second populations are capable of colonizing a different tissue within the agricultural plant.

In some embodiments, combinations of endophytes are selected for their ability to confer a benefit to the host plant at different points in the life cycle of said host plant. In one example, one endophyte can be selected to impart improved seedling vigor, and a second endophyte can be selected to improve soil nutrient acquisition by roots of the mature plant.

In still another embodiment, combinations of endophytes are selected for their ability to confer one or more distinct fitness traits on the inoculated agricultural plant, either individually or in synergistic association with other endophytes. In another embodiment, one endophyte may induce the colonization of a second endophyte. Alternatively, two or more endophytes may induce the colonization of a third endophyte. For example, the first population of endophytes or endophytic components is selected on the basis that it confers significant increase in biomass, while the second population promotes increased drought tolerance on the inoculated agricultural plant. Therefore, in one embodiment, the first population is capable of conferring at least one trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof. In another embodiment, the second population is capable of conferring a trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, and enhanced soil water retention. In still another embodiment, each of the first and second population is capable of conferring a different trait selected from the group consisting of thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, and enhanced soil water retention.

The combinations of endophytes can also be selected based on combinations of the above criteria. For example, the first population of endophytes can be selected on the basis of the compound it produces (e.g., its ability to fix nitrogen, thus providing a potential nitrogen source to the plant), while the second population can be selected on the basis of its ability to confer increased resistance of the plant to a pathogen (e.g., a fungal pathogen).

In some embodiments of the present invention, it is contemplated that combinations of endophytes can provide an increased benefit to the host plant, as compared to that conferred by a single endophyte, by virtue of additive effects. For example, one endophyte strain that induces a benefit in the host plant may induce such benefit equally well in a plant that is also colonized with a different endophyte strain that also induces the same benefit in the host plant. The host plant thus exhibits the same total benefit from the combination of different endophyte strains as the additive benefit to individual plants colonized with each individual endophyte of the combination. In one example, a plant is colonized with two different endophyte strains: one provides a 1× increase in biomass when associated with the plant, and the other provides a 2× increase in biomass when associated with a different plant. When both endophyte strains are associated with the same plant, that plant would experience a 3× (additive of 1×+2× single effects) increase in auxin biomass. Additive effects are a surprising embodiment of the present invention.

In some embodiments of the present invention, it is contemplated that a combination of endophytes can provide an increased benefit to the host plant, as compared to that conferred by a single endophyte, by virtue of synergistic effects. For example, one endophyte strain that induces a benefit in the host plant may induce such benefit beyond additive effects in a plant that is also colonized with a different endophyte strain that also induces that benefit in the host plant. The host plant thus exhibits the greater total benefit from the combination of different endophyte strains than could be seen from the additive benefit of individual plants colonized with each individual endophyte of the combination. In one example, a plant is colonized with two different endophyte strains: one provides a 1× increase in biomass when associated with a plant, and the other provides a 2× increase in biomass when associated with a different plant. When both endophyte strains are associated with the same plant, that plant would experience a 5× (greater than an additive of 1×+2× single effects) increase in biomass. Synergistic effects are a surprising embodiment of the present invention.

### Endophyte Selection: Compositions of the Invention

In some embodiments, the endophyte is selected from the genus Penicillium. In some embodiments, the endophyte comprises a nucleotide sequence that is at least 97% identical to SEQ ID NO: 1. In some embodiments, the endophyte comprises a nucleotide sequence that is at least 97% identical to SEQ ID NO: 2. In some embodiments, the endophyte comprises a nucleotide sequence that is at least 97% identical to SEQ ID NO: 3. In some embodiments, the endophyte comprises a nucleotide sequence that is at least 97% identical to SEQ ID NO: 4. In some embodiments, the endophyte comprises a nucleotide sequence that is at least 97% identical to SEQ ID NO: 5. In some embodiments, the endophyte comprises a nucleotide sequence that is at least 97% identical to SEQ ID NO: 6. In some embodiments, the endophyte comprises a nucleotide sequence that is at least 97% identical to SEQ ID NO: 7. In some embodiments, the endophyte comprises a nucleotide sequence that is at least 97% identical to SEQ ID NO: 8.

In some embodiments, the endophyte is at least 97% identical to a sequence selected from the group consisting of SEQ ID NO: 1-SEQ ID NO: 8. In some embodiments, the endophyte is between 97% and 98% identical, at least 98% identical, between 98% identical and 99% identical, or at least 99% identical to a sequence selected from the group consisting of SEQ ID NO: 1-SEQ ID NO: 8.

In some cases, the endophyte, or one or more components thereof, is of monoclonal origin, providing high genetic uniformity of the endophyte population in an agricultural formulation or within a synthetic plant element or plant combination with the endophyte.

In some embodiments, the endophyte can be cultured on a culture medium or can be adapted to culture on a culture medium.

The compositions provided herein are preferably stable. The endophyte may be shelf-stable, where at least 0.01%, of the CFUs are viable after storage in desiccated form (i.e., moisture content of 30% or less) for 1, 2, 3, 4, 5, 6, 7, 8, 9, 10 or greater than 10 weeks at 4° C. or at room temperature. Optionally, a shelf-stable formulation is in a dry formulation, a powder formulation, or a lyophilized formulation. In some embodiments, the formulation is formulated to provide stability for the population of endophytes. In an embodiment, the formulation is substantially stable at temperatures between about −20° C. and about 50° C. for at least about 1, 2, 3, 4, 5, or 6 days, or 1, 2, 3 or 4 weeks, or 1, 2, 3, 4, 5, 6, 7, 8, 9, 10, 11 or 12 months, or one or more years. In another embodiment, the formulation is substantially stable at temperatures between about 4° C. and about 37° C. for at least about 5, 10, 15, 20, 25, 30 or greater than 30 days.

Endophytes and Synthetic Combinations with Plants and Plant Elements

It is contemplated that the methods and compositions of the present invention may be used to improve any characteristic of any agricultural plant. The methods described herein can also be used with transgenic plants comprising one or more exogenous transgenes, for example, to yield additional trait benefits conferred by the newly introduced endophytic microbes. Therefore, in one embodiment, a plant element of a transgenic soybean plant is contacted with an endophytic microbe. In one embodiment, a plant element of a transgenic maize plant is contacted with an endophytic microbe.

For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

In one embodiment, it is contemplated that the plant of the present invention is soybean (Glycine max).

The primary uses for harvested soybean crops include: soybean oil, soybean meal, livestock feed, and uses for human consumption. All parts of a soy plant are utilized, including the starch, flours, oils, and proteins.

In one embodiment, it is contemplated that the plant of the present invention is maize (corn) (Zea mays).

The primary uses for harvested maize crops include: livestock feed, food for human consumption, biofuels, high fructose corn syrup, sweeteners, dry distiller grains, plastics, cosmetics, and textiles. All parts of a corn plant are utilized, including the starch, fiber, proteins, and oils.

The endophyte compositions and methods of the present invention are capable of providing improvements of agronomic interest agricultural plants, for example soybeans and maize.

In some embodiments, the present invention contemplates the use of endophytes that can confer a beneficial agronomic trait upon the plant element or resulting plant with which it is associated.

In some cases, the endophytes described herein are capable of moving from one tissue type to another. For example, the present invention's detection and isolation of endophytes within the mature tissues of plants after coating on the exterior of a plant element demonstrates their ability to move from the plant element into the vegetative tissues of a maturing plant. Therefore, in one embodiment, the population of endophytes is capable of moving from the plant element exterior into the vegetative tissues of a plant. In one embodiment, the endophyte that is coated onto the plant element of a plant is capable, upon germination of the plant element into a vegetative state, of localizing to a different tissue of the plant. For example, endophytes can be capable of localizing to any one of the tissues in the plant, including: the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In an embodiment, the endophyte is capable of localizing to the root and/or the root hair of the plant. In another embodiment, the endophyte is capable of localizing to the photosynthetic tissues, for example, leaves and shoots of the plant. In other cases, the endophyte is localized to the vascular tissues of the plant, for example, in the xylem and phloem. In still another embodiment, the endophyte is capable of localizing to the reproductive tissues (flower, pollen, pistil, ovaries, stamen, fruit) of the plant. In another embodiment, the endophyte is capable of localizing to the root, shoots, leaves and reproductive tissues of the plant. In still another embodiment, the endophyte colonizes a fruit or plant element tissue of the plant. In still another embodiment, the endophyte is able to colonize the plant such that it is present in the surface of the plant (i.e., its presence is detectably present on the plant exterior, or the episphere of the plant). In still other embodiments, the endophyte is capable of localizing to substantially all, or all, tissues of the plant. In certain embodiments, the endophyte is not localized to the root of a plant. In other cases, the endophyte is not localized to the photosynthetic tissues of the plant.

In some cases, endophytes are capable of replicating within the host plant and colonizing the plant.

As shown in the Examples section below, the endophyte populations described herein are capable of colonizing a host plant. Successful colonization can be confirmed by detecting the presence of the endophyte population within the plant. For example, after applying the fungi to the plant elements, high titers of the fungi can be detected in the roots and shoots of the plants that germinate from the plant elements. Detecting the presence of the endophyte inside the plant can be accomplished by measuring the viability of the endophyte after surface sterilization of the plant element or the plant: endophyte colonization results in an internal localization of the endophyte, rendering it resistant to conditions of surface sterilization. The presence and quantity of endophyte can also be established using other means known in the art, for example, immunofluorescence microscopy using microbe-specific antibodies, or fluorescence in situ hybridization (see, for example, Amann et al. (2001) Current Opinion in Biotechnology 12:231-236, incorporated herein by reference in its entirety). Alternatively, specific nucleic acid probes recognizing conserved sequences from an endophyte can be employed to amplify a region, for example by quantitative PCR, and correlated to CFUs by means of a standard curve.

In some cases, plants are inoculated with endophytes that are isolated from the same species of plant as the plant element of the inoculated plant. For example, an endophyte that is normally found in one variety of a plant is associated with a plant element of a plant of another variety of that plant that in its natural state lacks said endophyte. For example, an endophyte that is normally found in one variety of Glycine max (soybean) is associated with a plant element of a plant of another variety of Glycine max that in its natural state lacks said endophyte. In an embodiment, the endophyte is obtained from a plant of a related species of plant as the plant element of the inoculated plant. For example, an endophyte that is normally found in one species of a plant is applied to another species of the same genus, or vice versa. In some cases, plants are inoculated with endophytes that are heterologous to the plant element of the inoculated plant. In an embodiment, the endophyte is obtained from a plant of another species. For example, an endophyte that is normally found in dicots is applied to a monocot plant, or vice versa. In other cases, the endophyte to be inoculated onto a plant is obtained from a related species of the plant that is being inoculated. In one embodiment, the endophyte is obtained from a related taxon, for example, from a related species. The plant of another species can be an agricultural plant. In another embodiment, the endophyte is part of a designed composition inoculated into any host plant element.

In another embodiment, the endophyte is disposed, for example, on the surface of a reproductive element of an agricultural plant, in an amount effective to be detectable in the mature agricultural plant. In one embodiment, the endophyte is disposed in an amount effective to be detectable in an amount of at least about 100 CFU between 100 and 200 CFU, at least about 200 CFU, between 200 and 300 CFU, at least about 300 CFU, between 300 and 400 CFU, at least about 500 CFU, between 500 and 1,000 CFU, at least about 1,000 CFU, between 1,000 and 3,000 CFU, at least about 3,000 CFU, between 3,000 and 10,000 CFU, at least about 10,000 CFU, between 10,000 and 30,000 CFU, at least about 30,000 CFU, between 30,000 and 100,000 CFU, at least about 100,000 CFU or more in the mature agricultural plant.

In some cases, the endophyte is capable of colonizing particular plant elements or tissue types of the plant. In an embodiment, the endophyte is disposed on the plant element or seedling in an amount effective to be detectable within a target tissue of the mature agricultural plant selected from a fruit, a seed, a leaf, or a root, or portion thereof. For example, the endophyte can be detected in an amount of at least about 100 CFU, at least about 200 CFU, at least about 300 CFU, at least about 500 CFU, at least about 1,000 CFU, at least about 3,000 CFU, at least about 10,000 CFU, at least about 30,000 CFU, at least about 100,000 CFU or more, in the target tissue of the mature agricultural plant.

### Beneficial Attributes of Synthetic Combinations of Plant Elements and Endophytes

### Improved Plant Attributes Conferred by Endophytes

The present invention contemplates the establishment of a relationship between an endophyte and a plant element. In one embodiment, endophyte association results in a detectable change to the plant element, or the whole plant. The detectable change can be an improvement in a number of agronomic traits (e.g., improved general health, increased response to biotic or abiotic stresses, or enhanced properties of the plant or a plant element, including fruits and grains). Alternatively, the detectable change can be a physiological or biological change that can be measured by methods known in the art. The detectable changes are described in more detail in the sections below. As used herein, an endophyte is considered to have conferred an improved agricultural trait whether or not the improved trait arose from the plant, the endophyte, or the concerted action between the plant and endophyte. Therefore, for example, whether a beneficial hormone or chemical is produced by the plant or the endophyte, for purposes of the present invention, the endophyte will be considered to have conferred an improved agronomic trait upon the host plant, as compared to an isoline plant that has not been associated with said endophyte.

In some embodiments, provided herein, are methods for producing a plant element of a plant with a heritably altered trait. The trait of the plant can be altered without known genetic modification of the plant genome, and comprises the following steps. First, a preparation of an isolated endophyte that is heterologous to the plant element of the plant is provided, and optionally processed to produce an endophyte formulation. The endophyte formulation is then contacted with the plant. The plants are then allowed to go to seed, and the seeds are collected.

### Improved General Health

Also described herein are plants, and fields of plants, that are associated with beneficial endophytes, such that the overall fitness, productivity or health of the plant or a portion thereof, is maintained, increased and/or improved over a period of time. Improvement in overall plant health can be assessed using numerous physiological parameters including, but not limited to, height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof. Improved plant health, or improved field health, can also be demonstrated through improved resistance or response to a given stress, either biotic or abiotic stress, or a combination of one or more abiotic stresses, as provided herein.

### Other Abiotic Stresses

Disclosed herein are endophyte-associated plants with increased resistance to an abiotic stress. Exemplary abiotic stresses include, but are not limited to: drought, heat, salt content, metal content, low nutrient conditions, cold, excess water conditions.

Drought and Heat Tolerance.

In some cases, a plant resulting from seeds or other plant elements treated with an endophyte can exhibit a physiological change, such as a compensation of the stress-induced reduction in photosynthetic activity. Fv/Fm tests whether or not plant stress affects photosystem II in a dark adapted state. Fv/Fm is one of the most commonly used chlorophyll fluorescence measuring parameter. The Fv/Fm test is designed to allow the maximum amount of the light energy to take the fluorescence pathway. It compares the dark-adapted leaf pre-photosynthetic fluorescent state, called minimum fluorescence, or Fo, to maximum fluorescence called Fm. In maximum fluorescence, the maximum number of reaction centers have been reduced or closed by a saturating light source. In general, the greater the plant stress, the fewer open reaction centers available, and the Fv/Fm ratio is lowered. Fv/Fm is a measuring protocol that works for many types of plant stress. For example, there would be a difference in the Fv/Fm after exposure of an endophyte treated plant that had been subjected to heat shock or drought conditions, as compared to a corresponding control, a genetically identical plant that does not contain the endophytes grown in the same conditions. In some cases, the endophyte-associated plant as disclosed herein can exhibit an increased change in photosynthetic activity ΔFv(ΔFv/Fm) after heat-shock or drought stress treatment, for example 1, 2, 3, 4, 5, 6, 7 days or more after the heat-shock or drought stress treatment, or until photosynthesis ceases, as compared with corresponding control plant of similar developmental stage but not comprising endophytes. For example, a plant having an endophyte able to confer heat and/or drought-tolerance can exhibit a ΔFv/Fm of from about 0.1 to about 0.8 after exposure to heat-shock or drought stress or a ΔFv/Fm range of from about 0.03 to about 0.8 under one day, or 1, 2, 3, 4, 5, 6, 7, or over 7 days post heat-shock or drought stress treatment, or until photosynthesis ceases. In some embodiments, stress-induced reductions in photosynthetic activity can be compensated by at least about 0.25% (for example, at least about 0.5%, between 0.5% and 1%, at least about 1%, between 1% and 2%, at least about 2%, between 2% and 3%, at least about 3%, between 3% and 5%, at least about 5%, between 5% and 10%, at least about 8%, at least about 10%, between 10% and 15%, at least about 15%, between 15% and 20%, at least about 20%, between 20$ and 25%, at least about 25%, between 25% and 30%, at least about 30%, between 30% and 40%, at least about 40%, between 40% and 50%, at least about 50%, between 50% and 60%, at least about 60%, between 60% and 75%, at least about 75%, between 75% and 80%, at least about 80%, between 80% and 85%, at least about 85%, between 85% and 90%, at least about 90%, between 90% and 95%, at least about 95%, between 95% and 99%, at least about 99% or at least 100%) as compared to the photosynthetic activity decrease in a corresponding reference agricultural plant following heat shock conditions. Significance of the difference between endophyte-associated and reference agricultural plants can be established upon demonstrating statistical significance, for example at p<0.05 with an appropriate parametric or non-parametric statistic, e.g., Chi-square test, Student's t-test, Mann-Whitney test, or F-test based on the assumption or known facts that the endophyte-associated plant and reference agricultural plant have identical or near identical genomes (isoline comparison).

In some embodiments, the plants comprise endophytes able to increase heat and/or drought-tolerance in sufficient quantity, such that increased growth or improved recovery from wilting under conditions of heat or drought stress is observed. For example, an endophyte population described herein can be present in sufficient quantity in a plant, resulting in increased growth as compared to a plant that does not contain endophytes, when grown under drought conditions or heat shock conditions, or following such conditions. Increased heat and/or drought tolerance can be assessed with physiological parameters including, but not limited to, increased height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, wilt recovery, turgor pressure, or any combination thereof, as compared to a reference agricultural plant grown under similar conditions. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

In various embodiments, endophytes introduced into the plant can confer in the resulting plant thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased protein content, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof. A difference between the endophyte-associated plant and a reference agricultural plant can also be measured using other methods known in the art.

Salt Stress.

In other embodiments, endophytes able to confer increased tolerance to salinity stress can be introduced into plants. The resulting plants comprising endophytes can exhibit increased resistance to salt stress, whether measured in terms of survival under saline conditions, or overall growth during, or following salt stress. The physiological parameters of plant health recited above, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., isogenic plants without the endophytes) grown under identical conditions. For example, the endophyte may provide an improved benefit or tolerance to a plant that is of at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions. In other instances, endophyte-associated plants and reference agricultural plants can be grown in soil or growth media comprising different concentration of sodium to establish the inhibitory concentration of sodium (expressed, for example, as the concentration in which growth of the plant is inhibited by 50% when compared with plants grown under no sodium stress). Therefore, in another embodiment, a plant resulting from plant elements comprising an endophyte able to confer salt tolerance described herein exhibits an increase in the inhibitory sodium concentration by at least 10 mM, between 10 mM and 15 mM, for example at least 15 mM, between 15 mM and 20 mM, at least 20 mM, between 20 mM and 30 mM, at least 30 mM, between 30 mM and 40 mM, at least 40 mM, between 40 mM and 50 mM, at least 50 mM, between 50 mM and 60 mM, at least 60 mM, between 60 mM and 70 mM, at least 70 mM, between 70 mM and 80 mM, at least 80 mM, between 80 mM and 90 mM, at least 90 mM, between 90 mM and 100 mM, at least 100 mM or more, when compared with the reference agricultural plants.

High Metal Content.

Plants are sessile organisms and therefore must contend with the environment in which they are placed. Plants have adapted many mechanisms to deal with chemicals and substances that may be deleterious to their health. Heavy metals in particular represent a class of toxins that are highly relevant for plant growth and agriculture, because many of them are associated with fertilizers and sewage sludge used to amend soils and can accumulate to toxic levels in agricultural fields. Therefore, for agricultural purposes, it is important to have plants that are able to tolerate soils comprising elevated levels of toxic heavy metals. Plants cope with toxic levels of heavy metals (for example, nickel, cadmium, lead, mercury, arsenic, or aluminum) in the soil by excretion and internal sequestration. Endophytes that are able to confer increased heavy metal tolerance may do so by enhancing sequestration of the metal in certain compartments away from the seed or fruit and/or by supplementing other nutrients necessary to remediate the stress. Use of such endophytes in a plant would allow the development of novel plant-endophyte combinations for purposes of environmental remediation (also known as phytoremediation). Therefore, in one embodiment, the plant comprising endophytes shows increased metal tolerance as compared to a reference agricultural plant grown under the same heavy metal concentration in the soil.

Alternatively, the inhibitory concentration of the heavy metal can be determined for endophyte-associated plant and compared with a reference agricultural plant under the same conditions. Therefore, in one embodiment, the plants resulting from plant elements comprising an endophyte able to confer heavy metal tolerance described herein exhibit an increase in the inhibitory metal concentration by at least 0.1 mM, between 0.1 mM and 0.3 mM, for example at least 0.3 mM, between 0.3 mM and 0.5 mM, at least 0.5 mM, between 0.5 mM and 1 mM, at least 1 mM, between 1 mM and 2 mM, at least 2 mM, between 2 mM and 5 mM, at least 5 mM, between 5 mM and 10 mM, at least 10 mM, between 10 mM and 15 mM, at least 15 mM, between 15 mM and 20 mM, at least 20 mM, between 20 mM and 30 mM, at least 30 mM, between 30 mM and 50 mM, at least 50 mM or more, when compared with the reference agricultural plants.

Finally, plants inoculated with endophytes that are able to confer increased metal tolerance exhibit an increase in overall metal excretion by at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

Low Nutrient Stress.

Endophytes described herein may also confer to the plant an increased ability to grow in nutrient limiting conditions, for example by solubilizing or otherwise making available to the plants macronutrients or micronutrients that are complexed, insoluble, or otherwise in an unavailable form. In one embodiment, a plant is inoculated with an endophyte that confers increased ability to liberate and/or otherwise provide to the plant with nutrients selected from the group consisting of phosphate, nitrogen, potassium, iron, manganese, calcium, molybdenum, vitamins, or other micronutrients. Such a plant can exhibit increased growth in soil comprising limiting amounts of such nutrients when compared with reference agricultural plant. Differences between the endophyte-associated plant and reference agricultural plant can be measured by comparing the biomass of the two plant types grown under limiting conditions, or by measuring the physical parameters described above. Therefore, in one embodiment, the plant comprising endophyte shows increased tolerance to nutrient limiting conditions as compared to a reference agricultural plant grown under the same nutrient limited concentration in the soil, as measured for example by increased biomass or seed yield of at least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

Cold Stress.

In some cases, endophytes can confer to the plant the ability to tolerate cold stress. As used herein, cold stress refers to both the stress induced by chilling (0° C.-15° C.) and freezing (<0° C.). Some cultivars of agricultural plants can be particularly sensitive to cold stress, but cold tolerance traits may be multigenic, making the breeding process difficult. Endophytes able to confer cold tolerance can reduce the damage suffered by farmers on an annual basis. Improved response to cold stress can be measured by survival of plants, production of protectant substances such as anthocyanin, the amount of necrosis of parts of the plant, or a change in crop yield loss, as well as the physiological parameters used in other examples. Therefore, in an embodiment, the plant comprising endophytes shows increased cold tolerance exhibits as compared to a reference agricultural plant grown under the same conditions of cold stress. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

Biotic Stress.

In other embodiments, the endophyte protects the plant from a biotic stress, for example, insect infestation, nematode infestation, complex infection, fungal infection, bacterial infection, oomycete infection, protozoal infection, viral infection, and herbivore grazing, or a combination thereof. For example, the endophyte may provide an improved benefit or tolerance to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

Insect Herbivory.

There are an abundance of insect pest species that can infect or infest a wide variety of plants. Pest infestation can lead to significant damage. Insect pests that infest plant species are particularly problematic in agriculture as they can cause serious damage to crops and significantly reduce plant yields. A wide variety of different types of plant are susceptible to pest infestation including commercial crops such as cotton, soybean, wheat, barley, and corn (maize).

In some cases, endophytes described herein may confer upon the host plant the ability to repel insect herbivores. In other cases, endophytes may produce, or induce the production in the plant of, compounds which are insecticidal or insect repellant. The insect may be any one of the common pathogenic insects affecting plants, particularly agricultural plants.

The endophyte-associated plant can be tested for its ability to resist, or otherwise repel, pathogenic insects by measuring, for example, insect load, overall plant biomass, biomass of the fruit or grain, percentage of intact leaves, or other physiological parameters described herein, and comparing with a reference agricultural plant. In an embodiment, the endophyte-associated plant exhibits increased biomass as compared to a reference agricultural plant grown under the same conditions (e.g., grown side-by-side, or adjacent to, endophyte-associated plants). In other embodiments, the endophyte-associated plant exhibits increased fruit or grain yield as compared to a reference agricultural plant grown under the same conditions (e.g., grown side-by-side, or adjacent to, endophyte-associated plants).

Nematodes.

Nematodes are microscopic roundworms that feed on the roots, fluids, leaves and stems of more than 2,000 row crops, vegetables, fruits, and ornamental plants, causing an estimated $100 billion crop loss worldwide and accounting for 13% of global crop losses due to disease. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore parasitic on a large number of crop species. The cyst- and lesion-forming nematode species have a more limited host range, but still cause considerable losses in susceptible crops.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots infected by SCN are dwarfed or stunted. Nematode infestation also can decrease the number of nitrogen-fixing nodules on the roots, and may make the roots more susceptible to attacks by other soil-borne plant nematodes.

In an embodiment, the endophyte-associated plant has an increased resistance to a nematode when compared with a reference agricultural plant. As before with insect herbivores, biomass of the plant or a portion of the plant, or any of the other physiological parameters mentioned elsewhere, can be compared with the reference agricultural plant grown under the same conditions. Particularly useful measurements include overall plant biomass, biomass and/or size of the fruit or grain, and root biomass. In one embodiment, the endophyte-associated plant exhibits increased biomass as compared to a reference agricultural plant grown under the same conditions (e.g., grown side-by-side, or adjacent to, the endophyte-associated plants, under conditions of nematode challenge). In another embodiment, the endophyte-associated plant exhibits increased root biomass as compared to a reference agricultural plant grown under the same conditions (e.g., grown side-by-side, or adjacent to, the endophyte-associated plants, under conditions of nematode challenge). In still another embodiment, the endophyte-associated plant exhibits increased fruit or grain yield as compared to a reference agricultural plant grown under the same conditions (e.g., grown side-by-side, or adjacent to, the endophyte-associated plants, under conditions of nematode challenge).

Fungal Pathogens.

Fungal diseases are responsible for yearly losses of over $10 Billion on agricultural crops in the US, represent 42% of global crop losses due to disease, and are caused by a large variety of biologically diverse pathogens. Different strategies have traditionally been used to control them. Resistance traits have been bred into agriculturally important varieties, thus providing various levels of resistance against either a narrow range of pathogen isolates or races, or against a broader range. However, this involves the long and labor intensive process of introducing desirable traits into commercial lines by genetic crosses and, due to the risk of pests evolving to overcome natural plant resistance, a constant effort to breed new resistance traits into commercial lines is required. Alternatively, fungal diseases have been controlled by the application of chemical fungicides. This strategy usually results in efficient control, but is also associated with the possible development of resistant pathogens and can be associated with a negative impact on the environment. Moreover, in certain crops, such as barley and wheat, the control of fungal pathogens by chemical fungicides is difficult or impractical.

The present invention contemplates the use of endophytes that are able to confer resistance to fungal pathogens to the host plant. Increased resistance to fungal inoculation can be measured, for example, using any of the physiological parameters presented above, by comparing with reference agricultural plants. In an embodiment, the endophyte-associated plant exhibits increased biomass and/or less pronounced disease symptoms as compared to a reference agricultural plant grown under the same conditions (e.g., grown side-by-side, or adjacent to, the endophyte-associated plants, infected with the fungal pathogen). In still another embodiment, the endophyte-associated plant exhibits increased fruit or grain yield as compared to a reference agricultural plant grown under the same conditions (e.g., grown side-by-side, or adjacent to, the endophyte-associated plants, infected with the fungal pathogen). In another embodiment, the endophyte-associated plant exhibits decreased hyphal growth as compared to a reference agricultural plant grown under the same conditions (e.g., grown side-by-side, or adjacent to, the endophyte-associated plants, infected with the fungal pathogen). For example, the endophyte may provide an improved benefit to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions.

Viral Pathogens.

Plant viruses are estimated to account for 18% of global crop losses due to disease. There are numerous examples of viral pathogens affecting agricultural productivity. In an embodiment, the endophyte provides protection against viral pathogens such that the plant has increased biomass as compared to a reference agricultural plant grown under the same conditions. In still another embodiment, the endophyte-associated plant exhibits greater fruit or grain yield, when challenged with a virus, as compared to a reference agricultural plant grown under the same conditions. In yet another embodiment, the endophyte-associated plant exhibits lower viral titer, when challenged with a virus, as compared to a reference agricultural plant grown under the same conditions.

Complex Pathogens.

Likewise, bacterial pathogens are a significant problem negatively affecting agricultural productivity and accounting for 27% of global crop losses due to plant disease. In an embodiment, the endophyte described herein provides protection against bacterial pathogens such that the plant has greater biomass as compared to a reference agricultural plant grown under the same conditions. In still another embodiment, the endophyte-associated plant exhibits greater fruit or grain yield, when challenged with a complex pathogen, as compared to a reference agricultural plant grown under the same conditions. In yet another embodiment, the endophyte-associated plant exhibits lower complex count, when challenged with a bacterium, as compared to a reference agricultural plant grown under the same conditions.

### Improvement of Other Traits

In other embodiments, the endophyte can confer other beneficial traits to the plant. Improved traits can include an improved nutritional content of the plant or plant element used for human consumption. In one embodiment, the endophyte-associated plant is able to produce a detectable change in the content of at least one nutrient. Examples of such nutrients include amino acid, protein, oil (including any one of Oleic acid, Linoleic acid, Alpha-linoleic acid, Saturated fatty acids, Palmitic acid, Stearic acid and Trans fats), carbohydrate (including sugars such as sucrose, glucose and fructose, starch, or dietary fiber), Vitamin A, Thiamine (vit. B1), Riboflavin (vit. B2), Niacin (vit. B3), Pantothenic acid (B5), Vitamin B6, Folate (vit. B9), Choline, Vitamin C, Vitamin E, Vitamin K, Calcium, Iron, Magnesium, Manganese, Phosphorus, Potassium, Sodium, Zinc. In an embodiment, the endophyte-associated plant or part thereof contains at least one increased nutrient when compared with reference agricultural plants.

In other cases, the improved trait can include reduced content of a harmful or undesirable substance when compared with reference agricultural plants. Such compounds include those which are harmful when ingested in large quantities or are bitter tasting (for example, oxalic acid, amygdalin, certain alkaloids such as solanine, caffeine, nicotine, quinine and morphine, tannins, cyanide). As such, in one embodiment, the endophyte-associated plant or part thereof contains less of the undesirable substance when compared with reference agricultural plant. In a related embodiment, the improved trait can include improved taste of the plant or a part of the plant, including the fruit or plant reproductive element. In a related embodiment, the improved trait can include reduction of undesirable compounds produced by other endophytes in plants, such as degradation of Fusarium-produced deoxynivalenol (also known as vomitoxin and a virulence factor involved in Fusarium head blight of maize and wheat) in a part of the plant, including the fruit or plant reproductive element.

In other cases, the improved trait can be an increase in overall biomass of the plant or a part of the plant, including its fruit or plant reproductive element.

The endophyte-associated plant can also have an altered hormone status or altered levels of hormone production when compared with a reference agricultural plant. An alteration in hormonal status may affect many physiological parameters, including flowering time, water efficiency, apical dominance and/or lateral shoot branching, increase in root hair, and alteration in fruit ripening.

The association between the endophyte and the plant can also be detected using other methods known in the art. For example, the biochemical, metabolomics, proteomic, genomic, epigenomic and/or transcriptomic profiles of endophyte-associated plants can be compared with reference agricultural plants under the same conditions.

### Methods of Using Endophytes and Synthetic Combinations Comprising Endophytes

As described herein, purified endophyte populations and compositions comprising the same (e.g., formulations) can be used to confer beneficial traits to the host plant including, for example, one or more of the following: increased root biomass, increased root length, increased height, increased shoot length, increased leaf number, improved water use efficiency (drought tolerance), increased overall biomass, increase grain yield, increased photosynthesis rate, increased tolerance to drought, increased heat tolerance, increased salt tolerance, increased resistance to nematode stress, increased resistance to a fungal pathogen, increased resistance to a complex pathogen, increased resistance to a viral pathogen, a detectable modulation in the level of a metabolite, and a detectable modulation in the proteome relative to a reference plant. For example, in some embodiments, a purified endophyte population can improve two or more such beneficial traits, e.g., water use efficiency and increased tolerance to drought.

In some cases, the endophyte may produce one or more compounds and/or have one or more activities, e.g., one or more of the following: production of a metabolite, production of a phytohormone such as auxin, production of acetoin, production of an antimicrobial compound, production of a siderophore, production of a cellulase, production of a pectinase, production of a chitinase, production of a xylanase, nitrogen fixation, or mineral phosphate solubilization. For example, an endophyte can produce a phytohormone selected from the group consisting of an auxin, a cytokinin, a gibberellin, ethylene, a brassinosteroid, and abscisic acid. In one particular embodiment, the endophyte produces auxin (e.g., indole-3-acetic acid (IAA)). Production of auxin can be assayed as described herein. Many of the microbes described herein are capable of producing the plant hormone auxin indole-3-acetic acid (IAA) when grown in culture. Auxin plays a key role in altering the physiology of the plant, including the extent of root growth. Therefore, in another embodiment, the endophytic population is disposed on the surface or within a tissue of the seed or seedling in an amount effective to detectably increase production of auxin in the agricultural plant when compared with a reference agricultural plant. In one embodiment, the increased auxin production can be detected in a tissue type selected from the group consisting of the root, shoot, leaves, and flowers.

In some embodiments, the endophyte can produce a compound with antimicrobial properties. For example, the compound can have antibacterial properties, as determined by the growth assays provided herein. In one embodiment, the compound with antibacterial properties shows bacteriostatic or bactericidal activity against E. coli and/or Bacillus sp. In another embodiment, the endophyte produces a compound with antifungal properties, for example, fungicidal or fungistatic activity against S. cerevisiae and/or Rhizoctonia.

In some embodiments, the endophyte is a bacterium capable of nitrogen fixation, and is thus capable of producing ammonium from atmospheric nitrogen. The ability of a bacterium to fix nitrogen can be confirmed by testing for growth of the bacterium in nitrogen-free growth media, for example, LGI media, as described herein.

In some embodiments, the endophyte can produce a compound that increases the solubility of mineral phosphate in the medium, i.e., mineral phosphate solubilization, for example, using the growth assays described herein. In one embodiment, the endophyte produces a compound that allows the bacterium to grow in growth media comprising Ca₃HPO₄ as the sole phosphate source.

In some embodiments, the endophyte can produce a siderophore. Siderophores are small high-affinity iron chelating agents secreted by microorganisms that increase the bioavailability of iron. Siderophore production by the endophyte can be detected, for example, using any known method in the art.

In some embodiments, the endophyte can produce a hydrolytic enzyme. For example, in one embodiment, an endophyte can produce a hydrolytic enzyme selected from the group consisting of a cellulase, a pectinase, a chitinase and a xylanase. Hydrolytic enzymes can be detected using the methods known in the art.

In some embodiments, metabolites in plants can be modulated by making synthetic combinations of purified endophytic populations. For example, an endophyte described herein can cause a detectable modulation (e.g., an increase or decrease) in the level of various metabolites, e.g., indole-3-carboxylic acid, trans-zeatin, abscisic acid, phaseic acid, indole-3-acetic acid, indole-3-butyric acid, indole-3-acrylic acid, jasmonic acid, jasmonic acid methyl ester, dihydrophaseic acid, gibberellin A3, salicylic acid, upon colonization of a plant.

In some embodiments, the endophyte modulates the level of the metabolite directly (e.g., the microbe itself produces the metabolite, resulting in an overall increase in the level of the metabolite found in the plant). In other cases, the agricultural plant, as a result of the association with the endophytic microbe (e.g., an endophyte), exhibits a modulated level of the metabolite (e.g., the plant reduces the expression of a biosynthetic enzyme responsible for production of the metabolite as a result of the microbe inoculation). In still other cases, the modulation in the level of the metabolite is a consequence of the activity of both the microbe and the plant (e.g., the plant produces increased amounts of the metabolite when compared with a reference agricultural plant, and the endophytic microbe also produces the metabolite). Therefore, as used herein, a modulation in the level of a metabolite can be an alteration in the metabolite level through the actions of the microbe and/or the inoculated plant.

The levels of a metabolite can be measured in an agricultural plant, and compared with the levels of the metabolite in a reference agricultural plant, and grown under the same conditions as the inoculated plant. The uninoculated plant that is used as a reference agricultural plant is a plant that has not been applied with a formulation with the endophytic microbe (e.g., a formulation comprising a population of purified endophytes). The uninoculated plant used as the reference agricultural plant is generally the same species and cultivar as, and is isogenic to, the inoculated plant.

The metabolite whose levels are modulated (e.g., increased or decreased) in the endophyte-associated plant may serve as a primary nutrient (i.e., it provides nutrition for the humans and/or animals who consume the plant, plant tissue, or the commodity plant product derived therefrom, including, but not limited to, a sugar, a starch, a carbohydrate, a protein, an oil, a fatty acid, a mineral, or a vitamin). The metabolite can be a compound that is important for plant growth, development or homeostasis (for example, a phytohormone such as an auxin, cytokinin, gibberellin, a brassinosteroid, ethylene, or abscisic acid, a signaling molecule, or an antioxidant). In other embodiments, the metabolite can have other functions. For example, in one embodiment, a metabolite can have bacteriostatic, bactericidal, fungistatic, fungicidal or antiviral properties. In other embodiments, the metabolite can have insect-repelling, insecticidal, nematode-repelling, or nematicidal properties. In still other embodiments, the metabolite can serve a role in protecting the plant from stresses, may help improve plant vigor or the general health of the plant. In yet another embodiment, the metabolite can be a useful compound for industrial production. For example, the metabolite may itself be a useful compound that is extracted for industrial use, or serve as an intermediate for the synthesis of other compounds used in industry. In a particular embodiment, the level of the metabolite is increased within the agricultural plant or a portion thereof such that it is present at a concentration of at least 0.1 ug/g dry weight, between 0.1 ug/g to 0.3 ug/g, for example, at least 0.3 ug/g dry weight, between 0.3 ug/g to 1.0 ug/g, 1.0 ug/g dry weight, between 1 ug/g and 3 ug/g, 3.0 ug/g dry weight, between 3 ug/g and 10 ug/g, 10 ug/g dry weight, between 10 ug/g and 30 ug/g, 30 ug/g dry weight, between 30 ug/g and 100 ug/g, 100 ug/g dry weight, between 100 ug/g and 300 ug/g, 300 ug/g dry weight, between 300 ug/g and 1 mg/g, 1 mg/g dry weight, between 1 mg/g and 3 mg/g, 3 mg/g dry weight, between 3 mg/g and 10 mg/g, 10 mg/g dry weight, between 10 mg/g and 30 mg/g, 30 mg/g dry weight, between 30 mg/g and 100 mg/g, 100 mg/g dry weight or more, of the plant or portion thereof.

Likewise, the modulation can be a decrease in the level of a metabolite. The reduction can be in a metabolite affecting the taste of a plant or a commodity plant product derived from a plant (for example, a bitter tasting compound), or in a metabolite which makes a plant or the resulting commodity plant product otherwise less valuable (for example, reduction of oxalate content in certain plants, or compounds which are deleterious to human and/or animal health). The metabolite whose level is to be reduced can be a compound that affects quality of a commodity plant product (e.g., reduction of lignin levels).

In some embodiments, the endophyte is capable of generating a complex network in the plant or surrounding environment of the plant, which network is capable of causing a detectable modulation in the level of a metabolite in the host plant.

In a particular embodiment, the metabolite can serve as a signaling or regulatory molecule. The signaling pathway can be associated with a response to a stress, for example, one of the stress conditions selected from the group consisting of drought stress, salt stress, heat stress, cold stress, low nutrient stress, nematode stress, insect herbivory stress, fungal pathogen stress, complex pathogen stress, and viral pathogen stress.

The inoculated agricultural plant is grown under conditions such that the level of one or more metabolites is modulated in the plant, wherein the modulation is indicative of increased resistance to a stress selected from the group consisting of drought stress, salt stress, heat stress, cold stress, low nutrient stress, nematode stress, insect herbivory stress, fungal pathogen stress, complex pathogen stress, and viral pathogen stress. The increased resistance can be measured at about 10 minutes after applying the stress, between 10 minutes and 20 minutes, for example about 20 minutes, between 20 and 30 minutes, 30 minutes, between 30 and 45 minutes, about 45 minutes, between 45 minutes and 1 hour, about 1 hour, between 1 and 2 hours, about 2 hours, between 2 and 4 hours, about 4 hours, between 4 and 8 hours, about 8 hours, between 8 and 12 hours, about 12 hours, between 12 and 16 hours, about 16 hours, between 16 and 20 hours, about 20 hours, between 20 and 24 hours, about 24 hours, between 24 and 36 hours, about 36 hours, between 36 and 48 hours, about 48 hours, between 48 and 72 hours, about 72 hours, between 72 and 96 hours, about 96 hours, between 96 and 120 hours, about 120 hours, between 120 hours and one week, or about a week after applying the stress.

The metabolites or other compounds described herein can be detected using any suitable method including, but not limited to gel electrophoresis, liquid and gas phase chromatography, either alone or coupled to mass spectrometry, NMR, immunoassays (radioimmunoassays (MA) or enzyme-linked immunosorbent assays (ELISA)), chemical assays, spectroscopy and the like. In some embodiments, commercial systems for chromatography and NMR analysis are utilized.

In other embodiments, metabolites or other compounds are detected using optical imaging techniques such as magnetic resonance spectroscopy (MRS), magnetic resonance imaging (MRI), CAT scans, ultra sound, MS-based tissue imaging or X-ray detection methods (e.g., energy dispersive x-ray fluorescence detection).

Any suitable method may be used to analyze the biological sample (e.g., seed or plant tissue) in order to determine the presence, absence or level(s) of the one or more metabolites or other compounds in the sample. Suitable methods include chromatography (e.g., HPLC, gas chromatography, liquid chromatography), mass spectrometry (e.g., MS, MS-MS), LC-MS, enzyme-linked immunosorbent assay (ELISA), antibody linkage, other immunochemical techniques, biochemical or enzymatic reactions or assays, and combinations thereof. The levels of one or more of the recited metabolites or compounds may be determined in the methods of the present invention. For example, the level(s) of one metabolites or compounds, two or more metabolites, three or more metabolites, four or more metabolites, five or more metabolites, six or more metabolites, seven or more metabolites, eight or more metabolites, nine or more metabolites, ten or more metabolites, or compounds etc., including a combination of some or all of the metabolites or compounds including, but not limited to those disclosed herein may be determined and used in such methods.

In some embodiments, a synthetic combination of a plant and a formulation comprising at least one endophytic microbe will cause an increase in the level of a protein in the plant.

In some embodiments, a synthetic combination of a plant and a formulation comprising at least one endophytic microbe will cause a decrease in the level of a protein in the plant.

In some embodiments, a synthetic combination of a plant and a formulation comprising at least one endophytic microbe will cause an increase in the level of expression of a gene in the plant.

In some embodiments, a synthetic combination of a plant and a formulation comprising at least one endophytic microbe will cause a decrease in the level of expression of a gene in the plant.

In some embodiments, a synthetic combination of a plant and a formulation comprising at least one endophytic microbe will cause an increase in the level of a plant hormone.

In some embodiments, a synthetic combination of a plant and a formulation comprising at least one endophytic microbe will cause a modulation in the concentration or amount of a metabolite.

As shown in the Examples and otherwise herein, endophyte-inoculated plants display increased thermal tolerance, herbicide tolerance, drought resistance, insect resistance, fungus resistance, virus resistance, bacteria resistance, male sterility, cold tolerance, salt tolerance, increased yield, enhanced nutrient use efficiency, increased nitrogen use efficiency, increased protein content, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof.

Therefore, in an embodiment, the endophytic population is disposed on the surface or on or within a tissue of the seed or seedling in an amount effective to increase the biomass of the plant, or a part or tissue of the plant derived from the seed or seedling. The increased biomass is useful in the production of commodity products derived from the plant. Such commodity products include an animal feed, a fish fodder, a cereal product, a processed human-food product, a sugar or an alcohol. Such products may be a fermentation product or a fermentable product, one such exemplary product is a biofuel. The increase in biomass can occur in a part of the plant (e.g., the root tissue, shoots, leaves, etc.), or can be an increase in overall biomass when compared with a reference agricultural plant. Such increase in overall biomass can be under relatively stress-free conditions. In other cases, the increase in biomass can be in plants grown under any number of abiotic or biotic stresses, including drought stress, salt stress, heat stress, cold stress, low nutrient stress, nematode stress, insect herbivory stress, fungal pathogen stress, complex pathogen stress, and viral pathogen stress.

In another embodiment, the endophytic population is disposed on the surface or within a tissue of the seed or seedling in an amount effective to increase the rate of seed germination when compared with a reference agricultural plant.

In other cases, the microbe is disposed on the seed or seedling in an amount effective to increase the average biomass of the fruit or cob from the resulting plant when compared with a reference agricultural plant.

Plants inoculated with an endophytic population may also show an increase in overall plant height. Therefore, in an embodiment, the present invention provides for a seed comprising an endophytic population that is disposed on the surface or within a tissue of the seed or seedling in an amount effective to increase the height of the plant. For example, the endophytic population is disposed in an amount effective to result in an increase in height of the agricultural plant when compared with a reference agricultural plant. Such an increase in height can be under relatively stress-free conditions. In other cases, the increase in height can be in plants grown under any number of abiotic or biotic stresses, including drought stress, salt stress, heat stress, cold stress, low nutrient stress, nematode stress, insect herbivory stress, fungal pathogen stress, complex pathogen stress, or viral pathogen stress.

In another embodiment, the plant containing the endophyte is able to grown under nutrient stress conditions while exhibiting no difference in the physiological parameter compared to a plant that is grown without nutrient stress. In some embodiments, such a plant will exhibit no difference in the physiological parameter when grown with 2-5% less nitrogen than average cultivation practices on normal agricultural land, for example, at least 5-10% less nitrogen, at least 10-15% less nitrogen, at least 15-20% less nitrogen, at least 20-25% less nitrogen, at least 25-30% less nitrogen, at least 30-35% less nitrogen, at least 35-40% less nitrogen, at least 40-45% less nitrogen, at least 45-50% less nitrogen, at least 50-55% less nitrogen, at least 55-60% less nitrogen, at least 60-65% less nitrogen, at least 65-70% less nitrogen, at least 70-75% less nitrogen, at least 80-85% less nitrogen, at least 85-90% less nitrogen, at least 90-95% less nitrogen, or less, when compared with crop plants grown under normal conditions during an average growing season. In some embodiments, the microbe capable of providing nitrogen-stress tolerance to a plant is diazotrophic. In other embodiments, the microbe capable of providing nitrogen-stress tolerance to a plant is non-diazotrophic.

The host plants inoculated with the endophytic population may also show improvements in their ability to utilize water more efficiently. Water use efficiency is a parameter often correlated with drought tolerance. Water use efficiency (WUE) is a parameter often correlated with drought tolerance, and is the CO₂ assimilation rate per amount of water transpired by the plant. An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a low water potential, are more efficient and thereby are able to produce more biomass and economic yield in many agricultural systems. An increased water use efficiency of the plant relates in some cases to an increased fruit/kernel size or number.

Therefore, in one embodiment, the plants described herein exhibit an increased water use efficiency (WUE) when compared with a reference agricultural plant grown under the same conditions. For example, the endophyte may provide an increase in WUE to a plant that is of at least 3%, between 3% and 5%, at least 5%, between 5% and 10%, least 10%, between 10% and 15%, for example at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with uninoculated plants grown under the same conditions. Such an increase in WUE can occur under conditions without water deficit, or under conditions of water deficit, for example, when the soil water content is less than or equal to 60% of water saturated soil, for example, less than or equal to 50%, less than or equal to 40%, less than or equal to 30%, less than or equal to 20%, less than or equal to 10% of water saturated soil on a weight basis. In some embodiments, the plants inoculated with the endophytic population show increased yield under non-irrigated conditions, as compared to reference agricultural plants grown under the same conditions.

In a related embodiment, the plant comprising endophyte can have a higher relative water content (RWC), than a reference agricultural plant grown under the same conditions. Formulations for Agricultural Use

The endophyte populations described herein are intended to be useful in the improvement of agricultural plants, and as such, may be formulated with other compositions as part of an agriculturally compatible carrier. It is contemplated that such carriers can include applications such as, but not be limited to: seed treatment, root wash, seedling soak, foliar application, soil inocula, in-furrow application, sidedress application, soil pre-treatment, wound inoculation, drip tape irrigation, vector-mediation via a pollinator, injection, osmopriming, hydroponics, aquaponics, aeroponics. The carrier composition with the endophyte populations, may be prepared for agricultural application as a liquid, a solid, or a gas formulation. Application to the plant may be achieved, for example, as a powder for surface deposition onto plant leaves, as a spray to the whole plant or selected plant element, as part of a drip to the soil or the roots, or as a coating onto the plant element prior to planting. Such examples are meant to be illustrative and not limiting to the scope of the invention.

The formulation useful for these embodiments generally and typically include at least one member selected from the group consisting of a buffer, a tackifier, a microbial stabilizer, a fungicide, an anticomplex agent, an herbicide, a nematicide, an insecticide, a bactericide, a virucide, a plant growth regulator, a rodenticide, a desiccant, and a nutrient.

The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes the purified population (see, for example, U.S. Pat. No. 7,485,451, which is incorporated herein by reference in its entirety). Suitable formulations that may be prepared include wettable powders, granules, gels, agar strips or pellets, thickeners, biopolymers, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant elements, sugar cane bagasse, hulls or stalks from grain processing, ground plant material or wood from building site refuse, sawdust or small fibers from recycling of paper, fabric, or wood. Other suitable formulations will be known to those skilled in the art.

In an embodiment, the formulation can include a tackifier or adherent. Such agents are useful for combining the complex population of the invention with carriers that can contain other compounds (e.g., control agents that are not biologic), to yield a coating composition. Such compositions help create coatings around the plant or plant element to maintain contact between the endophyte and other agents with the plant or plant element. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, carragennan, PGA, other biopolymers, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. Other examples of adherent compositions that can be used in the synthetic preparation include those described in EP 0818135, CA 1229497, WO 2013090628, EP 0192342, WO 2008103422 and CA 1041788, each of which is incorporated herein by reference in its entirety.

It is also contemplated that the formulation may further comprise an anti-caking agent.

The formulation can also contain a surfactant, wetting agent, emulsifier, stabilizer, or anti-foaming agent. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision), polysorbate 20, polysorbate 80, Tween 20, Tween 80, Scattics, Alktest TW20, Canarcel, Peogabsorb 80, Triton X-100, Conco NI, Dowfax 9N, Igebapl CO, Makon, Neutronyx 600, Nonipol NO, Plytergent B, Renex 600, Solar NO, Sterox, Serfonic N, T-DET-N, Tergitol NP, Triton N, IGEPAL CA-630, Nonident P-40, Pluronic. In one embodiment, the surfactant is present at a concentration of between 0.01% v/v to 10% v/v. In another embodiment, the surfactant is present at a concentration of between 0.1% v/v to 1% v/v. An example of an anti-foaming agent would be Antifoam-C.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the population used, and should promote the ability of the endophyte population to survive application on the seeds and to survive desiccation. Examples of suitable desiccants include one or more of trehalose, sucrose, glycerol, and methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some cases, it is advantageous for the formulation to contain agents such as a fungicide, an anticomplex agent, an herbicide, a nematicide, an insecticide, a plant growth regulator, a rodenticide, a bactericide, a virucide, or a nutrient. Such agents are ideally compatible with the agricultural plant element or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In the liquid form, for example, solutions or suspensions, endophyte populations of the present invention can be mixed or suspended in water or in aqueous solutions. Suitable liquid diluents or carriers include water, aqueous solutions, petroleum distillates, or other liquid carriers.

Solid compositions can be prepared by dispersing the endophyte populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils (such as soybean oil, maize (corn) oil, and cottonseed oil), glycerol, ethylene glycol, polyethylene glycol, propylene glycol, polypropylene glycol, etc.

In an embodiment, the formulation is ideally suited for coating of a population of endophytes onto plant elements. The endophytes populations described in the present invention are capable of conferring many fitness benefits to the host plants. The ability to confer such benefits by coating the populations on the surface of plant elements has many potential advantages, particularly when used in a commercial (agricultural) scale.

The endophyte populations herein can be combined with one or more of the agents described above to yield a formulation suitable for combining with an agricultural plant element, seedling, or other plant element. Endophyte populations can be obtained from growth in culture, for example, using a synthetic growth medium. In addition, endophytes can be cultured on solid media, for example on petri dishes, scraped off and suspended into the preparation. Endophytes at different growth phases can be used. For example, endophytes at lag phase, early-log phase, mid-log phase, late-log phase, stationary phase, early death phase, or death phase can be used. Endophytic spores may be used for the present invention, for example but not limited to: arthospores, sporangispores, conidia, chlamadospores, pycnidiospores, endospores, zoospores.

The formulations comprising endophyte populations of the present invention typically contains between about 0.1 to 95% by weight, for example, between about 1% and 90%, between about 3% and 75%, between about 5% and 60%, between about 10% and 50% in wet weight of the population of the present invention. It is preferred that the formulation contains at least about 10̂3 CFU per ml of formulation, for example, at least about 10̂4, at least about 10̂5, at least about 10̂6, at least about 10̂7 CFU, at least about 10̂8 CFU per ml of formulation. It is preferred that the formulation be applied to the plant element at about 10̂2 CFU/seed, between 10̂2 and 10̂3 CFU, at least about 10̂3 CFU, between 10̂3 and 10̂4 CFU, at least about 10̂4 CFU, between 10̂4 and 10̂5 CFU, at least about 10̂5 CFU, between 10̂5 and 10̂6 CFU, at least about 10̂6 CFU, between 10̂6 and 10̂7 CFU, at least about 10̂7 CFU, between 10̂7 and 10̂8 CFU, or even greater than 10̂8 CFU per seed.

In some embodiments, fungal endophytes may be encapsulated in a fungal host, whether its native host or a heterologous host, before incorporation into a formulation.

### Populations of Plant Elements (PEs)

In another embodiment, the invention provides for a substantially uniform population of plant elements (PEs) comprising two or more PEs comprising the endophytic population, as described herein above. Substantial uniformity can be determined in many ways. In some cases, at least 10%, between 10% and 20%, for example, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the PEs in the population, contains the endophytic population in an amount effective to colonize the plant disposed on the surface of the PEs. In other cases, at least 10%, between 10% and 20%, for example, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the plant element s in the population, contains at least 1, between 10 and 10, 10, between 10 and 100, or 100 CFU on the plant element surface or per gram of plant element, for example, between 100 and 200 CFU, at least 200 CFU, between 200 and 300 CFU, at least 300 CFU, between 300 and 1,000 CFU, at least 1,000 CFU, between 1,000 and 3,000 CFU, at least 3,000 CFU, between 3,000 and 10,000 CFU, at least 10,000 CFU, between 10,000 and 30,000 CFU, at least 30,000 CFU, between 30,000 and 100,000 CFU, at least 100,000 CFU, between 100,000 and 300,000 CFU, at least 300,000 CFU, between 300,000 and 1,000,000 CFU, or at least 1,000,000 CFU per plant element or more.

In a particular embodiment, the population of plant elements is packaged in a bag or container suitable for commercial sale. Such a bag contains a unit weight or count of the plant elements comprising the endophytic population as described herein, and further comprises a label. In an embodiment, the bag or container contains at least 100 plant elements, between 100 and 1,000 plant elements, 1,000 plant elements, between 1,000 and 5,000 plant elements, for example, at least 5,000 plant elements, between 5,000 and 10,000 plant elements, at least 10,000 plant elements, between 10,000 and 20,000 plant elements, at least 20,000 plant elements, between 20,000 and 30,000 plant elements, at least 30,000 plant elements, between 30,000 and 50,000 plant elements, at least 50,000 plant elements, between 50,000 and 70,000 plant elements, at least 70,000 plant elements, between 70,000 and 80,000 plant elements, at least 80,000 plant elements, between 80,000 and 90,000, at least 90,000 plant elements or more. In another embodiment, the bag or container can comprise a discrete weight of plant elements, for example, at least 1 lb, between 1 and 2 lbs, at least 2 lbs, between 2 and 5 lbs, at least 5 lbs, between 5 and 10 lbs, at least 10 lbs, between 10 and 30 lbs, at least 30 lbs, between 30 and 50 lbs, at least 50 lbs, between 50 and 70 lmbs, at least 70 lbs or more. The bag or container comprises a label describing the plant elements and/or said endophytic population. The label can contain additional information, for example, the information selected from the group consisting of: net weight, lot number, geographic origin of the plant elements, test date, germination rate, inert matter content, and the amount of noxious weeds, if any. Suitable containers or packages include those traditionally used in plant seed commercialization. The invention also contemplates other containers with more sophisticated storage capabilities (e.g., with microbiologically tight wrappings or with gas- or water-proof containments).

In some cases, a sub-population of seeds comprising the endophytic population is further selected on the basis of increased uniformity, for example, on the basis of uniformity of microbial population. For example, individual plant elements of pools collected from individual cobs, individual plants, individual plots (representing plants inoculated on the same day) or individual fields can be tested for uniformity of microbial density, and only those pools meeting specifications (e.g., at least 80% of tested plant elements have minimum density, as determined by quantitative methods described elsewhere) are combined to provide the agricultural seed sub-population.

The methods described herein can also comprise a validating step. The validating step can entail, for example, growing some plant elements collected from the inoculated plants into mature agricultural plants, and testing those individual plants for uniformity. Such validating step can be performed on individual seeds collected from cobs, individual plants, individual plots (representing plants inoculated on the same day) or individual fields, and tested as described above to identify pools meeting the required specifications.

In some embodiments, methods described herein include planting a synthetic combination described herein. Suitable planters include an air seeder and/or fertilizer apparatus used in agricultural operations to apply particulate materials including one or more of the following, seed, fertilizer and/or inoculants, into soil during the planting operation. Seeder/fertilizer devices can include a tool bar having ground-engaging openers thereon, behind which is towed a wheeled cart that includes one or more containment tanks or bins and associated metering means to respectively contain and meter therefrom particulate materials.

In certain embodiments, a composition described herein may be in the form of a liquid, a slurry, a solid, or a powder (wettable powder or dry powder). In another embodiment, a composition may be in the form of a seed coating. Compositions in liquid, slurry, or powder (e.g., wettable powder) form may be suitable for coating plant elements. When used to coat plant elements, the composition may be applied to the plant elements and allowed to dry. In embodiments wherein the composition is a powder (e.g., a wettable powder), a liquid, such as water, may need to be added to the powder before application to a seed.

In still another embodiment, the methods can include introducing into the soil an inoculum of one or more of the endophyte populations described herein. Such methods can include introducing into the soil one or more of the compositions described herein. The inoculum(s) or compositions may be introduced into the soil according to methods known to those skilled in the art. Non-limiting examples include in-furrow introduction, spraying, coating seeds, foliar introduction, etc. In a particular embodiment, the introducing step comprises in-furrow introduction of the inoculum or compositions described herein.

In an embodiment, plant elements may be treated with composition(s) described herein in several ways but preferably via spraying or dripping. Spray and drip treatment may be conducted by formulating compositions described herein and spraying or dripping the composition(s) onto a seed(s) via a continuous treating system (which is calibrated to apply treatment at a predefined rate in proportion to the continuous flow of seed), such as a drum-type of treater. Batch systems, in which a predetermined batch size of seed and composition(s) as described herein are delivered into a mixer, may also be employed.

In another embodiment, the treatment entails coating plant elements. One such process involves coating the inside wall of a round container with the composition(s) described herein, adding plant elements, then rotating the container to cause the plant elements to contact the wall and the composition(s), a process known in the art as “container coating.” Plant elements can be coated by combinations of coating methods. Soaking typically entails using liquid forms of the compositions described. For example, plant elements can be soaked for about 1 minute to about 24 hours (e.g., for at least 1 min, between 1 and 5 min, 5 min, between 5 and 10 min, 10 min, between 10 and 20 min, 20 min, between 20 and 40 min, 40 min, between 40 and 80 min, 80 min, between 80 min and 3 hrs, 3 hrs, between 3 hrs and 6 hrs, 6 hr, between 6 hrs and 12 hrs, 12 hr, between 12 hrs and 24 hrs, 24 hrs).

### Population of Plants and Agricultural Fields

A major focus of crop improvement efforts has been to select varieties with traits that give, in addition to the highest return, the greatest homogeneity and uniformity. While inbreeding can yield plants with substantial genetic identity, heterogeneity with respect to plant height, flowering time, and time to seed, remain impediments to obtaining a homogeneous field of plants. The inevitable plant-to-plant variability is caused by a multitude of factors, including uneven environmental conditions and management practices. Another possible source of variability can, in some cases, be due to the heterogeneity of the endophyte population inhabiting the plants. By providing endophyte populations onto plant reproductive elements, the resulting plants generated by germinating the plant reproductive elements have a more consistent endophyte composition, and thus are expected to yield a more uniform population of plants.

Therefore, in another embodiment, the invention provides a substantially uniform population of plants. The population can include at least 10 plants, between 10 and 100 plants, for example, at least 100 plants, between 100 and 300 plants, at least 300 plants, between 300 and 1,000 plants, at least 1,000 plants, between 1,000 and 3,000 plants, at least 3,000 plants, between 3,000 and 10,000 plants, at least 10,000 plants, between 10,000 and 30,000 plants, at least 30,000 plants, between 30,000 and 100,000 plants, at least 100,000 plants or more. The plants are derived from plant reproductive elements comprising endophyte populations as described herein. The plants are cultivated in substantially uniform groups, for example in rows, groves, blocks, circles, or other planting layout.

The uniformity of the plants can be measured in a number of different ways. In one embodiment, there is an increased uniformity with respect to endophytes within the plant population. For example, in one embodiment, a substantial portion of the population of plants, for example at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the plant elements or plants in a population, contains a threshold number of an endophyte population. The threshold number can be at least 10 CFU, between 10 and 100 CFU, at least 100 CFU, between 100 and 300 CFU, for example at least 300 CFU, between 300 and 1,000 CFU, at least 1,000 CFU, between 1,000 and 3,000 CFU, at least 3,000 CFU, between 3,000 and 10,000 CFU, at least 10,000 CFU, between 10,000 and 30,000 CFU, at least 30,000 CFU, between 30,000 and 100,000 CFU, at least 100,000 CFU or more, in the plant or a part of the plant. Alternatively, in a substantial portion of the population of plants, for example, in at least 1%, between 1% and 10%, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 75%, at least 75%, between 75% and 80%, at least 80%, between 80% and 90%, at least 90%, between 90% and 95%, at least 95% or more of the plants in the population, the endophyte population that is provided to the seed or seedling represents at least 0.1%, between 0.1% and 1% at least 1%, between 1% and 5%, at least 5%, between 5% and 10%, at least 10%, between 10% and 20%, at least 20%, between 20% and 30%, at least 30%, between 30% and 40%, at least 40%, between 40% and 50%, at least 50%, between 50% and 60%, at least 60%, between 60% and 70%, at least 70%, between 70% and 80%, at least 80%, between 80% and 90%, at least 90%, between 90% and 95%, at least 95%, between 95% and 99%, at least 99%, between 99% and 100%, or 100% of the total endophyte population in the plant/seed.

In an embodiment, there is increased genetic uniformity of a substantial proportion or all detectable endophytes within the taxa, genus, or species of a component relative to an uninoculated control. This increased uniformity can be a result of the endophyte being of monoclonal origin or otherwise deriving from a population comprising a more uniform genome sequence and plasmid repertoire than would be present in the endophyte population a plant that derives its endophyte community largely via assimilation of diverse soil symbionts.

In another embodiment, there is an increased uniformity with respect to a physiological parameter of the plants within the population. In some cases, there can be an increased uniformity in the height of the plants when compared with a population of reference agricultural plants grown under the same conditions. For example, there can be a reduction in the standard deviation in the height of the plants in the population of at least 5%, between 5% and 10%, for example, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60% or more, when compared with a population of reference agricultural plants grown under the same conditions. In other cases, there can be a reduction in the standard deviation in the flowering time of the plants in the population of at least 5%, between 5% and 10%, for example, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60% or more, when compared with a population of reference agricultural plants grown under the same conditions.

### Commodity Plant Products

The present invention provides a commodity plant product, as well as methods for producing a commodity plant product, that is derived from a plant of the present invention. As used herein, a “commodity plant product” refers to any composition or product that is comprised of material derived from a plant, seed, plant cell, or plant element of the present invention. Commodity plant products may be sold to consumers and can be viable or nonviable. Nonviable commodity products include but are not limited to nonviable plant elements and grains; processed seeds, seed parts, and plant elements; dehydrated plant tissue, frozen plant tissue, and processed plant tissue; seeds and plant elements processed for animal feed for terrestrial and/or aquatic animal consumption, oil, meal, flour, flakes, bran, fiber, paper, tea, coffee, silage, crushed of whole grain, and any other food for human or animal consumption such as the fruit or other edible portion of the plant; and biomasses and fuel products; and raw material in industry.

Industrial uses of oils derived from the agricultural plants described herein include ingredients for paints, plastics, fibers, detergents, cosmetics, lubricants, and biodiesel fuel. Plant oils may be split, inter-esterified, sulfurized, epoxidized, polymerized, ethoxylated, or cleaved. Designing and producing plant oil derivatives with improved functionality and improved oliochemistry is a rapidly growing field. For example, a mixture of triglycerides is usually split and separated into pure fatty acids, which are then combined with petroleum-derived alcohols or acids, nitrogen, sulfonates, chlorine, or with fatty alcohols derived from fats and oils to produce the desired type of oil or fat. Commodity plant products also include industrial compounds, such as a wide variety of resins used in the formulation of adhesives, films, plastics, paints, coatings and foams.

Although the present invention has been described in detail with reference to examples below, it is understood that various modifications can be made without departing from the spirit of the invention. For instance, while the particular examples below may illustrate the methods and embodiments described herein using a specific plant, the principles in these examples may be applied to any agricultural crop. Therefore, it will be appreciated that the scope of this invention is encompassed by the embodiments of the inventions recited herein and the specification rather than the specific examples that are exemplified below.

All cited patents and publications referred to in this application are herein incorporated by reference in their entirety.

### EXAMPLES

### Example 1: Isolation and Identification of Penicillium Fungal Endophytes

Isolation and cultivation of endophytic microbes from agricultural plants was performed according to methods well known in the art. Microbial taxa found in agriculturally relevant communities were identified using high-throughput marker gene sequencing across several crops and numerous varieties of seeds.

Classification of fungal strains using ITS sequences was done by the following methodology.

Total genomic DNA was extracted from individual fungal isolates, using the Qiagen DNeasy Plant Mini Kit. Polymerase Chain Reaction (PCR) was used to amplify the nuclear ribosomal internal transcribed spacers (ITS) and the 5.8S gene (ITS ribosomal DNA [rDNA]) and when possible the first 600 bp of the large subunit (LSU rDNA) as a single fragment (ca. 1,000 to 1,200 bp in length) using the forward primer ITS1F (5′-CTTGGTCATTTAGAGGAAGTAA-3′) given as SEQ ID NO: 8, and the reverse primer ITS4 (5′-TCCTCCGCTTATTGATATGC-3′) given as SEQ ID NO: 9. Each 25 microliter-reaction mixture included 22.5 microliters of Invitrogen Platinum Taq supermix, 0.5 microliter of each primer (10 uM), and 1.5 microliters of DNA template (˜2-4 ng). Cycling reactions were run with MJ Research PTC thermocyclers and consisted of 94° C. for 5 min, 35 cycles of 94° C. for 30 s, 54° C. for 30 s, and 72° C. for 1 min, and 72° C. for 10 min. Sanger sequencing was performed using an ABI 3730x1 DNA Analyzers for capillary electrophoresis and fluorescent dye terminator detection.

PCR reactions were purified to remove primers, dNTPs, and other components by methods known in the art, for example by the use of commercially available PCR clean-up kits, or 3M sodium acetate and washed in absolute ethanol as described below, and resuspended in sterile water. DNA amplicons were then sequenced using methods known in the art, for example Sanger sequencing (Johnston-Monje and Raizada 2011, PLoS ONE 6(6): e20396) using one of the two primers used for amplification.

The resulting sequences were aligned as query sequences with publicly available databases such as GenBank nucleotide, UNITE (Abarenkov et al., 2010, New Phytologist 186(2): 281-285 and PlutoF (Abarenkov et al. 2010b, Evol Bioinform 189-196). UNITE and PlutoF are specifically compiled and used for identification of fungi. In all the cases, the isolates were identified to species level if their sequences were more than 95% similar to any identified accession from all databases analyzed (Zimmerman and Vitousek 2012, 109(32):13022-13027). When the similarity percentage was between 90-97%, the isolate was classified at genus, family, order, class, subdivision or phylum level depending on the information displayed in databases used. To support the molecular identification, fungal taxa were confirmed by inducing sporulation on PDA or V8 agar plates and using reported morphological criteria for identification of fruiting bodies structure and shape (Ainsworth et al. 2008, Ainsworth & Bisby's Dictionary of the Fungi 2008, CABI).

Strain A (Penicillium sp.) is described herein by its ITS sequence given as SEQ ID NO: 1.

Strain B (Penicillium SMCD2206) is described herein by its ITS sequence given as SEQ ID NO: 2. Strain B is deposited with International Depositary Authority of Canada (IDAC, National Microbiology Laboratory, Public Health Agency of Canada, 1015 Arlington Street, Winnipeg, Manitoba, Canada, R3E 3R2) as Deposit ID 081111-01.

The strain-specific primer pair for Strain B is given as SEQ ID NO: 10 for the forward primer and SEQ ID NO: 11 for the reverse primer. The amplicon resulting from sequencing using those primers is given as SEQ ID NO: 3.

Strain D (Penicillium olsonii) is described herein by its ITS sequence given as SEQ ID NO: 4.

Strain E (Penicillium griseofulvum) is described herein by its ITS sequence given as SEQ ID NO: 5.

Strain F (Penicillium janthinellum) is described herein by its ITS sequence given as SEQ ID NO: 6.

Strain G (Penicillium sp.) is described herein by its ITS sequence given as SEQ ID NO: 7.

The polynucleotide sequences for SEQ ID NOs: 1-11 are given in Table 1.

Based on performance in experiments assessing different agronomici traits in plants grown from seeds treated with each Penicillium endohyte strain in the present invention, Strains A, B, D, and E are described as beneficial Penicillium strains as compared to Penicillium Strains F and G.

### Example 2: In Vitro Testing and Characterization of Penicillium Fungal Endophytes

### Strains and Culture Preparations

Fungal endophyte strain Strain B was tested for various metabolic activities as described below.

To prepare the cultures as initial inocula for various assays, fungi were grown in one liter of Yeast Extract Peptone Dextrose (YEPD) broth in a 2.5-liter Ultra Yield and Fernbeck flasks (Thomson Instrument Company). The cultures were grown at 25° C. with continuous shaking at a speed of 130 revolutions per minute (rpm) for five days. The cultures were aliquoted into 50-mL Falcon tubes and were harvested by centrifugation at a speed of 3,500 rpm for 20 minutes. For each sample, one gram (g) of fresh biomass was first rinsed in 5 mL sterile water and resuspended in 15 mL of sterile water. In order to achieve homogeneity, samples were sonicated for 15 seconds continuously with probe intensity set to 3 using the Sonic Dismembrator Model 100 (Thermo Fisher Scientific, Waltham, Mass.). Strain purity was assessed by plating 100 microliter (uL) of fungal strain resuspension on PDA. After sonication, the cultures were allowed to sit at room temperature for 5-10 minutes before being used in in vitro assays.

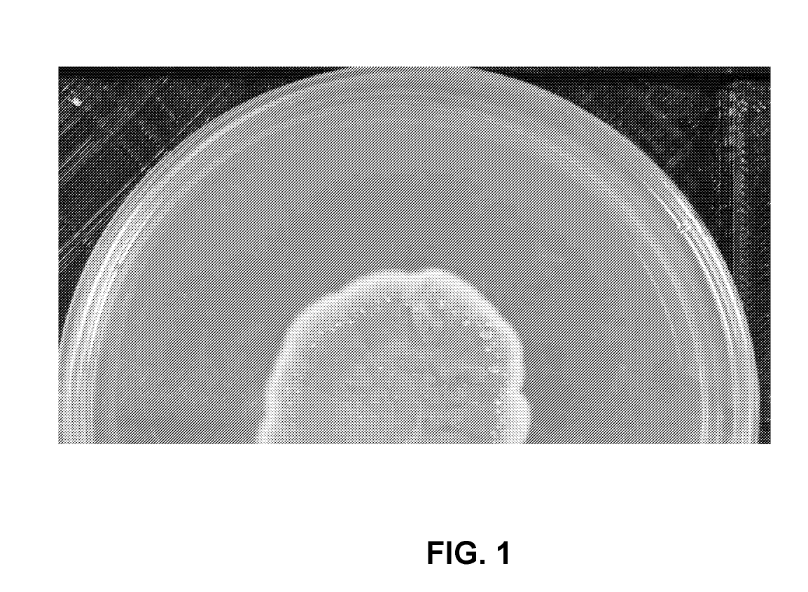

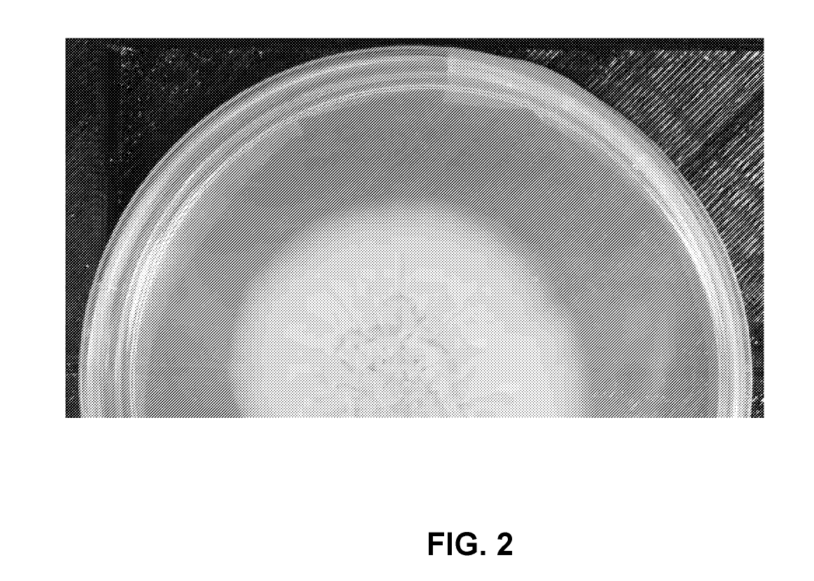

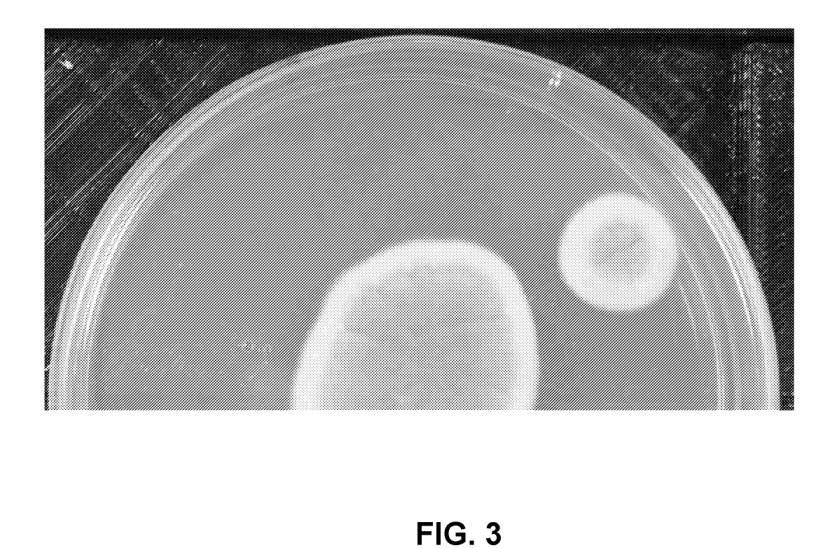

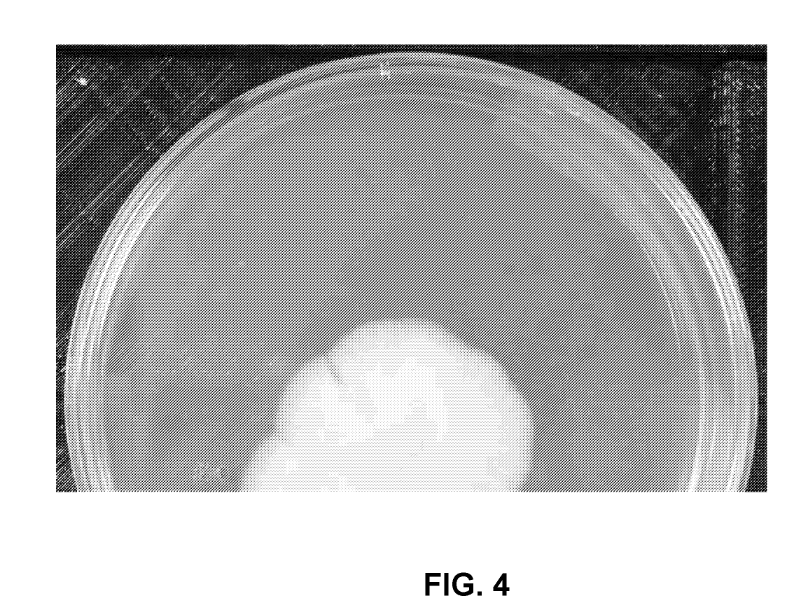

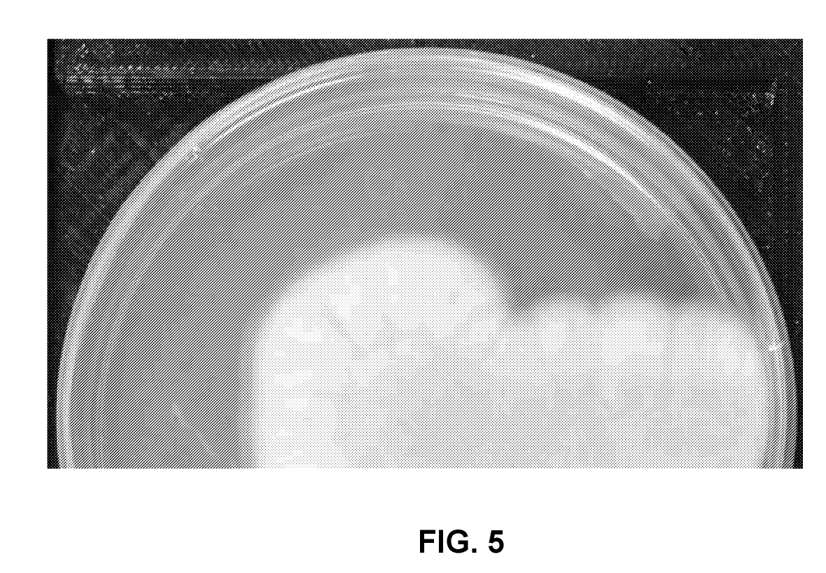

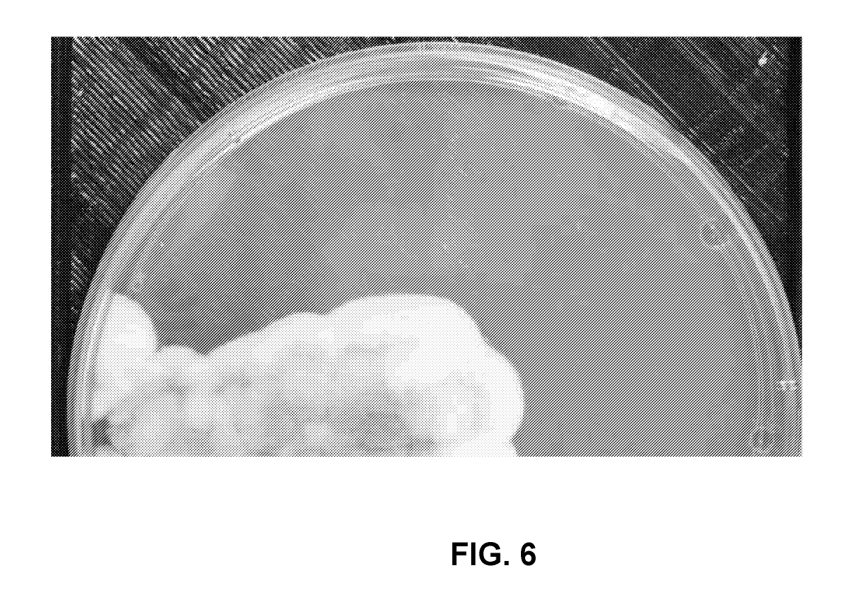

Photographs of the culture plates of Penicillium strains A, B, D, E, F, and G are given in FIGS. 1-6, respectively.

### Auxin Biosynthesis by Endophytes

To measure auxin levels, 100 microliters of fungal culture prepared as described above was inoculated into 1 mL of R2A broth supplemented with L-tryptophan (5 mM) in transparent flat bottom, 12-well tissue culture plates. Each culture was grown in three duplicates. The plates were sealed with a breathable membrane, wrapped in aluminum foil, and incubated at 25° C. on a shaker at a speed of 150 rpm in the dark for 3 days. After 3 days the OD600 nm and OD530 nm were measured on a plate reader to check for fungal growth. After measuring these ODs, the culture from each well was transferred into a 1.5 mL Eppendorf tube and briefly spun for 1 minute at top speed in a conventional centrifuge. An aliquot of 250 microliters of supernatant was transferred into each well of transparent flat bottom, 48-well tissue culture plates. 50 microliters of yellowish Salkowski reagent (0.01 M FeCl3 in 35% HClO4 (perchloric acid, #311421, Sigma) were added to each well and incubated in the dark for 30 minutes before measuring the OD540 nm in a plate reader to detect pink/red color. Images were also taken for qualitative scoring of the results later.

Auxin is an important plant hormone that can promote cell enlargement and inhibit branch development (meristem activity) in above ground plant tissues, while below ground it has the opposite effect, promoting root branching and growth. Additionally, auxin signaling pathway has been shown to interact with plant defense signaling pathways. Several microbes utilize the auxin-defense crosstalk to down-regulate the defense responses, therefore allowing harmonious co-existence of the microbe and plants.

Strain B was screened for the ability to produce auxin as a possible growth-promoting agent (Table 2).

### Acetoin and Diacetyl Production

The method was adapted from Phalip et al., (1994) J Basic Microbiol 34: 277-280. (incorporated herein by reference). 100 microliters of fungal culture prepared as described above was inoculated into 1 mL of R2A broth supplemented with 5% sterile glucose in transparent flat bottom, 12-well tissue culture plates. Each culture was grown in triplicates. The plates were sealed with a breathable membrane, wrapped in aluminum foil, and incubated at 25° C. on a shaker at a speed of 150 rpm in the dark for 3 days. After 3 days the OD600 nm and OD525 nm were measured on a plate reader to check for fungal growth. After measuring these ODs, the culture from each well was transferred into a 1.5 mL Eppendorf tube and briefly spun for 1 minute at top speed in a conventional centrifuge. An aliquot of 250 microliters of supernatant was transferred into each well of transparent flat bottom, 48-well tissue culture plates. 50 microliters per well was added of freshly blended Barritt's Reagents A and B [5 g/L creatine mixed 3:1 (v/v) with freshly prepared alpha-naphthol (75 g/L in 2.5 M sodium hydroxide)]. After 30 minutes, images were taken to score for red or pink coloration relative to a copper colored negative control and the absorption at 525 nm was measured using a plate reader to quantify the acetoin and diacetyl abundance.

Acetoin is a neutral, four-carbon molecule used as an external energy storage by a number of fermentive microbes. It is produced by the decarboxylation of alpha-acetolactate, a common precursor in the biosynthesis of branched-chain amino acids. Owing to its neutral nature, production and excretion of acetoin during exponential growth prevents overacidification of the cytoplasm and the surrounding medium that would result from accumulation of acidic metabolic products, such as acetic acid and citric acid. Once superior carbon sources are exhausted, and the culture enters stationary phase, acetoin can be used to maintain the culture density.

Strain B acetoin results are summarized in Table 2.

### Siderophore Production

To ensure no contaminating iron was carried over from previous experiments, all glassware was deferrated with 6 M HCl and water prior to media preparation [Cox (1994) Methods Enzymol 235: 315-329, incorporated herein by reference]. In this cleaned glassware, 1 mL of R2A broth media, which is iron limited, was aliquotted into each well of transparent flat bottom, 12-well tissue culture plates. 100 microliters of fungal culture prepared as described above were inoculated into each well. Each culture was grown in three replicates. The plates were sealed with a breathable membrane, wrapped in aluminum foil, and incubated at 25° C. on a shaker at a speed of 150 rpm in the dark for 3 days. After 3 days the OD600 nm and OD530 nm were measured on a plate reader to check for fungal growth. After measuring these ODs, the culture from each well was transferred into a 1.5 mL Eppendorf tube and briefly spun for 1 minute at top speed in a conventional centrifuge. An aliquot of 250 microliters of supernatant was transferred into each well of transparent flat bottom, 48-well tissue culture plates. After incubation, 100 microliters of 0-CAS preparation without gelling agent [Perez-Miranda et al. (2007), J Microbiol Methods 70: 127-131, incorporated herein by reference] was added into each well. One liter of 0-CAS reagent was prepared using the cleaned glassware by mixing 60.5 mg of chrome azurol S (CAS), 72.9 mg of hexadecyltrimethyl ammonium bromide (HDTMA), 30.24 g of finely crushed Piperazine-1,4-bis-2-ethanesulfonic acid (PIPES) with 10 mL of 1 mM FeCl3.6H2O in 10 mM HCl solvent. The PIPES had to be finely powdered and mixed gently with stirring (not shaking) to avoid producing bubbles, until a deep blue color was achieved. 30 minutes after adding the reagent to each well, images were taken and color change was scored by looking for purple halos (catechol type siderophores) or orange colonies (hydroxamate siderophores) relative to the deep blue of the O-CAS. Absorption at 420 nm was measured using a plate reader to quantify the abundance of siderophore.

Siderophore production by fungus on a plant surface or inside a plant may both show that a microbe is equipped to grow in a nutrient limited environment, and perhaps protect the plant environment from invasion by other, perhaps undesirable microbes. Strain B siderophore results are summarized in Table 2.

### Additional In Vitro Testing and Characterization of Penicillium Fungal Endophytes

Examples below are adapted from: Johnston-Monje and Raizada (2011), which is incorporated herein by reference in its entirety.

Assay for Growth on Nitrogen Free LGI Media.

All glassware is cleaned with 6 M HCl before media preparation. A new 96 deep-well plate (2 mL well volume) is filled with 1 mL/well of sterile LGI broth [per L, 50 g Sucrose, 0.01 g FeCl₃-6H₂O, 0.8 g K₃PO₄, 0.2 g MgSO₄-7H₂O, 0.002 g Na₂MoO₄-2H₂O, pH 7.5]. Bacteria are inoculated with a flame-sterilized 96 pin replicator. The plate is sealed with a breathable membrane, incubated at 25° C. with gentle shaking for 5 days, and OD₆₀₀ readings taken.

ACC Deaminase Activity Assay.

Microbes are assayed for growth with ACC as their sole source of nitrogen. Prior to media preparation all glassware is cleaned with 6 M HCl. A 2 M filter sterilized solution of ACC (#1373A, Research Organics, USA) is prepared in water. 1 ul/mL of this is added to autoclaved LGI broth (see above), and 1 mL aliquots are placed in a new 96 well plate. The plate is sealed with a breathable membrane, incubated at 25° C. with gentle shaking for 5 days, and OD600 readings taken. Only wells that are significantly more turbid than their corresponding nitrogen free LGI wells are considered to display ACC deaminase activity.

Mineral Phosphate Solubilization Assay.

Microbes are plated on tricalcium phosphate media. This is prepared as follows: 10 g/L glucose, 0.373 g/L NH₄NO₃, 0.41 g/L MgSO₄, 0.295 g/L NaCl, 0.003 FeCl₃, 0.7 g/L Ca₃HPO₄ and 20 g/L Agar, pH 6, then autoclaved and poured into 150 mm plates. After 3 days of growth at 25° C. in darkness, clear halos are measured around colonies able to solubilize the tricalcium phosphate.

RNAse Activity Assay.

1.5 g of torula yeast RNA (#R6625, Sigma) is dissolved in 1 mL of 0.1 M Na₂HPO₄ at pH 8, filter sterilized and added to 250 mL of autoclaved R2A agar media which is poured into 150 mm plates. The microbes are inoculated inoculated using a flame-sterilized 96 pin replicator, and incubated at 25° C. for 3 days. On day three, plates are flooded with 70% perchloric acid (#311421, Sigma) for 15 minutes and scored for clear halo production around colonies.

Pectinase Activity Assay.

Adapting a previous protocol 0.2% (w/v) of citrus pectin (#76280, Sigma) and 0.1% triton X-100 are added to R2A media, autoclaved and poured into 150 mm plates. Microbes are inoculated using a 96 pin plate replicator. After 3 days of culturing in the darkness at 25° C., pectinase activity is visualized by flooding the plate with Gram's iodine. Positive colonies are surrounded by clear halos.

Cellulase Activity Assay.

Adapting a previous protocol, 0.2% carboxymethylcellulose (CMC) sodium salt (#C5678, Sigma) and 0.1% triton X-100 are added to R2A media, autoclaved and poured into 150 mm plates. Microbes are inoculated using a 96 pin plate replicator. After 3 days of culturing in the darkness at 25° C., cellulose activity is visualized by flooding the plate with Gram's iodine. Positive colonies are surrounded by clear halos.

Oxidase and Catalase Activity Assays.

Oxidase and catalase activities are tested with 1% (w/v) tetramethyl-p-phenylene diamine and 3% (v/v) hydrogen peroxide solution, respectively. Gelatin and casein hydrolytic properties are analyzed by streaking fungal strains onto TSA plates from the stock culture. After incubation, trichloroacetic acid (TCA) is applied to the plates and an observation is made immediately for a period of at least 4 min (Medina and Baresi 2007, J Microbiol Methods 69:391-393). Chitinase activity of the isolates is determined as zones of clearing around colonies following the method of Chernin et al. (1998) J Bacteriol 180:4435-4441 (incorporated herein by rereference). Hemolytic activity is determined by streaking fungal isolates onto Columbia 5% sheep blood agar plates. Protease activity is determined using 1% skimmed milk agar plates, while lipase activity is determined on peptone agar medium. Formation of halo zone around colonies was used as indication of activity (Smibert and Krieg 1994, In: Gerhardt P, Murray R, Wood W, Krieg N (Eds) Methods for General and Molecular Bacteriology, ASM Press, Washington, D.C., pp 615-640, incorporated herein by reference). Pectinase activity is determined on nutrient agar supplemented with 5 g L⁻¹ pectin. After 1 week of incubation, plates are flooded with 2% hexadecyl trimethyl ammonium bromide solution for 30 min. The plates are washed with 1M NaCl to visualize the halo zone around the fungal growth (Mateos et al. 1992, Appl Environ Microbiol 58:1816-1822, incorporated herein by reference).

Assays for Poly-Hydroxybutyrate (PHB) and n-Acyl-Homoserine Lactone (AHL) Production.

The fungal isolates are tested for PHB production (qualitative) following the viable colony staining methods using Nile red and Sudan black B (Juan et al. 1998 Appl Environ Microbiol 64:4600-4602; Spiekermann et al. 1999, Arch Microbiol 171:73-80, each of which is incorporated by reference). The LB plates with overnight fungal growth are flooded with 0.02% Sudan black B for 30 min and then washed with ethanol (96%) to remove excess strains from the colonies. The dark blue coloured colonies are taken as positive for PHB production. Similarly, LB plates amended with Nile red (0.5 uL mL⁻¹) were exposed to UV light (312 nm) after appropriate fungal growth to detect PHB production. Colonies of PHA-accumulating strains fluoresce under ultraviolet light. The fungal strains were tested for AHL production following the method modified from Cha et al. (1998), Mol Plant-Microbe Interact 11:1119-1129. The LB plates containing 40 ug ml⁻¹ X-Gal are plated with reporter strains (A. tumefaciens NTL4.pZLR4). The LB plates are spot inoculated with 10 uL of fungal culture and incubated at 28±2° C. for 24 h. Production of AHL activity is indicated by a diffuse blue zone surrounding the test spot of culture. Agrobacterium tumefaciens NTL1 (pTiC58ΔaccR) is used as positive control and plate without reporter strain is considered as a negative control.

Antibiosis Assay.

Bacteria or fungi are inoculated using a 96 pin plate replicator onto 150 mm Petri dishes containing R2A agar, then grown for 3 days at 25° C. At this time, colonies of either E. coli DH5a (gram negative tester), Bacillus subtillus ssp. Subtilis (gram positive tester), or yeast strain AH109 (fungal tester) are resuspended in 1 mL of 50 mM Na₂HPO₄ buffer to an OD₆₀₀ of 0.2, and 30 ul of this is mixed with 30 mL of warm LB agar. This is quickly poured completely over a microbe array plate, allowed to solidify and incubated at 37° C. for 16 hours. Antibiosis is scored by looking for clear halos around fungal colonies.

Antagonistic activities against plant pathogenic bacteria, fungi and oomycetes. The antagonistic activities of fungal isolates are screened against plant pathogenic fungi (Fusarium caulimons, Fusarium graminarium, Fusarium oxysporum, Fusarium solani, Rhizoctonia solani, Thielaviopsis basicola) and oomycetes (Phytophthora infestans, Phytophthora citricola, Phytophthora cominarum).

Antagonistic activity of the fungal isolates against pathogenic fungi and oomycetes is tested by the dual culture technique on potato dextrose agar (PDA) and yeast malt agar (YMA) media (Dennis and Webster 1971, Trans Brit Mycol Soc 57:25-39, incorporated herein by reference). A small agar plug (5 mm in diameter) of target fungus/oomycetes is placed near the edge of petri dishes of both media, adjacent to an agar plug of the isolated fungus (as close as possible without touching). Plates are incubated for 14 days at 24° C. and radial growth or inhibition of it is measured.

### Biolog Assay

Fungal strains were maintained on potato dextrose agar (PDA) in dark at 25° C. and subcultured at regular intervals to maintain viability. Sterile cotton buds were used to gently scrape fungal spores and mycelial fragments from two-week old cultures that were resuspended in 5 mL Filamentous Fungi Inoculation Fluid (FF-IF) obtained from BIOLOG.

In order to achieve homogeneity, the fungal cell suspension was sonicated for 90 seconds continuously with probe intensity set to 3 using the Sonic Dismembrator Model 100 (Thermo Fisher Scientific, Waltham, Mass.). The homogenous cell suspension was measured using a microplate reader to achieve an absorbance range of 0.2-0.3 at 590 nm. A 48-fold dilution of the homogenous cell suspension was done using FF-IF, and 100 uL of the diluted sample was used per well in the 96-well Phenotype MicroArray (PM) 1 MicroPlate (Hayward, Calif.). The PM1 Microplate contained 95 carbon sources and one negative control. Each well contained a unique carbon substrate.

MicroPlates were sealed with Parafilm and incubated at 25° C. in an enclosed container for 72 hours. All MicroPlates were examined at intervals of 24 hours and assay results were recorded at 72 hours. The ability of each fungal strain to utilize the carbon substrates on the PM 1 Microplate was determined by measuring fungi cell growth/turbidity at 590 nm. All MicroPlates contained a negative control well (water only) that remained colorless until the end of each experiment indicating no or very little fungal cell growth.

The ability of a strain to utilize a specific carbon substrate in the BIOLOG PM MicroPlates could be determined by the increased turbidity due to cell growth in that particular well.

The following carbon substrates were utilized by Strain B: L-Arabinose, L-Proline, D-Xylose, L-Glutamic acid, D-Ribose, L-Asparagine, Sucrose, Tween 80, Adonitol, L-Alanine, L-Alanyl-Glycine, L-Galactonic-acid-γ-lactone, β-Methyl-D-glucoside, m-Inositol, D-Galactose, D-Trehalose, D-Glucuronic acid, D-Gluconic acid, D-Mannitol, D-L-Malic acid, α-D-Glucose, Maltose, D-Melibiose, Maltotriose, Pyruvic acid, D-Galacturonic acid, D-Mannose, L-Threonine, Inosine, L-Lyxose, D-Alanine, L-Lactic acid, D-Galactonic acid-γ-lactone, Uridine, α-Hydroxy Glutaric acid-γ-lactone, D-L-α-Glycerol phosphate.

Biolog assay results are summarized in Table 3.

### Example 3: Identification of Differentially Regulated Proteins in Penicillium Fungal Culture (Proteomics)

### Methods

Microbial Samples Preparation:

Microbes were cultivated in three biological replicates for each strain (Strain B and Strain F). Fungal strains were streaked on potato dextrose (PD) agar and individual plugs containing spores and mycelial tissues were used to initiate growth in 10 mL PD broth for 6 days. All strains were grown with agitation at room temperature. Microbial culture filtrate was harvested by centrifuging at 4500 RPM for 20 minutes in 15 mL Falcon tubes to allow culture separation and removal of the supernatant. Five mL of culture supernatant were used for secreted proteomics analysis. All steps were performed in sterile conditions. Culture filtrates were kept in dry ice after harvest at all times to preserve protein stability. Media-only samples consisting of PDB and R2A were tested independently to ensure the absence of intact proteins that may potentially interfere with the secreted microbial peptides.

Protein Purification and Visualization:

Samples were shipped to the vendor site (MS Bioworks, Ann Arbor, Mich.) for peptide purification and analysis. Each sample was concentrated on a Pall 3 kD MWCO MicroSep Spin Column (VWR Cat#89132-006) and quantified at 1:10 dilution by Qubit fluorometry (Life Technologies). Twelve ug of each sample was separated ˜1.5 cm on a 10% Bis-Tris Novex mini-gel (Invitrogen) using the MES buffer system. The gel was stained with Coomassie and each lane was excised into ten equally sized segments. Gel pieces were processed using a robot (ProGest, DigiLab) by washing with 25 mM ammonium bicarbonate followed by acetonitrile. The samples were subsequently reduced with 10 mM dithiothreitol at 60° C. followed by alkylation with 50 mM iodoacetamide at room temperature, digested with trypsin (Promega) at 37° C. for 4 hours and quenched with formic acid. The supernatant was analyzed directly without further processing.

Mass Spectrometry:

The digests were analyzed by nano LC/MS/MS with a Waters NanoAcquity HPLC system interfaced to a ThermoFisher Q Exactive. Peptides were loaded on a trapping column and eluted over a 75 um analytical column at 350 nL/min; both columns were packed with Proteo Jupiter resin (Phenomenex). A 30 min gradient was employed (5h total). The mass spectrometer was operated in data-dependent mode, with MS and MS/MS performed in the Orbitrap at 70,000 FWHM and 17,500 FWHM resolution, respectively. The fifteen most abundant ions were selected for MS/MS.

Data Acquisition and Processing:

Symbiota provided protein sequence data, KEGG annotations and corresponding protein mass spectrometry spectral count data to ABiL. All data were converted into file formats and a local database suitable for subsequent processing, analysis and parallelization.

Protein Ortholog Identification:

Pairs/groups of orthologous proteins were identified using a modified version of the OrthoMCL pipeline (Fischer, 2011). Orthologs were identified as reciprocal best BLASTP hits, and then clusters of orthologous proteins were defined using the modified OrthoMCL pipeline. This process was done independently for the within genera and the between genera analyses. BLASTP was run in parallel on the Georgia Tech PACE HPC environment.

Protein Functional Annotation:

KEGG annotations for individual proteins were provided by Symbiota. The program BLAST2GO (Conesa, 2005) was used to annotate proteins with gene ontology (GO) terms based on sequence similarity to previously annotated proteins.

Protein Expression Quantification and Normalization:

Individual protein expression levels were taken as the number of observed spectra (i.e. the spectra count) corresponding to each protein. Protein spectra counts were retrieved across three replicates for each species. Missing counts for any given ortholog or replicate were assigned values of 0. Individual protein expression levels (spectra counts) were then normalized by the total number of observed spectra for each replicate. This process was done independently for the three replicates corresponding to each member of the A-B pair of every species. Fold-change (FC) values for orthologous pairs/groups were computed as log 2 AB spectra counts for the purpose of functional enrichment analysis (below).

Protein Differential Expression Analysis:

Differential protein expression analysis was done for a) pairs of orthologous proteins from the within genera analysis and b) groups of orthologous proteins from the between genera analysis. Differential expression was quantified by comparing the within group normalized spectra count variation to the between group normalized spectra count variation using the Students t-test. A Benjamini-Hochberg False Discover Rate threshold of 0.2 was used to identify differentially abundant orthologous proteins.

Pathway and Functional Enrichment Analysis:

Enrichment analysis was done in parallel using both KEGG and GO annotations with the hypergeometric test and via Gene Set Enrichment Analysis (GSEA) (Huang, 2009; Subramanian, 2005). For the hypergeometric test, for any given functional annotation category (i.e. KEGG pathway or GO term), the number of proteins up-regulated in the beneficial member of the orthologous pair was compared to the total number of proteins up-regulated in the complete set of orthologs. For GSEA analysis, orthologous protein pairs/groups were ranked by FC values (as defined in #3 above) and the distribution of FC values was evaluated for a shift using the clusterprofiler R package (Yu, 2012).

### Results

Secreted fungal proteins as cataloged in this experiment were at the interface of the host-symbiont symbiosis, and play important roles in modulating the plant-microbe interaction due to the molecules having direct access to the plant host cell wall.

The in-culture secretomics analysis of the beneficial (Strain B) and control (Strain F) strains of the filamentous fungus Penicillium revealed a total of 71 secreted proteins of which 66 could be grouped in either Gene Ontology (GO) or Kyoto Encyclopedia of Genes and Genomes (KEGG).

Comparative analysis of the orthologous proteins expressed by the beneficial and control strains of Penicillium revealed a total of 13 orthologous proteins (12 could be categorized either in GO or KEGG) that were secreted in the beneficial strain culture only. (Table 4A). The proteins ranged between 12.3 to 5-fold difference (differential expression was quantified by comparing the within group normalized spectra count variation to the between group normalized spectra count variation using the Students t test).

Similar differential expression analysis of the secreted proteins showed that 43 (41 could be categorized either in GO or KEGG) orthologous proteins were detected only in the control strain (Table 4B). The expression levels of proteins in the beneficial strain relative to the neutral strain were found to range from −11.3 to −3.7 in fold difference.

In addition, 11 (9 could be categorized either in GO or KEGG) orthologous proteins were found to be present in higher fold changes (5.1 to 2.2) in the beneficial Penicillium strain relative to the control strain (Table 4C), and four orthologous proteins were detected at a lower expression level (−7.7 to −0.8) in the beneficial strain in comparison with the control fungal strain (Table 4D).

Overall, the small proteins found to be secreted in the fungal culture could be categorized into various biological categories based on Gene Ontology (GO) clustering. Striking differential expression patterns were observed for proteins within the following gene families.
(1) carbohydrate and cellulose metabolism: glucan 1,4-alpha-glucosidase activity, polysaccharide metabolic process; glucan 1,4-alpha-glucosidase activity, polysaccharide catabolic process, starch binding; carbohydrate metabolic process, cell wall macromolecule catabolic process, lysozyme activity, peptidoglycan catabolic process; arabinogalactan endo-1,4-beta-galactosidase activity, carbohydrate metabolic process, glucosidase activity; carbohydrate binding, carbohydrate metabolic process, hydrolase activity, hydrolyzing O-glycosyl compounds, integral component of membrane; cellulose 1,4-beta-cellobiosidase activity, cellulose binding, cellulose catabolic process, extracellular region, hydrolase activity, hydrolyzing O-glycosyl compounds; carbohydrate metabolic process, glucan endo-1,6-beta-glucosidase activity, glucosylceramidase activity, sphingolipid metabolic process; acetylxylan esterase activity, cellulose catabolic process, extracellular region, hydrolase activity, methylation, methyltransferase activity, xylan catabolic process; carbohydrate binding, carbohydrate metabolic process, cell wall, cell wall organization, DNA binding, hydrolase activity, hydrolyzing O-glycosyl compounds, nucleus, transcription, DNA-templated, transferase activity, zinc ion binding; (1->3)-beta-D-glucan metabolic process, 1,3-beta-glucanosyltransferase activity, anchored component of membrane, carbohydrate metabolic process, fungal-type cell wall, hydrolase activity, integral component of membrane, plasma membrane, transferase activity; carbohydrate binding, carbohydrate metabolic process, hydrolase activity; carbohydrate metabolic process, hydrolase activity, hydrolyzing O-glycosyl compounds; bglX, Cyanoamino acid metabolism, Phenylpropanoid biosynthesis, Starch and sucrose metabolism; Carbohydrate digestion and absorption, E3.2.1.1, amyA, malS, Starch and sucrose metabolism; Galactose metabolism, galM, GALM, Glycolysis/Gluconeogenesis; E3.2.1.58, Starch and sucrose metabolism; E3.2.1.58, Starch and sucrose metabolism; E3.2.1.22B, galA, rafA, Galactose metabolism, Glycerolipid metabolism, Glycosphingolipid biosynthesis—globo series, Sphingolipid metabolism; carbohydrate binding, carbohydrate metabolic process, hydrolase activity; carbohydrate binding, carbohydrate metabolic process, endoribonuclease activity, hydrolase activity, hydrolyzing O-glycosyl compounds, integral component of membrane, RNA phosphodiester bond hydrolysis, endonucleolytic, rRNA transcription; carbohydrate binding, carbohydrate metabolic process, cell wall, cell wall organization, hydrolase activity, hydrolyzing O-glycosyl compounds, integral component of membrane, transferase activity; carbohydrate binding, carbohydrate metabolic process, hydrolase activity, acting on glycosyl bonds; E3.2.1.4, Starch and sucrose metabolism; E3.2.1.58, Starch and sucrose metabolism; Galactose metabolism, malZ, Starch and sucrose metabolism.
(2) ATP binding and the mitochondrion: ADP binding, ATP binding, ATP hydrolysis coupled proton transport, ATP synthesis coupled proton transport, mitochondrial ATP synthesis coupled proton transport, mitochondrial proton-transporting ATP synthase, catalytic core, proton-transporting ATP synthase activity, rotational mechanism, proton-transporting ATP synthase complex, catalytic core F(1), proton-transporting ATPase activity, rotational mechanism; cytosol, electron carrier activity, heme binding, metal ion binding, mitochondrial ATP synthesis coupled electron transport, mitochondrion, nucleus, oxidation-reduction process, respiratory chain; mitochondrion, oxidation-reduction process, oxidoreductase activity; ATP binding, carbohydrate phosphorylation, cell, cellular glucose homeostasis, glucose binding, glycolytic process, hexokinase activity.
(3) metabolic activities including hydrolase activity and protein glycosylation: MAN1, N-Glycan biosynthesis, Protein processing in endoplasmic reticulum, Various types of N-glycan biosynthesis; hydrolase activity, metabolic process; hydrolase activity, acting on ester bonds, metabolic process; catalytic activity, hydrolase activity, acting on glycosyl bonds, metabolic process; Betalain biosynthesis, Isoquinoline alkaloid biosynthesis, Melanogenesis, Riboflavin metabolism, TYR, Tyrosine metabolism; E5.1.3.15, Glycolysis/Gluconeogenesis; dephosphorylation, phosphatase activity; cytoplasm, GTP binding, GTPase activity, translation elongation factor activity, translation initiation factor activity, translational elongation, translational initiation; E3.1.3.8, Inositol phosphate metabolism; hydrolase activity, metabolic process; flavin adenine dinucleotide binding, integral component of membrane, oxidation-reduction process, oxidoreductase activity, acting on CH—OH group of donors; flavin adenine dinucleotide binding, oxidation-reduction process, oxidoreductase activity, acting on CH—OH group of donors; flavin adenine dinucleotide binding, oxidation-reduction process, oxidoreductase activity, acting on CH—OH group of donors, UDP-N-acetylmuramate dehydrogenase activity; chitosanase activity, extracellular region, polysaccharide catabolic process; carbohydrate metabolic process, chitin catabolic process, chitinase activity; hydrolase activity, acting on ester bonds, metabolic process.
(4) regulation of transcription: nutrient reservoir activity, regulation of transcription, DNA-templated, sequence-specific DNA binding, transcription factor activity, sequence-specific DNA binding.
(5) transport: APE2.
(6) proteolysis: PEPD; aspartic-type endopeptidase activity, carbohydrate binding, extracellular region, proteolysis, zymogen activation; aspartic-type endopeptidase activity, extracellular region, pathogenesis, proteolysis; pepP; FMN binding, integral component of membrane, metalloendopeptidase activity, oxidation-reduction process, oxidoreductase activity, proteolysis; ATP binding, helicase activity, nucleic acid binding, proteolysis, serine-type peptidase activity; aspartic-type endopeptidase activity, ATP binding, methylation, methyltransferase activity, nucleic acid binding, proteolysis, ribosome, structural constituent of ribosome, tryptophan-tRNA ligase activity, tryptophanyl-tRNA aminoacylation.
(7) cellular membrane-associated: integral component of membrane; cytoplasm, regulation of catalytic activity, Rho GDP-dissociation inhibitor activity; anchored component of membrane, carbohydrate metabolic process, plasma membrane, transferase activity; anchored component of membrane, carbohydrate metabolic process, hydrolase activity, plasma membrane, transferase activity; anchored component of membrane, carbohydrate metabolic process, plasma membrane, transferase activity.
(8) amino acid biosynthesis and metabolism: 2-Oxocarboxylic acid metabolism, Alanine, aspartate and glutamate metabolism, Arginine and proline metabolism, Biosynthesis of amino acids, Carbon fixation in photosynthetic organisms, Carbon metabolism, Cysteine and methionine metabolism, GOT1, Isoquinoline alkaloid biosynthesis, Phenylalanine metabolism, Phenylalanine, tyrosine and tryptophan biosynthesis, Tropane, piperidine and pyridine alkaloid biosynthesis, Tyrosine metabolism; Biosynthesis of amino acids, Carbon fixation in photosynthetic organisms, Carbon metabolism, Pentose phosphate pathway, rpiA; Glutathione metabolism, GSR, gor, Thyroid hormone synthesis; Cysteine and methionine metabolism, E3.3.1.1, ahcY; AGXT, Alanine, aspartate and glutamate metabolism, Carbon metabolism, Glycine, serine and threonine metabolism, Glyoxylate and dicarboxylate metabolism, Methane metabolism, Peroxisome; Cysteine and methionine metabolism, E2.4.2.28, mtaP.
(9) response to oxidative stress: msrA Methionine sulfoxide reductase.

Many secreted proteins in the carbohydrate and cellulose metabolism category were associated with degradation of xylan, a polysaccharide found abundantly in plant cell walls. The proteins have also been implicated to be important in symbiotic fungi such as mycorrhiza because of their ability to acquire and utilize carbon photosynthates such as sucrose that is found in abundance within the plant cell walls. Secreted proteins in this category such as invertase potentially may enable the fungal endophyte to more efficiently access similar plant-derived sugars (Ceccaroli et al. 2011).

In addition, one of the most notable findings uncovered in this study is that proteins that are associated with cell wall degradation namely glucan 1,4-alpha-glucosidase were secreted in high abundance in the beneficial Penicillium strain (Strain B). Interestingly, two of the highest expressed proteins (12.3 and 7.5 fold changes) in the beneficial fungal strain were identified as a glucan 1,4-alpha-glucosidase. This protein was found to be present only in the culture of Strain B and was detected to have little to no expression in the beneficial strain relative to the beneficial one. This is especially intriguing in light of a recent report documenting an enzyme similarly identified as a glucan 1,4-alpha-glucosidase that was isolated from the fungus Botrytis cinerea and was observed to play a crucial role in conferring improved disease resistance against B. cinerea, Pseudomonas syringae pv. tomato DC3000 and tobacco mosaic virus in plants challenged with those pathogens (Zhang et al 2015).

Proteins that were involved in ATP binding and mitochondrial ATP synthesis group are primarily involved in energy and reactive oxidative species (ROS) generation. In living organisms, ROS are used in many important biological processes such as signaling pathways and defense mechanisms against pathogens (Rexroth et al. 2012).

Fungal secreted proteins that were associated in metabolic processes such as hydrolase activity and protein glycosylation are known to include enzymes that could be useful for breaking down biomass to smaller molecules that are then subsequently utilized as nutrition sources (Li et al. 2004). These enzymes may be useful in breaking down plant cell components that are known to consist of cellulose, hemicellulose, pectins, and wall-associated proteins (Di Pietro et al. 2009). Studies have also reported on the abundance of secreted proteins that are involved in degrading biomass produced by other filamentous fungus such as Trichoderma reesei (Foreman et al. 2003). The role of glycosylation as a form of post-translational modification in filamentous fungi is well studied, and members of this group are associated with stability, secretion and localization of proteins. In addition, there are also reports of glycoproteins that are localized within the membrane playing a role in trans-membrane communication (Despande et al. 2008).

A markedly higher expression of proteins that are involved in the hydrolase activity was observed in the control Penicillium strain (Strain F) in this study. For instance, in the beneficial Penicillium strain (Strain B), the total number of expressed proteins associated with hydrolase activity totaled eight. Three of those proteins were detected only in the beneficial strain (Strain B), and another three had lower expression than its control counterpart (Strain F). Interestingly, the total number of expressed proteins associated in this category in the control Penicillium strain was 19. Fourteen of the 19 proteins, (74%) had very little or no expression in the beneficial Penicillium strain, while two were expressed higher relative to the beneficial strain. Only three proteins implicated in hydrolase activity were expressed lower in the control strain relative to the beneficial strain.

Fungal proteins that are involved in the regulation of transcription have been documented extensively in filamentous fungi. For instance, it is well-known that fungi produce low-molecular mass compounds known as secondary metabolites that exert important roles not only in transcription but also, in development and intercellular communications within the cellular milieu (Brakhage, 2013). In a recent review of fungal secreted proteins, Lo Presti et al. (2015) highlighted the role of a symbiont fungal effector; a group that encompasses any secreted molecule that is involved in regulating the fungal-plant relationship, in interactions with a plant host ethylene-responsive transcription factor which in turn modulates the expression of genes involved in host defense.

Fungal proteins that grouped with the transport category have wide-ranging functions in modulating the plant-microbe symbiosis as reported by Dupont et al. (2015). The transport of sugars and amino acid were among the function of these membrane transport proteins.

Fungal proteins that clustered within the proteolysis category typically included proteases and have been known to exist in endophytic (Lindstrom et al. 1993), and other filamentous fungi (Suarez et al. 2005). One such proteolytic enzyme, a fungal serine protease has been reported to in a mutualistic fungal endophyte, and the authors speculated that the expression of similar proteases may be a general feature of endophytes (Reddy et al. 1996). The inventors herein determined that such proteases were unique to beneficial fungal endophytes of the genus Penicillium.

Proteins associated with cellular membranes have been implicated in several processes. One notable finding in this dataset was the presence of Rho GDP-dissociation inhibitor activity in the control Penicillium strain, with very little to no expression detected in the beneficial strain (−7.7 fold change). In a study that investigated the functional role of this protein in another filamentous fungi, it has been reported that proteins like those play a role in modifying the fungal morphology especially related to the actin cyctoskeleton, in addition to being implicated in some gene expression and cell growth signaling pathways (Menotta et al. 2008).

Secreted proteins that were grouped in this category are important due to their role in building and breaking down amino acid residues. One such protein, 2-Oxocarboxylic acid, was reported to be one of the most enriched pathways in the cucumber pathogen, Botrytis cinerea during infection of the plant (Kong et al. 2015). Interestingly, this protein was present in only in the control Penicillium strain and was detected to have little to no expression in the beneficial strain relative to the beneficial one (−8.2 fold change).

One interesting finding obtained from comparing the secreted protein dataset of the beneficial and control Penicillium strains was the presence of a methionine sulfoxide reductase (msrA) in the culture of the control strain only. Enzymes such as msrA play a role in repairing damages caused by reactive oxygen species (ROS) on sulfur-containing amino acid residues, and in Aspergillus nidulans msrA confers protection against detrimental cellular modifications that are caused by oxidative stress (Soriani et al. 2009). This secreted protein was found to be present only in the culture of Strain F and was detected to have little to no expression in the beneficial strain Strain B (−8.2 fold change).

In conclusion, it was observed that the Penicillium strain secreted proteins in culture fall into nine major GO/KEGG categories. Proteins that were exclusively secreted by the beneficial Penicillium strain were grouped into 12 GO/KEGG categories while proteins that are only detected in the control strain encompassed 41 categories indicating that a wider range of proteins were secreted by the control fungal strain.

In the beneficial fungal secretome, there was enrichment of proteins that fell into carbohydrate and cellulose metabolic processes. Nine out of the 12 GO/KEGG categories (75%) of the proteins that were expressed only in the beneficial strain were grouped under that category, and based on the normalized protein spectra counts for both Penicillium strains, these proteins were secreted in abundance by the beneficial strain.

This differed in the secretome of the control strain, Strain F where only 14 out of 41 total specific GO/KEGG categories (34%) of orthologous proteins that are unique to that strain were found related to carbohydrate and cellulose metabolic processes. Therefore, the Penicillium culture secretome data supported the higher expression of secreted proteins related to carbohydrate and cellulose metabolism in a beneficial fungal strain compared to a control strain within the same genus.

One of the most striking findings based on the data was that a secreted protein that is involved in response to oxidative stress was expressed at a high level in the control strain relative to little or no presence in the beneficial fungal strain.

The secretome of the neutral strain of Penicillium strain also showed a slightly higher presence and expression of proteolytic proteins (13%) relative to the beneficial strain (12%). Interestingly, there was no lyase proteins detected in the beneficial strain's secretome, there were two secreted lyase protein that were expressed at −6.8 and −7.2 fold change in the neutral Penicillium strain relative to the beneficial strain. Lyase proteins in fungi are typically associated with breaking down pectin and glycosaminoglycans (Zhao et al. 2014), two major components of the plant cell wall.

### Example 4: Coating of Seeds with Penicillium Endophyte Strains

The following protocol was used to coat seeds with fungal inocula for planting in greenhouse trials. The “sticker” (2% methylcellulose) was autoclaved and aliquoted into 50 mL Falcon tubes. Seeds were pre-weighed and placed into 50 mL Falcon tubes (2 replicate seed aliquots per treatment). Penicillium strains were prepared by centrifuging cultures (2500×g for 10 minutes), removing supernatant, washing pellets, resuspending in minimal water, normalized to 10̂4 spores per seed in 250 uL sterile H2O. 250 uL of the 2% methylcellulose sticker was pre-mixed with the liquid culture suspension, and this liquid was pipetted onto the pre-weighed seeds. The Falcon tube was closed and shaken to distribute the culture:sticker mixed solution evenly. 150 uL of FloRite flowability polymer was added to the Falcon tube with the coated seeds, and shaken. Seeds were transferred to a labeled envelope and kept at room temperature until sowing. For all treatments, 2 replicate seed treatments were performed and on-seed CFUs were assessed on both replicates.

The following protocol was used to coat seeds with fungal inocula for planting in field trials. First, 3% Sodium alginate (SA) was prepared and autoclaved in the following manner. Erlenmeyer flasks were filled with the appropriate amount of deionized water and warmed to about 50 degrees C. on a heat plate with agitation using a stirring bar. SA powder was poured slowly into the water until it all dissolved. The solution was autoclaved (121° C. @15 PSI for 30 minutes). Talcum powder was autoclaved in dry cycle (121° C. @15 PSI for 30 minutes) and aliquoted in Ziploc bags or 50 ml falcon tubes at a ratio of 15 g per kg of seed to be treated for formulation controls and 10 g per kg of seed for actual treatments.

The next day, seeds were treated with either powdered or liquid formulations.

For powdered formulations, 10 g per kg of seed was allocated to the seeds to be treated, according to the following procedure. Seeds were placed in large plastic container. 16.6 ml of 2% SA per Kg of seeds to be treated were poured on the seeds. The container was covered and shaken slowly in orbital motion for about 20 seconds to disperse the SA. Endophyte powder was mixed with an equal amount of talcum powder. The mix of endophyte and talc was added on top of the seeds, trying to disperse it evenly. The container was covered and seeds were shaken slowly in orbital motion for about 20 seconds. 13.3 ml of Flo-rite per kg of seed to be treated is poured on the seeds. Seeds were shaken again, slowly and in orbital motion.

For liquid formulations, 8.5 mL per seed was allocated to the seeds to be treated, according to the following procedure. Seeds were placed in large plastic container. 8.3 ml of 2% SA per kg of seed and the same amount of culture (8.3 ml per kg of seed) were poured on the seeds. The container was covered and shaken slowly in orbital motion for about 20 seconds to disperse the SA. 15 g of talcum powder per kg of seed were added, trying to disperse it evenly. The container was covered and seeds were shaken slowly in orbital motion for about 20 seconds. 13.3 ml of Flo-rite per kg of seed to be treated were poured on the seeds. Seeds were shaken again, slowly and in orbital motion.

### Example 5: Seedling Assays

### Seeds and Seed Sterilization

Seeds were surface-sterilized with chlorine gas and hydrochloric acid as follows: Seeds were placed in a 250 mL open glass bottle and placed inside a desiccator jar in a fume. The cap of the glass bottle was treated similarly. A beaker containing 100 mL of commercial bleach (8.25% sodium hypochlorite) was placed in the desiccator jar near the bottle containing the seeds. Immediately prior to sealing the jar, 3 mL of concentrated hydrochloric acid (34-37.5%) was carefully added to the bleach and the bottle gently shaken to mix both components. The sterilization was left to proceed for 16 hours. After sterilization, the bottle was closed with its sterilized cap, and reopened in a sterile laminar hood. The opened bottle was left in the sterile hood for a minimum of one hour, with occasional shaking and mixing to air out the seeds and remove chlorine gas leftover. The bottle was then closed and the seeds stored at room temperature in the dark until use.

### Seed Coating of Formulation

Coating of seeds with dry or liquid formulation was executed as described in Example 4. All endophytes were grown in UltraYields flasks. Besides non-treated seeds, seeds were also coated with liquid formulation and medium only, to serve as controls.

### Seed Germination Assay on Water Agar

Sterilized seeds were placed onto water agar plates (1.3% bacto agar) in a biosafety hood using flamed forceps. For each treatment, 4 plates were sowed with 8 seeds each plate. After sowing, plates were sealed with Parafilm, randomized to avoid position effects, and placed in a drawer at room temperature in the dark. Seed germination was monitored every day for 2-4 days. After 3 days, images were taken of each plate and the root length of each seedling is measured using the imaging software ImageJ. The percentage difference between the treated seedlings, the mock-treated seedlings, and non-treated seedlings was then calculated.

### Rolling Paper Assay for Evaluating Seed Germination and Seedling Drought Tolerance

Sterilized seeds were placed 1-inch apart from each other onto sterilized rolling paper pre-soaked with sterile diH20 in a biosafety hood. The seeds were placed about one inch below the top and about ten inches above the bottom of the rolling paper. After placing the seeds, another layer of pre-soaked rolling paper was covered onto the top and the paper was carefully and slowly rolled up. The paper roll with seeds was placed vertically into autoclaved glass jar and covered with the lid to hold water absorbed in rolling paper. The jars were kept in a growth chamber in the dark, at 22° C., 60% RH for 4 days. At day 4, the lids were opened and the jars placed at 22° C., 70% RH, 12 h day light (level 4, ˜300-350 microE) for 3 more days before scoring.

### Drought Tolerance Assay Using Vermiculite

After scoring the germination rate of seeds on water agar, seedlings of similar physiological status (i.e., similar radical and shoot lengths) were transferred onto autoclaved vermiculite loosely packed in test tubes (3-cm in diameter) in their natural position (i.e., root down and shoot up). Before seedling transfer, 1.5 ml of sterile diH20 was added onto the top of the vermiculite. After transfer, the seedlings were gently covered with surrounding vermiculite. Test tubes were covered with lid to keep moisture for seeding to recover from transplanting and incubated in a growth chamber in the dark with the settings described above. The lid was removed the next day and the growth of seedlings was monitored every day for drought tolerance.

### Results

As shown in Table 5, Penicillium Strain B promoted wheat root (radical) and shoot growth three days after sowing on water agar, under normal watering and water-limited conditions.

### Example 6: Greenhouse Characterization

### Setup and Watering Conditions

A sandy loam growth substrate was mixed in the greenhouse and consisting of 60% loam and 40% mortar sand (Northeast Nursery, Peabody, Mass.). Prior to mixing, loam was sifted through a ⅜″ square steel mesh screen to remove larger particles and debris.

For greenhouse experiments, half of the nitrogen fertilizer (urea) and all phosphate (monoammonium phosphate, MAP) and potash to be applied during the season were added to the soil mixture prior to sowing. The remaining urea was provided dissolved in irrigation water at the onset of the reproductive stages of development. For soybean the total applied nutrients were 440 lbs/acre of urea, 38 lbs/MAP, and 105 lbs/acre potash. Substrate surface area per pot was calculated based on pot diameter in order to approximate the “acreage” of individual pots. An equivalent volume of fertilized soil was then gently added to each pot in order to minimize compaction of the soil. The substrate was saturated with water 3-4 hours before sowing.

Commercially available soybean seeds were coated with microbial treatments using the formulation used for field trials and described herein. Treatments included microbial coatings with the Penicillium strains Strain B and Strain F, and at least one non-endophyte control (non-treated, or formulation only-treated).

Three seeds were sown evenly spaced at the points of a triangle. Soil was then overlaid atop the seeds (estimated average planting depth at 1.0 to 1.5 inches) and an additional 700 mL water was added to moisten the overlaying substrate. Post-planting, the seeds were watered with 125 mL water per day. Pots were thinned down to 1 best seedling at true leaves stage (approximately 2 weeks).

The transplanting protocol for the seeds was as follows: Transplanting occurred at the time of thinning, to replace pots with no emergence or damaged plants with transplanted healthy plants of the same treatment in new pots. Three liters of the identical soil mix was added to the new pot. One plant was carefully removed from a healthy pot of the same treatment and placed in the new pot. The new pot was filled with soil to 4 L, with gentle packing around the roots. The new pot was watered with 700 mL water immediately after adding soil to each transplant. Transplanted seedlings were monitored for wilt and/or stress symptoms and delayed development. The original pots were retained in case the transplant became unhealthy.

Plants treated to the normal watering condition regime were watered with 125 mL water per day.

### Drought Stress Testing

Plants were provided with water to ˜50% capacity of the substrate for the first 14 days after sowing at which point water was withheld from water-stress plants until visible signs of wilting in vegetative tissues (i.e. drooping leaves and petioles, leaf rolling, chlorosis). Water-stressed plants were then irrigated to 50% soil water capacity, after which another drought cycle was initiated. Such drought cycles were continued until plants reached maturity. Throughout the experiment, the greenhouse was maintained on a 14-hour photoperiod where they were provided with at least 800 microE m̂-2 ŝ-1, ˜21° C. daytime and ˜18° C. nighttime temperatures and a relative humidity of ˜20-40%.

The watering regime for the drought-exposed seedlings was conducted as follows: approximately half saturation of soil at first day of emergence, third day of emergence, and 1 week later (day of thinning), full saturation at 5 days after thinning to initiate drought, full saturation to end drought when severe drought symptoms are observed, half saturation of soil maintained evenly (not cycling) until harvest.

### Scoring

The first day of emergence and final emergence at the true leaf stage were recorded. As follows: by the soy scale every 7 days; wilt score every other day; early pod count at 45 days post planting (average stage of 2-3 pods per plant) with length of each plant's longest pod providing a better predictive measurement than pod length, which was not found to correlate to yield; leaf count at 45 days post planting (found to correlate strongly to yield), yield as measured by final pod count, seed count, and dry seed weight at harvest, nodule count on roots, final dry biomass of plants (separating stems from roots and washing roots), temperature during greenhouse growth periods.

Seedlings were scored as follows:
Final Emergence: seedlings emerged at 12-13 days post planting, out of 3 seeds planted per pot
Pod Count: pods per plant, counted weekly after flowering but before maturity
Seed Pre-Count: seeds per plant, counted inside pods weekly before maturity
Seed Count, Mature: seeds per plant, harvested, mature
Seed Count, Mature+Immature: seeds per plant, harvested, mature and immature
Percent of Seeds That Are Mature: calculated from treatment averages, not per plant
Seed Weight, Mature: dry grams of seed per plant (dried 3 days at 50 degrees C.; mature only)
Wilt Scores: scored visually on a scale from 0=no wilt to 4=unrecoverable;

### Midseason Measurements and Harvest

For soybean, emergence percentage was observed. Further, at various times through the growing season, plants were assessed for pod length, pod number, relative chlorophyll content (SPAD), and total yield as mature seeds produced and seed fresh and dry mass. Soy was harvested at the point of agronomical relevance: senescence of pods.

To compare treated plants to controls, a fully Bayesian robust t-test was performed (Gelman, et al. 2013; Kruschke, 2012). Briefly, R (R Core Team, 2015) was used with the BEST package (Kruschke and Meredith, 2014) and JAGS (Plummer, 2003) to perform a Markov Chain Monte Carlo estimation of the posterior distribution the likely differences between the two experimental groups. A 95% highest density interval (HDI) was overlayed onto this distribution to aid in the interpretation of whether the two biological groups truly differ.

### Results

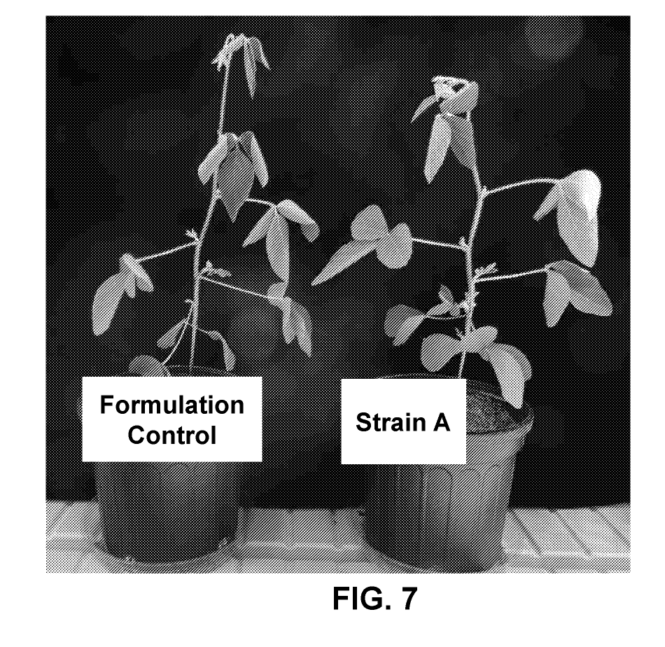

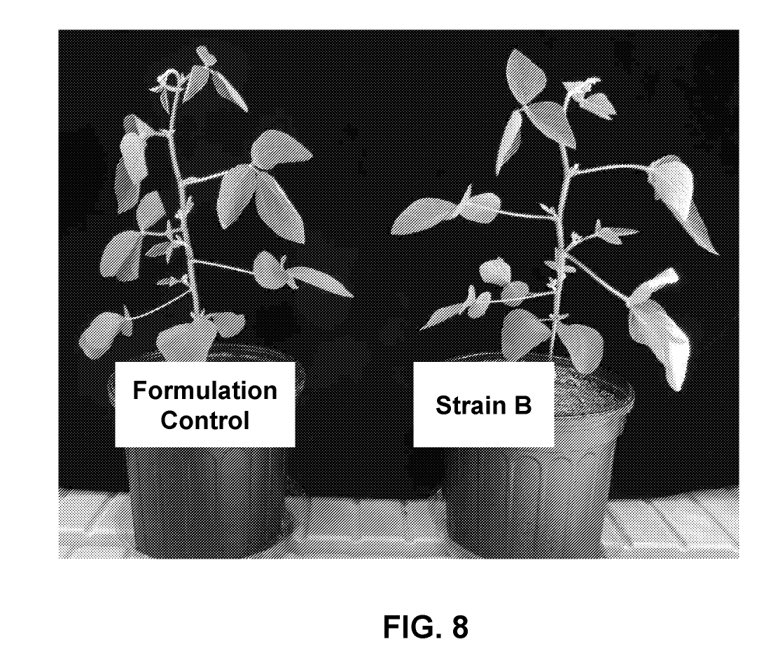

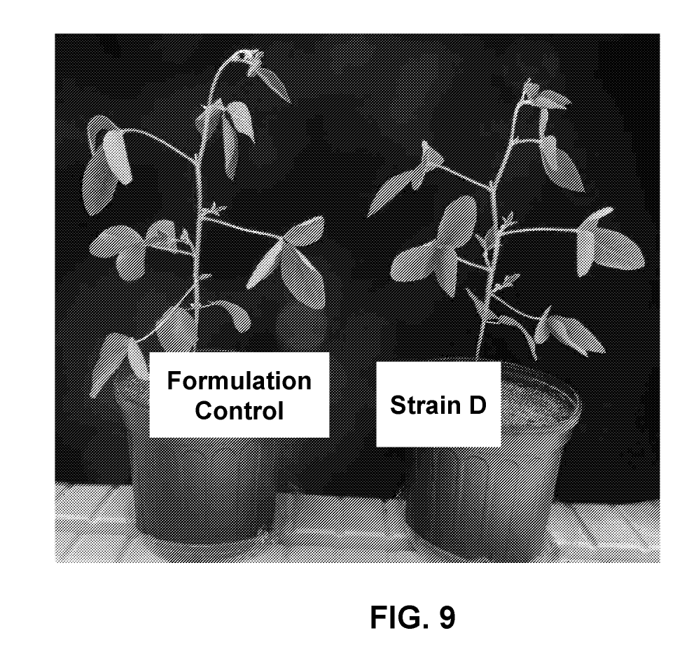

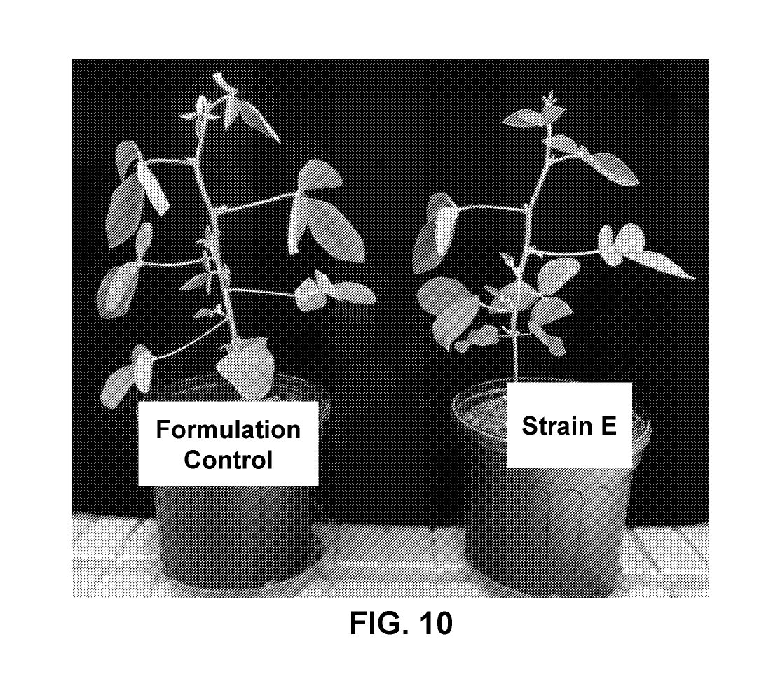

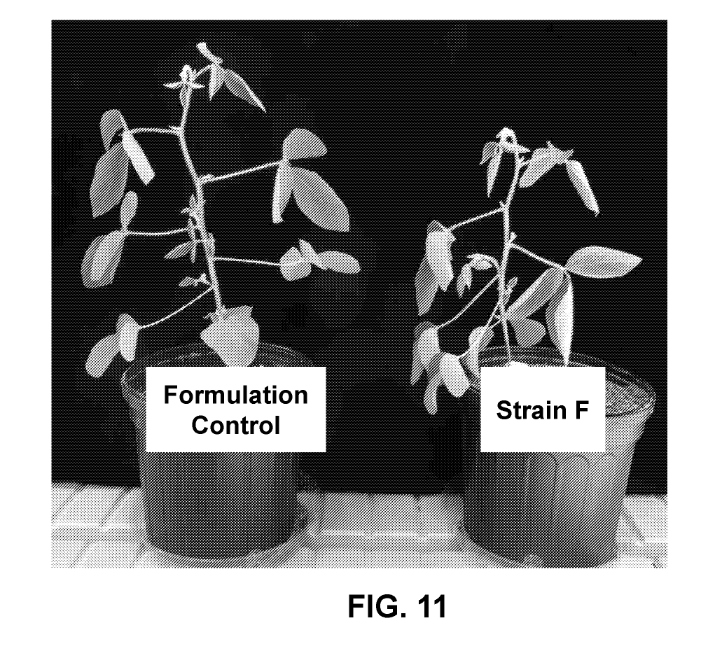

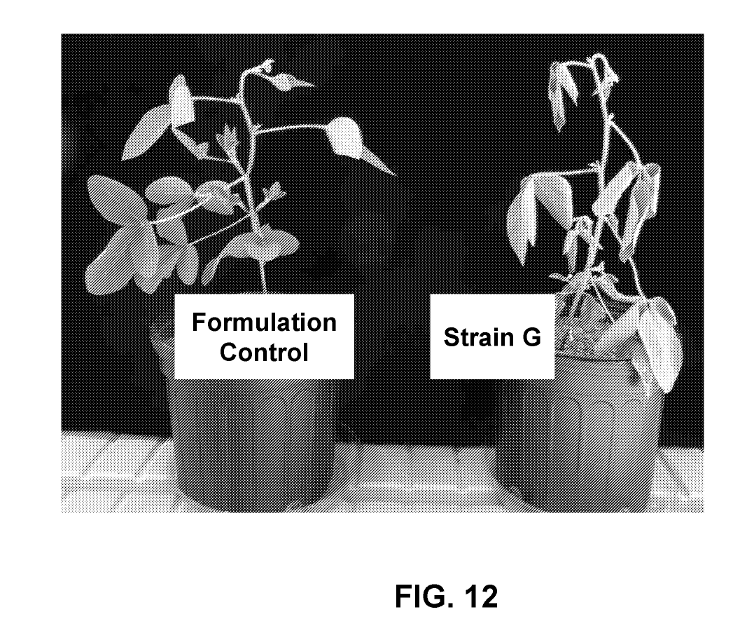

All results are shown in Table 6. Photographs of plants grown under water-limited conditions from seeds treated with the different Penicillium strains, as compared to plants grown from seed treated with the formulation control are shown in FIG. 7 (Strain A), FIG. 8 (Strain B), FIG. 9 (Strain D), FIG. 10 (Strain E), FIG. 11 (Strain F), and FIG. 12 (Strain G).

Plants grown from seeds treated with any of the following strains: Strain A, Strain B, Strain D, or Strain E, displayed the better measurable plant characteristics, including but not limited to better drought tolerance, increased pod counts, and final harvest yield, as compared to the plants grown from seeds treated with the Penicillium strains Strain F or Strain G.

Under normal watering (well watered) conditions as well as under water-limited (drought) conditions, Strain B imparted a number of improved agronomic characteristics to soybean plants grown from seeds that were inoculated with the Strain B formulation, vs. controls of isoline plants grown from seeds not inoculated with the fungal endophyte but instead treated with a formulation control (formulation components minus the fungal endophyte).

### Tissue Collection and Processing for Transcriptomics, Proteomics, Hormone, and Metabolomics Analysis

In order to assess the effects of Penicillium seed treatment on plant growth at the transcriptomic, proteomic, phytohormone, and metabolomic levels, soybean plants were harvested. Three pots from each treatment were selected. Once separated, the tissues (roots, stems, and leaves) from the three pots of each treatment were pooled. For collection, first all loosely attached substrate was removed from the roots by gently tapping and shaking the roots. Any adherent substrate was removed by submerging the roots in water and manually dislodging attached soil and debris. The roots were then blotted dry before being cut from the aerial tissue, followed by separating petioles and leaves from the stem. As tissues were removed from the plant they were immediately bagged and frozen in liquid nitrogen. All harvested tissues were kept in liquid nitrogen or stored at −80° C. until further processing.

To prepare for analyses, the tissues were ground with liquid nitrogen using a pre-chilled mortar and pestle. Approximately 100-200 micrograms of each ground sample pool was transferred to a chilled 1.5 mL microtube for RNA extraction and subsequent transcriptome, phytohormone and metabolite analysis. The remaining ground tissue was then transferred to a chilled 50 mL conical tube and stored in liquid nitrogen or at −80° C. until shipment for further analyses.

Transcriptomics analysis was performed as described in Example 8. Plant proteomics analysis was performed as described in Example 9. Hormone analysis was performed as described in Example 10. Metabolomics was performed as described in Example 11. Community sequencing microbiome profiles were analyzed as described in Example 12.

### Example 7: Assessment of Plant Colonization

The establishment of plant-microbe interactions is contingent on close proximity. The microbiome of the host plant consists of microorganisms inside tissues as well as those living on the surface and surrounding rhizosphere. The protocols described in this section allow confirmation of successful colonization of plants by endophytic fungi, for example by direct recovery of viable colonies from various tissues of the inoculated plant.

### Recovery of Viable Colonies

Seeds are surface-sterilized by exposing them to chlorine gas overnight, using the methods described elsewhere. Sterile seeds are then inoculated with submerged in 0.5 OD overnight cultures of fungi and allowed to briefly air dry. The seeds are then placed in tubes filled partially with a sterile sand-vermiculite mixture [(1:1 wt:wt)] and covered with 1 inch of the mixture, watered with sterile water, sealed and incubated in a greenhouse for 7 days. After incubation, various tissues of the plants are harvested and used as donors to isolate fungi by placing tissue section in a homogenizer (TSB 20%) and mechanical mixing. The slurry is then serially diluted in 10-fold steps to 10-3 and dilutions 1 through 10-3 are plated on TSA 20% plates (1.3% agar). Plates are incubated overnight and pictures are taken of the resulting plates as well as colony counts for CFU. Fungi are identified visually by colony morphotype and molecular methods described herein. Representative colony morphotypes are also used in colony PCR and sequencing for isolate identification via ribosomal gene sequence analysis as described herein. These trials are repeated twice per experiment, with 5 biological samples per treatment.

### Culture-Independent Methods to Confirm Colonization of the Plant or Seeds by Bacteria or Fungi

One way to detect the presence of endophytes on or within plants or seeds is to use quantitative PCR (qPCR). Internal colonization by the endophyte can be demonstrated by using surface-sterilized plant tissue (including seed) to extract total DNA, and isolate-specific fluorescent MGB probes and amplification primers are used in a qPCR reaction. An increase in the product targeted by the reporter probe at each PCR cycle therefore causes a proportional increase in fluorescence due to the breakdown of the probe and release of the reporter. Fluorescence is measured by a quantitative PCR instrument and compared to a standard curve to estimate the number of fungal or bacterial cells within the plant.

### EXPERIMENTAL DESCRIPTION

The design of both species-specific amplification primers, and isolate-specific fluorescent probes are well known in the art. Plant tissues (seeds, stems, leaves, flowers, etc.) are pre-rinsed and surface sterilized using the methods described herein.

Total DNA is extracted using methods known in the art, for example using commercially available Plant-DNA extraction kits, or the following method.

Tissue is placed in a cold-resistant container and 10-50 mL of liquid nitrogen is applied. Tissues are then macerated to a powder.

Genomic DNA is extracted from each tissue preparation, following a chloroform:isoamyl alcohol 24:1 protocol (Sambrook et al., 1989).

Quantitative PCR is performed essentially as described by Gao et al. (2010) with primers and probe(s) specific to the desired isolate using a quantitative PCR instrument, and a standard curve is constructed by using serial dilutions of cloned PCR products corresponding to the specie-specific PCR amplicon produced by the amplification primers. Data are analyzed using instructions from the quantitative PCR instrument's manufacturer software.

As an alternative to qPCR, Terminal Restriction Fragment Length Polymorphism, (TRFLP) can be performed, essentially as described in Johnston-Monje and Raizada (2011). Group specific, fluorescently labelled primers are used to amplify a subset of the microbial population, especially bacteria, especially fungi, especially archaea, especially viruses. This fluorescently labelled PCR product is cut by a restriction enzyme chosen for heterogeneous distribution in the PCR product population. The enzyme cut mixture of fluorescently labelled and unlabeled DNA fragments is then submitted for sequence analysis on a Sanger sequence platform such as the Applied Biosystems 3730 DNA Analyzer.

### Immunological Methods to Detect Microbes in Seeds and Vegetative Tissues

A polyclonal antibody is raised against specific fungal Penicillium strains via standard methods. A polyclonal antibody is also raised against specific GUS and GFP proteins via standard methods. Enzyme-linked immunosorbent assay (ELISA) and immunogold labeling is also conducted via standard methods, briefly outlined below.

Immunofluorescence microscopy procedures involve the use of semi-thin sections of seed or seedling or adult plant tissues transferred to glass objective slides and incubated with blocking buffer (20 mM Tris (hydroxymethyl)-aminomethane hydrochloride (TBS) plus 2% bovine serum albumin, pH 7.4) for 30 min at room temperature. Sections are first coated for 30 min with a solution of primary antibodies and then with a solution of secondary antibodies (goat anti-rabbit antibodies) coupled with fluorescein isothiocyanate (FITC) for 30 min at room temperature. Samples are then kept in the dark to eliminate breakdown of the light-sensitive FITC. After two 5-min washings with sterile potassium phosphate buffer (PB) (pH 7.0) and one with double-distilled water, sections are sealed with mounting buffer (100 mL 0.1 M sodium phosphate buffer (pH 7.6) plus 50 mL double-distilled glycerine) and observed under a light microscope equipped with ultraviolet light and a FITC Texas-red filter.

Ultrathin (50- to 70-nm) sections for TEM microscopy are collected on pioloform-coated nickel grids and are labeled with 15-nm gold-labeled goat anti-rabbit antibody. After being washed, the slides are incubated for 1 h in a 1:50 dilution of 5-nm gold-labeled goat anti-rabbit antibody in IGL buffer. The gold labeling is then visualized for light microscopy using a BioCell silver enhancement kit. Toluidine blue (0.01%) is used to lightly counterstain the gold-labeled sections. In parallel with the sections used for immunogold silver enhancement, serial sections are collected on uncoated slides and stained with 1% toluidine blue. The sections for light microscopy are viewed under an optical microscope, and the ultrathin sections are viewed by TEM.

### Example 8: Identification of Differentially Regulated Genes (Transcriptomics)

### Method: Qualitative Transcriptomics

For the first (qualitative) transcriptomics study, whole RNA was extracted from ground plant tissue over dry ice using the QIAgen Plant RNeasy mini kit (cat. no. 74904) per the manufacturer's instructions with minor modification. DNase treatment was performed on the column with the QIAgen RNase-free DNase kit (cat. no. 79254). The RW1 buffer wash was divided into two washes of half the buffer volume suggested by the manufacturer with the DNase treatment applied in between. After elution, RNA samples were kept on dry ice or at −20° C. until shipping. For transcriptome data acquisition, 1.5 micrograms of whole RNA was sent to Cofactor Genomics (St. Louis, Mo.).

To calculate expression values, transcript cDNA sequences were first aligned to the set of identified genes in the soy genome. Sequence read counts for each sample and gene were next normalized to account for differences in the number of reads per sample and differences in gene lengths. More specifically, raw sequence counts per gene were multiplied by a value representing the mean total number of reads aligned to the gene across all samples divided by the total number of aligned reads for a given sample. This value was then divided by the length of the gene it mapped to in order to eliminate gene length biases. The resulting values were considered to be the expression value.

The resulting expression values and their respective transcripts were filtered to reduce the influence of spurious observations. All observations with expression values lower than 10 were removed from downstream analysis. In addition, transcripts that mapped to genes without function information (i.e. ‘uncharacterized protein’) were not considered further. Fold changes between control and treated samples were calculated for each transcript by dividing the expression value from the treated sample by the expression value from the control sample. Gene ontology terms (functional categories) were determined for each transcript by referencing the Ensembl database (http://ensembl.gramene.org) using their respective genes.

### Method: Quantitative Transcriptomics

The second transcriptomics (quantitative) analyses were conducted on plants grown from seeds treated with a variety of Penicillium strains, and formulation control—treated plants. From this, up- and down-regulated transcripts in plants grown from seeds treated with each of the Penicillium strains were compared with the transcript profiles of plants grown from seeds treated the other strains and with the plants grown from seeds treated with only the formulation control.

The specific procedures used for the transcriptomics comparison analyses included the following parameters: FastQC v0.10.1 was run to verify quality of sequences (fastqc -o <Output directory> -t 4 <Sequence file>). TrimmomaticSE was run to remove TruSeq adapters (TrimmomaticSE -threads 4<Untrimmed filename> <Trimmed filename> ILLUMINACLIP:TruSeq3-SE.fa:2:30:10 LEADING:3 TRAILING:3 SLIDINGWINDOW:4:15 MINLEN:36). Quantification of reads mapped to each locus of the reference genome. The Glycine max Wm82.a2.v1 (Soybean) reference genome was download from Phytozome (phytozome.jgi.doe.gov). Prior to running STAR 2.5.lb_modified, a genome index was generated (STAR—runMode genomeGenerate—runThreadN 8—genomeDir <Output directory>—genomeFastaFiles Gmax_275_v2.0.fa—limitGenomeGenerateRAM 30000000000). Sequences were aligned to the reference genome using STAR 2.5.lb modified (STAR—genomeDir <Genome index directory>—runThreadN 40—readFilesIn <Trimmed seqs directory>—readFilesCommand zcat—outSAMtype BAM SortedByCoordinate—outFilterIntronMotifs RemoveNoncanonicalUnannotated). The .bam file was indexed using Samtools (samtools index <.bam file>). QC was performed on the .bam file using the RSeQC bam_stat.py utility (bam_stat.py -i <.bam file>><Output report file>). Reference genome annotation file Gmax_275_Wm82.a2.v1.gene_exons.gff was converted to a .gtf file containing just exon entries with gene_id parameter specifying the locus without specific transcript designation. This results in all reads mapping to the defined range being reported as part of this gene locus. htseq-count 0.6.1p1 was used to quantify the reads (htseq-count -f bam -s reverse <Mapped file from STAR> <.gtf file>> <counts.txt file>). Quantification of reads mapped to alternatively-spliced transcripts from the reference genome. Salmon 0.6.0 was run in quasi-mapping mode to quantify transcript-specific reads (salmon quant -i transcripts_index −1 SR -r <(gunzip -c <Sequence file>) -o <Quant file>). Differential expression analysis of reads mapped to each locus of the reference genome. Gene locus and transcript counts were run separately. Counts/Quant files for each sample were supplied to DESeq2, which generated log 2FoldChange values for each comparison between a rep and its formulation. Results with an absolute value of log 2FoldChange greater than 1.4 and a padj value less than 0.05 were considered high confidence hits.

To compare these results to qualitative results, the reference genome v2.0 gene was cross-referenced (using the Glyma_11 to Glyma_20 Correspondence_Full.csv file available at Soybase.org) to obtain the reference genome v1.1 gene. If this v1.1 gene was found in the qualitative results output (minus the transcript[.#] specification), the gene was flagged.

### Results: Transcriptomics Qualitative Analysis (Soy Normal Watering and Water-Limited Conditions)

All results are summarized in Table 7A.

The transcriptomic analysis of soybean plants inoculated with endophytic fungal strain Strain B grown under normal watering and water-limited regimes in the greenhouse revealed three major pathways that are modulated by the endophyte: symbiosis enhancement, growth promotion, and resistance against abiotic and biotic stresses.

The following transcripts were modulated in plants grown from seeds treated with Strain B under normal watering or water-limited conditions:

Transcriptomics Upregulated Root, normal watering regime: 18.5 kDa class I heat shock protein, Alcohol-dehydrogenase, Aldehyde dehydrogenase, Amidophosphoribosyltransferase (chloroplastic), Amine oxidase, Arginine decarboxylase, Asparagine synthetase, ATP synthase epsilon chain (chloroplastic), ATP synthase gamma chain, Beta-amyrin 24-hydroxylase, Beta-galactosidase, Calcium-binding EF-hand family protein, Calmodulin-2, Chalcone synthase 7, Chalcone—flavonone isomerase 1A, Chlorophyll a-b binding protein 2 (chloroplastic), Chlorophyll a-b binding protein 3 (chloroplastic), Cytochrome c oxidase subunit 1, DNA-directed RNA polymerase subunit, Early nodulin-36A, Early nodulin-70, Early nodulin-93, Ethylene-responsive element binding factor 4, Eukaryotic translation initiation factor 6, Ferritin, Ferritin-1 (chloroplastic), Flavonoid 4′-O-methyltransferase, Fructose-bisphosphate aldolase, Glutamine synthetase, Glutamine synthetase cytosolic isozyme 2, Glutathione peroxidase, Histone H4, HMG-Y-related protein A, Leghemoglobin A, Leghemoglobin C1, Leghemoglobin C2, Leghemoglobin C3, Lipase, Lipoxygenase, MLO-like protein, NAC domain protein, Nodulin-16, Nodulin-20, Nodulin-21, Nodulin-22, Nodulin-24, Nodulin-26, Nodulin-26B, Nodulin-44, Nodulin-051, Peptidyl-prolyl cis-trans isomerase, Phosphoribulokinase, photosystem I subunit F, Repetitive proline-rich cell wall protein 2, Ribulose bisphosphate carboxylase small chain 1 (chloroplastic), Ribulose bisphosphate carboxylase small chain 4 (chloroplastic), S-adenosylmethionine synthase, Serine hydroxymethyltransferase, Stress-induced protein SAM22, Superoxide dismutase, Thioredoxin.

Transcriptomics Upregulated Stem, normal watering regime: 18.5 kDa class I heat shock protein, 2-hydroxyisoflavanone synthase, Alcohol-dehydrogenase, Annexin, Asparagine synthetase, ATP synthase epsilon chain (chloroplastic), ATP synthase gamma chain, Calmodulin-2, Chalcone synthase 7, Chalcone—flavonone isomerase 1A, Chlorophyll a-b binding protein 2 (chloroplastic), Cytochrome P450 78A3, Cytochrome P450 monooxygenase CYP89H3, DNA-directed RNA polymerase, Early nodulin-36A, Ethylene-responsive element binding factor 4, Eukaryotic translation initiation factor 6, Ferritin, Ferritin-1 (chloroplastic), Ferritin-2 (chloroplastic), Glucose-1-phosphate adenylyltransferase, Glutamine synthetase, Glutathione peroxidase, Lipoxygenase, MLO-like protein, MYB transcription factor MYB187, NADPH—cytochrome P450 reductase, nine-cis-epoxycarotenoid dioxygenase 4, Non-specific lipid-transfer protein, S-adenosylmethionine synthase, Serine hydroxymethyltransferase, Serine/threonine-protein kinase, Thioredoxin, Tubulin beta-1 chain, UDP-glucose 6-dehydrogenase.

Transcriptomics Upregulated Leaf, normal watering regime: 2-hydroxyisoflavanone synthase, Alcohol-dehydrogenase, Amine oxidase, Annexin, Asparagine synthetase, Auxin-induced protein 15A, Beta-amyrin 24-hydroxylase, Beta-galactosidase, Calmodulin-2, Chalcone synthase 7, Chalcone—flavonone isomerase 1A, Cytochrome P450 77A3, Cytochrome P450 78A3, Cytochrome P450 monooxygenase CYP89H3, DNA-directed RNA polymerase, DNA-directed RNA polymerase subunit, Ethylene-responsive element binding factor 4, Eukaryotic translation initiation factor 6, Ferritin, Ferritin-1 (chloroplastic), Ferritin-2 (chloroplastic), Ferritin-4 (chloroplastic), Fructose-bisphosphate aldolase, Glucan endo-1,3-beta-glucosidase, Glutamate receptor, Lipoxygenase, Malic enzyme, MLO-like protein, Monosaccharide transporter, MYB transcription factor MYB187, NADPH—cytochrome P450 reductase, Non-specific lipid-transfer protein, Phospholipase D, Protein PsbN, Repetitive proline-rich cell wall protein 2, Repetitive proline-rich cell wall protein 3, S-adenosylmethionine synthase, Serine hydroxymethyltransferase, Signal recognition particle 9 kDa protein, Stress-induced protein SAM22, Superoxide dismutase, Thioredoxin, Tubulin beta-1 chain, Wound-induced protein.

Transcriptomics Downregulated Root, normal watering regime: 2-hydroxyisoflavanone synthase, 50S ribosomal protein L35, Amine oxidase, Annexin, Asparagine synthetase, Beta-galactosidase, Calmodulin-2, Cytochrome P450 78A3, Cytochrome P450 82A4, Cytochrome P450 93A3, DNA-directed RNA polymerase, Expansin, Ferritin-4 (chloroplastic), G2/mitotic-specific cyclin S13-6, G2/mitotic-specific cyclin S13-7, Histone H2A, Histone H2B, Histone H3, Histone H4, Lipase, Lipoxygenase, Malic enzyme, MLO-like protein, Monosaccharide transporter, NADPH—cytochrome P450 reductase, Non-specific lipid-transfer protein, Peptidyl-prolyl cis-trans isomerase, Peroxidase, Phospholipase D, Phosphomannomutase, Protein P21, Repetitive proline-rich cell wall protein 3, S-adenosylmethionine synthase, Serine hydroxymethyltransferase, Signal recognition particle 9 kDa protein, Stem 28 kDa glycoprotein, Stem 31 kDa glycoprotein, Superoxide dismutase, Thioredoxin, Tubulin beta-1 chain, UDP-glucose 6-dehydrogenase.

Transcriptomics Downregulated Stem, normal watering regime: 50S ribosomal protein L35, Aldehyde dehydrogenase, Amine oxidase, Annexin, Arginine decarboxylase, Asparagine synthetase, Auxin-induced protein 15A, Auxin-induced protein 6B, Auxin-induced protein X15, beta glucosidase 42, Beta-amyrin 24-hydroxylase, Beta-galactosidase, Calcium-binding EF-hand family protein, Chlorophyll a-b binding protein 2 (chloroplastic), Chlorophyll a-b binding protein 3 (chloroplastic), Cytochrome P450 77A3, DNA-directed RNA polymerase subunit, Ferritin, Ferritin-4 (chloroplastic), Fructose-bisphosphate aldolase, G2/mitotic-specific cyclin S13-6, G2/mitotic-specific cyclin S13-7, Histone H2A, Histone H2B, Histone H3, Histone H4, HMG-Y-related protein A, Lipase, Lipoxygenase, Malic enzyme, Monosaccharide transporter, Non-specific lipid-transfer protein, Pectinesterase, Peptidyl-prolyl cis-trans isomerase, Peptidyl-prolyl cis-trans isomerase 1, Peroxidase, Phosphomannomutase, Phosphoribulokinase, photosystem I subunit F, Repetitive proline-rich cell wall protein 2, Repetitive proline-rich cell wall protein 3, Ribulose bisphosphate carboxylase small chain 1 (chloroplastic), Ribulose bisphosphate carboxylase small chain 4 (chloroplastic), RuBisCO-associated protein, S-adenosylmethionine synthase, Stem 31 kDa glycoprotein, Stress-induced protein SAM22, Superoxide dismutase, Thioredoxin, Uracil-DNA glycosylase.

Transcriptomics Downregulated Leaf, normal watering regime: 18.5 kDa class I heat shock protein, 50S ribosomal protein L35, Aldehyde dehydrogenase, Arginine decarboxylase, Asparagine synthetase, ATP synthase epsilon chain (chloroplastic), ATP synthase gamma chain, Auxin-induced protein 15A, Auxin-induced protein 6B, Auxin-induced protein X15, beta glucosidase 42, Beta-galactosidase, Calcium-binding EF-hand family protein, Calmodulin-2, Chlorophyll a-b binding protein 2 (chloroplastic), Chlorophyll a-b binding protein 3 (chloroplastic), Early nodulin-36A, Fructose-bisphosphate aldolase, G2/mitotic-specific cyclin S13-7, Glutamine synthetase, Glutathione peroxidase, Histone H2A, Histone H2B, Histone H3, Histone H4, HMG-Y-related protein A, Lipoxygenase, nine-ci s-epoxycarotenoid dioxygenase 4, Non-specific lipid-transfer protein, Pectinesterase, Peptidyl-prolyl cis-trans isomerase, Peptidyl-prolyl cis-trans isomerase 1, Phosphomannomutase, Phosphoribulokinase, photosystem I subunit F, Protein P21, Ribulose bisphosphate carboxylase small chain, Ribulose bisphosphate carboxylase small chain 1 (chloroplastic), Ribulose bisphosphate carboxylase small chain 4 (chloroplastic), RuBisCO-associated protein, S-adenosylmethionine synthase, Serine hydroxymethyltransferase, Serine/threonine-protein kinase, Stem 31 kDa glycoprotein, Thioredoxin, UDP-glucose 6-dehydrogenase, Uracil-DNA glycosylase.

Transcriptomics Upregulated Root, water-limited watering regime: 50S ribosomal protein L35, Alcohol-dehydrogenase, Aldehyde dehydrogenase, Amine oxidase, Annexin, Asparagine synthetase, ATP synthase gamma chain, beta glucosidase 42, Beta-amyrin 24-hydroxylase, Beta-galactosidase, Calcium-binding EF-hand family protein, Calmodulin-2, Chlorophyll a-b binding protein 2 (chloroplastic), Chlorophyll a-b binding protein 3 (chloroplastic), Cytochrome P450 78A3, Cytochrome P450 93A3, Early nodulin-36A, Ethylene-responsive element binding factor 4, Flavonoid 4′-O-methyltransferase, Fructose-bisphosphate aldolase, Glutathione peroxidase, Histone H2A, Histone H2B, Histone H3, Histone H4, Lipase, Lipoxygenase, MLO-like protein, Monosaccharide transporter, NAC domain protein, nine-cis-epoxycarotenoid dioxygenase 4, Nodulin-26, Non-specific lipid-transfer protein, Pectinesterase, Peptidyl-prolyl cis-trans isomerase, Peptidyl-prolyl cis-trans isomerase 1, Peroxidase, Phospholipase D, Phosphoribulokinase, photosystem I subunit F, Repetitive proline-rich cell wall protein 2, Repetitive proline-rich cell wall protein 3, Ribulose bisphosphate carboxylase small chain 1 (chloroplastic), Ribulose bisphosphate carboxylase small chain 4 (chloroplastic), Serine hydroxymethyltransferase, Serine/threonine-protein kinase, Stress-induced protein SAM22, Superoxide dismutase, Thioredoxin, Tubulin beta-1 chain, UDP-glucose 6-dehydrogenase.

Transcriptomics Upregulated Stem, water-limited watering regime: 50S ribosomal protein L35, Alcohol-dehydrogenase, Aldehyde dehydrogenase, Amine oxidase, Annexin, Arginase, Arginine decarboxylase, ATP synthase epsilon chain (chloroplastic), Auxin-induced protein 15A, Auxin-induced protein 6B, Auxin-induced protein X15, beta glucosidase 42, Beta-amyrin 24-hydroxylase, Calcium-binding EF-hand family protein, Calmodulin-2, Chalcone—flavonone isomerase 1A, Chlorophyll a-b binding protein 2 (chloroplastic), Chlorophyll a-b binding protein 3 (chloroplastic), Cytochrome P450 78A3, Cytochrome P450 monooxygenase CYP89H3, DNA-directed RNA polymerase subunit, Early nodulin-36A, Ethylene-responsive element binding factor 4, Ferritin, Ferritin-1 (chloroplastic), Ferritin-2 (chloroplastic), Ferritin-4 (chloroplastic), Fructose-bisphosphate aldolase, G2/mitotic-specific cyclin 513-6, G2/mitotic-specific cyclin 513-7, Glutathione peroxidase, Histone H2A, Histone H2B, Histone H3, Histone H4, HMG-Y-related protein A, Malic enzyme, Monosaccharide transporter, Non-specific lipid-transfer protein, Peptidyl-prolyl cis-trans isomerase, Peptidyl-prolyl cis-trans isomerase 1, Peroxidase, Phenylalanine ammonia-lyase, photosystem I subunit F,Repetitive proline-rich cell wall protein 2, Ribulose bisphosphate carboxylase small chain 1 (chloroplastic), Ribulose bisphosphate carboxylase small chain 4 (chloroplastic), S-adenosylmethionine synthase, Signal recognition particle 9 kDa protein, Superoxide dismutase, Thioredoxin, Tubulin beta-1 chain, UDP-glucose 6-dehydrogenase, Uracil-DNA glycosylase.

Transcriptomics Upregulated Leaf, water-limited watering regime: 2-hydroxyisoflavanone synthase, 3-ketoacyl-CoA synthase, 50S ribosomal protein L35, Alcohol-dehydrogenase, Aldehyde dehydrogenase, Amine oxidase, Annexin, Arginase, Asparagine synthetase, beta glucosidase 42,Beta-amyrin 24-hydroxylase, Calcium-binding EF-hand family protein, Calmodulin-2, CASP-like protein 10, Chalcone synthase 7, Chalcone—flavonone isomerase 1A, Chlorophyll a-b binding protein 2 (chloroplastic), Chlorophyll a-b binding protein 3 (chloroplastic), Cytochrome P450 77A3, DNA-directed RNA polymerase subunit, Ethylene-responsive element binding factor 4, Eukaryotic translation initiation factor 6, Ferritin-1 (chloroplastic), Flavonoid 4′-O-methyltransferase, Fructose-bisphosphate aldolase, Glucan endo-1,3-beta-glucosidase, Glutathione peroxidase, Histone H2A, Histone H2B, Histone H3, Histone H4, HMG-Y-related protein A, Lipoxygenase, Malic enzyme, MLO-like protein, Monosaccharide transporter, MYB transcription factor MYB187, NAC domain protein, Non-specific lipid-transfer protein, Peptidyl-prolyl cis-trans isomerase, Phospholipase D, Phosphomannomutase, Phosphoribulokinase, Ribulose bisphosphate carboxylase small chain, Ribulose bisphosphate carboxylase small chain 1 (chloroplastic), Ribulose bisphosphate carboxylase small chain 4 (chloroplastic), S-adenosylmethionine synthase, Serine/threonine-protein kinase, Signal recognition particle 9 kDa protein, Stress-induced protein SAM22, Superoxide dismutase, Superoxide dismutase, Thioredoxin, Tubulin beta-1 chain, UDP-glucose 6-dehydrogenase, Uracil-DNA glycosylase.

Transcriptomics Downregulated Root, water-limited watering regime: 18.5 kDa class I heat shock protein, 2-hydroxyisoflavanone synthase, Arginine decarboxylase, Asparagine synthetase, ATP synthase epsilon chain (chloroplastic), Chalcone synthase 7, Chalcone—flavonone isomerase 1A, Cytochrome P450 82A4, DNA-directed RNA polymerase, DNA-directed RNA polymerase subunit, Eukaryotic translation initiation factor 6, Ferritin, Ferritin-1 (chloroplastic), Ferritin-2 (chloroplastic), Ferritin-4 (chloroplastic), G2/mitotic-specific cyclin S13-6, G2/mitotic-specific cyclin S13-7, Glutathione peroxidase, Histone H2A, Histone H2B, Histone H3, Histone H4, HMG-Y-related protein A, Lipase, Lipoxygenase, Malic enzyme, MLO-like protein, NADPH—cytochrome P450 reductase, Nodulin-16, Nodulin-20, Nodulin-21, Nodulin-22, Nodulin-24, Nodulin-44, Nodulin-C51, Peptidyl-prolyl cis-trans isomerase, Phosphomannomutase, S-adenosylmethionine synthase, Signal recognition particle 9 kDa protein, Superoxide dismutase, Superoxide dismutase, Thioredoxin.

Transcriptomics Downregulated Stem, water-limited watering regime: 18.5 kDa class I heat shock protein, 2-hydroxyisoflavanone synthase, 3-ketoacyl-CoA synthase, Aldehyde dehydrogenase, Amine oxidase, Annexin, Apocytochrome f, Asparagine synthetase, ATP synthase gamma chain, Beta-galactosidase, Chalcone synthase 7, Cytochrome P450 77A3, DNA-directed RNA polymerase, Eukaryotic translation initiation factor 6, Fructose-bisphosphate aldolase, Glutamine synthetase, Glutathione peroxidase, Lipoxygenase, MLO-like protein, MYB transcription factor MYB187, NADPH—cytochrome P450 reductase, nine-cis-epoxycarotenoid dioxygenase, Non-specific lipid-transfer protein, Pectinesterase, Peptidyl-prolyl cis-trans isomerase, Phosphomannomutase, Phosphoribulokinase, Photosystem II reaction center protein J, Photosystem II reaction center protein Z, Repetitive proline-rich cell wall protein 3, RuBisCO-associated protein, S-adenosylmethionine synthase, Serine hydroxymethyltransferase, Serine/threonine-protein kinase, Stress-induced protein SAM22, Superoxide dismutase, Thioredoxin.

Transcriptomics Downregulated Leaf water-limited watering regime: 18.5 kDa class I heat shock protein, Acetolactate synthase, Aldehyde dehydrogenase, Apocytochrome f, Arginine decarboxylase, Asparagine synthetase, ATP synthase epsilon chain (chloroplastic), ATP synthase gamma chain, Auxin-induced protein 15A, Auxin-induced protein 6B, Beta-galactosidase, Cytochrome b6, Cytochrome P450 78A3, Cytochrome P450 monooxygenase CYP89H3, DNA-directed RNA polymerase, Early nodulin-36A, Ferritin, Ferritin-2 (chloroplastic), Ferritin-4 (chloroplastic), Fructose-bisphosphate aldolase, Glutamate receptor, Glutamine synthetase, Lipoxygenase, MLO-like protein, NADPH—cytochrome P450 reductase, nine-cis-epoxycarotenoid dioxygenase 4,Nitrate reductase, Non-specific lipid-transfer protein, Peptidyl-prolyl cis-trans isomerase, Peptidyl-prolyl cis-trans isomerase 1, photosystem I subunit F,Photosystem II reaction center protein J, Repetitive proline-rich cell wall protein 2, Ribose-phosphate pyrophosphokinase, RuBisCO-associated protein, S-adenosylmethionine synthase, Serine hydroxymethyltransferase, Site-determining protein, Thioredoxin.

### Symbiosis Enhancement

### Nodulation

Nodulin-encoding genes are specifically expressed during the development of symbiotic root nodules (Legocki and Verma, 1980). Upon nodule formation bacteria differentiate into nitrogen-fixing bacteroids that are beneficial to the plants (Kereszt et al., 2011). Nodulin proteins serve transport and regulatory functions in symbiosis (Fortin et al., 1985). Under normal watering, nodulins 16, 20, 21, 22, 24, 26, 26B, 44, and C51, and early nodulins 36A, 70, and 93 were upregulated in roots. Under drought conditions, nodulins 16, 20, 22, 24, 44, and C51 were downregulated in roots.

Leghemoglobin biosynthesis genes are involved in nodulation, specifically in providing oxygen flux to the respiring bacteroids (Appleby, 1984). Under normal watering, leghemoglobins A, C1, C2, and C3 were upregulated in roots. Under drought conditions, leghemoglobins A, C1, C2, and C3 were downregulated in roots.

Flavonoids are secondary metabolites that have many functions in higher plants, including UV protection, fertility, antifungal defense and the recruitment of nitrogen-fixing bacteria (Dao et al., 2011). The chalcone precursor of the flavonoids is synthesized by chalcone synthase, and isomerized to yield a flavanone by chalcone flavanone isomerase, followed by further enzymatic modifications (Dao et al., 2011). Under normal watering, chalcone synthase 7 (CHS7) and 2-hydroxyisoflavanone synthase (IFS2) were upregulated in leaves, and flavonoid 4′-O-methyltransferase was upregulated in roots. Under drought conditions, chalcone-flavanone isomerase 1A (CHI-1A) and flavonoid 4′-O-methyltransferase were upregulated in leaves.

Chloroplastic amidophosphoribosyltransferase is the first enzyme in de novo purine biosynthesis (Ito et al., 1994). It is associated with maturation of nodules in soybean and moth-bean (Vigna aconitifolia) (Kim et al., 1995). Under normal watering, chloroplastic amidophosphoribosyltransferase (PURI) was upregulated in roots.

Malic enzyme has been shown to be important for carbon metabolism of bacteroids and free living bacteria by supplying acetyl-CoA for the TCA cycle or providing NADPH and pyruvate for various biosynthetic pathways (Dao et al., 2008). Soybean plants inoculated with a NAD(+)-dependent malic enzyme mutant formed small root nodules and exhibited significant nitrogen-deficiency symptoms (Dao et al., 2008). Under normal watering, malic enzyme was upregulated in leaves.

### Nitrogen Metabolism

In most legumes, asparagine is the principal assimilation product of symbiotic nitrogen fixation (Scott et al., 1976). In soybean, high asparagine synthetase transcript level in source leaves is positively correlated with protein concentration of seed (Wan et al., 2006), and in roots, is linked with increased levels of asparagine in xylem sap transported to the shoot (Antunes et al., 2008). Under normal watering, two asparagine synthetase transcripts were upregulated in stems and leaves. In a comparison of drought and normal watering, three other asparagine synthetase transcripts were more upregulated by Strain B in stems under drought than under normal watering.

Glutamine and glutamate synthetases are enzymes responsible for assimilation of fixed ammonia during nitrogen fixation (Lara et al., 1983). In common bean (Phaseolus vulgaris) and in soy, nodule-specific forms of glutamine synthetase are produced in rhizobia-colonized nodules (Lara et al., 1983; Sengupta-Gopalan and Pitas, 1986), highlighting the enzyme's role in symbiosis. Under normal watering, glutamine synthetase and glutamine synthetase cytosolic isozyme 2 (GSGME) were upregulated in roots.

### Growth Promotion

### Carbon Metabolism and Photosynthesis

S-adenosylmethionine synthase, which catalyzes synthesis of s-adenosylmethionine from methionine and ATP, functions as a primary methyl-group donor and as a precursor for metabolites such as ethylene, polyamines, and vitamin B1 (Hesse et al., 2004). Under normal watering, one S-adenosylmethionine synthase transcript was upregulated in roots. Under drought conditions, a second S-adenosylmethionine synthase transcript was upregulated in leaves. In a comparison of drought and normal watering, this second transcript was more upregulated by Strain B in leaves under drought than under normal watering. Under normal watering, S-adenosylmethionine synthase was downregulated in roots.

Beta-galactosidase is a key enzyme in carbohydrate metabolism (Seki et al., 2002). Under normal watering, beta-galactosidase was upregulated in roots. In a comparison of drought and normal watering, four other beta-galactosidase transcripts were more upregulated by Strain B in roots under drought than under normal watering.

Phosphorylase, an enzyme that catalyzes the addition of a phosphate group from an inorganic phosphate to an acceptor, is an important allosteric enzyme in carbohydrate metabolism (Madsen, N. B, 1986). Under normal watering, two transcripts for phosphorylase were upregulated in stems.

Fructose-bisphosphate aldolase is a glycolytic enzyme, induced by the plant hormone gibberellin, that may regulate the vacuolar H-ATPase-mediated control of cell elongation that determines root length (Konishi et al., 2005). Four fructose-bisphosphate aldolase transcripts were notably upregulated in a tissue- and condition-specific manner. Under normal watering, two fructose-bisphosphate aldolase transcripts were upregulated in roots. Under drought conditions, these two transcripts and one additional fructose-bisphosphate aldolase transcript were upregulated in roots. In a comparison of drought and normal watering, all three of these transcripts were more upregulated by Strain B in roots and a fourth fructose-bisphosphate aldolase transcripts was more upregulated by Strain B in leaves under drought than under normal watering.

Glucose-1-phosphate adenylyltransferase, a transferase that transfers phosphorous-containing nucleotide groups, is involved in starch and sucrose metabolism (Ghosh and Preiss, 1966). Five transcripts encoding for glucose-1-phosphate adenylyltransferase were highly upregulated in stems of Strain B-treated plants grown under normal watering regime.

A hallmark of vascular plants is that photosynthetic, green source tissues produce sugars and assimilate inorganic nitrogen, and transport an excess to non-photosynthetic sink tissues such as roots and reproductive organs (Giaquinta, 1983). Several sugar transporters were identified and characterized in plants (Sauer and Tanner, 1993). Monosaccharide transporters are primarily transcribed in sink tissues such as young leaves, storage tissues, and floral organs (Sauer and Tanner, 1993). The monosaccharide transporters typically function as high affinity monosaccharide proton co-transporters in plants, since they transport pentoses and various hexoses (Buttner, 2010). Under drought conditions, monosaccharide transporter 1 (MST1) was upregulated in leaves.

Phosphoribulokinase catalyzes the ATP-dependent phosphorylation of D-ribulose 5-phosphate to form D-ribulose 1,5-bisphosphate, the photosynthetic CO2 acceptor. In higher plants this enzyme is one of the target enzymes of the ferredoxin/thioredoxin system (Buchanan, 1980). Under drought conditions, two transcripts for phosphoribulokinase were upregulated in roots. In a comparison of drought and normal watering, these transcripts were more upregulated by Strain B in roots under drought than under normal watering.

In plants, serine hydroxymethyltransferase is a key respiratory enzyme in the mitochondria, converting glycine to serine (Ho and Saito, 2001). As glycine is a photorespiration product in C3 plants, serine hydroxymethyltransferase participates in the photorespiration pathway in the leaves (McClung et al., 2000; Prabhu et al., 1998), where it may contribute to resistance to biotic and abiotic stress (Moreno et al., 2005). Five serine hydroxymethyltransferase transcripts were notably upregulated in a tissue- and condition-specific manner. Under normal watering, one serine hydroxymethyltransferase transcript was upregulated in both stems and leaves, and another in leaves only. Under drought conditions, two other serine hydroxymethyltransferase transcripts were upregulated in roots. In a comparison of drought and normal watering, one of these latter was more upregulated by Strain B in roots and an additional fifth serine hydroxymethyltransferase transcript was more upregulated by Strain B in leaves under drought than under normal watering.

ATP synthase provides energy to the cell through the synthesis of adenosine triphosphate (ATP) from adenosine diphosphate (ADP) and inorganic phosphate (Pi). The ATP produced by the light reactions is then used by the dark reactions of photosynthesis to reduce CO2 to carbohydrates (McCarty, 1992). Changes in ATP synthase contents have been reported in response to changes in light intensity (Anderson et al., 1988), leaf age (Schottler et al., 2007), and drought stress (Kohzuma et al., 2009). Under drought conditions, ATP synthase gamma chain (ATPC) was upregulated in roots. In a comparison of drought and normal watering, this transcript was more upregulated by Strain B in roots under drought than under normal watering.

The chloroplastic ATP synthase epsilon chain generates ATP from ADP and inorganic phosphate using energy derived from a trans-thylakoidal electrochemical proton gradient (Cruz et al., 1995). Under normal watering, chloroplastic ATP synthase epsilon chain (ATPE) was upregulated in roots.

Chlorophyll a-b binding proteins (CABs) are protective components of the photosynthetic light harvesting system known to participate in drought responses (Fang and Xiong, 2014). Under both normal and drought conditions, chloroplastic chlorophyll a-b binding proteins 2 (CAB2/LHCB1-7) and 3 (CAB3) were upregulated in roots. In a comparison of drought and normal watering, both CAB transcripts were more upregulated by Strain B in roots and CAB2 was also more upregulated by Strain B in leaves under drought than under normal watering.

Photosystem I subunit F (PSAF) promotes efficiency of electron transfer from plastocyanin to P700 (Haldrup et al., 2000) and has been shown to be upregulated in response to cold stress (Batista-Santos et al., 2011). Under drought conditions, photosystem I subunit F (PSAF) was upregulated in roots. In a comparison of drought and normal watering, this transcript was more upregulated by Strain B in roots under drought than under normal watering.

The photosystem II complex initiates photosynthesis by catalyzing electron transfer from water to the electron transport chain (Suorsa et al., 2004). The low molecular weight photosystem II-associated trans-membrane protein N participates in assembly and photoinhibition repair of the photosystem II reaction center (Torabi et al., 2014). Under normal watering, photosystem II protein N (PsbN) was upregulated in leaves. Under drought conditions, the photosystem II reaction center protein J (PsbJ) was downregulated in stems and leaves, and photosystem II reaction center protein Z (PsbZ) was downregulated in stems.

The cytochrome b6f complex, highly conserved across plants, algae, and cyanobacteria (Sainz et al., 2000), is key to the electron transfer chain of photosynthesis (Allen, 2004). Under drought conditions, apocytochrome f, the precursor of cytochrome prior to heme binding (Kuras et al., 1995) and cytochrome b6 were downregulated in stems and leaves.

A non-enzymatic, narbonin-like RuBisCO complex protein (RCP) was reported to accumulate in leaves following pod removal, but there is no evidence that it shares narbonin's role in storage and its function remains unknown (Mahato et al., 2004; Staswick, 1997; Staswick et al., 1994). Under normal watering, this RuBisCO-associated protein was downregulated in stems.

Cytochrome c oxidase is a multimeric complex composed of several different subunits (Capaldi, 1990). Subunits I, II and III are encoded by the mitochondrial genome (Unseld et al., 1997), while the other subunits are encoded in the nuclear genome and imported into the mitochondria post-translationally (Newton, 1988). Cytochrome c oxidase complex catalyzes the transfer of electrons from cytochrome c to molecular oxygen (Michel et al., 1998). It has been shown that the genetic components of the cytochrome c-dependent pathway are similarly regulated by carbon and nitrogen sources (Curi et al., 2003) and that their expression can be tissue-specific (Smart et al., 1994; Ribichich et al., 2001). The mitochondrial gene for subunit I (cox1) from soybean has been previously isolated (Grabau, 1986). Our data demonstrates that under normal watering, cytochrome c oxidase subunit 1 (COX1) was upregulated in roots.

Ribulose-1,5-bisphosphate carboxylase catalyzes the carboxylation and hydrolytic cleavage of ribulose-1,5-bisphosphate, the primary event in carbon fixation. In vascular plants, the small subunit of ribulose-1,5-bisphosphate carboxylate (RuBPCss) is encoded by the nuclear genome while the large subunit is encoded in the chloroplast genome (Kawashima and Wildman, 1972). Ribulose bisphosphate carboxylase small chain 3A has been reported to contribute to rapid increases in the amount of cytoplasmic soluble sugars present at the early stage of cold exposure (Grimaud et al., 2013). Under both normal and drought conditions, chloroplastic ribulose bisphosphate carboxylase small chains 1 and 4 were upregulated in roots. In a comparison of drought and normal watering, these transcripts were more upregulated by Strain B in roots, and these transcripts and an additional ribulose bisphosphate carboxylase small chain transcript were more upregulated by Strain B in leaves under drought than under normal watering.

Chloroplastic 50S ribosomal proteins are subunits forming the ribosome, involved in synthesis of organelle-specific proteins in the chloroplast (Bartsch et al., 1982). In a comparison of drought and normal watering, 50S ribosomal protein L35 was more upregulated by Strain B in stems under drought than under normal watering.

### Cell Wall

Amine oxidase generates hydrogen peroxide that is important for lignification of cortical cell wall and xylem tissue under both stress and normal conditions (Angelini et al., 1993). Four amine oxidase transcripts were notably upregulated in a tissue- and condition-specific manner. Under normal watering, one anime oxidase transcript was upregulated in roots. Under both normal watering and drought conditions, a second amine oxidase transcript was upregulated in leaves. In a comparison of drought and normal watering, this second amine oxidase transcript was more upregulated by Strain B in leaves, a third transcript in stems, and a fourth in roots, under drought than under normal watering. Under drought conditions, amine oxidase was downregulated in stems.

Pectinesterase is an enzyme involved in cell wall modification during growth, biotic stress, and nodule formation (Caffall and Mohnen, 2009; Carvalho et al., 2013; Zhang et al., 2015). Under drought conditions, pectinesterase was upregulated in roots.

In plants, peroxidases are involved in cell wall lignification, usually associated with pathogen resistance (Bruce and West, 1989), abiotic stress (Huttova et al., 2006; Quiroga et al., 2001), or cell wall modification during growth (Van Hoof and Gaspar, 1976; Kukavica et al., 2012). In a comparison of drought and normal watering, peroxidase (GM-IPER1) was more upregulated by Strain B in stems under drought than under normal watering.

UDP-glucose 6-dehydrogenase is an enzyme that participates in cell wall formation and modification by providing UDP-glucuronate for polysaccharide biosynthesis (Cook et al., 2012). In a comparison of drought and normal watering, UDP-glucose 6-dehydrogenase was more upregulated by Strain B in roots under drought than under normal watering.

There is an evidence to suggest that CASP-like proteins build transmembrane scaffolds for localization of proteins and modification of the cell wall, important in directing the growth of some specialized tissues (Roppolo et al., 2014). In Arabidopsis, CASP-like proteins demonstrated tissue-specific expression in trichome cells, abscission zone cells, root cap cells, and xylem pole pericycle cells (Roppolo et al., 2014). Under drought conditions, CASP-like protein 10 was upregulated in leaves.

Expansin acts during growth to loosen the cell wall polysaccharide network (Cosgrove, 2000), permitting growth but increasing vulnerability to pathogen infection (Ding et al., 2008). Under normal watering, expansin was downregulated in roots.

### Development and Regulation

DNA-directed RNA polymerase is responsible for transcription of DNA sequences to mRNA transcripts (Azuma et al., 1991; Kollmar and Farnham, 1993). Under drought conditions, a DNA-directed RNA polymerase subunit was upregulated in leaves. Under drought conditions, a DNA-directed RNA polymerase was downregulated in leaves.

Eukaryotic initiation factor 6 is a regulator of ribosome biogenesis and protein translation (Guo et al., 2013). Recent studies have shown that this gene is predominately expressed in meristem and lateral roots, and it may play a critical role in growth and development through involvement in ribosome biogenesis in these tissues (Kato et al., 2010). Under normal watering, eukaryotic translation initiation factor 6 was upregulated in stems. G2/mitotic-specific cyclin S13 is essential for the control of the cell cycle at the G2/M (mitosis) transition (Hata et al., 1991). G2/mitotic-specific cyclin S13-6 was found to be upregulated in response to high temperature and humidity stress during soybean seed development (Wang et al., 2012). In a comparison of drought and normal watering, G2/mitotic-specific cyclin S13-6 and -7 were more upregulated by Strain B in stems under drought than under normal watering.

Histones are primarily involved in DNA packaging into chromatin, a process that modifies gene expression. Recent studies show that the developmental transition from a vegetative to a reproductive phase (i.e. flowering) is controlled by chromatin modifications (He, 2009). Under drought conditions, 3 histone H2A transcripts, 4 histone H3 transcripts, and 1 histone H4 transcript were upregulated in stems and 3 histone H2A transcripts, 2 histone H2B transcripts, 7 histone H3 transcripts, and 6 histone H4 transcripts were upregulated in leaves. In a comparison of drought and normal watering, all of these and an additional 55 histone transcripts were more upregulated by Strain B in stems and leaves under drought than under normal watering.

An HMG-Y-related protein A gene, related to the “high mobility group” (HMG) chromatin proteins involved in gene regulation via recognition and modulation of both DNA and chromatin structure (Bustin and Reeves, 1996), has been isolated in soy (Laux et al., 1991). The presence of both a HMG-Y-like DNA binding domain and a histone H1 domain suggests that it may interact with histones in chromatin (Laux et al., 1991). In a comparison of drought and normal watering, HMG-Y-related protein A was more upregulated by Strain B in stems and leaves under drought than under normal watering.

Uracil-DNA glycosylase, an enzyme present across eukaryotes, participates in DNA repair, specifically by excising uracil bases incorporated mistakenly during replication or due to damage (Cordoba-Cañero et al., 2010). In a comparison of drought and normal watering, uracil-DNA glycosylase was more upregulated by Strain B in stems under drought than under normal watering.

In higher plants, signal-recognition-particle (SRP) assembly has a crucial role in targeting chloroplast nuclear- and plastome-encoded proteins toward the proper cellular compartment in chloroplast (Halic et al., 2004; Ferro et al., 2010). In eukaryotes, the SRP contains six proteins (SRP9, SRP14, SRP19, SRP54, SRP68, SRP72) (ZWIEB et al., 2005). Our data shows that under drought conditions, signal recognition particle 9 kDa protein SRP9 was upregulated in leaves.

Auxin directs cell elongation by stimulating cell wall-loosening factors (Friml, 2003). This is consistent with reports that increased seed germination, shoot growth and seed production have been accompanied by increased production of auxin-like compounds (Friml, 2003). In addition, Nod-factor independent nodulation is mediated in legumes through control of development of nodule primordia by varying concentrations of the plant hormones auxins, cytokinin, and ethylene (Schultze and Kondorosi, 1998). Under drought conditions, auxin-induced proteins 15A and 6B were upregulated in stems. In a comparison of drought and normal watering, both auxin-induced protein transcripts were more upregulated by Strain B in stems under drought than under normal watering.

Tubulin beta-1 chain (TUBB1) is involved in plant cell growth (Takahashi et al., 1995) and has been shown to accumulate in roots (Oppenheimer et al., 1988). Under normal watering, tubulin beta-1 chain (TUBB1) was upregulated in leaves.

The nine-cis-epoxycarotenoid dioxygenase family of enzymes catalyze a key step in biosynthesis of abscisic acid, the plant hormone responsible for growth regulation under stress conditions (Priya and Siva, 2015). Under drought conditions, nine-cis-epoxycarotenoid dioxygenase 4 (NCED4) was upregulated in roots.

NAC domain proteins are homologous to well-known Arabidopsis transcription factors that regulate the differentiation of xylem vessels and fiber cells (Ooka et al., 2003). Under drought conditions, NAC domain protein 32 (NAC32) was upregulated in leaves.

The cytochrome P450 CYP78 family participates in biosynthesis of a growth factor associated with apical meristem development and flower and fruit size (Eriksson et al., 2010; Kazama et al., 2010). Under normal watering, cytochrome P450 78A3 (CYP78A3) was upregulated in leaves. Under drought conditions, cytochrome P450 78A3 (CYP78A3) was upregulated in stems.

Acetolactate synthase catalyzes formation of the precursors of branched-chain amino acids (Chipman et al., 1998). Sulfonylurea herbicides inhibit the action of acetolactate synthase (Walter et al., 2014). Under drought conditions, acetolactate synthase was downregulated in leaves.

Division of chloroplasts relies on a site-determining protein homologous to MinD in prokaryotes, which positions the plastid-division apparatus necessary to initiate binary fission (Colletti et al., 2000). Under drought conditions, site-determining protein was downregulated in leaves.

### Resistance to Abiotic and Biotic Stresses

### Abiotic Stresses

Plant protection against oxidative damage is regulated through enzymatic and non-enzymatic mechanisms. Superoxide dismutase (SOD) is a detoxification enzymes that catalyzes the dismutation of superoxide (O2-) to hydrogen peroxide (H2O2), which peroxidases (PDX) then reduce to water (Matamoros et al., 2003). SOD is specifically highly upregulated in plants grown under drought that show higher expression of nodulation genes. This is consistent with the literature showing that SOD plays a major role in maintaining nodule integrity via controlling ROS overproduction to prevent oxidative damage (Chihaoui et al., 2012). Under normal watering, superoxide dismutase (SODB2) and chloroplastic superoxide dismutase [Fe] (SODB) were upregulated in roots. Under drought conditions, two transcripts for superoxide dismutase [Cu-Zn] (CSD2) were upregulated in leaves. In a comparison of drought and normal watering, the two transcripts for superoxide dismutase [Cu-Zn] were more upregulated by Strain B in leaves under drought than under normal watering. Under normal watering, superoxide dismutase (SODB2) and chloroplastic superoxide dismutase [Fe] (SODB) were downregulated in stems.

In plants, alcohol dehydrogenase, a highly conserved enzyme, is induced by stress conditions, particularly during hypoxic response, to anaerobically supply NAD+ for metabolism (Chung and Ferl, 1999). Under normal watering, alcohol dehydrogenase (ADH-2) was upregulated in stems.

Aldehyde dehydrogenases are members of the NAD(P)(+)-dependent protein superfamily involved in the conversion of various aldehydes to their corresponding nontoxic carboxylic acids (Brocker et al., 2013). Aldehyde dehydrogenases are involved in a wide range of metabolic pathways including growth, development, seed storage, and environmental stress adaptation in higher plants (Rodrigues et al., 2006; Brocker et al., 2013). Under drought conditions, aldehyde dehydrogenase was upregulated in leaves. In a comparison of drought and normal watering, the same aldehyde dehydrogenase transcript was more upregulated by Strain B in leaves under drought than under normal watering. Under normal watering, aldehyde dehydrogenase was downregulated in leaves.

The first committed step in biosynthesis of very-long-chain fatty acids (20 or more carbons) is condensation of C2 units to acyl CoA by 3-ketoacyl CoA synthase (KCS) (Gilbert et al., 1981). Very-long-chain fatty acid derivatives act as protective barriers between plants and the environment, provide energy storage in seeds, and function as signaling molecules in membranes (Devaiah et al., 2006; Pollard et al., 2008). The expression of two Arabidopsis 3-ketoacyl CoA synthase genes increased in response to drought stress, salt, mannitol and ABA (Lee et al., 2009). Indeed our results show that two 3-ketoacyl CoA synthase transcripts were upregulated under drought in leaves of Strain B-treated plants. Under drought conditions, 3-ketoacyl-CoA synthase was downregulated in stems.

Lipid signaling has been implicated in epidermal stages of rhizobium-host interaction in Medicago truncatula (Pii et al., 2012). Phospholipase D is required for MtN5 induction in S. meliloti-inoculated roots (Pii et al., 2012). Furthermore, phospholipase D and its product, phosphatidic acid, have been implicated in multiple plant stress responses by functioning in signal transduction cascades and influencing the biophysical state of lipid membranes (Bargmann and Munnik, 2006). In a comparison of drought and normal watering, phospholipase D was more upregulated by Strain B in roots under drought than under normal watering.

Annexins, a multigene and a multifunctional family of Ca2+-dependent membrane-binding proteins, have been shown to potentially regulate the level and the extent of ROS accumulation and lipid peroxidation during stress responses (Jami et al., 2008). Under drought conditions, two annexin transcripts were upregulated in leaves. In a comparison of drought and normal watering, these two transcripts and two additional annexin transcripts were more upregulated by Strain B in leaves under drought than under normal watering.

The plant glutathione peroxidases are ubiquitous enzymes (Yang et al., 2005) that detoxify lipid hydroperoxides and other reactive molecules in a species-, organ- and stress-specific manner (Churn et al., 1999; Ramos et al., 2009). Under drought conditions, two transcripts for glutathione peroxidase were upregulated in leaves.

The cytochrome P450 CYP77 family hydroxylates and epoxidizes fatty acids, a step in production of precursors of cutin and suberin, biopolyesters that provide protection for the aerial parts and roots, respectively (Kolattukudy, 1980; Sauveplane et al., 2009). Under drought conditions, cytochrome P450 77A3 (CYP77A3) was upregulated in leaves.

The cytochrome P450 CYP93 family is involved in elicitor-inducible glyceollin biosynthesis (Nelson and Werck-Reichhart, 2011; Schopfer and Ebel, 1998). Under normal watering, beta-amyrin 24-hydroxylase (CYP93E1) was upregulated in roots. In a comparison of drought and normal watering, Cytochrome P450 93A3 (CYP93A3) was more upregulated by Strain B in roots under drought than under normal watering.

Recent reverse genetics studies in Arabidopsis revealed that besides their iron storage role, ferritins may be involved in mechanisms of action in oxidative stress pathways (Briat et al., 2010). Six ferritin transcripts were notably upregulated in a tissue- and condition-specific manner. Under normal watering, one ferritin transcript was upregulated in stems and leaves. Under drought conditions, this transcript, another ferritin transcript, and transcripts for chloroplastic ferritins 1, 2, and 4 were all upregulated in stems. In a comparison of drought and normal watering, all five of these transcripts and one additional ferritin transcript (FER2-1) were more upregulated by Strain B in stems under drought than under normal watering.

Thioredoxins are implicated in different aspects of plant life including development and adaptation to environmental changes and stresses. They act as antioxidants by facilitating the reduction of other proteins by cysteine thiol-disulfide exchange (Nordberg and Arnér, 2001). Under normal watering, one thioredoxin transcript was upregulated in roots. Under drought conditions, a second thioredoxin transcript was upregulated in leaves, a third in stems, and a fourth in roots. In a comparison of drought and normal watering, these last two transcripts were more upregulated by Strain B in stems and leaves, respectively, under drought than under normal watering.

Phosphomannomutase is related to biosynthesis of ascorbic acid, an antioxidant important in responses to oxidative stress (Qian et al., 2007). Under drought conditions, phosphomannomutase was upregulated in leaves. In a comparison of drought and normal watering, this transcript was more upregulated by Strain B in leaves under drought than under normal watering.

The EF-hand structural domain provides calcium-binding activity for proteins involved in calcium-signaling for stress response and developmental regulation, including calmodulin and proteins from the salt overly sensitive (SOS) pathway and SOS-like calcium binding proteins (SCaBP) family (Lin et al., 2009). Under drought conditions, an unidentified calcium-binding EF-hand family protein was upregulated in leaves. In a comparison of drought and normal watering, this transcript was more upregulated by Strain B in stems and leaves under drought than under normal watering.

Calmodulins mediate calcium signaling, with widely varied roles across tissues, developmental stages, and stimulus responses (McCormack et al., 2005). In Arabidopsis, calmodulin-2 binds to heat shock protein 70 during calcium signaling, potentially mediating temperature stress responses (Cha et al., 2012). Under drought conditions, calmodulin-2 was upregulated in leaves. In a comparison of drought and normal watering, calmodulin-2 was more upregulated by Strain B in leaves under drought than under normal watering.

The ethylene-responsive element binding factors are stress-inducible transcription factors, participating in genetic regulation of various stress responses (Seki et al., 2002). Under drought conditions, ethylene-responsive element binding factor 4 (ERF-4) was upregulated in roots. In a comparison of drought and normal watering, this transcript was more upregulated by Strain B in roots under drought than under normal watering.

MYB proteins are transcription factors present across eukaryotes, involved in growth, metabolism, and stress responses in plants (Li et al., 2015). Under drought conditions, MYB transcription factor 187 (MYB187) was upregulated in leaves. In a comparison of drought and normal watering, this transcript was more upregulated by Strain B in leaves under drought than under normal watering.

Peptidyl prolyl cis-trans isomerases are molecular chaperones that participate in protein folding and signal transduction (Aviezer-Hagai et al., 2006). In Arabidopsis, the two peptidyl prolyl cis-trans isomerases ROF1 and ROF2 possess different tissue-specific, developmentally regulated, and heat-inducible expression patterns (Aviezer-Hagai et al., 2006). ROF2 has also been shown to participate in intracellular pH homeostasis in Arabidopsis (Bissoli et al., 2012). Under drought conditions, one peptidyl-prolyl cis-trans isomerase was upregulated in leaves. In a comparison of drought and normal watering, this transcript and three others were more upregulated by Strain B in leaves and three additional peptidyl-prolyl cis-trans isomerase transcripts were more upregulated by Strain B in stems under drought than under normal watering.

Nitrate reductase is the enzyme responsible for production of nitric oxide in leaves to induce stomatal closure, in response to ABA signaling under water stress (Desikan et al., 2002). Under drought conditions, nitrate reductase was downregulated in leaves.

Ribose-phosphate pyrophosphokinase produces phosphoribosyldiphosphate, a precursor in biosynthesis of nucleotides, histidine, and tryptophan (Schomburg and Stephan, 1997), as well as NAD and NADP (Zakataeva et al., 2011) and amino sugar synthesis (Rashid et al., 1997). Possibly via NAD's role in cell regulation (Rongvaux et al., 2003), ribose-phosphate pyrophosphokinase has been implicated in iron deficiency response in rice (Chen et al., 2014) and stomatal control in faba bean (Khazaei et al., 2014). Under drought conditions, a ribose-phosphate pyrophosphokinase was downregulated in leaves.

### Biotic Stresses

Lipases are involved in plant resistance to pathogens (Shah, 2005). Under drought conditions, two lipases were upregulated in roots. In a comparison of drought and normal watering, these lipases was more upregulated by Strain B in roots under drought than under normal watering. Under normal watering, two transcripts of lipase were downregulated in roots.

Lipoxygenases catalyze the dioxygenation of polyunsaturated fatty acids in oxylipins, a group of lipids that include j asmonic acid (JA) and its derivatives, and which are involved in a number of developmental and stress response processes (Andersson et al., 2006). Oxilipins may exert protective activities either as signaling molecules in plants during development, wounding, insect and pathogen attack, or as direct anti-microbial substances that are toxic to the invader (Yan et al., 2013). Seven lipoxygenase transcripts were notably upregulated in a tissue- and condition-specific manner. Under normal watering, LOX10 was upregulated in roots and two additional lipoxygenase transcripts were upregulated in leaves. Under drought conditions, LOX9 was upregulated in roots and one additional lipoxygenase transcript was upregulated in leaves. In a comparison of drought and normal watering, these latter two transcripts and two additional lipoxygenase transcripts in roots were more upregulated by Strain B in the previously mentioned tissues under drought than under normal watering. Under drought conditions, one transcript of lipoxygenase was downregulated in stems and another in leaves.

Non-specific lipid transfer proteins (ns-LTPs) are ubiquitous, small, secreted proteins, able to bind to several classes of lipids in vitro (Carvalho and Gomes, 2007). They have been implicated in cutin biosynthesis during pollen development (Zhang et al., 2010), and in stress responses and signaling (Ge et al., 2003). In addition, lipid signaling has recently been implicated in epidermal stages of rhizobium-host interaction: in Medicago truncatula, lipid transfer protein MtN5 positively regulates nodulation (Pii et al., 2012). Twelve non-specific lipid-transfer protein transcripts were notably upregulated in a tissue-specific manner in drought. Under drought conditions, one non-specific lipid-transfer protein was upregulated in roots, another in stems and leaves, and ten more in leaves. In a comparison of drought and normal watering, all of these transcripts were more upregulated by Strain B in the previously mentioned tissues under drought than under normal watering. Under normal watering, one transcript of non-specific lipid-transfer protein was downregulated in stems and leaves, and another only in leaves. Under drought conditions, a different transcript of non-specific lipid-transfer protein was downregulated in stems.

Stress-induced protein SAM22 has been implicated in mechanisms of biotic stress response, including wounding, salicylic acid signaling, hydrogen peroxide signaling, and fungal elicitor response (Crowell et al., 1992). Under normal watering, stress-induced protein SAM22 (DD4/62) was upregulated in roots and leaves. Under drought conditions, the same transcript was upregulated in leaves. Under normal watering, stress-induced protein SAM22 was downregulated in stems.

Repetitive proline-rich cell wall proteins (PRPs), one of the five families of structural cell wall proteins (Carpita and Gibeaut, 1993) that are associated with early stages of legume root nodule formation (Franssen et al., 1987) and other plant developmental stages, may also contribute to defense response mechanisms against physical damage and pathogen infection (Bradley et al., 1992; Brisson et al., 1994). Under normal watering, repetitive proline-rich cell wall protein 2 (PRP2) was upregulated in roots, and PRP3 was upregulated in leaves. Under drought conditions, PRP3 was upregulated in roots. In a comparison of drought and normal watering, PRP3 was more upregulated by Strain B in roots under drought than under normal watering. Under drought conditions, repetitive proline-rich cell wall protein 2 was downregulated in leaves.

The cytochrome P450 CYP89 family has highly variable metabolic functions, leading to involvement in developmental regulation and plant-insect interactions (Christ et al., 2013). For example, CYP89A9 is involved in chlorophyll catabolism during senescence in Arabidopsis (Christ et al., 2013), CYP89H3 specifically is involved in ripening in grapes (Agudelo-Romero et al., 2013), and CYP89A35 is involved in pathogen response via SA and ABA in chili pepper (Kim et al., 2006; Nelson and Werck-Reichhart, 2011). Under normal watering, cytochrome P450 monooxygenase 89H3 (CYP89H3) was upregulated in stems. Under normal watering, cytochrome P450 82A4 was downregulated in roots.

Beta-glucosidase is a cellulase that catalyzes the hydrolysis of terminal non-reducing residues in beta-D-glucosides with release of glucose (Jeng et al., 2011). Beta-glucosidases have been shown to have an elicitor activity of plant defense response to herbivore injury (Mattiacci et al., 1995) or, more specifically, in soybean, they mediate the release of free isoflavones from their conjugates (Suzuki et al., 2006). Flavonoids and isoflavonoids are secondary metabolites that have many functions in higher plants that include UV protection, fertility, antifungal defense and the recruitment of nitrogen-fixing bacteria (Dao et al., 2011). In a comparison of drought and normal watering, beta glucosidase 42 (BGLU42) was more upregulated by Strain B in leaves under drought than under normal watering.

Soybean beta-1,3-endoglucanase releases elicitor-active carbohydrates from the cell walls of fungal pathogens, initiating phytoalexin accumulation in fungus-infected soybean plants (Takeuchi et al., 1990). Beta-1,3-endoglucanase is synonymouswith glucan endo-1,3-beta-glucosidase (Reference: http://www.uniprot.org/uniprot/P34742 see Names & Taxonomy, herein incorporated by reference). Under normal watering, glucan endo-1,3-beta-glucosidase, synonymous according to the UniProt protein database (UniProt Consortium, 2015a), was upregulated in leaves.

MLO-like proteins are transmembrane proteins known to bind to calmodulin and to participate in biotic stress responses (Elliott et al., 2005; Yu et al., 2005). Six MLO-like protein transcripts were notably upregulated in a tissue- and condition-specific manner. Under normal watering, one MLO-like protein was upregulated in roots. Under drought conditions, a second MLO-like protein was upregulated in leaves. In a comparison of drought and normal watering, four additional MLO-like proteins were more upregulated by Strain B in roots under drought than under normal watering.

Wound-induced proteins are those that are upregulated locally by mechanical wounding, which may include proteins active in cell wall modification, defense signaling, or other biotic stress responses (Siebertz et al., 1989; Yen et al., 2001). Under normal watering, a wound-induced protein was upregulated in leaves.

Phenylalanine ammonia lyase (PAL) is the first committed enzyme in the phenylpropanoid pathway leading to biosynthesis of the polyphenol compounds, whose multiple functions include providing mechanical support via lignification (Whetten and Sederoff, 1992), protecting against abiotic and biotic stress as antioxidants (Dixon and Paiva, 1995), and signaling with the flavonoid nodulation factors (Weisshaar and Jenkins, 1998). Under drought conditions, phenylalanine ammonia-lyase was upregulated in the stems.

Arginine decarboxylase is a key enzyme in plant polyamine biosynthesis (Hanfrey et al., 2001). Polyamines have been implicated in a wide range of biological processes including plant growth and development, senescence, environmental stress, and anti-fungal and antiviral effects in pathogen responses (Bais and Ravishankar, 2002). In a comparison of drought and normal watering, arginine decarboxylase was more upregulated by Strain B in stems under drought than under normal watering.

Arginase breaks down arginine, a major free and storage-protein-bound amino acid, to urea and ornithine (Polacco and Holland, 1993). Expression of arginase has been shown to be inducible in tomato leaves in response to various stresses including wounding and pathogen infection (Chen et al., 2004). Arginase was upregulated in leaves of Strain B-treated plants exposed to drought.

In soy, the glycoproteins VSPα and VSβ are vegetative storage proteins important in hypocotyls, young leaves, flowers, and pods, inducible by JA signaling, wounding, sugars, and light, and downregulated by phosphate and auxin (Berger et al., 1995). Under normal watering, VspA (28 kDa glycoprotein) and VspB (31 kDa glycoprotein) were downregulated in roots, and VspB was downregulated in stems and leaves.

S-receptor-like serine/threonine protein kinase, characterized in Glycine soj a, has been shown to play a key role as a positive regulator of plant tolerance to salt stress (Sun et al., 2013). Under normal watering, serine/threonine-protein kinase was downregulated in leaves.

Soy protein P21 (P25096) is a pathogenesis-related (PR) protein of the thaumatin family, involved in antifungal activity (UniProt Consortium, 2015b; Zhang and Shih, 2007). Under normal watering, protein P21 was downregulated in roots and leaves.

### Results: Transcriptomics Quantitative Analysis (Soy Water-Limited Conditions)

Quantitative transcriptomics analyses demonstrated significant conclusions in five areas, as described below.

Genes were Quantified as being Significantly Up/Down Regulated Plants Grown from Strain A or Strain B, Vs Formulation, that Confirm the Qualitative Analysis (Leaf and Root Tissues)

All results are summarized in Table 7B. Descriptions of genes are included in the Qualitative Transcriptomics results section.

Additional Genes that were Quantified as being Significantly Up/Down Regulated in Leaf or Root Tissues of Plants Grown from Seeds Treated with Strain a or Strain B

All results are summarized in Table 7C.

Avr4 promotes Cf-4 receptor-like protein association with the BAK1/SERK3 receptor-like kinase to initiate receptor endocytosis and plant immunity (Liebrand et al. 2014)

Receptor-like proteins (RLPs) are cell surface receptors involved in perceiving and responding to microbes, including HR and immunity (Liebrand et al. 2014). They often have a leucine-rich repeats domain (LRR)—possibly the same protein as “disease resistance family protein/LRR family protein.

Sodium/calcium exchanger family protein/calcium-binding EF hand family protein participates in the maintenance of Ca2+ homeostasis in Arabidopsis and may represent a new type of Ca2+ transporter in higher plants shown to be involved in salt stress in Arabidopis (Peng et al., 2012).

The AMP-dependent synthetase and ligase family protein is an enzyme involved in carbon metabolism, and has a role in degrading acetone to acetyl-CoA in an anaerobic microbe that uses nitrate as an electron acceptor.

Cysteine proteinases superfamily protein participate in developmental regulation, including accumulation and degradation of storage proteins, senescence, and programmed cell death (Schaller, 2004).

Uridine diphosphate glycosyltransferases (UGT) are a superfamily of regulatory enzymes that modify the activity, solubility, and transport of plant hormones, secondary metabolites, and xenobiotics, thus participating in plant developmental regulation, biotic stress responses, and detoxification of pollutants and herbicides (Ross et al., 2001; Wang 2009). Transgenic wheat expressing a barley UDP-glucosyltransferase detoxifies deoxynivalenol and provides high levels of resistance to Fusarium graminearum. A UDP-glucosyltransferase functions in both acylphloroglucinol glucoside and anthocyanin biosynthesis in strawberry (Fragaria×ananassa). UDP-glucosyltransferase UGT84A2/BRT1 is required for Arabidopsis nonhost resistance to the Asian soybean rust pathogen Phakopsora pachyrhizi. Transgenic Arabidopsis thaliana expressing a barley UDP-glucosyltransferase exhibit resistance to the mycotoxin deoxynivalenol. Indole-3-acetic acid UDP-glucosyltransferase from immature seeds of pea is involved in modification of glycoproteins.

The phosphoglycerate mutase (PGM) enzyme catalyzes the interconversion of 2- and 3-phosphoglycerate in the glycolytic/gluconeogenic pathways that are present in the majority of cellular organisms.

2-oxoglutarate (20G) and Fe(II)-dependent oxygenase superfamily protein catalyzes prolyl hydroxylation of RPS23 and is involved in translation control and stress granule formation (Singleton et al. 2014). The OGFOD proteins appear to participate in normal translation processes and gene regulation during translation, with some of these proteins modifying mRNA and tRNA.

Protein kinase protein with adenine nucleotide alpha hydrolases-like domain is involved in protein amino acid phosphorylation and response to stress (Mayer et al., 1999).

Cysteine proteases and proteinases participate in developmental regulation, including accumulation and degradation of storage proteins, senescence, and programmed cell death (Schaller, 2004).

Ferric reduction oxidase 2 facilitates iron nutrient uptake.

Expansin acts during growth to loosen the cell wall polysaccharide network (Cosgrove, 2000), permitting growth but increasing vulnerability to pathogen infection (Ding et al., 2008).

In plants, peroxidases are involved in cell wall lignification, usually associated with pathogen resistance (Bruce and West, 1989), abiotic stress (Huttová et al., 2006; Quiroga et al., 2001), or cell wall modification during growth (Arnaldos et al., 2002; G Martinez Pastur, 2001; Van Hoof and Gaspar, 1976; Kukavica et al., 2012).

Late embryogenesis abundant proteins (LEA) provide desiccation tolerance by changing their folding during drying, possibly creating a water shell under drought and stabilizing cellular components in the absence of water under full desiccation (Shih et al., 2008). The LEA gene HVA1 was successfully transferred from barley into rice to provide water deficit and salt stress tolerance (Xu et al., 1996).

Fructose-bisphosphate aldolase is a glycolytic enzyme, induced by the plant hormone gibberellin, that may regulate the vacuolar H-ATPase-mediated control of cell elongation that determines root length (Konishi et al., 2005).

Pectin lyases are a group of enzymes that are thought to contribute to many biological processes, such as the degradation of pectin. A comprehensive study done in Arabidopsis identified multiple genes involved in cellular metabolism, cellular transport and localization, and stimulus responses like wounding (Cao J., 2012)

Pectinesterase is an enzyme involved in cell wall modification during growth, biotic stress, and nodule formation (Caffall and Mohnen, 2009; Carvalho et al., 2013; Zhang et al., 2015).

NAC (No Apical Meristem) domain transcriptional regulator superfamily protein:

RhNAC3, a stress-associated NAC transcription factor, has a role in dehydration tolerance through regulating osmotic stress-related genes in rose petals. NAC domain protein genes are homologous to well-known Arabidopsis transcription factors that regulate the differentiation of xylem vessels and fiber cells (Ooka et al., 2003).

Cytochrome P450, family 81, subfamily D, polypeptide 3 is involved in secondary metabolite production.

CAP160 protein is a stress-related transporter, and may function in daytime soybean transcriptome fluctuations during water deficit stress

RhNAC3, a stress-associated NAC transcription factor, has a role in dehydration tolerance through regulating osmotic stress-related genes in rose petals

SAUR genes are induced rapidly and transiently by auxin, localized to the membrane and cytoplasm, and related to developmental processes, especially tissue elongation.

The tetratricopeptide repeat is a conserved motif that supports protein-protein interactions, most often found in multiprotein complexes and involved in a variety of essential functions (Blatch and Lassie, 1999). Proteins containing tetratricopeptide repeats have been associated with chloroplast development (Hu et al., 2014), root growth and auxin signaling (Zhang et al., 2015), gibberellin signaling (Jacobsen et al., 1996), heat shock protein function in abiotic stress responses (Prodromou et al., 1999).

Bifunctional inhibitor/lipid-transfer protein/seed storage 2S albumin superfamily protein is involved in seed quality—storing protein and/or lipid.

Ber e 1 protein is the versatile major allergen from Brazil nut seeds (Alcocer et al. 2012).

2S albumin proteins are the proteins that get stored in seeds.

The Arabidopsis NIMIN proteins affect NPR1 differentially (Hermann et al. 2013). NPR receives SA and triggers the PR genes to initiate SAR. NIM-interacting proteins (NIMIN) and TGA transcription factors are part of the transcription complex that carries out NPR's mechanism.

BR hormones promote growth in balance/crosstalk with immune response. BRI1 is the receptor, “reading” brassinosteroids. “Brassinosteroids (BRs) are a group of phytohormones that regulate various biological processes in plants. Interactions and crosstalk between BRs and other plant hormones control a broad spectrum of physiological and developmental processes.”

WRKY transcription factors are involved in biotic and abiotic stress responses and development.

Amino acid kinase family protein regulates nitrogen metabolism. An example, aspartate kinase, receives feedback control and catalyzes the first step: adding a phosphate group to an amino acid (here, aspartate).

VIP1 is a transcription factor that transclocates to the nucleus in response to mechanical touch and hypo-osmotic conditions, which mimic mechanical stimuli. VIP1 appears to suppress touch-induced responses (Tsugama et al. 2016). VIP was first studied in relation to Agrobacterium, which apparently uses VIP1's movement into the nucleus to “hitch a ride” and transfer its own DNA into the plant cell's nucleus (Gelvin 2010).

Glycosyl hydrolases family 32 protein is the key enzyme in decomposing lignocellulose/plant cell walls (Kanokratana et al. 2015, Mori et al. 2014). Likely used by plants to remodel cell walls during stress, and possibly in relation to microbial colonization.

The RWP-RK family are transcription factors that regulate responses to nitrogen availability, including nodule formation and rhizobial colonization. They were first found in legumes, but homologs exist in all vascular plants, green algae, and slime molds.

Late embryogenesis abundant proteins (LEA) provide desiccation tolerance by changing their folding during drying, possibly creating a water shell under drought and stabilizing cellular components in the absence of water under full desiccation (Shih et al., 2008). The LEA gene HVA1 was successfully transferred from barley into rice to provide water deficit and salt stress tolerance (Xu et al., 1996).

CYP71 family previously identified in up/down regulation during stress responses to drought, phenol (Cerekovic et al. 2015, Xu et al. 2012). Suggests a close relative enzyme (CYP71A22 (cytochrome P450, family 71, subfamily A, polypeptide 22) participates in the pathway to synthesize furanocoumarins, defensive secondary metabolites, in grapefruit (Chen et al. 2014).

The heat-shock proteins are molecular chaperones expressed under various stresses to stabilize proteins (De Maio, 1999).

Up/Down Regulated Genes that are Unique to Plants Grown from Seeds Treated with Strain A or Strain B (Genes in Leaf or Root Tissues) as Compared to Plants Grown from Seeds Treated with the Formulation Control, that are not Found Significantly Up- or Down-Regulated in Plants Grown from Seeds Treated with Strain F

All results are summarized in Table 7D.

Lectin receptor kinases are signaling molecules involved in a wide range of plant activities, including nodulation and symbiosis in legumes, detection and response to herbivory and pathogens, abiotic stress tolerance, and certain aspects of plant development (Vaid et al., 2012).

In plants, the FW2.2-like (FWL) genes containing the PLAC8 conserved motif determine cell count in vegetative and reproductive structures, contributing directly to yield (Guo et al., 2010). Fruit size in tomato (Nesbitt and Tanksley, 2001), plant and organ size in corn (Guo et al., 2010), and organogenesis in corn and soybean (Libault and Stacey, 2010) have been attributed to FWL genes.

Glycerol acyltransferases are a group of enzymes involved in synthesizing triacylglycerol, which accumulates in the seed, permitting normal seed embryo development (Zhang et al., 2009) and providing the main component of soybean oil (Wang et al., 2006).

The ribosome is essential for translation of mRNA into proteins in all living organisms. In eukaryotes, the ribosome is composed of a large 60S subunit and a small 40S subunit (Ben-Shem et al., 2011).

Embryo defective 2737 (Emb2737) is a putative membrane protein, essential to embryonic development (Simm et al., 2013).

In plants the Armadillo (ARM) repeat region, which is a motif involved in protein-protein interactions, is preceded by a E3 ubiquitin ligase motif called the U-box and thus can result in proteasomal degradation or, alternately, regulate other processes such as transcription and DNA repair (Samuel et al. 2006). In Arabidopsis, an Arm Repeat protein interacts with a transcriptional regulation of Abscisic Acid-Responsive genes, making plants sensitive to high osmolarity during germination and insensitive to salt during subsequent seedling growth (Kim et al. 2004)

Pleiotropic drug resistance 1 is a plasma membrane ABC transporter involved in tolerance against accumulation of the deoxynivalenol (DON), one of the toxins produced by Fusarium pathogens during spike invasion as first step in the progression of the head blight disease in wheat (Gottwald et al. 2012).

The enzyme encoded by histidinol phosphate aminotransferase 1 gene catalyzes the synthesis of 3-(imidazol-4-yl)-2-oxopropyl phosphate and is involved in histidine metabolism. This is a gene associated with symbiotic nitrogen fixation in soybean, upregulated under drought conditions (Dhanapal et al. 2015)

The tetratricopeptide repeat is a conserved motif that supports protein-protein interactions, most often found in multiprotein complexes and involved in a variety of essential functions (Blatch and Lassie, 1999). Proteins containing tetratricopeptide repeats have been associated with chloroplast development (Hu et al., 2014), root growth and auxin signaling (Zhang et al., 2015), gibberellin signaling (Jacobsen et al., 1996), heat shock protein function in abiotic stress responses (Prodromou et al., 1999).

AGC kinases are regulatory proteins involved in many key functions and pathways, including growth and development and biotic stress responses (Garcia et al., 2012).

K+ uptake permeases (KUP) are K+/H+ symporters embedded in the plasma membrane, responsible for transporting the essential nutrient potassium. Upregulation of a KUP gene was detected in a low-potassium-tolerant variety of soybean, suggesting a mechanism for tolerance (Wang et al., 2012).

Phloem protein 2-A1 in Arabidopsis was found to act as a molecular chaperone and provide antifungal activity (Lee et al., 2014). In citrus, the phloem protein 2 family is associated with phloem plugging in response to bacterial and viral infections (Albrigo et al., 2014; Hajeri et al., 2014).

The development and cell death (DCD) domain regulates programmed cell death via the hypersensitive response in plants, and is shared by numerous plant proteins involved in hormone response, embryo development, and hypersensitive response to pathogens and abiotic stress (Tenhaken et al., 2005).

Abiotic stresses and pathogen attack can result in accumulated DNA damage and eventual genotoxic stress, necessitating coordination of DNA repair in response to environmental stress (Dona et al., 2013). The RAD family proteins are dedicated to DNA repair, with numerous homologs in plants (Heitzeberg et al., 2004; Liu et al., 2000).

LMBR-like proteins are integral membrane proteins involved in developmental regulation, with homologs across eukaryote taxa (Kelsey et al., 2012).

Lectin receptor kinases are signaling molecules involved in a wide range of plant activities, including nodulation and symbiosis in legumes, detection and response to herbivory and pathogens, abiotic stress tolerance, and certain aspects of plant development (Vaid et al., 2012).

Helicases are involved in basic cellular functions, including DNA replication and repair, RNA splicing, translation, and the cell cycle (Isono et al., 1999).

Increases in organic acid metabolism support a shift in carbon metabolism that accommodates increased nitrate assimilation (Scheible et al., 2000). Phosphoenolpyruvate carboxylase is a metabolic enzyme involved in organic acid metabolism, regulated by its related kinase (Scheible et al., 2000).

Amino acid permeases transport nitrogen across cell membranes in the form of amino acids, contributing to the dynamics of nitrogen fixation and assimilation, and regulating growth and development (Tegeder, 2012). For example, in Arabidopsis, seed count and normal seed development depend absolutely on the amino acid permease AAP8 (Schmidt et al. 2007).

Transcription factors are proteins that bind to specific sequences of DNA, usually as part of a multi-protein complex, in order to regulate which genes are expressed (Tripathi et al., 2013). Transcription factors also participate in DNA repair processes (Malewicz and Perlmann, 2014).

TRF-like genes are a subgroup of the MYB transcription factors (Du et al., 2013), involved in growth, metabolism, and stress responses in plants (Li et al., 2015). In maize, TRF-like genes were found to be more highly expressed under drought stress and in seeds (Du et al., 2013).

Up/Down Regulated Genes that are Significantly Represented in Plants Grown from Seeds Treated with Strain a or Strain B Versus Plants Grown from Seeds Treated with Strain F

All results are summarized in Table 7E.

Receptor-like proteins (RLPs) are cell surface receptors involved in perceiving and responding to microbes, including HR and immunity (Liebrand et al. 2014). They often have a leucine-rich repeats domain (LRR)—possibly the same protein as “disease resistance family protein/LRR family protein.

The tetratricopeptide repeat is a conserved motif that supports protein-protein interactions, most often found in multiprotein complexes and involved in a variety of essential functions (Blatch and Lassie, 1999). Proteins containing tetratricopeptide repeats have been associated with chloroplast development (Hu et al., 2014), root growth and auxin signaling (Zhang et al., 2015), gibberellin signaling (Jacobsen et al., 1996), heat shock protein function in abiotic stress responses (Prodromou et al., 1999).

AMP-dependent synthetase and ligase family protein is an enzyme involved in carbon metabolism. The link below describes this enzyme's role in degrading acetone to acetyl-CoA in an anaerobic microbe that uses nitrate as an electron acceptor. In the proposed acetone degradation pathway, an acetone carboxylase converts acetone to acetoacetate, an AMP-dependent synthetase/ligase converts acetoacetate to acetoacetyl-CoA, and an acetyl-CoA acetyltransferase cleaves acetoacetyl-CoA to two acetyl-CoA (Oosterkamp et al. 2015).

VIP1 is a transcription factor that transclocates to the nucleus in response to mechanical touch and hypo-osmotic conditions, which mimic mechanical stimuli. VIP1 appears to suppress touch-induced responses (Tsugama et al. 2016). VIP was first studied in relation to Agrobacterium, which apparently uses VIP1's movement into the nucleus to “hitch a ride” and transfer its own DNA into the plant cell's nucleus (Gelvin 2010).

The RWP-RK family are transcription factors that regulate responses to nitrogen availability, including nodule formation and rhizobial colonization. They were first found in legumes, but homologs exist in all vascular plants, green algae, and slime molds.

UTP-glucose-1-phosphate uridylyltransferase provides UDP-glucose, an important substrate for cell wall polysaccharide biosynthesis (Cook et al., 2012; Hertzberg et al., 2001).

UDP-glucose 6-dehydrogenase is an enzyme that participates in cell wall formation and modification by providing UDP-glucuronate for polysaccharide biosynthesis (Cook et al., 2012).

CYP71 family previously identified in up/down regulation during stress responses to drought, phenol (Cerekovic et al. 2015, Xu et al. 2012). Suggests a close relative enzyme (CYP71A22 (cytochrome P450, family 71, subfamily A, polypeptide 22) participates in the pathway to synthesize furanocoumarins, defensive secondary metabolites, in grapefruit (Chen et al. 2014).

Glycosyl hydrolases family 32 protein is a key enzyme in decomposing lignocellulose/plant cell walls (Kanokratana et al. 2015, Mori et al. 2014). Likely used by plants to remodel cell walls during stress, and possibly in relation to microbial colonization.

The tetratricopeptide repeat is a conserved motif that supports protein-protein interactions, most often found in multiprotein complexes and involved in a variety of essential functions (Blatch and Lassie, 1999). Proteins containing tetratricopeptide repeats have been associated with chloroplast development (Hu et al., 2014), root growth and auxin signaling (Zhang et al., 2015), gibberellin signaling (Jacobsen et al., 1996), heat shock protein function in abiotic stress responses (Prodromou et al., 1999).

Leucine-rich repeat receptor kinases (LRR-RKs) perform cell surface signaling to register environmental information, including microbe recognition (Belkhadir et al. 2014).

The Snf1-related protein kinases SnRK2. 4 and SnRK2. 10 are involved in maintenance of root system architecture during salt stress (McLoughlin et al. 2012)

A family of kinases involved in osmotic stress signaling, possibly related to abscisic acid signaling.

WRKY DNA-binding protein 40 is a family of transcription factors involved in biotic and abiotic stress responses and development.

SAUR genes are induced rapidly and transiently by auxin, localized to the membrane and cytoplasm, and related to developmental processes, especially tissue elongation.

The OGFOD proteins participate in normal translation processes and gene regulation during translation, with some of these proteins modifying mRNA and tRNA.

UDP-glucosyl transferase 88A1 can be applied in transgenic crops for pathogen resistance; produces glucosides and detoxifies microbial products. Uridine diphosphate glycosyltransferases (UGT) are a superfamily of regulatory enzymes that modify the activity, solubility, and transport of plant hormones, secondary metabolites, and xenobiotics, thus participating in plant developmental regulation, biotic stress responses, and detoxification of pollutants and herbicides (Ross et al., 2001; Wang 2009).

Cysteine proteases and proteinases participate in developmental regulation, including accumulation and degradation of storage proteins, senescence, and programmed cell death (Schaller, 2004).

CAP160 protein gene has been found up/down regulated before in drought stress in soy, as well as dehydration tolerance in roses and dessication tolerance in germinated Arabidopsis seeds. It is a stress-related transporter.

NmrA-like negative transcriptional regulator family protein performs as a nitrogen metabolite repressor, meaning ignoring a non-preferred nitrogen source because a preferred source is available. The superfamily includes the short-chain dehydrogenase/reductases (SDR), involved in metabolism and sometimes signaling, but NmrA is unlikely to act as a dehydrogenase. It is associated with a beneficial response to nanoparticle stress in soy (aluminum oxide nanoparticle treatment in flooded 2-day-old soy seedlings; 5-fold upregulation of this protein/transcript associated with enhanced growth).

The tetratricopeptide repeat is a conserved motif that supports protein-protein interactions, most often found in multiprotein complexes and involved in a variety of essential functions (Blatch and Lassie, 1999). Proteins containing tetratricopeptide repeats have been associated with chloroplast development (Hu et al., 2014), root growth and auxin signaling (Zhang et al., 2015), gibberellin signaling (Jacobsen et al., 1996), heat shock protein function in abiotic stress responses (Prodromou et al., 1999).

Up/Down Regulated Transcripts that are Significantly Represented in Plants Grown from Seeds Treated with Strain a or Strain B Vs. Plants Grown from Seeds Treated with Strain F

All results are summarized in Table 7F.

In plants the Armadillo (ARM) repeat region, which is a motif involved in protein-protein interactions, is preceded by a E3 ubiquitin ligase motif called the U-box and thus can result in proteasomal degradation or, alternately, regulate other processes such as transcription and DNA repair (Samuel et al. 2006). In Arabidopsis, an Arm Repeat protein interacts with a transcriptional regulation of Abscisic Acid-Responsive genes, making plants sensitive to high osmolarity during germination and insensitive to salt during subsequent seedling growth (Kim et al. 2004)

The bHLH family includes the MYC family transcription factors, involved in jasmonic acid defense signaling, and also the growth-promoting brassinosteroids.

Some members of the calcineurin-like metallo-phosphoesterase superfamily protein control the seed coat in soy (Sun et al., 2015).

WD40 domains allow proteins to bind with other proteins to form scaffolding and complexes, or to mediate other proteins' interactions. They are important in development and stress signaling.

NOT2_3_5 domain-containing proteins are involved in generating miRNA for post-transcriptional regulation of gene expression. Also includes Vire2-Interacting Protein2, above, involved in touch and hypo-osmotic signaling.

The development and cell death (DCD) domain regulates programmed cell death via the hypersensitive response in plants, and is shared by numerous plant proteins involved in hormone response, embryo development, and hypersensitive response to pathogens and abiotic stress (Tenhaken et al., 2005).

Abiotic stresses and pathogen attack can result in accumulated DNA damage and eventual genotoxic stress, necessitating coordination of DNA repair in response to environmental stress (Dona et al., 2013). The RAD family proteins are dedicated to DNA repair, with numerous homologs in plants (Heitzeberg et al., 2004; Liu et al., 2000).

LMBR-like proteins are integral membrane proteins involved in developmental regulation, with homologs across eukaryote taxa (Kelsey et al., 2012).

G protein alpha subunit 1 is involved in signal transduction—from cell surface receptors to transcription factors or other effector proteins.

the adaptor protein Yae1 binds to Rli1 and then recruits it to the CIA machinery via interactions that it makes with the deca-GX 3 motif on Lto1

P-loop containing nucleoside triphosphate hydrolases superfamily protein is a very large family, whose function is to interact with the triphosphate tail of bound nucleotides. Involved in just about everything: transcription, translation, replication, DNA repair, signaling, protein transport, chromosome structure, membrane transport, metabolism.

CAP160 protein is a stress-related protein.

MLO-like proteins are transmembrane proteins known to bind to calmodulin and to participate in biotic stress responses (Elliott et al., 2005; Yu et al., 2005).

One of the detoxification enzymes, superoxide dismutase (SOD), catalyses the dismutation of superoxide (O2-) to hydrogen peroxide (H202) that gets reduced to water by peroxidases (PDX) (Matamoros et al., 2003). SOD is specifically highly upregulated in plants grown under drought that show higher expression of nodulation genes. This is consistent with the literature showing that SOD plays a major role in maintaining nodule integrity via controlling ROS overproduction (Chihaoui et al., 2012).

XB3 ortholog 1 is involved with Hhypersensitive response (HR) signaling. A ubiquitin ligase, it binds to XA21, which is a receptor kinase that erforms microbial recognition. In rice, downregulating XB3 reduces the plant's ability to resist disease.

### Example 9: Identification of Differentially Regulated Proteins (Proteomics)

### Sample Preparation for Proteomics Analysis

1 mL of 5% SDS 1 mM DTT was added to 1 mL of homogenized tissue and the samples were boiled for 5 m. The samples were cooled on ice and 2 mL of 8M urea solution was added. The samples were spun for 20 m at 14,000 rpm and the soluble phase recovered. A 25% volume of 100% TCA solution was added to the soluble phase, left on ice for 20 m and centrifuged for 10 m at 14,000 rpm. The protein pellet was washed twice with ice-cold acetone and solubilized in 125 uL 0.2M NaOH and neutralized with 125 uL of 1M Tris-Cl pH 8.0. Protein solutions were diluted in THE (50 mM Tris-Cl pH 8.0, 100 mM NaCl, 1 mM EDTA) buffer. RapiGest SF reagent (Waters Corp., Milford, Mass.) was added to the mix to a final concentration of 0.1% and samples were boiled for 5 min. TCEP (Tris (2-carboxyethyl) phosphine) was added to 1 mM (final concentration) and the samples were incubated at 37° C. for 30 min. Subsequently, the samples were carboxymethylated with 0.5 mg/ml of iodoacetamide for 30 min at 37° C. followed by neutralization with 2 mM TCEP (final concentration). Proteins samples prepared as above were digested with trypsin (trypsin:protein ratio—1:50) overnight at 37° C. RapiGest was degraded and removed by treating the samples with 250 mM HCl at 37° C. for 1h followed by centrifugation at 14,000 rpm for 30 min at 4° C. The soluble fraction was then added to a new tube and the peptides were extracted and desalted using Aspire RP30 desalting columns (Thermo Scientific). The trypsinized samples were labeled with isobaric tags (iTRAQ, ABSCIEX, Ross et al 2004), where each sample was labeled with a specific tag to its peptides.

### Mass Spectrometry Analysis

Each set of experiments (samples 1-6; 7, 8; 9-12; 13-16; 17-20) was then pooled and fractionated using high pH reverse phase chromatography (HPRP-Xterra C18 reverse phase, 4.6 mm×10 mm 5 um particle (Waters)). The chromatography conditions were as follows: the column was heated to 37° C. and a linear gradient from 5-35% B (Buffer A-20 mM ammonium formate pH10 aqueous, Buffer B-20 mM ammonium formate pH10 in 80% ACN-water) was applied for 80 min at 0.5 ml/min flow rate. A total of 30 fractions of 0.5 ml volume where collected for LC-MS/MS analysis. Each of these fractions was analyzed by high-pressure liquid chromatography (HPLC) coupled with tandem mass spectroscopy (LC-MS/MS) using nano-spray ionization. The nanospray ionization experiments were performed using a TripleT of 5600 hybrid mass spectrometer (AB SCIEX Concord, Ontario, Canada)) interfaced with nano-scale reversed-phase HPLC (Tempo, Applied Biosystems (Life Technologies), CA, USA) using a 10 cm-180 micron ID glass capillary packed with 5 um C18 Zorbax™ beads (Agilent Technologies, Santa Clara, Calif.). Peptides were eluted from the C18 column into the mass spectrometer using a linear gradient (5-30%) of ACN (Acetonitrile) at a flow rate of 550 μl min-1 for 100 min. The buffers used to create the ACN gradient were: Buffer A (98% H2O, 2% ACN, 0.2% formic acid, and 0.005% TFA) and Buffer B (100% ACN, 0.2% formic acid, and 0.005% TFA). MS/MS data were acquired in a data-dependent manner in which the MS1 data was acquired for 250 ms at m/z of 400 to 1250 Da and the MS/MS data was acquired from m/z of 50 to 2,000 Da. For Independent data acquisition (IDA) parameters MS1-TOF 250 ms, followed by 50 MS2 events of 25 ms each. The IDA criteria, over 200 counts threshold, charge state +2-4 with 4 s exclusion. Finally, the collected data were analyzed using Protein Pilot 4.0 (AB SCIEX) for peptide identifications and quantification.

### Results

The proteomic analysis of soybean plants inoculated with endophytic fungal strain Strain B grown under drought and normal watering regime in the greenhouse revealed three major pathways that are modulated by the endophyte: symbiosis enhancement, growth promotion and resistance against abiotic and biotic stresses. All results are summarized in Table 8.

Proteomics upregulated, normal watering conditions, in root tissue: cysteine proteinase RD21a-like, isocitrate dehydrogenase [NADP], bifunctional aspartate aminotransferase and glutamate/aspartate-prephenate aminotransferase-like, proteasome subunit alpha type-5-like, THO complex subunit 4-like isoform X1, asparagine synthetase, root [glutamine-hydrolyzing]-like, mitochondrial dicarboxylate/tricarboxylate transporter DTC-like, DNA repair and recombination protein RAD26-like, peptidyl-prolyl cis-trans isomerase CYP20-2, chloroplastic-like isoform 1, photosystem I P700 apoprotein A2, histone H4-like, 1-deoxy-D-xylulose 5-phosphate reductoisomerase, chloroplastic-like, thylakoid lumenal 16.5 kDa protein, chloroplastic-like isoform X1, beta-amylase precursor, cytochrome b6-f complex iron-sulfur subunit, chloroplastic-like, thioredoxin M4, chloroplastic-like.

Proteins downregulated, normal watering conditions, in root tissue: magnesium-chelatase subunit ChlI, chloroplastic, endoplasmin homolog isoform X2, chloroplast stem-loop binding protein of 41 kDa b, chloroplastic-like isoform X2, leucine aminopeptidase 3, chloroplastic-like, peroxisomal (S)-2-hydroxy-acid oxidase GLO1-like isoform X1, glutamate—glyoxylate aminotransferase 2-like isoform 2, alcohol dehydrogenase 1, patellin-3-like isoform X3, alcohol dehydrogenase class-3, NADP-dependent malic enzyme-like, glutamine synthetase precursor isoform X1, UTP—glucose-1-phosphate uridylyltransferase-like, enolase-phosphatase E1-like, luminal-binding protein, chloroplast stem-loop binding protein of 41 kDa a, chloroplastic-like, ketol-acid reductoisomerase, chloroplastic-like, fructose-bisphosphate aldolase 1, chloroplastic-like, stromal 70 kDa heat shock-related protein, chloroplastic-like, putative glucose-6-phosphate 1-epimerase-like, cucumisin-like isoform X2.

Proteins upregulated, normal watering conditions, in leaf tissue: photosystem II protein H, thylakoid lumenal 16.5 kDa protein, chloroplastic-like isoform X1, CDGSH iron-sulfur domain-containing protein NEET-like, lipoxygenase L-5, photosystem II 44 kDa protein, histone H4-like, thioredoxin-like protein CDSP32, chloroplastic-like, dirigent protein 1-like, 1-deoxy-D-xylulose 5-phosphate reductoisomerase, chloroplastic-like, probable 60S ribosomal protein L14-like, thylakoid lumenal 29 kDa protein, chloroplastic-like, stromal 70 kDa heat shock-related protein, chloroplastic-like, thioredoxin M4, chloroplastic-like, fructose-bisphosphate aldolase 1, chloroplastic-like, ruBisCO-associated protein, cucumisin-like isoform X2, LOW QUALITY PROTEIN: 50S ribosomal protein L11, chloroplastic-like, chloroplast stem-loop binding protein of 41 kDa a, chloroplastic-like, methionine synthase, ATP synthase CF1 alpha subunit, sedoheptulose-1,7-bisphosphatase, chloroplastic-like isoform 1.

Proteins downregulated, normal watering conditions, in leaf tissue: inositol-3-phosphate synthase, protease Do-like 2, chloroplastic-like, T-complex protein 1 subunit gamma-like, cell division protein FtsZ homolog 1, chloroplastic-like, protein CURVATURE THYLAKOID 1A, chloroplastic-like, magnesium-chelatase subunit ChlI, chloroplastic, THO complex subunit 4-like isoform X1, bifunctional aspartate aminotransferase and glutamate/aspartate-prephenate aminotransferase-like, beta-amylase precursor, UTP—glucose-1-phosphate uridylyltransferase-like, putative dihydroxy-acid dehydratase, mitochondrial-like, 3-ketoacyl-CoA thiolase 2, peroxisomal-like isoform X1, ketol-acid reductoisomerase, chloroplastic-like, putative lactoylglutathione lyase-like isoform X2.

Proteins upregulated, water-limited conditions, in root tissue: alcohol dehydrogenase class-3, enolase-phosphatase E1-like, DNA-damage-repair/toleration protein DRT100-like precursor, heat shock protein 90-1, 26S protease regulatory subunit 10B homolog A-like, glutamate—glyoxylate aminotransferase 2-like isoform 2, ketol-acid reductoisomerase, chloroplastic-like, lipoxygenase-9, 3-ketoacyl-CoA thiolase 2, peroxisomal-like isoform X1, luminal-binding protein, protein CURVATURE THYLAKOID 1A, chloroplastic-like, 50S ribosomal protein L6, chloroplastic-like isoform 1, aldehyde dehydrogenase family 3 member I1, chloroplastic-like, beta-amylase precursor, glutamine synthetase precursor isoform X1, peptidyl-prolyl cis-trans isomerase, chloroplastic isoform 1, UTP—glucose-1-phosphate uridylyltransferase-like, NADP-dependent malic enzyme-like, isocitrate dehydrogenase [NADP], fructose-bisphosphate aldolase 1, chloroplastic-like, stromal 70 kDa heat shock-related protein, chloroplastic-like, cell division protein FtsZ homolog 1, chloroplastic-like, peroxisomal (S)-2-hydroxy-acid oxidase GLO1-like isoform X1, lipoxygenase, lipoxygenase L-5.

Proteins downregulated, water-limited conditions, in root tissue: photosystem I P700 apoprotein A2, putative dihydroxy-acid dehydratase, mitochondrial-like, calcium-transporting ATPase 4, plasma membrane-type-like, abscisic stress ripening-like protein, 3′-hydroxy-N-methyl-(S)-coclaurine 4′-O-methyltransferase-like, sulfite reductase [ferredoxin], chloroplastic, THO complex subunit 4-like isoform X1, 60S ribosomal protein L4-like isoform 1, isocitrate dehydrogenase [NAD] catalytic subunit 5, mitochondrial-like, transketolase, chloroplastic, histone H4-like, peroxidase 50-like, thylakoid lumenal 29 kDa protein, chloroplastic-like, cysteine proteinase RD21a-like, bifunctional aspartate aminotransferase and glutamate/aspartate-prephenate aminotransferase-like, T-complex protein 1 subunit gamma-like, thioredoxin-like protein CDSP32, chloroplastic-like, aconitate hydratase 2, mitochondrial-like, delta-1-pyrroline-5-carboxylate synthase-like, asparagine synthetase, root [glutamine-hydrolyzing]-like, mitochondrial dicarboxylate/tricarboxylate transporter DTC-like.

Proteins upregulated, water-limited conditions, in leaf tissue: ruBisCO-associated protein, thioredoxin-like protein CDSP32, chloroplastic-like, methionine synthase, vicianin hydrolase-like, peroxidase 50-like, chloroplast stem-loop binding protein of 41 kDa a, chloroplastic-like, LOW QUALITY PROTEIN: 50S ribosomal protein L11, chloroplastic-like, isocitrate dehydrogenase [NADP], alcohol dehydrogenase 1, cucumisin-like isoform X2, aspartate aminotransferase glyoxysomal isozyme AAT1 precursor, aconitate hydratase 2, mitochondrial-like, lipoxygenase, cytochrome b6-f complex iron-sulfur subunit, chloroplastic-like, ATP synthase CF1 alpha subunit, cytochrome f, sedoheptulose-1,7-bisphosphatase, chloroplastic-like isoform 1, glutamate—glyoxylate aminotransferase 2-like isoform 2, luminal-binding protein, fructose-bisphosphate aldolase 1, chloroplastic-like, inositol-3-phosphate synthase, lipoxygenase-9, photosystem I P700 apoprotein A2, DNA-damage-repair/toleration protein DRT100-like precursor, DNA repair and recombination protein RAD26-like, mitochondrial dicarboxylate/tricarboxylate transporter DTC-like.

Proteins downregulated, water-limited conditions, in leaf tissue: protein CURVATURE THYLAKOID 1A, chloroplastic-like, putative glucose-6-phosphate 1-epimerase-like, 3-ketoacyl-CoA thiolase 2, peroxisomal-like isoform X1, probable histone H2B.3, ornithine carbamoyltransferase, chloroplastic-like, chaperonin CPN60-like 2, mitochondrial-like, putative lactoylglutathione lyase-like isoform X2, delta-1-pyrroline-5-carboxylate synthase-like, ATP synthase CF1 epsilon subunit, asparagine synthetase, root [glutamine-hydrolyzing]-like, putative dihydroxy-acid dehydratase, mitochondrial-like, cell division protein FtsZ homolog 1, chloroplastic-like, protease Do-like 2, chloroplastic-like, THO complex subunit 4-like isoform X1.

### Symbiosis Enhancement

### Nodulation

Malic enzyme has been shown to be important for carbon metabolism of bacteroids and free living bacteria by supplying acetyl-CoA for the TCA cycle or providing NADPH and pyruvate for various biosynthetic pathways (Dao et al., 2008). Soybean plants inoculated with a NAD(+)-dependent malic enzyme mutant formed small root nodules and exhibited significant nitrogen-deficiency symptoms (Dao et al., 2008). Under normal watering, predicted NADP-dependent malic enzyme-like was downregulated in roots.

### Nitrogen Metabolism

In most legumes, asparagine is the principal assimilation product of symbiotic nitrogen fixation (Scott et al., 1976). In soybean, high asparagine synthetase transcript level in source leaves is positively correlated with protein concentration of seed (Wan et al., 2006), and in roots, is linked with increased levels of asparagine in xylem sap transported to the shoot (Antunes et al., 2008). Under normal watering, predicted asparagine synthetase, root [glutamine-hydrolyzing]-like was upregulated in roots.

Glutamine and glutamate synthetases are enzymes responsible for assimilation of fixed ammonia during nitrogen fixation (Lara et al., 1983). In common bean (Phaseolus vulgaris) and in soy, nodule-specific forms of glutamine synthetase are produced in rhizobia-colonized nodules (Lara et al., 1983; Sengupta-Gopalan and Pitas, 1986), highlighting the enzyme's role in symbiosis. Under drought conditions, predicted glutamine synthetase precursor was upregulated in roots.

After assimilation, aspartate is the principal nitrogen transport compound (Robinson et al., 1994; Schultz et al., 1998). Aspartate aminotransferase, responsible for synthesis of aspartate from glutamate, is highly expressed in rhizobia-colonized root nodules of alfalfa (Robinson et al., 1994). Under normal watering, predicted bifunctional aspartate aminotransferase and glutamate/aspartate-prephenate aminotransferase-like was upregulated in roots. Under drought conditions, aspartate aminotransferase glyoxysomal isozyme AAT1 precursor was upregulated in leaves.

Methionine is an amino acid in the aspartate family (Hesse et al., 2004), important for nitrogen transport especially in plants hosting nitrogen-fixing bacteria (Robinson et al., 1994; Schultz et al., 1998). Under drought conditions, methionine synthase was upregulated in leaves.

In most legumes, asparagine is the principal assimilation product of symbiotic nitrogen fixation (Scott et al., 1976). In soybean, high asparagine synthetase transcript level in source leaves is positively correlated with protein concentration of seed (Wan et al., 2006), and in roots, is linked with increased levels of asparagine in xylem sap transported to the shoot (Antunes et al., 2008). Under drought conditions, predicted asparagine synthetase, root [glutamine-hydrolyzing]-like was downregulated in leaves.

### Growth Promotion

### Carbon Metabolism and Photosynthesis

Fructose-bisphosphate aldolase is a glycolytic enzyme, induced by the plant hormone gibberellin, that may regulate the vacuolar H-ATPase-mediated control of cell elongation that determines root length (Konishi et al., 2005). Under normal conditions, predicted fructose-bisphosphate aldolase 1, chloroplastic-like was upregulated in leaves.

Along with fructose-1,6-bisphosphatase, sedoheptulose-1,7-bisphosphatase participates in carbon metabolism in the reductive pentose phosphate cycle, with photoactivation via the ferredoxin/thioredoxin system (Nishizawa and Buchanan, 1981). Transgenic overexpression of sedoheptulose-1,7-bisphosphatase in tobacco resulted in enhanced photosynthetic efficiency and increased growth (Miyagawa et al., 2001). Under drought conditions, predicted sedoheptulose-1,7-bisphosphatase, chloroplastic-like isoform 1 was upregulated in leaves.

ATP synthase provides energy to the cell through the synthesis of adenosine triphosphate (ATP) from adenosine diphosphate (ADP) and inorganic phosphate (Pi). The ATP produced by the light reactions is then used by the dark reactions of photosynthesis to reduce CO2 to carbohydrates (McCarty, 1992). Changes in ATP synthase contents have been reported in response to changes in light intensity (Anderson et al., 1988), leaf age (Schottler et al., 2007a), and drought stress (Kohzuma et al., 2009). Under drought conditions, ATP synthase CF1 alpha subunit was upregulated in leaves.

Beta-amylase is a starch-hydrolyzing enzyme (Ishikawa et al., 2007). Starch metabolism is important for grain filling: for example, in rice, starch accumulated in leaf sheaths contributes 30% of the grain yield (Ishikawa et al., 1993; Hirose et al., 2006). Under drought conditions, beta-amylase precursor was upregulated in roots.

Ketol-acid reductoisomerase is involved in biosynthesis of branched-chain amino acids necessary for plant growth (Wong et al., 2012). Under drought conditions, predicted ketol-acid reductoisomerase, chloroplastic-like was upregulated in roots.

The photosystem II complex initiates photosynthesis by catalyzing electron transfer from water to the electron transport chain (Suorsa et al., 2004). Photosystem II protein H is a small, hydrophilic subunit of the PSII complex, possibly associated with the pigment proteins (Levey et al., 2014). Under normal watering, two PRI subunits, photosystem II protein H and a 44 kDa photosystem II protein, were upregulated in leaves.

The cytochrome b6f complex, highly conserved across plants, algae, and cyanobacteria (Sainz et al., 2000), is key to the electron transfer chain of photosynthesis (Allen, 2004). Under drought conditions, cytochrome f and predicted cytochrome b6-f complex iron-sulfur subunit, chloroplastic-like were upregulated in leaves.

A non-enzymatic, narbonin-like RuBisCO complex protein (RCP) was reported to accumulate in leaves following pod removal, but there is no evidence that it shares narbonin's role in storage and its function remains unknown (Mahato et al., 2004; Staswick, 1997; Staswick et al., 1994). Under drought conditions, this RuBisCO-associated protein was upregulated in leaves.

In plants, glyoxylate aminotransferases in the peroxisome participate in photorespiration, a pathway that salvages byproduct from RuBisCO's oxygenase activity (Liepman and Olsen, 2003). Under drought conditions, predicted glutamate-glyoxylate aminotransferase 2-like isoform 2 was upregulated in roots and leaves.

Increases in organic acid metabolism support a shift in carbon metabolism that accommodates increased nitrate assimilation (Scheible et al., 2000). Isocitrate dehydrogenase is a metabolic enzyme in organic acid metabolism (Scheible et al., 2000), and dicarboxylate/tricarboxylate transporters are responsible for compartmentation of organic acids necessary to maintain cytosolic enzyme functions (Regalado et al., 2012). Under normal watering, predicted isocitrate dehydrogenase [NADP] and predicted mitochondrial dicarboxylate/tricarboxylate transporter DTC-like were upregulated in roots. Under drought conditions, predicted isocitrate dehydrogenase [NADP] was upregulated in leaves.

Aconitate hydratase, or aconitase, is a key enzyme converts citrate to isocitrate, participating in the cytosolic glyoxylate cycle, related to photorespiration, in cytosolic citrate metabolism, related to the balance of nitrate and organic acid metabolism, and in the mitochondrial tricarboxylic acid cycle (Arnaud et al., 2007; Peyret et al., 1995; Scheible et al., 2000; Terol et al., 2010). Under drought conditions, predicted aconitate hydratase 2, mitochondrial-like was upregulated in leaves.

Chloroplast stem-loop binding proteins have been shown to regulate mRNA stability of chloroplast precursor mRNAs via pre-processing and degradation (Monde et al., 2000; Yang et al., 1996). Under drought conditions, predicted chloroplast stem-loop binding protein of 41 kDa a, chloroplastic-like was upregulated in leaves.

CURVATURE THYLAKOID 1A (CURT1A) is active in the chloroplast, modifying thylakoid architecture and interacting with vesicle transport (Lindquist and Aronsson, 2014). Under drought conditions, predicted protein CURVATURE THYLAKOID 1A, chloroplastic-like was upregulated in roots.

Thylakoid luminal proteins are those localized to the lumen between thylakoid membranes in the chloroplast, participating in various functions (Konishi et al., 1993). Under normal watering, predicted thylakoid luminal 16.5 kDa and 29 kDa proteins, chloroplastic-like were upregulated in leaves.

ATP synthase provides energy to the cell through the synthesis of adenosine triphosphate (ATP) from adenosine diphosphate (ADP) and inorganic phosphate (Pi). The ATP produced by the light reactions is then used by the dark reactions of photosynthesis to reduce CO2 to carbohydrates (McCarty, 1992). Changes in ATP synthase contents have been reported in response to changes in light intensity (Anderson et al., 1988), leaf age (Schottler et al., 2007a), and drought stress (Kohzuma et al., 2009). Under drought conditions, ATP synthase CF1 epsilon subunit was downregulated in leaves.

Glucose-6-phosphate 1-epimerase is involved in ATP production via glycolysis and gluconeogenesis (Sun et al., 2014; Wurster and Hess, 1972). Under drought conditions predicted putative glucose-6-phosphate 1-epimerase-like was downregulated in leaves.

Chloroplast transketolase participates in carbon metabolism via the Calvin-Benson-Bassham cycle and the oxidative pentose phosphate pathway, and may have a role in regulating carbon allocation under light and dark conditions (Rocha et al., 2014). Under drought conditions, a predicted chloroplastic transketolase was downregulated in roots.

The photosystem I complex completes photosynthesis by catalyzing oxidation of plastocyanin and reduction of ferredoxin, the final steps of electron transport (Schottler et al., 2007b). Within photosystem I, the P700 chlorophyll a protein is a chlorophyll-binding protein that accumulates in response to light via translational regulation (Kreuz et al., 1986). Under drought conditions, photosystem I P700 apoprotein A2 was downregulated in roots.

In plants, glyoxylate aminotransferases in the peroxisome participate in photorespiration, a pathway that salvages byproduct from RuBisCO's oxygenase activity (Liepman and Olsen, 2003). Under normal watering, predicted glutamate—glyoxylate aminotransferase 2-like isoform 2 was downregulated in roots.

Increases in organic acid metabolism support a shift in carbon metabolism that accommodates increased nitrate assimilation (Scheible et al., 2000). Isocitrate dehydrogenase is a metabolic enzyme in organic acid metabolism (Scheible et al., 2000), and dicarboxylate/tricarboxylate transporters are responsible for compartmentation of organic acids necessary to maintain cytosolic enzyme functions (Regalado et al., 2012). Under drought conditions, predicted isocitrate dehydrogenase [NAD] catalytic subunit 5, mitochondrial-like was downregulated in roots.

Chloroplast stem-loop binding proteins have been shown to regulate mRNA stability of chloroplast precursor mRNAs via pre-processing and degradation (Monde et al., 2000; Yang et al., 1996). Under normal watering, predicted chloroplast stem-loop binding protein of 41 kDa b, chloroplastic-like isoform X2 was downregulated in roots.

CURVATURE THYLAKOID 1A (CURT1A) is active in the chloroplast, modifying thylakoid architecture and interacting with vesicle transport (Lindquist and Aronsson, 2014). Under normal watering, predicted protein CURVATURE THYLAKOID 1A, chloroplastic-like was downregulated in leaves. Under drought conditions, this protein was downregulated in leaves.

Magnesium chelatase is the first unique enzyme in chlorophyll biosynthesis, and may be regulated by abscisic acid in some species (Müller and Hansson, 2009; Shen et al., 2006). Under normal watering, a predicted chloroplastic magnesium-chelatase subunit (ChlI) was downregulated in roots.

### Cell Wall

In plants, peroxidases are involved in cell wall lignification, usually associated with pathogen resistance (Bruce and West, 1989), abiotic stress (Huttova et al., 2006; Quiroga et al., 2001), or cell wall modification during growth (Van Hoof and Gaspar, 1976; Kukavica et al., 2012). Under drought conditions, predicted peroxidase 50-like was upregulated in leaves.

UTP-glucose-1-phosphate uridylyltransferase provides UDP-glucose, an important substrate for cell wall polysaccharide biosynthesis (Cook et al., 2012; Hertzberg et al., 2001). Under drought conditions predicted UTP-glucose-1-phosphate uridylyltransferase-like was upregulated in roots.

Dirigent proteins have a chaperone-like role in selecting the stereochemistry or regiospecificity in enzyme reactions involving radical-radical coupling, including the formation of lignans, norlignans, and ellagitannins important in vasculature and plant defense (Davin and Lewis, 2005). Under normal watering, predicted dirigent protein 1-like was upregulated in leaves.

### Developmental Regulation

The THO complex is a factor composed of four polypeptides that associates with RNA and DNA to facilitate transcription elongation (Jimeno et al., 2002; Rondon et al., 2003). Under normal watering, predicted THO complex subunit 4-like isoform X1 was upregulated in roots.

The ribosome is essential for translation of mRNA into proteins in all living organisms. In eukaryotes, the ribosome is composed of a large 60S subunit and a small 40S subunit; in bacteria and plastids, these are smaller 50S and 30S subunits, respectively (Ben-Shem et al., 2011). Chloroplastic 50S ribosomal proteins are involved in synthesis of organelle-specific proteins in the chloroplast (Bartsch et al., 1982). Under normal watering, predicted probable 60S ribosomal protein L14-like was upregulated in leaves. Under drought conditions predicted 50S ribosomal protein L6, chloroplastic-like isoform 1 was upregulated in roots, and predicted 50S ribosomal protein L11, chloroplastic-like was upregulated in leaves.

Histones are primarily involved in DNA packaging into chromatin, a process that modifies gene expression. Recent studies show that the developmental transition from a vegetative to a reproductive phase (i.e. flowering) is controlled by chromatin modifications (He, 2009). Under normal watering, predicted histone H4-like was upregulated in leaves.

The proteasome is a complex of multiple subunits, including protease and regulatory subunits, that degrades proteins in response to regulatory signaling, including ubiquitination (Smalle et al., 2002). Under normal watering, predicted proteasome subunit alpha type-5-like was upregulated in roots. Under drought conditions, predicted 26S protease regulatory subunit 10B homolog A-like was upregulated in roots.

Cucumisin is a protease in the family of subtilases, which carry out regulatory roles as well as protein degradation in plants, including breakdown of storage proteins, xylem differentiation, and pathogen defense (Schaller, 2004; Yamagata et al., 1994). Under drought conditions, predicted cucumisin-like isoform X2 was upregulated in leaves.

Cysteine proteases and proteinases participate in developmental regulation, including accumulation and degradation of storage proteins, senescence, and programmed cell death (Schaller, 2004). Under normal watering, predicted cysteine proteinase RD21a-like was upregulated in roots.

Enolase-phosphatase is a highly conserved, bifunctional enzyme participating in the methionine salvage pathway associated with biosynthesis of methionine and ethylene, maintaining efficiency of those processes by recovering their byproducts (Albers, 2009). Under drought conditions, predicted enolase-phosphatase E1-like was upregulated in roots.

CDGSH iron-sulfur domain-containing proteins (CISDs), in the NEET family, are highly conserved proteins capable of both electron accepting and electron donating, targeted to the mitochondrion or the chloroplast, and involved in developmental and homeostatic regulation (Su et al., 2013; Tamir et al., 2015). Under normal watering, predicted CDGSH iron-sulfur domain-containing protein NEET-like was upregulated in leaves.

The THO complex is a factor composed of four polypeptides that associates with RNA and DNA to facilitate transcription elongation (Jimeno et al., 2002; Rondón et al., 2003). Under drought conditions, predicted THO complex subunit 4-like isoform X1 was downregulated in roots.

The ribosome is essential for translation of mRNA into proteins in all living organisms. In eukaryotes, the ribosome is composed of a large 60S subunit and a small 40S subunit; in bacteria and plastids, these are smaller 50S and 30S subunits, respectively (Ben-Shem et al., 2011). Chloroplastic 50S ribosomal proteins are involved in synthesis of organelle-specific proteins in the chloroplast (Bartsch et al., 1982). Under drought conditions, predicted 60S ribosomal protein L4-like isoform 1 was downregulated in roots.

Histones are primarily involved in DNA packaging into chromatin, a process that modifies gene expression. Recent studies show that the developmental transition from a vegetative to a reproductive phase (i.e. flowering) is controlled by chromatin modifications (He, 2009). Under drought conditions, predicted probable histone H2B.3 was downregulated in leaves.

Leucine aminopeptidases (LAPs) are enzymes involved in protein turnover and, in some classes of LAPs, wounding responses (Scranton et al., 2013; Waditee-Sirisattha et al., 2011). Under normal watering, a predicted chloroplastic leucine aminopeptidase 3 was downregulated in roots.

Molecular chaperones have significant functional conservation across all domains of life. These proteins are implicated in facilitating proper folding of nascent polypeptides through binding of nonnative proteins to inhibit aggregation with other proteins and cellular structures. The CPN60 family of chaperonins include HSP60 chaperones found in bacteria, mitochondria and chloroplasts and the TCP-1 complex of chaperones deployed by eukaryotes and archaea (Fink, 1999). Under drought conditions, predicted chaperonin CPN60-like 2, mitochondrial-like was downregulated in leaves.

Endoplasmin is heat-shock protein 90-B1 (HSP90B1) (Cawthorn et al., 2012), a molecular chaperone in a heat shock protein family involved in signal transduction, cell cycle control, and protein folding, transport, and degradation (Chen et al., 2006). In Arabidopsis, HSP90.1 was found to participate in RPS2-mediated disease resistance (Takahashi et al., 2003). Under normal watering predicted endoplasmin homolog isoform X2 was downregulated in roots.

T-complex proteins are molecular chaperones involved in assembly and quality control of other protein (Mori et al., 1992). Under normal watering, predicted T-complex protein 1 subunit gamma-like was downregulated in leaves.

Ornithine carbamoyltransferase (OCT) is one of several enzymes required for the metabolism of arginine in plants. Arginine is an important and abundant constituent of storage proteins that are present in legume seeds. OCT activity varies with time during seed development and also following germination in pea and fava bean plants (Kollöffel and Stroband, 1973; Ruiter and Kollöffel, 1982). Under drought conditions, predicted ornithine carbamoyltransferase, chloroplastic-like was downregulated in leaves.

Calcium signaling is important for regulating many aspects of plant development and stress responses, and calcium pumps are essential to this regulatory activity (Boursiac et al., 2010). Under drought conditions, predicted calcium-transporting ATPase 4, plasma membrane-type-like was downregulated in roots.

Dihydroxy-acid dehydratase is the third enzyme in biosynthesis of branched-chain amino acids key to plant growth, including valine, leucine, isoleucine, and CoA (Flint and Emptage, 1988), and may be regulated by ROS and redox signaling via the ferredoxin/thioredoxin system (Balmer et al., 2006). Under drought conditions, predicted putative dihydroxy-acid dehydratase, mitochondrial-like was downregulated in roots and leaves.

Sulfur is required for the biosynthesis of many important biological molecules including amino acids and metalloproteins. Sulfite reductases are integral in the assimilation of sulfur into cysteine by catalyzing the reduction of sulfite to sulfide. In maize and pea, sulfite reductase has implicated in nucleoid-compaction; specifically in pea this compaction causes a reduction in transcriptional activity in plastid nucleoids (Sekine et al., 2007). Under drought conditions, a predicted chloroplastic sulfite reductase was downregulated in roots.

The bacterial cell division protein FtsZ is an ancestral tubulin involved in cell division of plastids in plants (Strepp et al., 1998). Under normal watering, predicted cell division protein FtsZ homolog 1, chloroplastic-like was downregulated in leaves.

### Resistance to Abiotic and Biotic Stresses

### Abiotic Stresses

In plants, alcohol dehydrogenase, a highly conserved enzyme, is induced by stress conditions, particularly during hypoxic response, to anaerobically supply NAD+ for metabolism (Chung and Ferl, 1999). Under drought conditions, predicted alcohol dehydrogenase 1 was upregulated in leaves, while predicted alcohol dehydrogenase class-3

Aldehyde dehydrogenases are members of the NAD(P)(+)-dependent protein superfamily involved in the conversion of various aldehydes to their corresponding nontoxic carboxylic acids (Brocker et al., 2013). Aldehyde dehydrogenases are involved in a wide range of metabolic pathways including growth, development, seed storage, and environmental stress adaptation in higher plants (Rodrigues et al., 2006; Brocker et al., 2013). Under drought conditions, predicted aldehyde dehydrogenase family 3 member I1, chloroplastic-like were upregulated in roots.

Peroxisomal 3-ketoacyl-CoA thiolase functions in fatty acid β-oxidation with broad substrate specificity, important particularly in seedlings for accessing stored lipids for carbon and energy during early growth (Germain et al., 2001). Derivatives of very-long-chain fatty acids (20 or more carbons) act as protective barriers between plants and the environment, provide energy storage in seeds, and function as signaling molecules in membranes (Devaiah et al., 2006; Pollard et al., 2008). Under drought conditions, predicted 3-ketoacyl-CoA thiolase 2, peroxisomal-like isoform X1 was upregulated in roots.

Thioredoxins are implicated in different aspects of plant life including development and adaptation to environmental changes and stresses. They act as antioxidants by facilitating the reduction of other proteins by cysteine thiol-disulfide exchange (Nordberg and Arnér, 2001). Under normal watering, predicted thioredoxin M4, chloroplastic-like and predicted thioredoxin-like protein CDSP32, chloroplastic-like were upregulated in leaves. Under drought conditions, predicted thioredoxin-like protein CDSP32, chloroplastic-like was upregulated in leaves.

Peptidyl prolyl cis-trans isomerases are molecular chaperones that participate in protein folding and signal transduction (Aviezer-Hagai et al., 2006). In Arabidopsis, the two peptidyl prolyl cis-trans isomerases ROF1 and ROF2 possess different tissue-specific, developmentally regulated, and heat-inducible expression patterns (Aviezer-Hagai et al., 2006). ROF2 has also been shown to participate in intracellular pH homeostasis in Arabidopsis (Bissoli et al., 2012). Under normal watering, predicted peptidyl-prolyl cis-trans isomerase CYP20-2, chloroplastic-like isoform 1 was upregulated in roots. Under drought conditions, predicted chloroplastic peptidyl-prolyl cis-trans isomerase was upregulated in roots.

Abiotic stresses and pathogen attack can result in accumulated DNA damage and eventual genotoxic stress, necessitating coordination of DNA repair in response to environmental stress (Doná et al., 2013). RAD26 is a nucleotide excision repair protein first identified in yeast (van Gool et al., 1994), with homologs later identified in plants (Heitzeberg et al., 2004; Liu et al., 2000). DRT-100 is a DNA-damage-repair/toleration gene associated with UV damage (Hays and Pang, 1994; Pang et al., 1993). Under normal watering, predicted DNA repair and recombination protein RAD26-like was upregulated in roots. Under drought conditions, DNA-damage-repair/toleration protein DRT100-like precursor was upregulated in roots.

The heat-shock proteins are molecular chaperones expressed under various stresses to stabilize proteins (De Maio, 1999). Under normal watering, predicted stromal 70 kDa heat shock-related protein, chloroplastic-like was upregulated in leaves. Under drought conditions, heat shock protein 90-1 was upregulated in roots.

The luminal binding proteins (BiP) are molecular chaperones in the endoplasmic reticulum, participating in protein folding and quality control processes (Valente et al., 2009). Studies in Arabidopsis, tobacco, and soy have shown that BiP protects against heat and drought stress (Koizumi, 1996; Valente et al., 2009). Under drought conditions, predicted luminal-binding protein was upregulated in roots.

In plants, alcohol dehydrogenase, a highly conserved enzyme, is induced by stress conditions, particularly during hypoxic response, to anaerobically supply NAD+ for metabolism (Chung and Ferl, 1999). Under normal watering, predicted alcohol dehydrogenase 1 and predicted alcohol dehydrogenase class-3 were downregulated in roots.

Peroxisomal 3-ketoacyl-CoA thiolase functions in fatty acid β-oxidation with broad substrate specificity, important particularly in seedlings for accessing stored lipids for carbon and energy during early growth (Germain et al., 2001). Derivatives of very-long-chain fatty acids (20 or more carbons) act as protective barriers between plants and the environment, provide energy storage in seeds, and function as signaling molecules in membranes (Devaiah et al., 2006; Pollard et al., 2008). Under drought conditions, predicted 3-ketoacyl-CoA thiolase 2, peroxisomal-like isoform X1 was downregulated in leaves.

The Do proteases, also called Deg and HtrA (Ponting, 1997), are ATP-independent serine endopeptidases common across all domains of organisms (Schuhmann and Adamska, 2012). In plants, Deg proteases conduct protein turnover and cellular regulation primarily in the chloroplast to combat photodamage, but also in the peroxisome to support β-oxidation processes (Schuhmann and Adamska, 2012). Under normal watering, predicted protease Do-like 2, chloroplastic-like was downregulated in leaves.

Myo-inositol 3-phosphate synthase is the first enzyme in myo-inositol biosynthesis, and its overexpression has been shown to increase salt tolerance in multiple plant species via activation of basal metabolism, inositol metabolism, glycolysis, the pentose phosphate pathway, and the tricarboxylic acid cycle (Kusuda et al., 2015). Under normal watering, predicted inositol-3-phosphate synthase was downregulated in leaves.

Lactoylglutathione lyase (glyoxalase I) participates in the glyoxalase pathway to detoxify methylglyoxal, a cytotoxic molecule associated with abiotic stresses (Mustafiz et al., 2011). Overexpression of lactoylglutathione lyase has been shown to provide tolerance to salinity and heavy metal stress (Mustafiz et al., 2011). Under drought conditions, predicted putative lactoylglutathione lyase-like isoform X2 was downregulated in leaves.

Delta-1-pyrroline-5-carboxylate synthase, the first enzyme in biosynthesis of the osmoprotectant proline, is induced by salt stress and associated with protection from osmotic stress (Ginzberg et al., 1998; Hong et al., 2000). Under drought conditions, predicted delta-1-pyrroline-5-carboxylate synthase-like was downregulated in leaves.

Abscisic acid stress ripening proteins (Asr), a DNA-binding protein that improves drought and salinity tolerance, is upregulated by water stress, salt stress, and the hormone abscisic acid, as well as by developmental regulation (Goldgur et al., 2007; Kalifa et al., 2004). Under drought conditions, abscisic stress ripening-like protein was downregulated in roots.

### Biotic Stresses

Lipoxygenases catalyze the dioxygenation of polyunsaturated fatty acids in oxylipins, a group of lipids that include j asmonic acid (JA) and its derivatives, and which are involved in a number of developmental and stress response processes (Andersson et al., 2006). Oxilipins may exert protective activities either as signaling molecules in plants during development, wounding, insect and pathogen attack, or as direct anti-microbial substances that are toxic to the invader (Yan et al., 2013). Under normal watering, one lipoxygenase was upregulated in leaves. Under drought conditions, a second lipoxygenase was upregulated in leaves, and a third in roots.

1-deoxy-D-xylulose 5-phosphate reductoisomerase is an enzyme in the plastidial, nonmevalonate pathway for biosynthesis of isoprenoids, including the terpenoids, which serve key roles in plant defense (Lange and Croteau, 1999). Under normal watering, predicted 1-deoxy-D-xylulose 5-phosphate reductoisomerase, chloroplastic-like was upregulated in leaves.

Patellins contain a Sec14 domain, implicated in lipid signaling, lipid metabolism, and membrane trafficking, and they have been shown to be involved in Arabidopsis root cell division and interference with viral movement proteins (MPs) that coordinate inter- and intracellular viral localization (Peiro et al., 2014). Under normal watering, predicted patellin-3-like isoform X3 was downregulated in roots.

(S)-2-hydroxy-acid oxidase, also called glycolate oxidase (Schomburg and Stephan, 1995), participates in photorespiration and in defense responses to pathogens (Chern et al., 2013). Interestingly, downregulation of GLO genes in rice resulted in greater pathogen resistance, possibly due to induction of basal resistance (Chern et al., 2013). Under normal watering, predicted peroxisomal (S)-2-hydroxy-acid oxidase GLO1-like isoform X1 was downregulated in roots.

3′-hydroxy-N-methyl-(S)-coclaurine 4′-O-methyltransferase participates in alkaloid biosynthesis and synthesis of cysteine and adenosine, by producing the intermediates (S)-reticuline and S-adenosyl-L-homocysteine (Frenzel and Zenk, 1990). Under drought conditions, 3′-hydroxy-N-methyl-(S)-coclaurine 4′-O-methyltransferase-like, an enzyme in this pathway, was downregulated in roots.

### Example 10: Identification of Differentially Regulated Hormones

### Methods

For hormone analysis, 100±10 mg tissue was measured into microtubes (chilled with liquid nitrogen), and sent on dry ice to the lab of Dr. Michael Kolomiets in the Department of Plant Pathology and Microbiology at Texas A&M University. Plant hormone analysis was performed per Christiansen et al. (2014) with slight modification. Briefly, hormones were extracted from 100±10 mg of frozen tissue and tissue weights were recorded for quantification. A mixture containing 10 microliters of 2.5 microMolar internal standards and 500 microliters of extraction buffer [1-propanol/H20/concentrated HCl (2:1:0.002, vol/vol/vol) was added to each sample and vortexed until thawed. Samples were agitated for 30 min at 4° C., then 500 microliters of dichloromethane (CH2C12) were added. Samples were agitated again for 30 min at 4° C., and then centrifuged at 13,000×g for 5 min. in darkness. The lower organic layer was removed into a glass vial and the solvent was evaporated by drying samples for 30-40 min under a N2 stream. Samples were re-solubilized in 150 microliters of MeOH, shaken for 1 min and centrifuged at 14,000×g for 2 min. A supernatant of 90 microliters was transferred into the autosampler vial and hormones were analyzed by ultraperformance liquid chromatography, coupled to mass spectrometry (UPLC-MS/MS). Ascentis Express C-18 Column (3 cm×2.1 mm, 2.7 cm) connected to an API 3200 using electrospray ionization-tandem mass spectrometry (MS/MS) with scheduled multiple reaction monitoring (SMRM). The injection volume was 5 microliters and had a 300 microliters/min mobile phase consisting of Solution A (0.05% acetic acid in water) and Solution B (0.05% acetic acid in acetonitrile) with a gradient consisting of (time-% B): 0.3—1%, 2—45%, 5—100%, 8—100%, 9—1%, 11—stop. Quantitation was carried out with Analyst software (AB Sciex), using the internal standards as a reference for extraction recovery. Leaf and root tissue was saved in −62° C. and saved for subsequent gene expression analysis.

Mass spectra of 8 plant hormones were obtained: jasmonic acid (JA), jasmonic acid-isoleucine (JA-Ile), salicylic acid (SA), abscisic acid (ABA), 12-oxo-phytodienoic acid (OPDA), 10-oxo-11 phytoenoic acid (OPEA), traumatic acid (TA) and cinnaminic acid (CA). Fold changes between control and treated samples were calculated by dividing the mass spectrum value from the treated sample by the value from the control sample.

### Results

All results are summarized in Table 9.

The plant hormone analysis of soybean plants inoculated with endophytic fungal strain Strain B grown under normal and water-limiting conditions in the greenhouse revealed that Strain B augmented and modified hormone levels in different tissue types and growth conditions in planta.

Our data shows that the levels of the plant hormone abscisic acid (ABA) were decreased in Strain B-treated plants compared to plants grown from seed treated with formulation only, grown under normal and water-limiting conditions, in all three tissue types except stem tissue under normal watering regime. ABA is involved in regulation of developmental processes such as seed maturation and dormancy (Baker et al., 1988), responses to environmental stresses (Shinozaki and Yamaguchi-Shinozaki, 2000) including stomatal closure (McAinsh, 1990) and expression of stress-related genes (Urao et al., 1993). Thus, plants treated with compositions such as Strain B may have an improved ability to cope with the stresses associated with normal and water-limited conditions, via modulation of expression of ABA.

Salicylic acid (SA) and cinnamic acid (CA) are upregulated in roots under normal condition and stems and leaves under drought, and CA is upregulated in stems under normal condition. They are down-regulated in leaves under normal condition and roots under drought and SA is downregulated in stems under normal condition. SA is considered one of the key endogenous component involved in local and systemic defense responses in plants (Shah and Klessig, 1999). At the infection site, plant triggers localized programmed cell death, a phenomenon known as the hypersensitive response (Caplan et al., 2008), followed by accumulation of SA, and an induction of pathogenesis-related proteins in distal tissues to protect plants from secondary infections. This type of protection is called systemic acquired resistance (SAR) and it provides broad-spectrum resistance against pathogenic fungi, oomycetes, bacteria and viruses (Shah and Klessig, 1999). SAR is associated with significant transcriptional reprogramming, which is dependent on the transcription cofactor NPR1 and its associated transcription factors (Dong, 2004). Protective effect of SAR can last for months, and possibly even throughout the whole growing season (Kuc, 1987). SA is synthesized through phenylpropanoid pathway from cinnamic acid (CA) via two possible pathways (Klambt, 1962; el-Basyouni et al., 1964). Cinnamic acid is a precursor for biosynthesis of the polyphenol compounds (Lee et al., 1995) that have multiple functions, such as providing mechanical support (lignins) (Whetten and Sederoff, 1992), protection against abiotic and biotic stress (antioxidants) (Dixon and Paiva, 1995), and signaling with the flavonoid nodulation factors (Weisshaar and Jenkins, 1998). The upregulated levels of SA and CA under both growth conditions is consistent with recent studies showing that plants respond to endophytic colonization by local defense responses (Compant et al., 2005), but the levels of expression are much lower than when plants are challenged with the pathogen (Bordiec et al., 2011), allowing the endophyte to systemically colonize the plant (Reinhold-Hurek and Hurek, 2011). Thus, plants treated with compositions such as Strain B may have an improved ability to cope with the stresses associated with normal and water-limited conditions, via modulation of expression of SA and/or CA.

Jasmonic acid (JA) and its derivative jasmonic acid isoleucine (JA-Ile) are up-regulated in leaves of Strain B-treated plants grown normal watering condition and in roots of plants grown under water-limited conditions and JA-Ile in stems under the same condition. JA and JA-Ile are down-regulated in roots and stems of Strain B-treated plants grown normal watering condition and in leaves of plants grown under water-limited conditions and JA in stems under the same condition. Jasmonates (JAs) are formed by the enzymatic action of 13-LOX on linolenic acid that enables production of 12-oxo-phytodienoic acid (OPDA) and its downstream products such as free JA, MeJA, cis-jasmone and JA-Ile (Göbel and Feussner, 2009). JAs are a type of oxylipins, that are involved in a number of developmental or stress response processes (Andersson et al., 2006) and they exert protective activities either as signaling molecules in plants during development, wounding, insect and pathogen attack, or direct anti-microbial substances that are toxic to the invader (Yan Y et al., 2013). The opposite pattern of expression under both normal and drought conditions of JA and JA-Ile with SA and CA is in line with well documented literature that SA and JA act antagonistically (Beckers and Spoel, 2006). Thus, plants treated with compositions such as Strain B may have an improved ability to cope with the stresses associated with normal and water-limited conditions, via modulation of expression of JA and/or JA-Ile.

12-oxo-phytodienoic acid (OPDA) levels are downregulated in all 3 tissue types in Strain B-treated plants grown under normal watering conditions and upregulated in all 3 tissue types in Strain B-treated plants grown under water-limited conditions. OPDA is a type of oxylipins, that are involved in a number of developmental or stress response processes (Andersson et al., 2006) and they exert protective activities either as signaling molecules in plants during development, wounding, insect and pathogen attack, or direct anti-microbial substances that are toxic to the invader (Yan Y et al., 2013). Thus, plants treated with compositions such as Strain B may have an improved ability to cope with the stresses associated with normal and water-limited conditions, via modulation of expression of OPDA.

10-oxo-11-phytoenoic acid (OPEA) is upregulated in roots and leaves under normal and roots and stems under water-limited conditions and downregulated in stems under normal and leaves under water-limited conditions. OPEA is produced in a pathway involving 9-LOX activity on linoleic acid. Despite structural similarity to jasmonates, physiological roles for OPEA is not well understood. This hormone is highly induced at the site of pathogen infection and it can suppress the growth of mycotoxigenic fungi suggesting more specialized roles in local defense reactions (Christensen et al., 2015). Thus, plants treated with compositions such as Strain B may have an improved ability to cope with the stresses associated with normal and water-limited conditions, via modulation of expression of OPEA.

Levels of traumatic acid (TA) are down-regulated in roots, stems and leaves of Strain B-treated plants grown under normal watering conditions and are downregulated in stems of Strain B-treated plants grown under water-limited conditions. TA is upregulated in roots and leaves of Strain B-treated plants grown under water-limited conditions. Traumatic acid, which is produced from both linoleic acid and linolenic acids, is a plant wound hormone associated with cell proliferation in plants (Vick and Zimmerman, 1987) and causes abscission in cotton buds (Strong and Kruitwagen, 1967). Thus, plants treated with compositions such as Strain B may have an improved ability to cope with the stresses associated with normal and water-limited conditions, via modulation of expression of TA.

### Example 11: Identification of Differentially Regulated Metabolites (Metabolomics)

### Methods

For metabolite analysis, 150±10 mg of each sample was transferred into 1.5 mL microtubes (chilled in liquid nitrogen) and sent on dry ice to the Proteomics and Metabolomics Facility at Colorado State University. Metabolomics data acquisition was performed per the following methods provided by Dr. Corey Broeckling at CSU. To prepare the samples for analysis, phytohormones were extracted from ground plant material using a biphasic protocol. One mL of a methyl tert-butyl ether (MTBE): methanol:water mixture (6:3:1) was added to each sample then shaken for 1 hour. Next, 250 microliters cold water and a mix of internal standards was added to each sample to promote phase separation. Samples were shaken again for 5 minutes. Samples were then centrifuged at 2,095×g at 4° C. for 15 minutes. The organic top phase was removed for hormone analysis, dried under an inert nitrogen environment, then re-suspended in 400 microliters of 50% acetonitrile. Extracts were then directly analyzed by LC-MS.

For GC-MS, the polar (lower phase) extract was dried using a speedvac, resuspended in 50 microliters of pyridine containing 50 mg/mL of methoxyamine hydrochloride, incubated at 60° C. for 45 min, sonicated for 10 min, and incubated for an additional 45 min at 60° C. Next, 25 microliters of N-methyl-N-trimethylsilyltrifluoroacetamide with 1% trimethylchlorosilane (MSTFA+1% TMCS, Thermo Scientific) was added and samples were incubated at 60° C. for 30 min, centrifuged at 3000×g for 5 min, cooled to room temperature, and 80 microliters of the supernatant was transferred to a 150 microliters glass insert in a GC-MS autosampler vial. Metabolites were detected using a Trace GC Ultra coupled to a Thermo ISQ mass spectrometer (Thermo Scientific). Samples were injected in a 1:10 split ratio twice in discrete randomized blocks. Separation occurred using a 30 m TG-5MS column (Thermo Scientific, 0.25 mm i.d., 0.25 micrometer film thickness) with a 1.2 mL/min helium gas flow rate, and the program consisted of 80° C. for 30 sec, a ramp of 15° C. per min to 330° C., and an 8 min hold. Masses between 50-650 m/z were scanned at 5 scans/sec after electron impact ionization. The ionization source was cleaned and retuned and the injection liner replaced between injection replicates. Analysis for plant hormones was performed by UPLC-MS/MS as follows.

Over 1250 metabolites were detected and mass spectra annotated by comparing to libraries of known spectra including an in-house database of ˜1200 compounds at CSU (LC-MS only), the National Institute of Standards and Technology databases, Massbank MS database, and the Golm Metabolite Database. Initial annotation was automated, followed by manual validation of annotations. Following annotation, approximately 160 compounds were identified. After removal of technical artifacts (e.g. siloxane), and ambiguous or vague annotations (e.g. carbohydrate or saccharide), 145 identified compounds remained for analysis. These compounds were assessed for fold change over control plants. Metabolites were grouped by pathways (e.g. carbohydrate metabolism or alkaloid biosynthesis) and the KEGG database and literature were manually referenced to identify pertinent shifts in metabolic patterns in plants treated with microbes. Any compound without an appreciable shift compared to that observed in control plants was removed from further analysis

### Results

All results are summarized in Table 10A (normal watering conditions) and Table 10B (water-limited conditions).

An important metabolic system in plants involves the production of phenylpropanoid compounds. The production of a wide variety of phenylpropanoids is induced under stress conditions and important plant signaling molecules, e.g. salicylic acid, are derived from phenylpropanoid precursors (Dixon and Paiva, 1995). The shikimic acid pathway sits atop many of these mechanisms as it produces the cyclic amino acids that constitute the raw materials for many defense compounds that can be induced upon biotic or abiotic challenge. The stresses that can induce production of various stress-related phenylpropanoid pathways include, but are not limited to, pathogen challenge (phytoalexins), wounding (lignins), and nutrient deficiency (flavonoids and isoflavonoids). Beneficial Penicillium endophyte treatments showed modulation of phenylpropanoid production under well-watered and drought conditions, often in a tissue-specific manner, as well as causing alterations in the levels of aromatic amino acid precursors (phenylalanine, tyrosine, tryptophan) that feed into these pathways. Lignin, for example, is an important structural component in plants, second in abundance only to cellulose. There is some evidence that lignin biosynthesis genes are upregulated in the root tissue of plants under various stages of drought stress (Moura et al., 2010), the rationale being root growth will be stimulated when plants are at a water deficit. Under the conditions tested there was a reduction in the quantity of lignin precursors (ferulic acid, caffeic acid) in well-watered plant roots treated with Strain B (relative to control). By contrast there was an increase in these metabolites in plants subjected to drought and treated with the same microbe.

Another diverse group of plant metabolites, the alkaloids, may be constitutively synthesized in the plant or may be produced de novo in response to biotic or abiotic challenge. Although alkaloids can be synthesized in response to stresses such as wounding, they are also transiently produced in early stages of plant development (Cheong et al., 2002). In this case, specific alkaloid synthetic pathways may become active in rapidly dividing cells in apical regions of root tissue (De Luca and St Pierre, 2000). Beneficial Penicillium endophyte treatments elicited a variety of alterations in alkaloid biosynthetic pathways under well-watered and drought (water-limited) conditions. 2-piperidinecarboxylic acid (pipecolic acid), accumulates in plants in response to pathogen attack, and has been shown to accumulate in halotolerant species (Navarova et al., 2012, Moulin et al., 2006). Pipecolic acid, a degradation product of the amino acid lysine, is an intermediary of tropane alkaloid biosynthesis and was observed to accumulate differentially in leaf tissue of plants grown from seeds treated with beneficial Penicillium endophytes, when compared to non-inoculated control plants.

Flavonoid and isoflavonoids compounds are exuded by plant roots into the rhizosphere in response to nutrient stress in order to recruit compatible nitrogen-fixing bacteria. These signals are perceived by N-fixing rhizobia, which then begin production of nodulation factors that stimulate the development of nodules in the roots of the host plant (Gibson et al., 2008). Indeed, one study showed Rhizobium leguminosarum cells pretreated with plant-produced hesperetin stimulate increased nodulation in the host compared to bacteria that are not pretreated (Begum et al., 2001). In addition to playing a role in symbiosis development, these compounds may also function in pathogen response. Daidzein, in particular, accumulates in soybean plants in response to invasion by pathogenic Pseudomonas (Osman and Fett, 1982). Strain B treatments caused a relative decrease in daidzein in root and stem tissue of well-watered plants with a contrasting increase in leaf tissue. Additionally, beneficial Penicillium endophyte treatment induced the accumulation of hesperetin in stem tissue in the drought condition.

A variety of other metabolites were modulated by beneficial Penicillium endophyte treatment. For instance, a direct precursor to brassinosteroids, campesterol, was to reduced relative to control in leaf tissue of water-stressed plants treated with Strain B. Additionally, campesterol is increased relative to control in leaves of well-watered plants treated with Strain B. Brassinosteroid hormones are important in plant growth and development, as shown through the relatively high concentrations measured in reproductive and developing tissues (Khripach et al., 2000). Lumichrome, a degradation product of riboflavin, has been shown to be produced by root-associated Rhizobia. Lumichrome has the ability to affect plant root respiration, transpiration rates, as well as stomatal conductance in a variety agrinomically relevant plants (Phillips et al., 1999, Matiru and Dakora, 2005). In addition to production by members of the Rhizobia, it has been shown that soil microbes such as Pseudomonas can degrade riboflavin to lumichrome in rhizosphere systems (Yanagita and Foster, 1956). Further, lumichrome can promote plant growth, perhaps through its ability to stimulate increases in photosynthetic rates (Matiru and Dakora, 2005; Khan et al., 2008). In the current experiment, lumichrome levels remained unchanged or were reduced relative to control in well-watered plants. However under drought conditions there was an observed accumulation of lumichrome in various tissues, including stem (Strain B). As lumichrome affects stomatal conductance and photosynthetic rates, it is possible that it may provide a benefit to plants in both water deficit and excess water conditions. Allantoin, a product of urea metabolism, can constitute a large percentage of the soluble nitrogen in plant sap and may be integral in nitrogen transport in nodulated soybean plants (Reinbothe and Mothes, 1962; McClure and Israel, 1979). When a variety of legumes were examined for allantoin accumulation under water-deficit conditions and its associated effect on nitrogen-fixation, more drought tolerant genotypes were observed to maintain low levels of allantoin while drought-sensitive varieties tended to accumulate greater amounts of allantoin (Serraj et al., 1999). The reduction in allantoin in drought-tolerant varieties correlated with these plants' ability to fix nitrogen in the face of water deprivation. Further, laboratory and field experiments both show similar results, as ureides such as allantoin accumulate in soy plants, particularly in shoot tissue, under drought stress while N-fixation is inhibited (Sinclair and Serraj, 1995; Serraj et al., 1999). The beneficial Penicillium endophyte treatments tested caused increased accumulation of allantoin in stem tissue under normal conditions relative to control plants, while both also exhibited a reduction in allantoin when challenged with drought stress.

In addition to the specific compounds and pathways listed above, beneficial Penicillium endophyte treatments caused significant modulation in the levels of free amino acids and nitrogenous compounds under both watering regimes, suggesting microbe-mediated shifts in nitrogen metabolism. Strain B caused an elevation of many amino acids in leaf tissue while having more variable, but a generally depressive effect, on amino acid levels in stem tissue. Further, it stimulated the accumulation in stem tissue of nearly all amino acids detected and despite a general decrease in free amino acids detected in root tissues of plants treated with both microbes. Of particular note, certain amino acids including alanine, are found to accumulate in plant cells adapted to water stress (Handa et al., 1983). Under the conditions tested, relative increase in alanine levels occurred in stem tissue of drought-stressed plants grown from seeds treated with Strain B. In addition to alanine, Handa et al. (1983) describe drought-mediated increases in several other amino acids such as valine, histidine, serine, isoleucine, and leucine. Beneficial Penicillium endophyte variously modulated these amino acids in response to water stress in a seemingly tissue-specific manner. For example, Strain B was capable of inducing the accumulation of histidine, serine, leucine, and valine in leaf tissue of plants exposed to drought.

The metabolism of carbohydrates and lipids also shifted with Strain B treatment. Sucrose, for example, accumulated in both root and stem tissue of well-watered plants treated with Strain B, which may correspond to relative increases in photosynthetic carbon assimilation compared to control plants. Galactose was also shown to increase relative to controls in stem and leaf tissue of well-watered plants treated with Strain B. In the drought condition, both microbes displayed a generally depressive effect on relative carbohydrate levels, although in many cases there is little to no change observed. Fatty acids may serve as precursors to lipid-based hormones such as the jasmonates. These important signaling molecules fill developmental roles as well as contributing to the plant's protective arsenal where their synthesis may be stimulated by herbivory and exposure to pathogens or UV and wounding (Wasternack and Kombrink, 2009). Strain B treatment affected lipid metabolism as shown by modulation in the levels of a variety of fatty acids (hexadecanoic acid) as well as other precursors to lipid biosynthesis (ethanolamine, sphingosine).

### Example 12: Microbial Community Sequencing of Plants

### Methods

Cultivation-independent analysis of microbial taxa based on marker gene high-throughput sequencing was performed as follows.

Leaf and root tissue was obtained from soybean plants grown from seeds treated with beneficial and control Penicillium endophyte strains grown under water-stressed conditions (seed treatment and growth conditions described above). Whole leaves and roots were collected from 4 biological replicates per treatment. For each treatment and tissue, the biological replicates were processed independently. The roots were cleaned in successive water baths, with manual disaggregation and removal of larger pieces of material. Tissues were flash frozen in liquid nitrogen, then ground using a mortar and pestle treated with 95% ethanol and RNAse Away (Life Technologies, Inc., Grand Island, N.Y.) to remove contaminant RNA and DNA. DNA was extracted from the ground tissues using the DNeasy DNA extraction kit (Qiagen, Hilden, Germany) according to the manufacturer's instructions. Marker genes were amplified and sequenced from the extracted DNA. For the bacterial and archaeal analyses, the V4 hypervariable region of the 16S rRNA gene was targeted (primers 515f, 806r), and for fungi, the second internal transcribed spacer (ITS2) region of the rRNA operon (primers fITS7, ITS4) was targeted. The two marker genes were PCR amplified separately using 35 cycles, and staggered 9-bp barcoded primers specific to each sample were used to facilitate combining of samples. To reduce the amplification of chloroplast and mitochondrial DNA, PNA clamps specific to the rRNA genes in these organelles were used. PCR reactions to amplify 16S rRNA and ITS regions followed the protocol of Kozich et al. (2013) (Kozich, Westcott, Baxter, Highlander, & Schloss, 2013).

PCR products were cleaned with Agencourt AMPure XP beads at a 0.7:1 bead-to-library ratio (Beckman Coulter), quantified using the PicoGreen assay (Life Technologies, Inc., Grand Island, N.Y.) and pooled in equimolar concentrations. The final library was quantified by qPCR using the KAPA Library quantification kit (KAPA Biosystems) and diluted to 4 nM. In preparation for cluster generation and sequencing, pooled libraries were denatured with NaOH, diluted with hybridization buffer, and then heat denatured before MiSeq sequencing (Illumina). Each run included a minimum of 2.5% PhiX to serve as an internal control.

### OTU Assignment

For ITS2 sequences, the raw sequence data were reassigned to distinct samples based on barcode sequences introduced during library prep, and quality filtering and OTU (i.e. operational taxonomic unit) clustering was conducted using the UPARSE pipeline (Edgar 2013). Each endophyte was assigned to an Operational Taxonomic Unit (OTU). OTU clustering (Rideout et al, 2014) was performed using a cascading approach, comparing the sequences against the Greengenes (McDonald et al., 2012) and SILVA (Quast et al., 2013) and UNITE (Abarenkov et al., 2010) reference databases, which are provided with full-length clustering at various widths. Sequences were compared to the combined Greengenes 99% OTU representative sequences and SILVA non-redundant sequences. Sequences without a 99% match to the combined reference 99% OTUs but having a 97% match were assigned to 97% OTUs with the best match representative sequence from the 99% reference sequences. Fungal sequences were compared to the UNITE Dynamic OTU representative sequences, where dynamic represents values between 97% and 99% depending on the OTU. Sequences that did not match the UNITE Dynamic OTUs at the appropriate clustering level, but did have a 97% match were assigned to 97% OTUs with best match representative sequence from the Dynamic OTUs. The remaining sequences that did not match any of the three reference databases, Greengenes, SILVA, or UNITE, but were present at a level of at least 10 reads across the samples, were de novo clustered using UPARSE (independently for the bacterial and fungal sequences). Sequences that did not match a reference sequence were mapped to the de novo OTUs at 97%. Remaining sequences that did not match either a reference or de novo OTU were removed from this analysis.

### Identification of Differences Between Treatments

Only samples having at least 1000 reads after quality filtering were retained, and only OTUs with a mean relative abundance of 0.1% within a tissue/treatment were included in this analysis. Community differences at the genus and family level were computed by summing the relative abundance of OTUs by their taxonomic assignments at the genus and family levels across all biological replicates of the tissue/treatment using the phyloseq package in R (McMurdie and Holmes (2013)) (Figures CSGen1-4 and Figures CSFam1-4). For each tissue, we identified OTUs found in all biological replicates of beneficial microbial treatment and not in microbial treatments with negative or neutral affects or in untreated controls (Tables CSUOTU1-3). OTUs with significant differences in abundance between treatments/tissues were identified using the R package DESeq2 (Love et al. 2014). Raw read counts per OTU for biological replicates of different microbial treatments and untreated controls were used as inputs to DESeq2, the log 2 fold change and adjusted p-value of each contrast are included in Tables CSDE1-2 as are the average, normalized abundance of each OTU (as counts per million) in each treatment.

### Results

All results are summarized in Table 11.

In all treatments, Enterobacteriaceae was the most abundant family of bacteria in soybean leaves and Escherichia-Shigella the most abundant bacterial genera. Seeds treated with Penicillium sp. reduced the average abundance of members of the Enterobacteriaceae family and the Escherichia-Shigella genera.

Plants grown from seeds treated with Penicillium demonstrated reduced average abundance of members of the Enterobacteriaceae family and the Escherichia-Shigella genera.

Seed treatment with Strain B increased the abundance of the arbuscular mycorrhizal (AM) fungi in roots of plants grown from said seeds. The family Glomeraceae was enriched in root tissue of plants grown from seeds treated with Strain B, and showed an increase in average abundance of 55% relative to untreated controls and an increase of 70% relative to Strain F. Glomeraceae contains several genera of AM fungi including Rhizophagus and Glomus.

The communities of plants grown from seeds treated with Strain B were enriched in OTUs belonging to the genus Rhizophagus, as compared to plants grown from seeds treated with Strain F or formulation control. On average, the members of the genus Rhizophagus made up more than 30% of the total fungal communities in samples of plants grown from seeds treated with Strain B, a 214% increase over the average abundance in untreated samples and 211% increase over the average abundance in samples of plants grown from seeds treated with Strain F. The Rhizophagus OTU F1.0|SYM97_ITS2|1601 was found within all biological replicates of soybean roots of plants grown from seeds treated with Strain B, but not in in samples of plants grown from seeds treated with Strain F or formulation control. The Rhizophagus OTUs F1.0|SYM97_ITS2|11548 and F1.0|SYM97_ITS2|1518 were significantly differentially abundant between Strain B treatment and the untreated control plants.

F1.0|SYM97_ITS2|1548 was also differentially abundant in samples of plants grown from seeds treated with Strain B.

### Example 13: Field Trials

Seeds from soybean were treated with Strain B as well as the formulation control as described in Example 4. Seeds were sown in at leaste two different growing regions for efficacy testing. Trials consisted of ten replicate plots for each treatment and control respectively arranged in a spatially balanced randomized complete block design (Van Es et al. 2007). The plot area was well-maintained and kept weed-, insect- and disease-free In addition to measuring total yield, metrics such as seedling emergence, normalized difference vegetation index (NDVI) and time to flowering were assessed. Trials were conducted during non-irrigated conditions.

All results are shown in Table 12.

Soybean trials under were conducted at one location using two soybean varieties in the Midwest region of the United States during 2015. Field conditions during the trial were particularly wet: field conditions did not constitute drought or water-limited conditions even though they were non-irrigated. No negative impacts on any measured variable was seen for plants grown from seeds treated with Strain B as compared to plants grown from seeds treated with the formulation control only. Parity was achieved for yield (bushels per acre), percent moisture (% per plot), and seed weight (pounds per bushel). No yield drag was observed under normal watering conditions.

Maize trials were conducted at two different locations using two soybean varieties in South America during 2015. Field conditions during the trial were particularly wet: field conditions did not constitute drought or water-limited conditions even though they were non-irrigated. No negative impacts on any measured variable was seen for plants grown from seeds treated with Strain B as compared to plants grown from seeds treated with the formulation control only. Parity was achieved for combine percent moisture, and combine test weight.

Having illustrated and described the principles of the present invention, it should be apparent to persons skilled in the art that the invention can be modified in arrangement and detail without departing from such principles. We claim all modifications that are within the spirit and scope of the appended claims. All publications and published patent documents cited in this specification are incorporated herein by reference to the same extent as if each individual publication or patent application is specifically and individually indicated to be incorporated herein by reference. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 1
Selected sequences of the present invention

| SEQ | | | | | | | | |
|---|---|---|---|---|---|---|---|---|
| ID | | | | | | | | |
| NO: | Kingdom | Phylum | Class | Order | Family | Genus | Species | Sequence |
| 1 | Fungi | Ascomy- | Euroto | Euro- | Tricho- | Peni- | | ATTACCGAGTGAGGGCCCTTTGGGTCCAACCTCCCACCCGTGTTTATTTTACCTTGTTGCTTCGGCGGGCCCGCCTTT |
| | | cota | Eurotio- | tiales | comaceae | cillium | | ACTGGCCGCCGGGGGGCTTCACGCCCCCGGGCCCGCGCCCGCCGAAGACACCCTCGAACTCTGTCTGAAGATTGAAGT |
| | | | mycetes | | | | | CTGAGTGAAAATATAAATTATTTAAAACTTTCAACAACGGATCTCTTGGTTCCGGCATCGATGAAGAACGCAGCGAAA |
| | | | | | | | | TGCGATACGTAATGTGAATTGCAAATTCAGTGAATCATCGAGTCTTTGAACGCACATTGCGCCCCCTGGTATTCCGGG |
| | | | | | | | | GGGCATGCCTGTCCGAGCGTCATTGCTGCCCTCAAGCCCGGCTTGTGTGTTGGGCCCCGTCCTCCGATTCCGGGGGAC |
| | | | | | | | | GGGCCCGAAAGGCAGCGGCGGCACCGCGTCCGGTCCTCGAGCGTATGGGGCTTTGTCACCCGCTCCGTAGGCCCGGCC |
| | | | | | | | | GGCGCTTGCCGATCAACCCAAATTTTTATCCAGGTTGACCTCGGATCAGGTAGGGATACCCGCTGAACTTAAGC |
| 2 | Fungi | Ascomy- | Euroto | Euro- | Tricho- | Peni- | Strain B | GTGAACCTGCGGAAGGATCATTACCGAGTGAGGGCCCTCTGGGTCCAACCTCCCACCCGTGTTTATTTTACCTTGTTGCTT |
| | | cota | Eurotio- | tiales | comaceae | cillium | | CGGCGGGCCCGCCTTAACTGGCCGCCGGGGGGCTTACGCCCCCGGGCCCGCGCCCGCCGAAGACACCCTCGAACTCTGTCT |
| | | | mycetes | | | | | GAAGATTGTAGTCTGAGTGAAAATATAAATTATTTAAAACTTTCAACAACGGATCTCTTGGTTCCGGCATCGATGAAGAAC |
| | | | | | | | | GCAGCGAAATGCGATACGTAATGTGAATTGCAAATTCAGTGAATCATCGAGTCTTTGAACGCACATTGCGCCCCCTGGTAT |
| | | | | | | | | TCCGGGGGGCATGCCTGTCCGAGCGTCATTGCTGCCCTCAAGCACGGCTTGTGTGTTGGGCCCCGTCCTCCGATCCCGGGG |
| | | | | | | | | GACGGGCCCGAAAGGCAGCGGCGGCACCGCGTCCGGTCCTCGAGCGTATGGGGCTTTGTCACCCGCTCTGTAGGCCCGGCC |
| | | | | | | | | GGCGCTTGCCGATCAACCCAAATTTTTATCCAGGTTGACCTCGGATCAGGTAGGGATACCCGCTGAACTTAAGCATAT |
| 3 | Fungi | Ascomy- | Euroto | Euro- | Tricho- | Peni- | chryso- | CGCCCTATCCTAGTTTCTCGGTTATTTCTAGGTCTCTATAGACCTCTATCGTCTCTCTAGCGATACCGCCTAGACTCT |
| | | cota | Eurotio- | tiales | comaceae | cillium | genum | CTCGCCGAGTCCTAGCTCTTAGCGCG |
| | | | mycetes | | | | | |
| 4 | Fungi | Ascomy- | Euroto | Euro- | Tricho- | Peni- | olsonii | AGGTGACCTGCGGAAGGATCATTACCGAGTGAGGGCCCTCTGGGTCCAACCTCCCACCCGTGTTTATTTTACCTTGTTGCT |
| | | cota | Eurotio- | tiales | comaceae | cillium | | TCGGCGAGCCTGCCTTCGGGCTGCCGGGGGGCATCTGCCCCCGGGTCCGCGCTCGCCGGAGACACCTTGAACTCTGTCTGA |
| | | | mycetes | | | | | AGATTGTAGTCTGAGACAAAATATAAATTATTTAAAACTTTCAACAACGGATCTCTTGGTTCCGGCATCGATGAAGAACGC |
| | | | | | | | | AGCGAAATGCGATACGTAATGTGAATTGCAGAATTCAGTGAATCATCGAGTCTTTGAACGCACATTGCGCCCTCTGGTATT |
| | | | | | | | | CCGGAGGGCATGCCTGTCCGAGCGTCATTGCTGCCCTCAAGCACGGCTTGTGTGTTGGGCTCCGTCCTCCTTCTGGGGGGA |
| | | | | | | | | CGGGCCCGAAAGGCAGCGGCGGCACCGCGTCCGGTCCTCGAGCGTATGGGGCTTTGTCACCCGCTCTGTAGGACTGGCCGG |
| | | | | | | | | CGCCTGCCGATCAACCAAACTTTTTTCCAGGTTGACCTCGGATCAGGTAGGGATACCCGCTGAACTTAAGCATATCAATAA |
| | | | | | | | | GCGGAGGAA |
| 5 | Fungi | Ascomy- | Euroto | Euro- | Tricho- | Peni- | griseo- | ATTACTGAGTGAGGGCCCTCTGGGTCCAACCTCCCACCCGTGTTTATTTACCTTGTTGCTTCGGCGGGCCCGCCTTAA |
| | | cota | Eurotio- | tiales | comaceae | cillium | fulvum | CTGGCCGCCGGGGGGCTTACGCCCCCGGGCCCGCGCCCGCCGAAGACACCCTCGAACTCTGTCTGAAGATTGTAGTCT |
| | | | mycetes | | | | | GAGTGAAAATATAAATTATTTAAAACTTTCAACAACGGATCTCTTGGTTCCGGCATCGATGAAGAACGCAGCGAAATG |
| | | | | | | | | CGATACGTAATGTGAATTGCAAATTCAGTGAATCATCGAGTCTTTGAACGCACATTGCGCCCCCTGGTATTCCGGGGG |
| | | | | | | | | GCATGCCTGTCCGAGCGTCATTGCTGCCCTCAAGCACGGCTTGTGTGTTGGGCCCCGTCCTCCGATTCCGGGGGACGG |
| | | | | | | | | GCCCGAAAGGCAGCGGCGGCACCGCGTCCGGTCCTCGAGCGTATGGGGCTTTGTCACCCGCTCTGTAGGCCCGGCCGG |
| | | | | | | | | CGCTTGCCGATCAACCCAAATTTTTATCCAGGTTGACCTCGGATCAGGTAGGGATACCCGCTGAACTTAAGCA |
| 6 | Fungi | Ascomy- | Euroto | Euro- | Tricho- | Peni- | janthi- | GACCTGCGGAAGGATCATTACCGAGTGAGGGCCCTCTGGGTCCAACCTCCCACCCGTGTTTATCATACCTAGTTGCTT |
| | | cota | Eurotio- | tiales | comaceae | cillium | nellum | CGGCGGGCCCGCCGTCATGGCCGCCGGGGGGCATCCGCCCCCGGGCCCGCGCCCGCCGAAGCCCCCCCTGAACGCTGT |
| | | | mycetes | | | | | CTGAAGATTGCAGTCTGAGCGATTAGCTAAATCAGTTAAAACTTTCAACAACGGATCTCTTGGTTCCGGCATCGATGA |
| | | | | | | | | AGAACGCAGCGAAATGCGATAAGTAATGTGAATTGCAGAATTCAGTGAATCATCGAGTCTTTGAACGCACATTGCGCC |
| | | | | | | | | CCCTGGTATTCCGGGGGGCATGCCTGTCCGAGCGTCATTGCTGCCCTCAAGCACGGCTTGTGTGTTGGGCCCCCGCCC |
| | | | | | | | | CCCGGCTCCCGGGGGGCGGGCCCGAAAGGCAGCGGCGGCACCGCGTCCGGTCCTCGAGCGTATGGGGCTTCGTCACCC |
| | | | | | | | | GCTCTGTAGGCCCGGCCGGCGCCCGCCGGCGACCCCCCTCAATCTTTCTCAGGTTGACCTCGGATCAGGTAGGGATAC |
| | | | | | | | | CCGCTGAACTTAAGCATATCAATAAGCGGAGGAA |
| 7 | Fungi | Ascomy- | Euroto | Euro- | Tricho- | Peni- | | CATTACCGAGTGAGGGCCCTCTGGGTCCAACCTCCCACCCGTGTTTATTTTACCTTGTTGCTTCGGCGGGCCCGCCTT |
| | | cota | Eurotio- | tiales | comaceae | cillium | | TACAGGCCGCCGGGGGGCTCACGCCCCCGGGCCCGCGCCCGCCGAAGACACCCTCGAACTCTGTCTGAAGATTGAAGT |
| | | | mycetes | | | | | CTGAGTGAAAATATAAATTATTTAAAACTTTCAACAACGGATCTCTTGGTTCCGGCATCGATGAAGAACGCAGCGAAA |
| | | | | | | | | TGCGATACGTAATGTGAATTGCAAATTCAGTGAATCATCGAGTCTTTGAACGCACATTGCGCCCCCTGGTATTCCGGG |
| | | | | | | | | GGGCATGCCTGTCCGAGCGTCATTGCTGCCCTCAAGCCCGGCTTGTGTGTTGGGCCCCGTCCTCCGATTTCCGGGGGA |
| | | | | | | | | CGGGCCCGAAAGGCAGCGGCGGCACCGCGTCCGGTCCTCGAGCGTATGGGGCTTTGTCACCCGCTCTGTAGGCCCGGC |
| | | | | | | | | CGGCGCTTGCCGATCAACCCAAATTTTTATCCAGGTTGACCTCGGATCAGGTAGGGATACCCGCTGAACTTAAGC |
| 8 | | | | | | | | CTTGGTCATTTAGAGGAAGTAA |
| 9 | | | | | | | | TCCTCCGCTTATTGATATGC |
| 10 | | | | | | | | AGCGGCCTCTATAATCCGGTA |
| 11 | | | | | | | | AGATATCGCTATCCGCGAAC |

TABLE 2
Auxin, acetoin, and siderophore production by the beneficial Penicillium
endophyte Strain B

| | auxin | | acetoin | | siderophore | |
|---|---|---|---|---|---|---|
| | Average | SE | Average | SE | Average | SE |
| (+) Control | 0.0825 | 0.0022 | 3.8513 | 0.0848 | 0.2450 | 0.0956 |
| Strain B | 0.0283 | 0.0009 | 0.0303 | 0.0013 | 0.2373 | 0.0976 |

TABLE 3
Biolog Assay Data
Utilization of sole carbon sources by fungal strain
Strain B using BIOLOG Phenotype MicroArray 1,
as recorded at 72 hours post-inoculation.

| Substrate | Strain B |
|---|---|
| Negative control | N/A |
| L-Arabinose | + |
| L-Proline | + |
| D-Xylose | + |
| L-Glutamic acid | + |
| D-Ribose | + |
| L-Asparagine | + |
| Sucrose | + |
| Tween 80 | + |
| Adonitol | + |
| L-Alanine | + |
| L-Alanyl-Glycine | + |
| L-Galactonic-acid-γ-lactone | + |
| β-Methyl-D-glucoside | + |
| m-Inositol | + |
| D-Galactose | + |
| D-Trehalose | + |
| D-Glucuronic acid | + |
| D-Gluconic acid | + |
| D-Mannitol | + |
| D-L-Malic acid | + |
| α-D-Glucose | + |
| Maltose | + |
| D-Melibiose | + |
| Maltotriose | + |
| Pyruvic acid | + |
| D-Galacturonic acid | + |
| D-Mannose | + |
| L-Threonine | + |
| Inosine | + |
| L-Lyxose | + |
| D-Alanine | + |
| L-Lactic acid | + |
| D-Galactonic acid-γ-lactone | + |
| Uridine | + |
| α-Hydroxy Glutaric acid-γ-lactone | + |
| D-L-α-Glycerol phosphate | + |
| D-Saccharic acid | − |
| Glycerol | − |
| Tween 20 | − |
| Tween 40 | − |
| L-glutamine | − |
| Glycyl-L-Aspartic acid | − |
| Fumaric acid | − |
| Glycyl-L-Glutamic acid | − |
| L-Serine | − |
| Glycyl-L-Proline | − |
| Citric acid | − |
| D-Malic acid | − |
| N-Acetyl-D-Glucosamine | − |
| Succinic acid | − |
| D-Sorbitol | − |
| D-Fructose | − |
| α-D-Lactose | − |
| Lactulose | − |
| Methyl Pyruvate | − |
| Dulcitol | − |
| L-Rhamnose | − |
| α-Methyl-D-Galactoside | − |
| m-Tartaric acid | − |
| D-Cellobiose | − |
| Mono Methyl Succinate | − |
| Adenosine | − |
| Bromo succinic acid | − |
| 2-Aminoethanol | − |
| p-Hydroxy Phenyl acetic acid | − |
| Phenylethyl-amine | − |
| L-Malic acid | − |
| L-Aspartic acid | − |
| Glyoxylic acid | − |
| L-Fucose | − |
| 1,2-Propanediol | − |
| D-Psicose | − |
| Acetic acid | − |
| Propionic acid | − |
| D-Glucosaminic acid | − |
| α-Keto-Glutaric acid | − |
| D-Glucose-1-Phosphate | − |
| 2-Deoxy adenosine | − |
| D-Threonine | − |
| Mucic acid | − |
| Glycolic acid | − |
| Tyramine | − |
| D-Serine | − |
| Formic acid | − |
| D-Glucose-6-Phosphate | − |
| Thymidine | − |
| D-Aspartic acid | − |
| α-Keto-Butyric acid | − |
| D-Fructose-6-Phosphate | − |
| α-Hydroxy Butyric acid | − |
| Tricarballylic acid | − |
| Acetoacetic acid | − |
| N-acetyl-β-D-Mannosamine | − |
| m-Hydroxy Phenyl Acetic acid | − |
| Glucuronamide | − |

### Table 4: Proteomics Analysis of Penicillium Culture Secretome

TABLE 4A
Proteins expressed in the culture of the Penicillium strain Strain B but not in that of the Penicillium strain Strain
F. Results are shown by KEGG pathway, by Gene Ontology (GO) description, means of levels for each strain (normalized spectra counts),
and the fold-change computed as log₂ StrainB/StrainF spectra count (normalized spectra counts of StrainB/StrainF).

| | | | Strain | Strain | |
|---|---|---|---|---|---|
| | | | B | F | Fold- |
| SEQID | KEGG | GO | mean | mean | change |
| 67 | E3.—.—.—, LCMT1 | acetylxylan esterase activity, cellulose catabolic process, extracellular | 0.031 | 0.000 | 5.0 |
| | | region, hydrolase activity, methylation, methyltransferase activity, | | | |
| | | xylan catabolic process | | | |
| 42 | Alzheimer's disease, ATPeF1B, ATP5B, | ADP binding, ATP binding, ATP hydrolysis coupled proton transport, | 0.056 | 0.000 | 5.8 |
| | ATP2, Huntington's disease, Oxidative | ATP synthesis coupled proton transport, mitochondrial ATP synthesis | | | |
| | phosphorylation, Parkinson's disease | coupled proton transport, mitochondrial proton-transporting ATP | | | |
| | | synthase, catalytic core, proton-transporting ATP synthase activity, | | | |
| | | rotational mechanism, proton-transporting ATP synthase complex, | | | |
| | | catalytic core F(1), proton-transporting ATPase activity, rotational | | | |
| | | mechanism | | | |
| 33 | None | anchored component of membrane, carbohydrate metabolic process, | 0.997 | 0.000 | 10.0 |
| | | plasma membrane, transferase activity | | | |
| 15 | E3.2.1.89 | arabinogalactan endo-1,4-beta-galactosidase activity, carbohydrate | 0.552 | 0.000 | 9.1 |
| | | metabolic process, glucosidase activity | | | |
| 47 | Aminoacyl-tRNA biosynthesis, Ribosome, | aspartic-type endopeptidase activity, ATP binding, methylation, | 0.051 | 0.000 | 5.7 |
| | RP-S10, MRPS10, rpsJ, WARS, trpS | methyltransferase activity, nucleic acid binding, proteolysis, ribosome, | | | |
| | | structural constituent of ribosome, tryptophan-tRNA ligase activity, | | | |
| | | tryptophanyl-tRNA aminoacylation | | | |
| 38 | None | carbohydrate binding, carbohydrate metabolic process, hydrolase | 0.312 | 0.000 | 8.3 |
| | | activity, hydrolyzing O-glycosyl compounds, integral component of | | | |
| | | membrane | | | |
| 75 | E3.2.1.— | carbohydrate metabolic process, cell wall macromolecule catabolic | 1.331 | 0.000 | 10.4 |
| | | process, lysozyme activity, peptidoglycan catabolic process | | | |
| 60 | GBA, srfJ, Lysosome, Other glycan | carbohydrate metabolic process, glucan endo-1,6-beta-glucosidase | 0.053 | 0.000 | 5.8 |
| | degradation, Sphingolipid metabolism | activity, glucosylceramidase activity, sphingolipid metabolic process | | | |
| 61 | E3.2.1.— | cellulose 1,4-beta-cellobiosidase activity, cellulose binding, cellulose | 0.066 | 0.000 | 6.1 |
| | | catabolic process, extracellular region, hydrolase activity, hydrolyzing | | | |
| | | O-glycosyl compounds | | | |
| 34 | E3.2.1.3, Starch and sucrose metabolism | glucan 1,4-alpha-glucosidase activity, polysaccharide catabolic process, | 0.174 | 0.000 | 7.5 |
| | | starch binding | | | |
| 28 | E3.2.1.3, Starch and sucrose metabolism | glucan 1,4-alpha-glucosidase activity, polysaccharide metabolic process | 4.965 | 0.000 | 12.3 |
| 58 | None | nutrient reservoir activity, regulation of transcription, DNA-templated, | 0.101 | 0.000 | 6.7 |
| | | sequence-specific DNA binding, transcription factor activity, sequence- | | | |
| | | specific DNA binding | | | |

TABLE 4B
Proteins not expressed in the culture of the Penicillium strain Strain B but were expressed in that of the Penicillium strain
Strain F. Results are shown by KEGG pathway, by Gene Ontology (GO) description, means of levels for each strain (normalized spectra counts),
and the fold-change computed as log₂ StrainB/StrainF spectra count (normalized spectra counts of StrainB/StrainF).

| | | | Strain | Strain | |
|---|---|---|---|---|---|
| | | | B | F | Fold- |
| SEQID | KEGG | GO | mean | mean | change |
| 72 | None | (1−>3)-beta-D-glucan metabolic process, 1,3-beta- | 0.000 | 1.287 | −10.3 |
| | | glucanosyltransferase activity, anchored component of | | | |
| | | membrane, carbohydrate metabolic process, fungal-type cell | | | |
| | | wall, hydrolase activity, integral component of membrane, | | | |
| | | plasma membrane, transferase activity | | | |
| 57 | E3.1.3.8, Inositol phosphate metabolism | acid phosphatase activity, dephosphorylation | 0.000 | 0.078 | −6.3 |
| 27 | Cysteine and methionine metabolism, E3.3.1.1, | adenosylhomocysteinase activity, one-carbon metabolic | 0.000 | 0.183 | −7.5 |
| | ahcY | process | | | |
| 48 | Carbohydrate digestion and absorption, E3.2.1.1, | alpha-amylase activity, calcium ion binding, carbohydrate | 0.000 | 0.195 | −7.6 |
| | amyA, malS, Starch and sucrose metabolism | catabolic process, carbohydrate metabolic process, hydrolase | | | |
| | | activity, hydrolyzing O-glycosyl compounds | | | |
| 18 | E3.2.1.22B, galA, rafA, Galactose metabolism, | alpha-galactosidase activity, carbohydrate metabolic process, | 0.000 | 0.078 | −6.3 |
| | Glycerolipid metabolism, Glycosphingolipid | extracellular region, polysaccharide catabolic process, raffinose | | | |
| | biosynthesis - globo series, Sphingolipid metabolism | alpha-galactosidase activity | | | |
| 55 | APE2 | amino acid transmembrane transport, amino acid | 0.000 | 0.428 | −8.7 |
| | | transmembrane transporter activity, aminopeptidase activity, | | | |
| | | integral component of membrane, metallopeptidase activity, | | | |
| | | proteolysis, zinc ion binding | | | |
| 36 | PEPD | aminopeptidase activity, manganese ion binding, | 0.000 | 0.578 | −9.2 |
| | | metallopeptidase activity, proteolysis | | | |
| 73 | pepP | aminopeptidase activity, metal ion binding, metallopeptidase | 0.000 | 0.170 | −7.4 |
| | | activity, proteolysis | | | |
| 45 | None | anchored component of membrane, carbohydrate metabolic | 0.000 | 0.561 | −9.1 |
| | | process, hydrolase activity, plasma membrane, transferase | | | |
| | | activity | | | |
| 32 | Amino sugar and nucleotide sugar metabolism, | ATP binding, carbohydrate phosphorylation, cell, cellular | 0.000 | 0.389 | −8.6 |
| | Butirosin and neomycin biosynthesis, Carbohydrate | glucose homeostasis, glucose binding, glycolytic process, | | | |
| | digestion and absorption, Carbon metabolism, | hexokinase activity | | | |
| | Central carbon metabolism in cancer, Fructose and | | | | |
| | mannose metabolism, Galactose metabolism, | | | | |
| | Glycolysis/Gluconeogenesis, HIF-1 signaling | | | | |
| | pathway, HK, Insulin signaling pathway, Starch and | | | | |
| | sucrose metabolism, Streptomycin biosynthesis, | | | | |
| | Type II diabetes mellitus | | | | |
| 70 | None | ATP binding, helicase activity, nucleic acid binding, proteolysis, | 0.000 | 0.021 | −4.4 |
| | | serine-type peptidase activity | | | |
| 30 | bglX, Cyanoamino acid metabolism, | beta-glucosidase activity, cellulose catabolic process | 0.000 | 0.219 | −7.8 |
| | Phenylpropanoid biosynthesis, Starch and sucrose | | | | |
| | metabolism | | | | |
| 14 | 2-Oxocarboxylic acid metabolism, Alanine, | biosynthetic process, cellular amino acid metabolic process, L- | 0.000 | 0.301 | −8.2 |
| | aspartate and glutamate metabolism, Arginine and | aspartate: 2-oxoglutarate aminotransferase activity, L- | | | |
| | proline metabolism, Biosynthesis of amino acids, | phenylalanine: 2-oxoglutarate aminotransferase activity, | | | |
| | Carbon fixation in photosynthetic organisms, | pyridoxal phosphate binding, transaminase activity | | | |
| | Carbon metabolism, Cysteine and methionine | | | | |
| | metabolism, GOT1, Isoquinoline alkaloid | | | | |
| | biosynthesis, Phenylalanine metabolism, | | | | |
| | Phenylalanine, tyrosine and tryptophan | | | | |
| | biosynthesis, Tropane, piperidine and pyridine | | | | |
| | alkaloid biosynthesis, Tyrosine metabolism | | | | |
| 53 | MAN1, N-Glycan biosynthesis, Protein processing | calcium ion binding, extracellular region, mannosyl- | 0.000 | 2.382 | −11.2 |
| | in endoplasmic reticulum, Various types of | oligosaccharide 1,2-alpha-mannosidase activity, membrane, | | | |
| | N-glycan biosynthesis | metabolic process, protein glycosylation | | | |
| 77 | E3.2.1.— | carbohydrate binding, carbohydrate metabolic process, cell | 0.000 | 2.037 | −11.0 |
| | | wall, cell wall organization, DNA binding, hydrolase activity, | | | |
| | | hydrolyzing O-glycosyl compounds, nucleus, transcription, | | | |
| | | DNA-templated, transferase activity, zinc ion binding | | | |
| 29 | None | carbohydrate binding, carbohydrate metabolic process, | 0.000 | 0.032 | −5.0 |
| | | endoribonuclease activity, hydrolase activity, hydrolyzing O- | | | |
| | | glycosyl compounds, integral component of membrane, RNA | | | |
| | | phosphodiester bond hydrolysis, endonucleolytic, rRNA | | | |
| | | transcription | | | |
| 40 | None | carbohydrate binding, carbohydrate metabolic process, | 0.000 | 0.730 | −9.5 |
| | | hydrolase activity | | | |
| 37 | None | carbohydrate binding, carbohydrate metabolic process, | 0.000 | 0.047 | −5.6 |
| | | hydrolase activity | | | |
| 43 | Galactose metabolism, malZ, Starch and sucrose | carbohydrate binding, carbohydrate metabolic process, | 0.000 | 0.360 | −8.5 |
| | metabolism | hydrolase activity, hydrolyzing O-glycosyl compounds | | | |
| 56 | E5.1.3.15, Glycolysis/Gluconeogenesis | carbohydrate binding, carbohydrate metabolic process, | 0.000 | 0.208 | −7.7 |
| | | hydrolase activity, isomerase activity | | | |
| 76 | Galactose metabolism, galM, GALM, Glycolysis/ | carbohydrate binding, carbohydrate metabolic process, | 0.000 | 0.174 | −7.5 |
| | Gluconeogenesis | isomerase activity | | | |
| 39 | None | carbohydrate metabolic process, hydrolase activity, hydrolyzing | 0.000 | 0.287 | −8.2 |
| | | O-glycosyl compounds | | | |
| 62 | None | catalytic activity, hydrolase activity, acting on glycosyl bonds, | 0.000 | 0.282 | −8.1 |
| | | metabolic process | | | |
| 52 | Glutathione metabolism, GSR, gor, Thyroid | cell, cell redox homeostasis, flavin adenine dinucleotide | 0.000 | 0.239 | −7.9 |
| | hormone synthesis | binding, glutathione metabolic process, glutathione-disulfide | | | |
| | | reductase activity, NADP binding, oxidation-reduction process | | | |
| 54 | msrA Methionine sulfoxide reductase | cellular response to hydrogen peroxide, cytosol, L-methionine | 0.000 | 0.291 | −8.2 |
| | | biosynthetic process from methionine sulphoxide, L- | | | |
| | | methionine-(S)-S-oxide reductase activity, nucleus, oxidation- | | | |
| | | reduction process, peptide-methionine (S)-S-oxide reductase | | | |
| | | activity, protein repair, response to oxidative stress | | | |
| 16 | EEF1A, Legionellosis, RNA transport | cytoplasm, GTP binding, GTPase activity, translation elongation | 0.000 | 0.104 | −6.7 |
| | | factor activity, translation initiation factor activity, translational | | | |
| | | elongation, translational initiation | | | |
| 63 | ARHGDI, RHOGDI, Neurotrophin signaling | cytoplasm, regulation of catalytic activity, Rho GDP-dissociation | 0.000 | 0.201 | −7.7 |
| | pathway, Vasopressin-regulated water reabsorption | inhibitor activity | | | |
| 20 | Alzheimer's disease, Amyotrophic lateral sclerosis | cytosol, electron carrier activity, heme binding, metal ion | 0.000 | 1.256 | −10.3 |
| | (ALS), Apoptosis, Colorectal cancer, CYC, | binding, mitochondrial ATP synthesis coupled electron | | | |
| | Hepatitis B, Herpes simplex infection, | transport, mitochondrion, nucleus, oxidation-reduction | | | |
| | Huntington's disease, Influenza A, Legionellosis, | process, respiratory chain | | | |
| | Non-alcoholic fatty liver | | | | |
| | disease (NAFLD), p53 signaling pathway, | | | | |
| | Parkinson's disease, Pathways in cancer, Small | | | | |
| | cell lung cancer, Sulfur metabolism, | | | | |
| | Toxoplasmosis, Tuberculosis, Two-component | | | | |
| | system, Viral myocarditis | | | | |
| 44 | None | dephosphorylation, phosphatase activity | 0.000 | 0.138 | −7.1 |
| 21 | ECE | FMN binding, integral component of membrane, | 0.000 | 0.082 | −6.4 |
| | | metalloendopeptidase activity, oxidation-reduction process, | | | |
| | | oxidoreductase activity, proteolysis | | | |
| 51 | Ether lipid metabolism, Glycerophospholipid | hydrolase activity, acting on ester bonds, metabolic process | 0.000 | 0.745 | −9.5 |
| | metabolism, Inositol phosphate metabolism, plcC, | | | | |
| | Thyroid hormone signaling pathway | | | | |
| 66 | None | hydrolase activity, metabolic process | 0.000 | 0.829 | −9.7 |
| 35 | None | hydrolase activity, metabolic process | 0.000 | 0.012 | −3.7 |
| 64 | None | integral component of membrane | 0.000 | 2.459 | −11.3 |
| 71 | Betalain biosynthesis, Isoquinoline alkaloid | integral component of membrane, metal ion binding, oxidation- | 0.000 | 0.263 | −8.0 |
| | biosynthesis, Melanogenesis, Riboflavin | reduction process, oxidoreductase activity | | | |
| | metabolism, TYR, Tyrosine metabolism | | | | |
| 68 | E3.2.1.58, Starch and sucrose metabolism | lyase activity, metabolic process | 0.000 | 0.145 | −7.2 |
| 69 | E3.2.1.58, Starch and sucrose metabolism | lyase activity, metabolic process | 0.000 | 0.114 | −6.8 |
| 46 | AGXT, Alanine, aspartate and glutamate | metabolic process, transaminase activity | 0.000 | 0.171 | −7.4 |
| | metabolism, Carbon metabolism, Glycine, serine | | | | |
| | and threonine metabolism, Glyoxylate and | | | | |
| | dicarboxylate metabolism, Methane metabolism, | | | | |
| | Peroxisome | | | | |
| 25 | None | mitochondrion, oxidation-reduction process, oxidoreductase | 0.000 | 0.531 | −9.1 |
| | | activity | | | |
| 23 | Cysteine and methionine metabolism, E2.4.2.28, | nucleoside metabolic process, S-methyl-5-thioadenosine | 0.000 | 0.076 | −6.3 |
| | mtaP | phosphorylase activity | | | |
| 17 | Biosynthesis of amino acids, Carbon fixation in | pentose-phosphate shunt, non-oxidative branch, ribose-5- | 0.000 | 0.264 | −8.0 |
| | photosynthetic organisms, Carbon metabolism, | phosphate isomerase activity | | | |
| | Pentose phosphate pathway, rpiA | | | | |

TABLE 4C
Proteins expressed in the culture of the Penicillium strain Strain B at a higher level than were expressed in the culture of the Penicillium
strain Strain F. Results are shown by KEGG pathway, by Gene Ontology (GO) description, means of levels for each strain (normalized spectra counts),
and the fold-change computed as log₂ StrainB/StrainF spectra count (normalized spectra counts of StrainB/StrainF).

| | | | Strain | Strain | |
|---|---|---|---|---|---|
| | | | B | F | Fold- |
| SEQID | KEGG | GO | mean | mean | change |
| 12 | None | flavin adenine dinucleotide binding, integral component of membrane, | 0.201 | 0.012 | 3.9 |
| | | oxidation-reduction process, oxidoreductase activity, acting on CH—OH | | | |
| | | group of donors | | | |
| 13 | None | flavin adenine dinucleotide binding, oxidation-reduction process, | 0.685 | 0.117 | 2.5 |
| | | oxidoreductase activity, acting on CH—OH group of donors | | | |
| 19 | None | carbohydrate binding, carbohydrate metabolic process, cell wall, cell wall | 0.634 | 0.048 | 3.7 |
| | | organization, hydrolase activity, hydrolyzing O-glycosyl compounds, | | | |
| | | integral component of membrane, transferase activity | | | |
| 22 | E3.2.1.4, Starch and sucrose metabolism | carbohydrate metabolic process, cellulose binding, extracellular region, | 0.881 | 0.170 | 2.4 |
| | | hydrolase activity, hydrolyzing O-glycosyl compounds | | | |
| 24 | None | flavin adenine dinucleotide binding, oxidation-reduction process, | 1.442 | 0.122 | 3.5 |
| | | oxidoreductase activity, acting on CH—OH group of donors, UDP-N- | | | |
| | | acetylmuramate dehydrogenase activity | | | |
| 26 | E3.4.23.— | aspartic-type endopeptidase activity, carbohydrate binding, extracellular | 6.126 | 0.183 | 5.1 |
| | | region, proteolysis, zymogen activation | | | |
| 49 | E3.4.23.18 | aspartic-type endopeptidase activity, extracellular region, pathogenesis, | 5.499 | 0.303 | 4.2 |
| | | proteolysis | | | |
| 50 | Amino sugar and nucleotide sugar | chitosanase activity, extracellular region, polysaccharide catabolic process | 8.011 | 0.511 | 4.0 |
| | metabolism, csn | | | | |
| 59 | None | carbohydrate binding, carbohydrate metabolic process, hydrolase activity, | 0.304 | 0.065 | 2.2 |
| | | acting on glycosyl bonds | | | |

TABLE 4D
Proteins expressed in the culture of the Penicillium strain Strain B at a lower level than were expressed in the culture of the Penicillium
strain Strain F. Results are shown by KEGG pathway, by Gene Ontology (GO) description, means of levels for each strain (normalized spectra counts),
and the fold-change computed as log₂ StrainB/StrainF spectra count (normalized spectra counts of StrainB/StrainF).

| | | | Strain | Strain | |
|---|---|---|---|---|---|
| | | | B | F | Fold- |
| SEQID | KEGG | GO | mean | mean | change |
| 74 | E3.2.1.58, Starch and sucrose | carbohydrate metabolic process, hydrolase activity, hydrolyzing O- | 0.017 | 3.833 | −7.7 |
| | metabolism | glycosyl compounds, transferase activity | | | |
| 65 | Amino sugar and nucleotide sugar | carbohydrate metabolic process, chitin catabolic process, chitinase | 0.027 | 0.293 | −3.4 |
| | metabolism, E3.2.1.14 | activity | | | |
| 41 | None | anchored component of membrane, carbohydrate metabolic process, | 0.138 | 0.495 | −1.8 |
| | | plasma membrane, transferase activity | | | |
| 31 | Ether lipid metabolism, | hydrolase activity, acting on ester bonds, metabolic process | 0.314 | 0.547 | −0.8 |
| | Glycerophospholipid metabolism, | | | | |
| | Inositol phosphate metabolism, plcC, | | | | |
| | Thyroid hormone signaling pathway | | | | |

Table 5: Wheat radical length under normal conditions

TABLE 5A
Wheat Radical Length of seedlings grown from seeds treated with Strain
B, vs. non-treated plants, under normal watering conditions

radical length

| | Average (cm) | SE |
|---|---|---|
| Non-Treated | 2.117 | 0.185 |
| Formulation Control | 2.590 | 0.327 |
| Strain B | 2.717 | 0.185 |

TABLE 5B
Wheat Shoot Length of seedlings grown from seeds treated with Strain
B, vs. non-treated plants, under water-limited conditions

shoot length

| | Average (cm) | SE |
|---|---|---|
| Non-Treated | 1.082 | 0.216 |
| Formulation Control | 1.792 | 0.226 |
| Strain B | 2.079 | 0.202 |

### Table 6: Greenhouse Soybean Plant Yield Characteristics

TABLE 6A
Greenhouse soybean plant yield characteristics under normal (non-
water limited) watering and water-limited conditions. Soybean plants
grown from seeds treated with Strain B show improved phenotypes under
normal watering and water-limited (drought) conditions. Asterisk
indicates significance at alpha level = 0.05. P values were calculated
using a Fisher exact test (R package “stats”), one-tailed for the
beneficial effect of treatment. Asterisk indicates Bayesian significance
at posterior probability = 95%, as calculated using Bayesian
high-density interval (R package “BEST)”. Bayesian posterior
probability of a beneficial effect quantifies the posterior belief
placed on the percent improvement being beneficial, i.e. the treatment
mean being different than the control mean in the direction reported.

| | |
|---|---|
| | Percent Change |
| Traits (per plant), at days post | Strain B vs |
| planting (dpp) | Formulation Control |

Soy: Normal

| | |
|---|---|
| Dry weight of mature seeds (0% moisture), | 18% |
| harvest | |
| Fresh weight of mature seeds, harvest | 15% |
| Number of mature seeds, harvest | 10% |
| SPAD measurement of chlorophyll, 87 dpp | −19% |
| Number of pods, 77 dpp | 6% |
| Lengths of pods, 46 dpp | −2% |

Soy: Drought

| | |
|---|---|
| Dry weight of mature seeds (0% moisture), | 18% |
| harvest | |
| Fresh weight of mature seeds, harvest | 21% |
| Number of mature seeds, harvest | 18% |
| SPAD measurement of chlorophyll, 87 dpp | 16% * |
| Number of pods, 77 dpp | 27% |
| Lengths of pods, 48 dpp | 18% |
| Percent of leaves scored 3 = severe | −63% * |
| wilting, 32 dpp | |
| Percent of leaves scored 0 = no | 188% * |
| wilting, 32 dpp | |

TABLE 6B
Beneficial Penicillium endophyte Strain B imparts improved plant characteristics under
water-limited conditions in the greenhouse compared to Penicillium strain Strain F.

| | Formulation | Strain | Strain | Strain | Strain | Strain | Strain | |
|---|---|---|---|---|---|---|---|---|
| TREATMENT | Control | A | E | B | G | D | F | unit |
| Final Emergence | 2.61 | 2.44 | 2.53 | 2.56 | 2.56 | 2.18 | 2.50 | seedlings, |
| (Jan27, 12dpp) | | | | | | | | out of 3 |
| | | | | | | | | planted per |
| | | | | | | | | pot |
| Pod Count (Mar18, | 15.79 | 16.93 | 15.38 | 14.83 | 14.08 | 16.00 | 15.07 | pods per |
| 63dpp) | | | | | | | | plant |
| Seed Pre-Count | 31.14 | 32.86 | 30.23 | 30.17 | 28.54 | 31.69 | 30.50 | seeds/plant, |
| (Mar25, 70dpp) | | | | | | | | counted |
| | | | | | | | | inside pods |
| Seed Count, | 23.79 | 24.07 | 23.92 | 24.33 | 22.07 | 23.38 | 22.36 | seeds per |
| Mature (Apr27, | | | | | | | | plant, |
| 103dpp) | | | | | | | | harvested, |
| | | | | | | | | mature |
| Seed Count, | 31.14 | 32.21 * | 30.38 | 29.33 | 28.36 | 31.23 | 28.57 | seeds per |
| Mature + Immature | | | | | | | | plant, |
| (Apr28, 104dpp) | | | | | | | | harvested, |
| | | | | | | | | mature + |
| | | | | | | | | immature |
| Percent of Seeds | 76.38% | 74.72% | 78.73% | 82.95% | 77.83% | 74.88% | 78.25% | % of seeds |
| That Are Mature | | | | | | | | matured |
| (Apr27, 103dpp) | | | | | | | | |
| Seed Weight, | 3.98 | 4.00 | 4.00 | 3.91 | 3.75 | 3.96 | 3.77 | dry grams |
| Mature (Apr27, | | | | | | | | of mature |
| 103dpp) | | | | | | | | seed per |
| | | | | | | | | plant |
| Wilt Score (Feb17, | 0.89 | 0.56 | 0.88 | 0.69 | 0.78 | 0.94 | 0.89 | score (0 = no |
| 33dpp) | | | | | | | | wilt, 4 = max |
| | | | | | | | | wilt) |
| Wilt Score (Feb24, | 0.83 | 0.44 | 0.41 | 0.44 | 0.94 | 0.76 | 0.89 | score (0 = no |
| 40dpp) | | | | | | | | wilt, 4 = max |
| | | | | | | | | wilt) |
| Wilt Score (Feb25, | 1.57 | 1.00 | 1.31 | 0.83 | 1.50 | 1.23 | 1.43 | score (0 = no |
| 41dpp) | | | | | | | | wilt, 4 = max |
| | | | | | | | | wilt) |
| Wilt Score (Feb26, | 2.64 | 2.29 | 2.54 | 1.75 | 2.21 | 2.31 | 2.36 | score (0 = no |
| 42dpp) | | | | | | | | wilt, 4 = max |
| | | | | | | | | wilt) |

* indicates statistically significant values vs. all other treatments/control groups.
Bold number values indicates instances where a particular strain performed better than any other Penicillium strain or control

### Table 7: Transcriptomics Results

TABLE 7A
Qualititative transcript analysis of upregulated and downregulated genes in leaf,
root, and stem tissues of plants grown from seeds treated with Strain B, under
normal (well watered, non-water limied) and water-limited (drought) growth conditions.
“+” and “−” denote a relative increase or decrease, respectively,
when compared to control plants grown in similar conditions (formulation control).

| | Plants grown from seeds treated with | | | | | |
|---|---|---|---|---|---|---|
| | Strain B change vs. Formulation Control | | | | | |
| | Normal | | | Drought | | |
| Gene Description | root | stem | leaf | root | stem | leaf |
| 18.5 kDa class I heat shock protein | + | + | − | − | − | − |
| 2-hydroxyisoflavanone synthase | − | + | + | − | − | + |
| 3-ketoacyl-CoA synthase | − | − | − | − | − | + |
| 3-ketoacyl-CoA synthase | − | − | − | − | − | + |
| 3-ketoacyl-CoA synthase | − | − | − | − | − | + |
| 3-ketoacyl-CoA synthase | − | − | − | − | − | − |
| 50S ribosomal protein L35 | − | − | − | + | + | + |
| Acetolactate synthase | − | − | − | − | − | − |
| Alcohol-dehydrogenase | + | + | + | + | + | + |
| Aldehyde dehydrogenase | + | − | − | + | + | − |
| Aldehyde dehydrogenase | + | − | − | + | + | − |
| Aldehyde dehydrogenase | − | − | − | − | − | + |
| Amidophosphoribosyltransferase (chloroplastic) | + | − | − | − | − | − |
| Amine oxidase | − | − | − | − | + | − |
| Amine oxidase | − | − | − | + | − | − |
| Amine oxidase | − | − | + | − | − | + |
| Amine oxidase | − | − | + | − | − | + |
| Amine oxidase | + | − | − | + | − | − |
| Annexin | − | + | + | + | + | + |
| Annexin | − | + | + | + | − | + |
| Annexin | − | + | + | + | + | + |
| Annexin | − | − | − | + | − | − |
| Annexin | − | − | − | + | − | − |
| Annexin | − | − | − | + | − | − |
| Annexin | − | − | − | + | − | − |
| Annexin | − | − | + | + | − | + |
| Annexin | − | − | + | + | − | + |
| Apocytochrome f | − | − | − | − | − | − |
| Arginase | − | − | − | − | + | + |
| Arginine decarboxylase | + | − | − | − | + | − |
| Asparagine synthetase | + | + | + | − | − | − |
| Asparagine synthetase | + | + | + | − | − | − |
| Asparagine synthetase | + | + | + | − | − | − |
| Asparagine synthetase | − | − | + | + | − | − |
| Asparagine synthetase | − | − | + | + | − | − |
| Asparagine synthetase | + | + | + | − | − | − |
| Asparagine synthetase | − | − | − | + | − | + |
| Asparagine synthetase | − | − | − | + | − | + |
| Asparagine synthetase | − | − | − | + | − | + |
| ATP synthase epsilon chain (chloroplastic) | + | + | − | − | + | − |
| ATP synthase gamma chain | + | + | − | + | − | − |
| Auxin-induced protein 15A | − | − | − | − | + | − |
| Auxin-induced protein 15A | − | − | + | − | + | − |
| Auxin-induced protein 6B | − | − | − | − | + | − |
| Auxin-induced protein X15 | − | − | − | − | + | − |
| beta glucosidase 42 | − | − | − | + | + | + |
| Beta-amyrin 24-hydroxylase | + | − | + | + | + | + |
| Beta-galactosidase | − | − | − | + | − | − |
| Beta-galactosidase | − | − | − | + | − | − |
| Beta-galactosidase | − | − | − | + | − | − |
| Beta-galactosidase | − | − | − | + | − | − |
| Beta-galactosidase | + | − | − | + | − | − |
| Beta-galactosidase | + | − | − | + | − | − |
| Beta-galactosidase | − | − | + | + | − | − |
| Calcium-binding EF-hand family protein | + | − | − | + | + | + |
| Calcium-binding EF-hand family protein | + | − | − | + | + | + |
| Calmodulin-2 | − | − | − | + | + | + |
| Calmodulin-2 | + | + | + | − | + | + |
| CASP-like protein 10 | − | − | − | − | − | + |
| Chalcone synthase 7 | + | + | + | − | − | + |
| Chalcone--flavonone isomerase 1A | + | + | + | − | + | + |
| Chalcone--flavonone isomerase 1A | + | + | + | − | + | + |
| Chlorophyll a-b binding protein 2 (chloroplastic) | + | + | − | + | + | + |
| Chlorophyll a-b binding protein 2 (chloroplastic) | + | − | − | + | + | + |
| Chlorophyll a-b binding protein 3 (chloroplastic) | + | − | − | + | + | + |
| Cytochrome b6 | − | − | − | − | − | − |
| Cytochrome c oxidase subunit 1 | + | − | − | − | − | − |
| Cytochrome P450 77A3 | − | − | + | − | − | + |
| Cytochrome P450 78A3 | − | + | + | + | + | − |
| Cytochrome P450 82A4 | − | − | − | − | − | − |
| Cytochrome P450 93A3 | − | − | − | + | − | − |
| Cytochrome P450 monooxygenase CYP89H3 | − | + | + | − | + | − |
| DNA-directed RNA polymerase | − | + | + | − | − | − |
| DNA-directed RNA polymerase | − | + | + | − | − | − |
| DNA-directed RNA polymerase | − | − | − | − | − | − |
| DNA-directed RNA polymerase subunit | + | − | + | − | + | + |
| Early nodulin-36A | + | + | − | + | + | − |
| Early nodulin-70 | + | − | − | − | − | − |
| Early nodulin-93 | + | − | − | − | − | − |
| Ethylene-responsive element binding factor 4 | + | + | + | + | + | + |
| Eukaryotic translation initiation factor 6 | + | + | + | − | − | + |
| Eukaryotic translation initiation factor 6 | + | + | + | − | − | + |
| Expansin | − | − | − | − | − | − |
| Ferritin | + | + | + | − | + | − |
| Ferritin | + | − | + | − | + | − |
| Ferritin | + | − | + | − | + | − |
| Ferritin | − | + | + | − | + | − |
| Ferritin | − | + | + | − | + | − |
| Ferritin-1 (chloroplastic) | + | + | + | − | + | + |
| Ferritin-1 (chloroplastic) | + | + | + | − | + | + |
| Ferritin-2 (chloroplastic) | − | + | + | − | + | − |
| Ferritin-4 (chloroplastic) | − | − | + | − | + | − |
| Flavonoid 4′-O-methyltransferase | + | − | − | + | − | + |
| Fructose-bisphosphate aldolase | − | − | − | − | − | + |
| Fructose-bisphosphate aldolase | + | − | − | + | − | − |
| Fructose-bisphosphate aldolase | + | − | − | + | − | − |
| Fructose-bisphosphate aldolase | + | − | − | + | − | − |
| Fructose-bisphosphate aldolase | + | − | + | + | − | + |
| Fructose-bisphosphate aldolase | + | − | − | + | + | − |
| Fructose-bisphosphate aldolase | + | − | − | + | − | − |
| Fructose-bisphosphate aldolase | + | − | − | + | − | − |
| G2/mitotic-specific cyclin S13-6 | − | − | − | − | + | − |
| G2/mitotic-specific cyclin S13-7 | − | − | − | − | + | − |
| Glucan endo-1,3-beta-glucosidase (beta-1,3- | − | − | + | − | − | + |
| endoglucanase) | | | | | | |
| Glucose-1-phosphate adenylyltransferase | − | + | − | − | − | − |
| Glucose-1-phosphate adenylyltransferase | − | + | − | − | − | − |
| Glucose-1-phosphate adenylyltransferase | − | + | − | − | − | − |
| Glucose-1-phosphate adenylyltransferase | − | + | − | − | − | − |
| Glucose-1-phosphate adenylyltransferase | − | + | − | − | − | − |
| Glucose-1-phosphate adenylyltransferase | − | + | − | − | − | − |
| Glutamate receptor | − | − | + | − | − | − |
| Glutamine synthetase | + | + | − | − | − | − |
| Glutamine synthetase cytosolic isozyme 2 | + | − | − | − | − | − |
| Glutathione peroxidase | + | + | − | − | + | + |
| Glutathione peroxidase | + | + | − | − | + | + |
| Glutathione peroxidase | + | + | − | − | + | + |
| Glutathione peroxidase | + | + | − | + | − | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | − | + | + |
| Histone H2A | − | − | − | + | + | + |
| Histone H2A | − | − | − | + | + | + |
| Histone H2A | − | − | − | − | + | + |
| Histone H2B | − | − | − | − | + | + |
| Histone H2B | − | − | − | − | + | + |
| Histone H2B | − | − | − | − | + | + |
| Histone H2B | − | − | − | − | + | + |
| Histone H2B | − | − | − | + | + | + |
| Histone H2B | − | − | − | − | + | + |
| Histone H3 | − | − | − | − | + | + |
| Histone H3 | − | − | − | − | + | + |
| Histone H3 | − | − | − | + | + | + |
| Histone H3 | − | − | − | − | + | + |
| Histone H3 | − | − | − | − | + | + |
| Histone H3 | − | − | − | + | + | + |
| Histone H3 | − | − | − | − | + | + |
| Histone H3 | − | − | − | + | + | + |
| Histone H3 | − | − | − | + | + | + |
| Histone H3 | − | − | − | + | + | + |
| Histone H3 | − | − | − | + | + | + |
| Histone H3 | − | − | − | − | + | + |
| Histone H3 | − | − | − | − | + | + |
| Histone H4 | − | − | − | − | + | + |
| Histone H4 | − | − | − | − | + | + |
| Histone H4 | − | − | − | − | + | + |
| Histone H4 | − | − | − | − | + | + |
| Histone H4 | + | − | − | − | + | + |
| Histone H4 | − | − | − | + | + | + |
| Histone H4 | − | − | − | − | + | + |
| Histone H4 | − | − | − | − | + | + |
| Histone H4 | − | − | − | + | + | + |
| Histone H4 | − | − | − | − | + | + |
| Histone H4 | − | − | − | − | + | + |
| Histone H4 | − | − | − | − | + | + |
| Histone H4 | − | − | − | − | + | + |
| Histone H4 | − | − | − | − | + | + |
| Histone H4 | − | − | − | − | + | + |
| Histone H4 | − | − | − | − | + | + |
| HMG-Y-related protein A | + | − | − | − | + | + |
| HMG-Y-related protein A | + | − | − | − | + | + |
| Leghemoglobin A | + | − | − | − | − | − |
| Leghemoglobin C1 | + | − | − | − | − | − |
| Leghemoglobin C2 | + | − | − | − | − | − |
| Leghemoglobin C3 | + | − | − | − | − | − |
| Lipase | − | − | − | + | − | − |
| Lipase | + | − | − | − | − | − |
| Lipase | − | − | − | + | − | − |
| Lipoxygenase | − | − | − | + | − | − |
| Lipoxygenase | − | − | − | + | − | − |
| Lipoxygenase | − | − | + | + | − | + |
| Lipoxygenase | − | − | + | + | − | + |
| Lipoxygenase | − | − | − | − | − | − |
| Lipoxygenase | − | − | − | + | − | + |
| Lipoxygenase | − | − | − | + | − | − |
| Lipoxygenase | + | − | + | + | − | − |
| Lipoxygenase | + | − | + | + | − | − |
| Lipoxygenase | − | − | − | − | − | + |
| Lipoxygenase | + | − | + | − | − | + |
| Lipoxygenase | + | − | + | − | − | + |
| Lipoxygenase | + | − | + | − | − | + |
| Lipoxygenase | − | + | − | + | − | − |
| Lipoxygenase | − | + | − | + | − | − |
| Lipoxygenase | − | − | + | + | − | + |
| Lipoxygenase | + | − | − | − | − | − |
| Lipoxygenase | − | − | + | − | − | − |
| Malic enzyme | − | − | + | − | + | + |
| Malic enzyme | − | − | + | − | + | + |
| MLO-like protein | − | − | − | + | − | − |
| MLO-like protein | − | − | − | + | − | − |
| MLO-like protein | + | − | + | − | − | − |
| MLO-like protein | − | − | − | + | − | − |
| MLO-like protein | − | − | + | − | − | + |
| MLO-like protein | − | + | + | + | − | − |
| Monosaccharide transporter | − | − | + | + | + | + |
| MYB transcription factor MYB187 | − | + | + | − | − | + |
| NAC domain protein | + | − | − | + | − | + |
| NADPH--cytochrome P450 reductase | − | + | + | − | − | − |
| nine-cis-epoxycarotenoid dioxygenase 4 | − | + | − | + | − | − |
| Nitrate reductase | − | − | − | − | − | − |
| Nodulin-16 | + | − | − | − | − | − |
| Nodulin-16 | + | − | − | − | − | − |
| Nodulin-20 | + | − | − | − | − | − |
| Nodulin-21 | + | − | − | − | − | − |
| Nodulin-22 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-24 | + | − | − | − | − | − |
| Nodulin-26 | + | − | − | + | − | − |
| Nodulin-26B | + | − | − | − | − | − |
| Nodulin-26B | + | − | − | − | − | − |
| Nodulin-26B | + | − | − | − | − | − |
| Nodulin-44 | + | − | − | − | − | − |
| Nodulin-C51 | + | − | − | − | − | − |
| Nodulin-C51 | + | − | − | − | − | − |
| Non-specific lipid-transfer protein | − | − | − | − | − | + |
| Non-specific lipid-transfer protein | − | − | + | − | − | + |
| Non-specific lipid-transfer protein | − | + | − | − | − | + |
| Non-specific lipid-transfer protein | − | − | − | − | − | + |
| Non-specific lipid-transfer protein | − | − | − | − | + | + |
| Non-specific lipid-transfer protein | − | − | + | + | − | − |
| Non-specific lipid-transfer protein | − | − | − | − | − | + |
| Non-specific lipid-transfer protein | − | − | − | − | − | + |
| Non-specific lipid-transfer protein | − | − | + | − | − | + |
| Non-specific lipid-transfer protein | − | + | − | − | − | + |
| Non-specific lipid-transfer protein | − | − | − | − | − | + |
| Non-specific lipid-transfer protein | − | − | + | − | − | + |
| Pectinesterase | − | − | − | + | − | − |
| Peptidyl-prolyl cis-trans isomerase | − | − | − | + | + | − |
| Peptidyl-prolyl cis-trans isomerase | + | − | − | + | + | + |
| Peptidyl-prolyl cis-trans isomerase | − | − | − | − | + | + |
| Peptidyl-prolyl cis-trans isomerase | − | − | − | − | − | + |
| Peptidyl-prolyl cis-trans isomerase | − | − | − | − | − | + |
| Peptidyl-prolyl cis-trans isomerase | − | − | − | − | − | + |
| Peptidyl-prolyl cis-trans isomerase | − | − | − | + | + | − |
| Peptidyl-prolyl cis-trans isomerase 1 | − | − | − | + | + | − |
| Peroxidase | − | − | − | + | + | − |
| Phenylalanine ammonia-lyase | − | − | − | − | + | − |
| Phospholipase D | − | − | + | + | − | + |
| Phosphomannomutase | − | − | − | − | − | + |
| Phosphoribulokinase | + | − | − | + | − | − |
| Phosphoribulokinase | + | − | − | + | − | + |
| Phosphorylase | + | + | + | − | − | − |
| Phosphorylase | + | + | + | − | − | − |
| photosystem I subunit F | + | − | − | + | + | − |
| Photosystem II reaction center protein J | − | − | − | − | − | − |
| Photosystem II reaction center protein Z | − | − | − | − | − | − |
| Photosystem II reaction center protein Z | − | − | − | − | − | − |
| Protein P21 | − | − | − | − | − | − |
| Protein PsbN | − | − | + | − | − | − |
| Repetitive proline-rich cell wall protein 2 | + | − | + | + | + | − |
| Repetitive proline-rich cell wall protein 3 | − | − | + | + | − | − |
| Ribose-phosphate pyrophosphokinase | − | − | − | − | − | − |
| Ribulose bisphosphate carboxylase small chain | − | − | − | − | − | + |
| Ribulose bisphosphate carboxylase small chain 1 | + | − | − | + | + | + |
| (chloroplastic) | | | | | | |
| Ribulose bisphosphate carboxylase small chain 4 | + | − | − | + | + | + |
| (chloroplastic) | | | | | | |
| Ribulose bisphosphate carboxylase small chain 4 | + | − | − | + | + | + |
| (chloroplastic) | | | | | | |
| Ribulose bisphosphate carboxylase small chain 4 | + | − | − | + | + | + |
| (chloroplastic) | | | | | | |
| Ribulose bisphosphate carboxylase small chain 4 | + | − | − | + | + | + |
| (chloroplastic) | | | | | | |
| RuBisCO-associated protein | − | − | − | − | − | − |
| S-adenosylmethionine synthase | − | − | − | − | − | + |
| S-adenosylmethionine synthase | + | + | + | − | + | − |
| Serine hydroxymethyltransferase | + | + | − | + | − | − |
| Serine hydroxymethyltransferase | − | + | + | + | − | − |
| Serine hydroxymethyltransferase | − | + | + | − | − | − |
| Serine hydroxymethyltransferase | − | − | + | − | − | − |
| Serine/threonine-protein kinase | − | − | − | + | − | − |
| Serine/threonine-protein kinase | − | + | − | − | − | + |
| Serine/threonine-protein kinase | − | + | − | − | − | + |
| Serine/threonine-protein kinase | − | + | − | − | − | + |
| Signal recognition particle 9 kDa protein | − | − | + | − | + | + |
| Site-determining protein | − | − | − | − | − | − |
| Stem 28 kDa glycoprotein | − | − | − | − | − | − |
| Stem 28 kDa glycoprotein | − | − | − | − | − | − |
| Stem 31 kDa glycoprotein | − | − | − | − | − | − |
| Stem 31 kDa glycoprotein | − | − | − | − | − | − |
| Stress-induced protein SAM22 | + | − | + | + | − | + |
| Stress-induced protein SAM22 | + | − | + | + | − | + |
| Stress-induced protein SAM22 | + | − | + | + | − | + |
| Superoxide dismutase | − | − | + | + | + | + |
| Superoxide dismutase | − | − | + | + | + | + |
| Superoxide dismutase | − | − | + | + | + | + |
| Superoxide dismutase | − | − | + | − | + | + |
| Superoxide dismutase | + | − | + | − | − | + |
| Superoxide dismutase | + | − | + | − | − | + |
| Superoxide dismutase | + | − | + | − | − | + |
| Thioredoxin | − | + | + | + | − | + |
| Thioredoxin | − | − | − | + | + | − |
| Thioredoxin | + | − | − | + | − | − |
| Thioredoxin | + | − | − | − | − | − |
| Thioredoxin | − | − | − | + | + | + |
| Thioredoxin | − | − | − | + | + | + |
| Thioredoxin | − | − | − | + | + | − |
| Thioredoxin | − | − | − | + | − | − |
| Tubulin beta-1 chain | − | + | + | + | + | + |
| UDP-glucose 6-dehydrogenase | − | + | − | + | + | + |
| Uracil-DNA glycosylase | − | − | − | − | + | + |
| Uracil-DNA glycosylase | − | − | − | − | + | + |
| Wound-induced protein | − | − | + | − | − | − |

TABLE 7B
Quantification of up- and down- regulated genes identified in qualitative transcriptomics studies, in plants grown
from seeds treated with Strain B, as compared to plants grown from seeds treated with the formulation control.

| | | Qualitative Plant Transcriptomics | | Quantitative Plant Transcriptomics | | |
|---|---|---|---|---|---|---|
| | | | Up/Down | | Up/Down | Fold |
| Tissue | Plant GeneName | Gene | Regulated | Gene | Regulated | Chang [symbol] |
| Root | Glyma.09G229200 | Purple acid phosphatase | + | purple acid phosphatase 10 | + | 1.84 |
| Root | Glyma.11G130900 | Histone H3 | + | Histone superfamily protein | + | 1.67 |
| Root | Glyma.11G035300 | Putative uncharacterized protein | + | 2-oxoglutarate (2OG) and Fe(II)- | + | 1.54 |
| | | | | dependent oxygenase superfamily | | |
| | | | | protein | | |
| Root | Glyma.15G237800 | Annexin | + | annexin 8 | + | 1.53 |
| Root | Glyma.08G189500 | Lipoxygenase | + | lipoxygenase 1 | + | 1.52 |
| Leaf | Glyma.18G255300 | Thioredoxin | + | thioredoxin H-type 5 | + | 1.51 |
| Leaf | Glyma.16G165500 | Chlorophyll a-b binding protein 2, | + | light-harvesting chlorophyll-protein | + | 1.50 |
| | | chloroplastic | | complex II subunit B1 | | |
| Leaf | Glyma.11G053400 | Soyasapogenol B glucuronide | + | UDP-glucosyl transferase 73B1 | + | 1.47 |
| | | galactosyltransferase | | | | |
| Root | Glyma.13G199500 | Annexin | + | annexin 8 | + | 1.45 |
| Leaf | Glyma.08G181000 | Soyasaponin III rhamnosyltransferase | + | UDP-Glycosyltransferase superfamily | + | 1.44 |
| | | | | protein | | |
| Leaf | Glyma.05G007100 | Carbonic anhydrase | + | carbonic anhydrase 1 | + | 1.43 |
| Root | Glyma.15G032300 | Histone H3 | + | Histone superfamily protein | + | 1.43 |
| Leaf | Glyma.03G179200 | MLO-like protein | + | Seven transmembrane MLO family | + | 1.42 |
| | | | | protein | | |
| Root | Glyma.03G028000 | Arginase | + | Arginase/deacetylase superfamily | + | 1.41 |
| | | | | protein | | |
| Leaf | Glyma.08G350800 | Beta-amyrin 24-hydroxylase | + | cytochrome P450, family 93, | + | 1.39 |
| | | | | subfamily D, polypeptide 1 | | |
| Root | Glyma.16G080100 | MLO-like protein | + | Seven transmembrane MLO family | + | 1.38 |
| | | | | protein | | |
| Root | Glyma.13G307000 | Putative uncharacterized protein | + | Peroxidase superfamily protein | + | 1.38 |
| Root | Glyma.10G292200 | Chalcone--flavonone isomerase 1B-2 | + | Chalcone-flavanone isomerase family | + | 1.37 |
| | | | | protein | | |
| Root | Glyma.05G222300 | Cytidine deaminase | + | cytidine deaminase 1 | + | 1.36 |
| Leaf | Glyma.13G347700 | Linoleate 9S-lipoxygenase-4 | + | lipoxygenase 1 | + | 1.35 |
| Root | Glyma.04G015700 | MLO-like protein | + | Seven transmembrane MLO family | + | 1.35 |
| | | | | protein | | |
| Leaf | Glyma.05G208900 | Cytochrome P450 monooxygenase | + | cytochrome P450, family 86, | + | 1.34 |
| | | CYP86A24 | | subfamily A, polypeptide 8 | | |
| Leaf | Glyma.07G001300 | Beta-amyrin synthase | + | Terpenoid cyclases family protein | + | 1.34 |
| Leaf | Glyma.07G185400 | Malate dehydrogenase | + | peroxisomal NAD-malate dehydrogenase | + | 1.32 |
| | | | | 2 | | |
| Leaf | Glyma.16G205200 | Putative uncharacterized protein | + | light harvesting complex of photosystem | + | 1.30 |
| | | | | II 5 | | |
| Leaf | Glyma.13G046200 | Ribulose bisphosphate carboxylase | + | Ribulose bisphosphate carboxylase (small | + | 1.30 |
| | | small chain 1, chloroplastic | | chain) family protein | | |
| Leaf | Glyma.19G046800 | Ribulose bisphosphate carboxylase | + | Ribulose bisphosphate carboxylase (small | + | 1.30 |
| | | small chain 4, chloroplastic | | chain) family protein | | |
| Leaf | Glyma.15G213600 | Serine/threonine-protein kinase | + | S-locus lectin protein kinase family | + | 1.29 |
| | | | | protein | | |
| Leaf | Glyma.05G201300 | Acyl carrier protein | + | acyl carrier protein 4 | + | 1.29 |
| Leaf | Glyma.11G096600 | Putative uncharacterized protein | + | NAC domain containing protein 36 | + | 1.29 |
| Leaf | Glyma.09G210900 | Phosphoribulokinase | + | phosphoribulokinase | + | 1.28 |
| Leaf | Glyma.19G046600 | Ribulose bisphosphate carboxylase | + | Ribulose bisphosphate carboxylase (small | + | 1.28 |
| | | small chain 4, chloroplastic | | chain) family protein | | |
| Leaf | Glyma.10G159700 | Cysteine synthase | + | cysteine synthase D1 | + | 1.27 |
| Leaf | Glyma.19G212600 | Pectinesterase | + | Plant invertase/pectin methylesterase | + | 1.27 |
| | | | | inhibitor superfamily | | |
| Leaf | Glyma.14G010900 | Fructose-bisphosphate aldolase | + | Aldolase superfamily protein | + | 1.27 |
| Leaf | Glyma.03G028000 | Arginase | + | Arginase/deacetylase superfamily | + | 1.27 |
| | | | | protein | | |
| Leaf | Glyma.19G007700 | Carbonic anhydrase | + | carbonic anhydrase 1 | + | 1.27 |
| Leaf | Glyma.12G198200 | Serine/threonine-protein kinase | + | S-locus lectin protein kinase family | + | 1.26 |
| | | | | protein | | |
| Leaf | Glyma.13G210800 | Glutamine synthetase | + | glutamine synthetase 2 | + | 1.26 |
| Leaf | Glyma.07G266000 | Putative uncharacterized protein | + | Cystatin/monellin superfamily protein | + | 1.25 |
| Leaf | Glyma.07G034800 | Lipoxygenase | + | lipoxygenase 1 | + | 1.24 |
| Root | Glyma.13G284200 | Putative uncharacterized protein | − | flavodoxin-like quinone reductase 1 | − | −1.20 |
| Leaf | Glyma.13G208200 | Putative uncharacterized protein | − | Eukaryotic aspartyl protease family | − | −1.20 |
| | | | | protein | | |
| Root | Glyma.17G072400 | Heat shock 70 kDa protein | − | heat shock protein 70B | − | −1.20 |
| Root | Glyma.12G102900 | Beta-amylase | − | beta-amylase 5 | − | −1.20 |
| Root | Glyma.01G162100 | Phospholipase D | − | phospholipase D delta | − | −1.20 |
| Root | Glyma.13G028200 | Photosystem Q(B) protein | − | photosystem II reaction center protein A | − | −1.20 |
| Root | Glyma.06G011700 | Glucose-1-phosphate | − | Glucose-1-phosphate | − | −1.21 |
| | | adenylyltransferase | | adenylyltransferase family protein | | |
| Root | Glyma.19G000700 | Pyruvate kinase | − | Pyruvate kinase family protein | − | −1.21 |
| Leaf | Glyma.18G202600 | Omega-3 fatty acid desaturase, | − | fatty acid desaturase 8 | − | −1.21 |
| | | chloroplastic | | | | |
| Root | Glyma.20G196900 | Superoxide dismutase | − | Fe superoxide dismutase 2 | − | −1.21 |
| Root | Glyma.04G210000 | NAD-dependent protein deacetylase | − | sirtuin 2 | − | −1.21 |
| Root | Glyma.14G182100 | NADPH--cytochrome P450 reductase | − | P450 reductase 1 | − | −1.21 |
| Leaf | Glyma.10G201100 | Pyruvate kinase | − | Pyruvate kinase family protein | − | −1.22 |
| Root | Glyma.09G037400 | DNA-directed RNA polymerase subunit | − | RNA polymerases M/15 Kd subunit | − | −1.23 |
| Root | Glyma.05G161600 | Glutathione S-transferase GST 14 | − | glutathione S-transferase tau 7 | − | −1.23 |
| Leaf | Glyma.17G153200 | Ribose-phosphate pyrophosphokinase | − | Phosphoribosyltransferase family protein | − | −1.24 |
| Root | Glyma.12G042400 | Glucose-1-phosphate | − | Glucose-1-phosphate | − | −1.24 |
| | | adenylyltransferase | | adenylyltransferase family protein | | |
| Leaf | Glyma.09G073600 | Sucrose synthase | − | sucrose synthase 4 | − | −1.25 |
| Leaf | Glyma.04G248500 | Aldehyde dehydrogenase | − | aldehyde dehydrogenase 3H1 | − | −1.26 |
| Root | Glyma.07G200200 | 18.5 kDa class I heat shock protein | − | HSP20-like chaperones superfamily | − | −1.27 |
| | | | | protein | | |
| Root | Glyma.04G011900 | Glucose-1-phosphate | − | Glucose-1-phosphate | − | −1.28 |
| | | adenylyltransferase | | adenylyltransferase family protein | | |
| Leaf | Glyma.01G129400 | Peroxisomal aminotransferase | − | alanine:glyoxylate aminotransferase 3 | − | −1.28 |
| Root | Glyma.10G193500 | Superoxide dismutase | − | Fe superoxide dismutase 2 | − | −1.28 |
| Root | Glyma.07G139700 | Probable glutathione S-transferase | − | glutathione S-transferase TAU 8 | − | −1.28 |
| Leaf | Glyma.11G170300 | Asparagine synthetase | − | glutamine-dependent asparagine | − | −1.28 |
| | | | | synthase 1 | | |
| Root | Glyma.18G080400 | Cytochrome P450 71D9 | − | cytochrome P450, family 71, | − | −1.29 |
| | | | | subfamily B, polypeptide 34 | | |
| Root | Glyma.05G000700 | Pyruvate kinase, cytosolic isozyme | − | Pyruvate kinase family protein | − | −1.33 |
| Leaf | Glyma.17G192000 | Putative uncharacterized protein | − | amino acid permease 6 | − | −1.34 |
| Leaf | Glyma.13G176100 | Polyubiquitin Ubiquitin | − | polyubiquitin 10 | − | −1.35 |
| Leaf | Glyma.05G161600 | Glutathione S-transferase GST 14 | − | glutathione S-transferase tau 7 | − | −1.36 |
| Leaf | Glyma.14G177600 | Putative uncharacterized protein | − | Cupredoxin superfamily protein | − | −1.37 |
| Root | Glyma.20G241700 | Chalcone-flavonone isomerase 2-A | − | Chalcone-flavanone isomerase family | − | −1.39 |
| | | | | protein | | |
| Leaf | Glyma.02G228100 | Asparagine synthetase | − | glutamine-dependent asparagine | − | −1.44 |
| | | | | synthase 1 | | |

[symbol]

indicates data missing or illegible when filed

TABLE 7C
Additional top up- and down- regulated genes in plants grown from seeds treated with Strain C, as compared to plants grown
from seeds treated with the formulation control, that were not identified in the qualitative transcriptomics studies.

| | | Quantitative Plant Transcriptomics | | |
|---|---|---|---|---|
| | | | Up/Down | |
| Tissue | Plant Gene Name | Gene | Regulated | Fold Change |
| Root | Glyma.06G031600.1 | AMP-dependent synthetase and ligase family protein | + | 2.54 |
| Leaf | Glyma.16G169400.1 | receptor like protein 19 | + | 2.18 |
| Root | Glyma.20G047000.1 | Protein kinase protein with adenine nucleotide alpha hydrolases- | + | 2.11 |
| | | like domain | | |
| Root | Glyma.12G130700.1 | Cysteine proteinases superfamily protein | + | 2.04 |
| Leaf | Glyma.13G272300.1 | sodium/calcium exchanger family protein/calcium-binding EF hand | + | 1.92 |
| | | family protein | | |
| Root | Glyma.10G231700.1 | ferric reduction oxidase 2 | + | 1.92 |
| Root | Glyma.13G336600.1 | expansin A4 | + | 1.91 |
| Root | Glyma.03G039800.1 | Peroxidase superfamily protein | + | 1.85 |
| Root | Glyma.13G281200.1 | Late embryogenesis abundant (LEA) protein-related | + | 1.84 |
| Root | Glyma.07G113500.1 | H(+)-ATPase 11 | + | 1.83 |
| Root | Glyma.10G125000.1 | Pectin lyase-like superfamily protein | + | 1.81 |
| Root | Glyma.14G218100.1 | Putative lysine decarboxylase family protein | + | 1.81 |
| Root | Glyma.04G073500.1 | potassium channel in Arabidopsis thaliana 3 | + | 1.79 |
| Root | Glyma.16G120300.1 | SGNH hydrolase-type esterase superfamily protein | + | 1.79 |
| Root | Glyma.04G180400.1 | BURP domain-containing protein | + | 1.78 |
| Leaf | Glyma.14G209600.1 | AMP-dependent synthetase and ligase family protein | + | 1.78 |
| Root | Glyma.06G284900.1 | Basic-leucine zipper (bZIP) transcription factor family protein | + | 1.77 |
| Root | Glyma.19G251100.1 | Protein kinase superfamily protein | + | 1.75 |
| Root | Glyma.06G273400.1 | Cysteine proteinases superfamily protein | + | 1.75 |
| Root | Glyma.01G020000.1 | ABC-2 type transporter family protein | + | 1.75 |
| Root | Glyma.11G024300.1 | C2 calcium/lipid-binding plant phosphoribosyltransferase family protein | + | 1.74 |
| Root | Glyma.03G113400.1 | H(+)-ATPase 11 | + | 1.73 |
| Root | Glyma.01G211000.1 | Glycosyl hydrolases family 32 protein | + | 1.73 |
| Leaf | Glyma.07G205400.1 | Cysteine proteinases superfamily protein | + | 1.73 |
| Root | Glyma.18G125200.1 | Integrase-type DNA-binding superfamily protein | + | 1.73 |
| Root | Glyma.08G323400.1 | MBOAT (membrane bound O-acyl transferase) family protein | + | 1.72 |
| Root | Glyma.11G056900.1 | Acyl transferase/acyl hydrolase/lysophospholipase superfamily protein | + | 1.71 |
| Root | Glyma.13G178700.1 | Pollen Ole e 1 allergen and extensin family protein | + | 1.71 |
| Root | Glyma.17G057300.1 | Auxin efflux carrier family protein | + | 1.71 |
| Root | Glyma.02G142400.1 | indoleacetic acid-induced protein 16 | + | 1.69 |
| Root | Glyma.12G027200.1 | glycosyl hydrolase 9C1 | + | 1.69 |
| Root | Glyma.03G092800.1 | non-specific phospholipase C3 | + | 1.69 |
| Root | Glyma.14G123500.1 | phosphate transporter 1;1 | + | 1.69 |
| Root | Glyma.02G046800.1 | Glycosyl hydrolase family 38 protein | + | 1.68 |
| Root | Glyma.13G342600.1 | AMP-dependent synthetase and ligase family protein | + | 1.68 |
| Root | Glyma.20G199400.1 | | + | 1.68 |
| Root | Glyma.13G132500.1 | nodulin MtN21/EamA-like transporter family protein | + | 1.68 |
| Leaf | Glyma.16G172100.1 | disease resistance family protein/LRR family protein | + | 1.67 |
| Root | Glyma.10G119900.1 | glycerol-3-phosphate acyltransferase 6 | + | 1.67 |
| Root | Glyma.06G123900.1 | glycosyl hydrolase 9A1 | + | 1.67 |
| Leaf | Glyma.16G175500.1 | UDP-glucosyl transferase 88A1 | + | 1.67 |
| Leaf | Glyma.07G254600.1 | UDP-Glycosyltransferase superfamily protein | + | 1.67 |
| Leaf | Glyma.16G174600.1 | disease resistance family protein/LRR family protein | + | 1.66 |
| Root | Glyma.19G212400.1 | Plant invertase/pectin methylesterase inhibitor superfamily | + | 1.66 |
| Root | Glyma.12G185800.1 | germin-like protein 10 | + | 1.66 |
| Root | Glyma.17G179400.1 | Disease resistance protein (CC-NBS-LRR class) family | + | 1.65 |
| Root | Glyma.15G086000.1 | FASCICLIN-like arabinogalactan-protein 12 | + | 1.65 |
| Root | Glyma.08G167700.1 | Myzus persicae-induced lipase 1 | + | 1.65 |
| Leaf | Glyma.16G171800.1 | receptor like protein 15 | + | 1.64 |
| Leaf | Glyma.10G104700.1 | UDP-Glycosyltransferase superfamily protein | + | 1.64 |
| Leaf | Glyma.15G221300.1 | UDP-glucosyl transferase 73B3 | + | 1.62 |
| Leaf | Glyma.14G015500.1 | receptor like protein 6 | + | 1.62 |
| Leaf | Glyma.20G047400.1 | Cofactor-independent phosphoglycerate mutase | + | 1.61 |
| Leaf | Glyma.16G197500.1 | 2-oxoglutarate (2OG) and Fe(II)-dependent oxygenase superfamily protein | + | 1.59 |
| Leaf | Glyma.12G206500.1 | | + | 1.59 |
| Leaf | Glyma.13G183200.1 | | + | 1.58 |
| Leaf | Glyma.06G324300.1 | cellulose synthase like G1 | + | 1.57 |
| Leaf | Glyma.19G030800.1 | HXXXD-type acyl-transferase family protein | + | 1.56 |
| Leaf | Glyma.16G187100.1 | disease resistance family protein/LRR family protein | + | 1.55 |
| Leaf | Glyma.16G171200.1 | disease resistance family protein/LRR family protein | + | 1.55 |
| Leaf | Glyma.19G133300.1 | 2-oxoglutarate (2OG) and Fe(II)-dependent oxygenase superfamily protein | + | 1.54 |
| Leaf | Glyma.04G233800.1 | | + | 1.54 |
| Leaf | Glyma.03G215100.1 | Protein of unknown function, DUF538 | + | 1.53 |
| Leaf | Glyma.17G134200.1 | cytochrome P450, family 706, subfamily A, polypeptide 4 | + | 1.52 |
| Leaf | Glyma.16G211700.1 | Kunitz family trypsin and protease inhibitor protein | + | 1.52 |
| Leaf | Glyma.01G198100.1 | basic helix-loop-helix (bHLH) DNA-binding superfamily protein | + | 1.52 |
| Leaf | Glyma.15G160600.1 | Ankyrin repeat family protein | + | 1.51 |
| Leaf | Glyma.16G174800.1 | disease resistance family protein/LRR family protein | + | 1.51 |
| Leaf | Glyma.03G141600.1 | Leucine-rich repeat protein kinase family protein | + | 1.51 |
| Leaf | Glyma.06G056400.1 | Leucine-rich receptor-like protein kinase family protein | + | 1.50 |
| Leaf | Glyma.19G202900.1 | alpha/beta-Hydrolases superfamily protein | + | 1.50 |
| Leaf | Glyma.16G169900.1 | disease resistance family protein/LRR family protein | + | 1.49 |
| Leaf | Glyma.16G170700.1 | disease resistance family protein/LRR family protein | + | 1.49 |
| Leaf | Glyma.01G160100.1 | basic chitinase | + | 1.49 |
| Leaf | Glyma.05G044000.1 | Pectin lyase-like superfamily protein | + | 1.48 |
| Leaf | Glyma.20G154700.1 | Pyridoxal phosphate (PLP)-dependent transferases superfamily protein | + | 1.48 |
| Leaf | Glyma.18G106300.1 | rhamnose biosynthesis 1 | + | 1.48 |
| Leaf | Glyma.01G192400.1 | | + | 1.48 |
| Leaf | Glyma.19G000900.1 | actin-11 | + | 1.48 |
| Leaf | Glyma.05G176700.1 | | + | 1.47 |
| Leaf | Glyma.11G218400.1 | | − | −1.45 |
| Leaf | Glyma.12G207400.1 | Thioredoxin superfamily protein | − | −1.46 |
| Leaf | Glyma.02G127300.1 | Peroxidase superfamily protein | − | −1.46 |
| Leaf | Glyma.05G063600.1 | ethylene responsive element binding factor 1 | − | −1.46 |
| Leaf | Glyma.16G100100.1 | Bifunctional inhibitor/lipid-transfer protein/seed storage 2S albumin | − | −1.46 |
| | | superfamily protein | | |
| Leaf | Glyma.20G168500.1 | Integrase-type DNA-binding superfamily protein | − | −1.47 |
| Leaf | Glyma.12G116900.1 | Zinc finger C-x8-C-x5-C-x3-H type family protein | − | −1.47 |
| Leaf | Glyma.16G164800.1 | Integrase-type DNA-binding superfamily protein | − | −1.47 |
| Leaf | Glyma.11G062600.1 | cytochrome P450, family 71, subfamily B, polypeptide 34 | − | −1.47 |
| Leaf | Glyma.16G188800.1 | Bifunctional inhibitor/lipid-transfer protein/seed storage 2S albumin | − | −1.47 |
| | | superfamily protein | | |
| Leaf | Glyma.19G038600.1 | ATPase E1-E2 type family protein/haloacid dehalogenase-like hydrolase | − | −1.48 |
| | | family protein | | |
| Root | Glyma.08G068700.1 | HSP20-like chaperones superfamily protein | − | −1.48 |
| Leaf | Glyma.06G157400.1 | NAC (No Apical Meristem) domain transcriptional regulator superfamily | − | −1.48 |
| | | protein | | |
| Root | Glyma.20G188700.1 | RNI-like superfamily protein | − | −1.48 |
| Root | Glyma.15G106200.1 | adenosine/AMP deaminase family protein | − | −1.48 |
| Root | Glyma.16G162600.1 | chlorophyll A/B binding protein 1 | − | −1.48 |
| Leaf | Glyma.18G197400.1 | Laccase/Diphenol oxidase family protein | − | −1.48 |
| Root | Glyma.03G114300.1 | homeobox-leucine zipper protein 3 | − | −1.48 |
| Root | Glyma.03G101200.1 | Fatty acid hydroxylase superfamily | − | −1.48 |
| Leaf | Glyma.06G195000.1 | expansin A15 | − | −1.48 |
| Leaf | Glyma.12G221500.1 | NAC domain containing protein 3 | − | −1.48 |
| Leaf | Glyma.13G279900.1 | NAC domain containing protein 3 | − | −1.48 |
| Leaf | Glyma.05G108200.1 | basic leucine-zipper 5 | − | −1.49 |
| Leaf | Glyma.04G097900.1 | MATE efflux family protein | − | −1.49 |
| Leaf | Glyma.05G126800.1 | | − | −1.49 |
| Root | Glyma.11G095100.1 | Glycosyl hydrolase superfamily protein | − | −1.49 |
| Root | Glyma.02G058000.1 | Major facilitator superfamily protein | − | −1.49 |
| Leaf | Glyma.06G154200.1 | cation/hydrogen exchanger 15 | − | −1.49 |
| Leaf | Glyma.10G192900.1 | glutathione S-transferase TAU 15 | − | −1.49 |
| Leaf | Glyma.16G158600.1 | SNF1-related protein kinase 2.9 | − | −1.50 |
| Leaf | Glyma.08G244800.1 | UDP-Glycosyltransferase superfamily protein | − | −1.50 |
| Root | Glyma.08G342000.1 | kunitz trypsin inhibitor 1 | − | −1.50 |
| Root | Glyma.01G219300.1 | root hair specific 8 | − | −1.50 |
| Root | Glyma.16G141000.1 | Major facilitator superfamily protein | − | −1.51 |
| Leaf | Glyma.10G007000.1 | ethylene response factor 1 | − | −1.51 |
| Leaf | Glyma.02G224800.1 | Laccase/Diphenol oxidase family protein | − | −1.51 |
| Leaf | Glyma.14G081300.1 | phospholipase A 2A | − | −1.51 |
| Root | Glyma.09G210500.1 | | − | −1.51 |
| Root | Glyma.07G200500.1 | HSP20-like chaperones superfamily protein | − | −1.51 |
| Root | Glyma.14G106700.1 | Protein of unknown function (DUF1677) | − | −1.51 |
| Root | Glyma.18G129800.1 | | − | −1.52 |
| Root | Glyma.16G163300.1 | | − | −1.52 |
| Leaf | Glyma.14G103100.1 | WRKY DNA-binding protein 40 | − | −1.52 |
| Leaf | Glyma.05G184500.1 | WRKY DNA-binding protein 51 | − | −1.52 |
| Root | Glyma.11G024100.1 | glutathione peroxidase 6 | − | −1.52 |
| Leaf | Glyma.11G180700.1 | BRI1-associated receptor kinase | − | −1.52 |
| Root | Glyma.12G180000.1 | magnesium (Mg) transporter 10 | − | −1.52 |
| Root | Glyma.16G159300.1 | Disease resistance protein (TIR-NBS-LRR class) family | − | −1.52 |
| Leaf | Glyma.10G010100.1 | NIM1-interacting 1 | − | −1.53 |
| Root | Glyma.16G187500.1 | disease resistance family protein/LRR family protein | − | −1.53 |
| Leaf | Glyma.02G009500.1 | NIM1-interacting 1 | − | −1.53 |
| Leaf | Glyma.18G190000.1 | Bifunctional inhibitor/lipid-transfer protein/seed storage 2S albumin | − | −1.53 |
| | | superfamily protein | | |
| Leaf | Glyma.17G147600.1 | expansin-like B1 | − | −1.53 |
| Root | Glyma.18G094600.1 | heat shock protein 21 | − | −1.54 |
| Root | Glyma.09G111900.1 | fatty acid desaturase 2 | − | −1.54 |
| Leaf | Glyma.19G136700.1 | UDP-glycosyltransferase 73B4 | − | −1.55 |
| Leaf | Glyma.16G195900.1 | Tetratricopeptide repeat (TPR)-like superfamily protein | − | −1.58 |
| Root | Glyma.01G081600.1 | Major facilitator superfamily protein | − | −1.58 |
| Root | Glyma.09G070500.1 | strictosidine synthase-like 4 | − | −1.59 |
| Leaf | Glyma.01G137500.1 | SAUR-like auxin-responsive protein family | − | −1.59 |
| Leaf | Glyma.16G189900.1 | CAP160 protein | − | −1.60 |
| Root | Glyma.16G195700.1 | Tetratricopeptide repeat (TPR)-like superfamily protein | − | −1.61 |
| Root | Glyma.16G188800.1 | Bifunctional inhibitor/lipid-transfer protein/seed storage 2S albumin | − | −1.61 |
| | | superfamily protein | | |
| Leaf | Glyma.18G231500.1 | | − | −1.61 |
| Leaf | Glyma.11G051800.1 | cytochrome P450, family 81, subfamily D, polypeptide 3 | − | −1.61 |
| Leaf | Glyma.12G149100.1 | NAC (No Apical Meristem) domain transcriptional regulator superfamily | − | −1.61 |
| | | protein | | |
| Root | Glyma.04G082300.1 | tocopherol cyclase, chloroplast/vitamin E deficient 1 (VTE1)/sucrose | − | −1.61 |
| | | export defective 1 (SXD1) | | |
| Root | Glyma.16G159100.1 | Disease resistance protein (TIR-NBS-LRR class) family | − | −1.62 |
| Root | Glyma.12G132600.1 | Protein kinase superfamily protein | − | −1.62 |
| Root | Glyma.14G063700.1 | HSP20-like chaperones superfamily protein | − | −1.64 |
| Root | Glyma.16G195600.1 | cytochrome P450, family 71, subfamily A, polypeptide 26 | − | −1.65 |
| Root | Glyma.15G062400.1 | basic pathogenesis-related protein 1 | − | −1.65 |
| Root | Glyma.13G291800.1 | late embryogenesis abundant domain-containing protein/LEA domain- | − | −1.65 |
| | | containing protein | | |
| Root | Glyma.16G173000.1 | chitinase A | − | −1.71 |
| Root | Glyma.16G182400.1 | Plant regulator RWP-RK family protein | − | −1.83 |
| Root | Glyma.16G159200.1 | Disease resistance protein (TIR-NBS-LRR class) family | − | −1.86 |
| Root | Glyma.16G175800.1 | Glycosyl hydrolases family 32 protein | − | −1.90 |
| Root | Glyma.12G132500.1 | UDP-Glycosyltransferase superfamily protein | − | −1.90 |
| Root | Glyma.16G168400.1 | VIRE2-interacting protein 1 | − | −1.92 |
| Root | Glyma.05G095600.1 | Amino acid kinase family protein | − | −1.96 |

TABLE 7D
Transcripts that are significantly up- or down- regulated in soybean plants grown
from seeds treated with Strain A or Strain B but that are not found to be significantly up- or
down- regulated in soybean plants grown from seeds treated with Strain F. Fold change is
expressed as Strain A (or Strain B) versus formulation control.

| | | | Fold | |
|---|---|---|---|---|
| Tissue | Transcript | label | Change | Transcript Name |
| Root | Glyma.15G019200.2 | StrainA vs Formulation | 7.09 | pleiotropic drug resistance 1 |
| Leaf | Glyma.07G144800.2 | StrainA vs Formulation | 6.03 | Transducin/WD40 repeat-like |
| | | | | superfamily protein |
| Root | Glyma.16G155800.1 | StrainA vs Formulation | 5.92 | histidinol phosphate |
| | | | | aminotransferase 1 |
| Root | Glyma.15G234500.1 | StrainA vs Formulation | 5.61 | |
| Leaf | Glyma.15G167100.7 | StrainA vs Formulation | 4.98 | |
| Root | Glyma.08G196700.6 | StrainA vs Formulation | 4.76 | |
| Root | Glyma.13G152800.2 | StrainA vs Formulation | 4.59 | |
| Root | Glyma.05G018500.5 | StrainA vs Formulation | 4.37 | galacturonosyltransferase 11 |
| Root | Glyma.16G105400.1 | StrainA vs Formulation | 4.13 | tetratricopeptide repeat (TPR)- |
| | | | | containing protein |
| Root | Glyma.13G124800.1 | StrainA vs Formulation | 4.09 | AGC (cAMP-dependent, cGMP- |
| | | | | dependent and protein kinase C) |
| | | | | kinase family protein |
| Root | Glyma.08G196700.6 | StrainB vs Formulation | 4.07 | |
| Leaf | Glyma.12G088600.5 | StrainA vs Formulation | 3.71 | |
| Leaf | Glyma.15G111700.1 | StrainA vs Formulation | 3.66 | Ribosomal protein L4/L1 family |
| Root | Glyma.08G288500.1 | StrainA vs Formulation | 3.51 | K+ uptake permease 6 |
| Leaf | Glyma.12G225400.2 | StrainA vs Formulation | 3.51 | embryo defective 2737 |
| Root | Glyma.16G040900.3 | StrainA vs Formulation | 3.44 | adenosine-5\′-phosphosulfate (APS) |
| | | | | kinase 3 |
| Root | Glyma.06G107200.2 | StrainA vs Formulation | 3.40 | electron carriers; protein disulfide |
| | | | | oxidoreductases |
| Root | Glyma.12G142100.1 | StrainB vs Formulation | 3.31 | S-locus lectin protein kinase family |
| | | | | protein |
| Root | Glyma.20G220200.1 | StrainA vs Formulation | 3.23 | phloem protein 2-B10 |
| Root | Glyma.13G152800.2 | StrainB vs Formulation | 3.16 | |
| Leaf | Glyma.04G152200.7 | StrainA vs Formulation | 3.15 | |
| Root | Glyma.08G005500.2 | StrainA vs Formulation | 3.05 | |
| Root | Glyma.10G035200.6 | StrainA vs Formulation | 3.03 | alpha/beta-Hydrolases superfamily |
| | | | | protein |
| Root | Glyma.05G018500.5 | StrainB vs Formulation | 2.95 | galacturonosyltransferase 11 |
| Root | Glyma.16G123800.10 | StrainB vs Formulation | 2.93 | PLAC8 family protein |
| Root | Glyma.10G083600.6 | StrainA vs Formulation | 2.89 | |
| Leaf | Glyma.12G040400.13 | StrainA vs Formulation | 2.76 | ARM repeat superfamily protein |
| Root | Glyma.12G163500.6 | StrainB vs Formulation | 2.68 | Phospholipid/glycerol |
| | | | | acyltransferase family protein |
| Root | Glyma.04G128700.3 | StrainB vs Formulation | −2.73 | SCP1-like small phosphatase 4 |
| Leaf | Glyma.12G211300.3 | StrainA vs Formulation | −2.77 | Protein of unknown function |
| | | | | (DUF688) |
| Root | Glyma.09G135200.5 | StrainA vs Formulation | −2.83 | NAD(P)-binding Rossmann-fold |
| | | | | superfamily protein |
| Leaf | Glyma.01G099800.2 | StrainA vs Formulation | −2.83 | delta 1-pyrroline-5-carboxylate |
| | | | | synthase 2 |
| Root | Glyma.04G185500.2 | StrainA vs Formulation | −3.07 | |
| Leaf | Glyma.01G013600.1 | StrainA vs Formulation | −3.08 | basic region/leucine zipper |
| | | | | transcription factor 16 |
| Leaf | Glyma.05G207400.3 | StrainA vs Formulation | −3.08 | Protein kinase protein with |
| | | | | tetratricopeptide repeat domain |
| Leaf | Glyma.04G165100.1 | StrainA vs Formulation | −3.13 | Plant protein of unknown function |
| | | | | (DUF863) |
| Root | Glyma.12G142100.2 | StrainB vs Formulation | −3.16 | S-locus lectin protein kinase family |
| | | | | protein |
| Root | Glyma.15G176500.2 | StrainA vs Formulation | −3.18 | growth-regulating factor 5 |
| Root | Glyma.04G105000.2 | StrainA vs Formulation | −3.22 | tRNA modification GTPase, putative |
| Root | Glyma.03G244500.9 | StrainB vs Formulation | −3.28 | Transducin/WD40 repeat-like |
| | | | | superfamily protein |
| Leaf | Glyma.17G126200.1 | StrainA vs Formulation | −3.72 | Transducin/WD40 repeat-like |
| | | | | superfamily protein |
| Root | Glyma.11G232900.1 | StrainA vs Formulation | −3.76 | P-loop containing nucleoside |
| | | | | triphosphate hydrolases superfamily |
| | | | | protein |
| Root | Glyma.13G337100.2 | StrainA vs Formulation | −3.83 | TRF-like 2 |
| Root | Glyma.14G098000.3 | StrainB vs Formulation | −4.00 | G protein alpha subunit 1 |
| Root | Glyma.01G122100.4 | StrainB vs Formulation | −4.59 | LMBR1-like membrane protein |
| Root | Glyma.10G243200.1 | StrainB vs Formulation | −4.65 | DNA repair (Rad51) family protein |
| Leaf | Glyma.20G156500.2 | StrainA vs Formulation | −5.14 | Homeobox-leucine zipper family |
| | | | | protein/lipid-binding START |
| | | | | domain-containing protein |
| Root | Glyma.14G098000.3 | StrainA vs Formulation | −5.20 | G protein alpha subunit 1 |
| Leaf | Glyma.17G170900.7 | StrainA vs Formulation | −5.38 | PLC-like phosphodiesterases |
| | | | | superfamily protein |
| Root | Glyma.02G157700.3 | StrainB vs Formulation | −5.51 | binding |
| Root | Glyma.13G116800.1 | StrainB vs Formulation | −6.61 | Alkaline-phosphatase-like family |
| | | | | protein |
| Root | Glyma.13G332300.3 | StrainA vs Formulation | −6.95 | sequence-specific DNA |
| | | | | binding; sequence-specific DNA |
| | | | | binding transcription factors |
| Root | Glyma.13G031600.3 | StrainA vs Formulation | −8.39 | amino acid permease 2 |
| Root | Glyma.04G201400.2 | StrainB vs Formulation | −9.04 | DCD (Development and Cell Death) |
| | | | | domain protein |
| Root | Glyma.06G098900.2 | StrainA vs Formulation | −9.31 | phosphoenolpyruvate carboxylase- |
| | | | | related kinase 2 |
| Root | Glyma.02G282700.2 | StrainA vs Formulation | −14.60 | helicase in vascular tissue and |
| | | | | tapetum |

TABLE 7E
Genes that are up- or down- regulated at least 1.5 fold higher or lower in the beneficial Penicillium
strains Strain A or Strain B as compared to the control Penicillium strain Strain F.

| | | | | Strain | Strain | | |
|---|---|---|---|---|---|---|---|
| | | | | B/ | A/ | | |
| | Strain | Strain | Strain | Strain | Strain | | |
| Tissue | B | A | F | F | F | Gene Name | Gene Description |
| Leaf | 2.18 | 2.51 | 1.05 | 2.07 | 2.38 | Glyma.16G169400 | receptor like protein 19 |
| Leaf | −1.58 | −2.11 | −1.01 | 1.56 | 2.09 | Glyma.16G195900 | Tetratricopeptide repeat (TPR)-like superfamily protein |
| Leaf | 1.66 | 2.08 | 1.03 | 1.61 | 2.02 | Glyma.16G174600 | disease resistance family protein/LRR family protein |
| Leaf | 1.40 | 2.02 | 1.13 | 1.24 | 1.79 | Glyma.16G172600 | multidrug resistance-associated protein 14 |
| Leaf | −1.03 | −1.76 | −1.01 | 1.03 | 1.75 | Glyma.U005800 | |
| Leaf | 1.51 | 1.78 | 1.02 | 1.48 | 1.74 | Glyma.16G174800 | disease resistance family protein/LRR family protein |
| Leaf | 1.64 | 1.72 | 1.01 | 1.62 | 1.70 | Glyma.16G171800 | receptor like protein 15 |
| Leaf | 1.18 | 1.79 | 1.06 | 1.11 | 1.68 | Glyma.16G138400 | RAD-like 6 |
| Leaf | −1.43 | −1.70 | −1.02 | 1.40 | 1.67 | Glyma.16G168400 | VIRE2-interacting protein 1 |
| Leaf | 1.55 | 1.72 | 1.03 | 1.51 | 1.67 | Glyma.16G187100 | disease resistance family protein/LRR family protein |
| Leaf | −1.08 | −1.67 | −1.01 | 1.08 | 1.66 | Glyma.16G174200 | P-loop containing nucleoside triphosphate hydrolases |
| | | | | | | | superfamily protein |
| Leaf | 1.49 | 1.68 | 1.02 | 1.47 | 1.65 | Glyma.16G169900 | disease resistance family protein/LRR family protein |
| Leaf | 1.28 | 1.64 | 1.01 | 1.26 | 1.62 | Glyma.16G174000 | receptor like protein 1 |
| Leaf | 1.43 | 1.67 | 1.04 | 1.38 | 1.61 | Glyma.16G158400 | Laccase/Diphenol oxidase family protein |
| Leaf | 1.40 | 1.63 | 1.02 | 1.37 | 1.60 | Glyma.17G182200 | receptor like protein 30 |
| Leaf | −1.01 | −1.66 | −1.05 | 0.97 | 1.59 | Glyma.01G163400 | |
| Leaf | −1.41 | −1.77 | −1.12 | 1.26 | 1.58 | Glyma.16G195800 | cytochrome P450, family 71, subfamily A, polypeptide 22 |
| Leaf | −1.52 | −1.88 | −1.19 | 1.28 | 1.58 | Glyma.05G184500 | WRKY DNA-binding protein 51 |
| Leaf | 1.01 | 1.87 | 1.18 | 0.85 | 1.58 | Glyma.03G015800 | BON association protein 2 |
| Leaf | 1.17 | 1.61 | 1.03 | 1.14 | 1.57 | Glyma.16G169600 | receptor like protein 6 |
| Leaf | 1.10 | 1.76 | 1.12 | 0.98 | 1.57 | Glyma.02G101300 | Cytochrome b561/ferric reductase transmembrane protein |
| | | | | | | | family |
| Leaf | −1.31 | −1.68 | −1.07 | 1.22 | 1.56 | Glyma.16G173000 | chitinase A |
| Leaf | 1.45 | 1.61 | 1.03 | 1.41 | 1.56 | Glyma.16G170800 | disease resistance family protein/LRR family protein |
| Leaf | 1.14 | 1.58 | 1.02 | 1.11 | 1.55 | Glyma.13G321100 | terpene synthase 03 |
| Leaf | 1.29 | 1.59 | 1.03 | 1.26 | 1.54 | Glyma.16G168700 | receptor like protein 19 |
| Leaf | −1.10 | −1.64 | −1.07 | 1.03 | 1.53 | Glyma.01G126600 | Disease resistance-responsive (dirigent-like protein) family |
| | | | | | | | protein |
| Leaf | 1.33 | 1.81 | 1.18 | 1.13 | 1.53 | Glyma.U037600 | disease resistance family protein/LRR family protein |
| Leaf | −1.01 | −1.62 | −1.06 | 0.96 | 1.53 | Glyma.09G164000 | |
| Leaf | 1.16 | 1.92 | 1.26 | 0.92 | 1.53 | Glyma.16G178400 | Heavy metal transport/detoxification superfamily protein |
| Leaf | −1.03 | −1.62 | −1.07 | 0.96 | 1.52 | Glyma.05G084500 | |
| Leaf | 1.04 | 1.69 | 1.12 | 0.93 | 1.52 | Glyma.06G135900 | |
| Leaf | 1.49 | 1.60 | 1.06 | 1.41 | 1.52 | Glyma.16G170700 | disease resistance family protein/LRR family protein |
| Leaf | −1.01 | −1.62 | −1.07 | 0.95 | 1.52 | Glyma.08G269900 | |
| Leaf | 1.67 | 1.69 | 1.12 | 1.50 | 1.51 | Glyma.16G172100 | disease resistance family protein/LRR family protein |
| Leaf | 1.24 | 1.55 | 1.03 | 1.21 | 1.51 | Glyma.16G185800 | disease resistance family protein/LRR family protein |
| Leaf | 1.06 | −1.61 | 1.07 | 1.00 | −1.50 | Glyma.U007200 | |
| Leaf | 1.38 | 1.52 | −1.01 | −1.37 | −1.51 | Glyma.16G156100 | leucine-rich repeat transmembrane protein kinase family |
| | | | | | | | protein |
| Leaf | 1.02 | −1.55 | 1.01 | 1.01 | −1.53 | Glyma.07G079100 | |
| Leaf | −1.52 | −1.60 | 1.03 | −1.48 | −1.56 | Glyma.14G103100 | WRKY DNA-binding protein 40 |
| Leaf | 1.02 | −1.60 | 1.01 | 1.01 | −1.58 | Glyma.14G063700 | HSP20-like chaperones superfamily protein |
| Leaf | −1.50 | −1.70 | 1.05 | −1.42 | −1.61 | Glyma.16G158600 | SNF1-related protein kinase 2.9 |
| Leaf | −1.27 | −1.66 | 1.03 | −1.23 | −1.62 | Glyma.16G175000 | disease resistance family protein/LRR family protein |
| Leaf | −1.02 | −1.72 | 1.03 | −0.99 | −1.66 | Glyma.13G023200 | |
| Leaf | 1.59 | 1.70 | −1.00 | −1.59 | −1.69 | Glyma.16G197500 | 2-oxoglutarate (2OG) and Fe(II)-dependent oxygenase |
| | | | | | | | superfamily protein |
| Leaf | 1.26 | 1.72 | −1.01 | −1.25 | −1.71 | Glyma.16G169700 | receptor like protein 27 |
| Leaf | 1.55 | 1.72 | −1.00 | −1.55 | −1.72 | Glyma.16G171200 | disease resistance family protein/LRR family protein |
| Leaf | 2.18 | 2.51 | 1.05 | 2.07 | 2.38 | Glyma.16G169400 | receptor like protein 19 |
| Leaf | −1.58 | −2.11 | −1.01 | 1.56 | 2.09 | Glyma.16G195900 | Tetratricopeptide repeat (TPR)-like superfamily protein |
| Leaf | 1.66 | 2.08 | 1.03 | 1.61 | 2.02 | Glyma.16G174600 | disease resistance family protein/LRR family protein |
| Leaf | 1.64 | 1.72 | 1.01 | 1.62 | 1.70 | Glyma.16G171800 | receptor like protein 15 |
| Leaf | 1.55 | 1.72 | 1.03 | 1.51 | 1.67 | Glyma.16G187100 | disease resistance family protein/LRR family protein |
| Leaf | −1.59 | −1.34 | 1.04 | −1.53 | −1.29 | Glyma.01G137500 | SAUR-like auxin-responsive protein family |
| Leaf | 1.59 | 1.70 | −1.00 | −1.59 | −1.69 | Glyma.16G197500 | 2-oxoglutarate (2OG) and Fe(II)-dependent oxygenase |
| | | | | | | | superfamily protein |
| Leaf | 1.55 | 1.72 | −1.00 | −1.55 | −1.72 | Glyma.16G171200 | disease resistance family protein/LRR family protein |
| Root | −1.92 | −2.08 | −1.03 | 1.87 | 2.02 | Glyma.16G168400 | VIRE2-interacting protein 1 |
| Root | −1.83 | −2.21 | −1.03 | 1.77 | 2.14 | Glyma.16G182400 | Plant regulator RWP-RK family protein |
| Root | −1.65 | −1.52 | −1.01 | 1.63 | 1.50 | Glyma.16G195600 | cytochrome P450, family 71, subfamily A, polypeptide 26 |
| Root | −1.71 | −1.82 | −1.10 | 1.55 | 1.65 | Glyma.16G173000 | chitinase A |
| Root | 1.56 | 1.70 | 1.03 | 1.52 | 1.66 | Glyma.16G199800 | |
| Root | −1.61 | −1.93 | −1.13 | 1.43 | 1.71 | Glyma.16G195700 | Tetratricopeptide repeat (TPR)-like superfamily protein |
| Root | −1.42 | −1.75 | −1.04 | 1.37 | 1.69 | Glyma.16G187200 | disease resistance family protein/LRR family protein |
| Root | −1.54 | −1.72 | −1.14 | 1.36 | 1.51 | Glyma.09G111900 | fatty acid desaturase 2 |
| Root | 1.42 | 1.83 | 1.06 | 1.34 | 1.73 | Glyma.16G169700 | receptor like protein 27 |
| Root | −1.46 | −2.07 | −1.09 | 1.34 | 1.89 | Glyma.16G195800 | cytochrome P450, family 71, subfamily A, polypeptide 22 |
| Root | −1.37 | −1.63 | −1.04 | 1.33 | 1.57 | Glyma.17G126300 | Cysteine proteinases superfamily protein |
| Root | −1.47 | −1.95 | −1.11 | 1.32 | 1.75 | Glyma.19G213100 | Integrase-type DNA-binding superfamily protein |
| Root | −1.61 | −2.02 | −1.21 | 1.32 | 1.66 | Glyma.16G188800 | Bifunctional inhibitor/lipid-transfer protein/seed storage 2S |
| | | | | | | | albumin superfamily protein |
| Root | −1.42 | −1.67 | −1.08 | 1.32 | 1.55 | Glyma.18G072300 | Chaperone DnaJ-domain superfamily protein |
| Root | −1.39 | −1.66 | −1.06 | 1.31 | 1.57 | Glyma.16G188000 | disease resistance family protein/LRR family protein |
| Root | 1.40 | 1.85 | 1.07 | 1.31 | 1.73 | Glyma.16G174600 | disease resistance family protein/LRR family protein |
| Root | 1.33 | 1.91 | 1.02 | 1.31 | 1.87 | Glyma.16G168900 | cytochrome P450, family 707, subfamily A, polypeptide 3 |
| Root | −1.28 | −1.87 | −1.01 | 1.27 | 1.86 | Glyma.06G031000 | |
| Root | −1.37 | −1.98 | −1.10 | 1.25 | 1.81 | Glyma.18G212500 | AZA-guanine resistant1 |
| Root | 1.32 | 1.64 | 1.06 | 1.25 | 1.55 | Glyma.07G266600 | |
| Root | 1.34 | 1.72 | 1.07 | 1.24 | 1.60 | Glyma.07G074000 | Cellulase (glycosyl hydrolase family 5) protein |
| Root | 1.40 | 1.82 | 1.12 | 1.24 | 1.62 | Glyma.18G044900 | acyl activating enzyme 12 |
| Root | 1.29 | 1.68 | 1.04 | 1.24 | 1.61 | Glyma.10G154400 | glycoside hydrolase family 2 protein |
| Root | 1.39 | 1.82 | 1.13 | 1.22 | 1.61 | Glyma.18G147000 | basic helix-loop-helix (bHLH) DNA-binding superfamily |
| | | | | | | | protein |
| Root | 1.49 | 1.84 | 1.22 | 1.22 | 1.51 | Glyma.13G262400 | Carbohydrate-binding X8 domain superfamily protein |
| Root | 1.56 | 1.93 | 1.28 | 1.22 | 1.50 | Glyma.03G029400 | Plant invertase/pectin methylesterase inhibitor superfamily |
| Root | 1.24 | 1.76 | 1.02 | 1.21 | 1.71 | Glyma.16G129300 | disease resistance family protein/LRR family protein |
| Root | 1.32 | 1.69 | 1.10 | 1.20 | 1.53 | Glyma.11G004700 | Cupredoxin superfamily protein |
| Root | −1.22 | −1.65 | −1.03 | 1.18 | 1.60 | Glyma.06G042200 | |
| Root | 1.22 | 1.58 | 1.04 | 1.18 | 1.52 | Glyma.08G076200 | Vacuolar iron transporter (VIT) family protein |
| Root | 1.23 | 1.59 | 1.05 | 1.18 | 1.51 | Glyma.08G120300 | receptor-like kinase in flowers 1 |
| Root | 1.26 | 1.75 | 1.08 | 1.16 | 1.62 | Glyma.19G133400 | 2-oxoglutarate (2OG) and Fe(II)-dependent oxygenase |
| | | | | | | | superfamily protein |
| Root | 1.23 | 1.59 | 1.06 | 1.16 | 1.50 | Glyma.19G258000 | RAB GTPase homolog A3 |
| Root | 1.26 | 1.77 | 1.09 | 1.16 | 1.62 | Glyma.05G225400 | Protein of unknown function (DUF1005) |
| Root | 1.26 | 1.68 | 1.09 | 1.16 | 1.54 | Glyma.05G138100 | DNAse I-like superfamily protein |
| Root | 1.32 | 1.72 | 1.14 | 1.15 | 1.50 | Glyma.07G081700 | gibberellin 20 oxidase 2 |
| Root | 1.22 | 1.76 | 1.06 | 1.15 | 1.66 | Glyma.20G110000 | TRICHOME BIREFRINGENCE-LIKE 36 |
| Root | 1.33 | 1.78 | 1.16 | 1.15 | 1.54 | Glyma.16G189200 | Heavy metal transport/detoxification superfamily protein |
| Root | −1.20 | −1.60 | −1.04 | 1.15 | 1.53 | Glyma.08G139800 | |
| Root | 1.67 | 2.27 | 1.46 | 1.14 | 1.56 | Glyma.06G123900 | glycosyl hydrolase 9A1 |
| Root | −1.20 | −2.94 | −1.05 | 1.14 | 2.80 | Glyma.05G114100 | Protein kinase superfamily protein |
| Root | 1.20 | 1.65 | 1.05 | 1.14 | 1.57 | Glyma.06G187100 | formin 8 |
| Root | 1.24 | 1.80 | 1.09 | 1.14 | 1.65 | Glyma.06G074600 | potassium channel in Arabidopsis thaliana 3 |
| Root | 1.16 | 1.66 | 1.02 | 1.14 | 1.63 | Glyma.17G009600 | RPM1 interacting protein 4 |
| Root | 1.18 | 1.58 | 1.05 | 1.13 | 1.50 | Glyma.12G091200 | NAC domain containing protein 90 |
| Root | 1.28 | 1.79 | 1.14 | 1.12 | 1.56 | Glyma.04G123000 | |
| Root | 1.17 | 1.78 | 1.05 | 1.11 | 1.69 | Glyma.13G328900 | ROP interactive partner 5 |
| Root | 1.32 | 1.88 | 1.19 | 1.11 | 1.59 | Glyma.05G188000 | |
| Root | 1.15 | 1.65 | 1.04 | 1.11 | 1.59 | Glyma.18G040600 | O-Glycosyl hydrolases family 17 protein |
| Root | 1.21 | 1.70 | 1.09 | 1.11 | 1.56 | Glyma.06G323100 | cellulase 3 |
| Root | 1.15 | 1.57 | 1.04 | 1.11 | 1.51 | Glyma.01G221200 | PLAC8 family protein |
| Root | 1.17 | 1.67 | 1.07 | 1.10 | 1.57 | Glyma.20G043400 | serine carboxypeptidase-like 17 |
| Root | 1.24 | 1.77 | 1.13 | 1.10 | 1.57 | Glyma.06G119000 | |
| Root | −1.15 | −1.62 | −1.06 | 1.08 | 1.53 | Glyma.03G143500 | myb-like HTH transcriptional regulator family protein |
| Root | 1.16 | 1.65 | 1.08 | 1.08 | 1.54 | Glyma.20G023000 | Transducin/WD40 repeat-like superfamily protein |
| Root | 1.36 | 2.07 | 1.28 | 1.07 | 1.62 | Glyma.13G042900 | white-brown complex homolog protein 11 |
| Root | 1.13 | 1.66 | 1.06 | 1.07 | 1.57 | Glyma.01G196500 | |
| Root | 1.10 | 1.66 | 1.07 | 1.03 | 1.55 | Glyma.14G203500 | ovate family protein 17 |
| Root | 1.19 | 1.81 | 1.17 | 1.02 | 1.55 | Glyma.09G280300 | Uncharacterised protein family (UPF0497) |
| Root | 1.22 | 1.84 | 1.21 | 1.01 | 1.52 | Glyma.16G054800 | Protein of unknown function (DUF 3339) |
| Root | 1.11 | 1.66 | 1.10 | 1.00 | 1.51 | Glyma.16G178000 | Heavy metal transport/detoxification superfamily protein |
| Root | 1.28 | 1.93 | 1.29 | 1.00 | 1.50 | Glyma.05G125300 | |
| Root | 1.27 | 2.10 | 1.28 | 0.99 | 1.64 | Glyma.03G010300 | |
| Root | 1.11 | 1.72 | 1.12 | 0.99 | 1.53 | Glyma.02G067500 | nudix hydrolase homolog 12 |
| Root | −1.09 | −1.72 | −1.11 | 0.98 | 1.55 | Glyma.16G038100 | |
| Root | 1.00 | −1.56 | 1.04 | 0.97 | −1.50 | Glyma.09G075100 | RNA-binding CRS1/YhbY (CRM) domain-containing protein |
| Root | 1.04 | 1.77 | 1.08 | 0.97 | 1.64 | Glyma.04G228900 | |
| Root | 1.02 | 1.63 | 1.07 | 0.95 | 1.53 | Glyma.05G072600 | myb domain protein 40 |
| Root | −1.03 | −1.77 | −1.09 | 0.95 | 1.63 | Glyma.05G009000 | early nodulin-related |
| Root | 1.25 | 2.07 | 1.32 | 0.94 | 1.56 | Glyma.09G072000 | Integrase-type DNA-binding superfamily protein |
| Root | 1.04 | 1.71 | 1.11 | 0.94 | 1.54 | Glyma.08G248000 | O-methyltransferase family protein |
| Root | 1.06 | 1.76 | 1.17 | 0.91 | 1.51 | Glyma.13G130300 | Ankyrin repeat family protein |
| Root | 1.16 | 2.32 | 1.34 | 0.87 | 1.73 | Glyma.15G180000 | Integrase-type DNA-binding superfamily protein |
| Root | −1.01 | 1.61 | 1.06 | −0.96 | 1.52 | Glyma.19G084200 | lysine histidine transporter 1 |
| Root | −1.05 | 1.70 | 1.06 | −0.99 | 1.61 | Glyma.16G178400 | Heavy metal transport/detoxification superfamily protein |
| Root | 1.02 | 1.55 | −1.03 | −1.00 | −1.51 | Glyma.13G357600 | |
| Root | −1.03 | 1.68 | 1.01 | −1.01 | 1.65 | Glyma.06G135900 | |
| Root | 1.06 | −1.55 | −1.01 | −1.04 | 1.53 | Glyma.07G232500 | CRT (chloroquine-resistance transporter)-like transporter 3 |
| Root | 1.11 | 1.76 | −1.06 | −1.04 | −1.66 | Glyma.06G215200 | Cytochrome P450 superfamily protein |
| Root | 1.19 | 1.55 | −1.01 | −1.18 | −1.54 | Glyma.16G174400 | Leucine-rich repeat transmembrane protein kinase family |
| | | | | | | | protein |
| Root | −1.20 | −1.66 | 1.01 | −1.19 | −1.64 | Glyma.16G169200 | receptor like protein 6 |
| Root | 1.29 | 1.54 | −1.02 | −1.25 | −1.51 | Glyma.04G214900 | Transducin/WD40 repeat-like superfamily protein |
| Root | −1.34 | −1.57 | 1.01 | −1.32 | −1.55 | Glyma.U002100 | disease resistance family protein/LRR family protein |
| Root | −1.43 | −2.12 | 1.06 | −1.35 | −1.99 | Glyma.16G175500 | UDP-glucosyl transferase 88A1 |
| Root | −1.40 | −1.67 | 1.02 | −1.37 | −1.63 | Glyma.16G185900 | CAP160 protein |
| Root | −1.47 | −1.99 | 1.02 | −1.44 | −1.95 | Glyma.16G195900 | Tetratricopeptide repeat (TPR)-like superfamily protein |
| Root | −1.52 | −2.25 | 1.04 | −1.46 | −2.16 | Glyma.16G159300 | Disease resistance protein (TIR-NBS-LRR class) family |
| Root | −1.86 | −1.81 | 1.07 | −1.74 | −1.69 | Glyma.16G159200 | Disease resistance protein (TIR-NBS-LRR class) family |
| Root | −1.92 | −2.08 | −1.03 | 1.87 | 2.02 | Glyma.16G168400 | VIRE2-interacting protein 1 |
| Root | −1.83 | −2.21 | −1.03 | 1.77 | 2.14 | Glyma.16G182400 | Plant regulator RWP-RK family protein |
| Root | −1.90 | −1.36 | −1.11 | 1.72 | 1.23 | Glyma.12G132500 | UDP-Glycosyltransferase superfamily protein |
| Root | 2.54 | 2.03 | 1.49 | 1.71 | 1.37 | Glyma.06G031600 | AMP-dependent synthetase and ligase family protein |
| Root | −1.65 | −1.52 | −1.01 | 1.63 | 1.50 | Glyma.16G195600 | cytochrome P450, family 71, subfamily A, polypeptide 26 |
| Root | −1.90 | −1.42 | −1.17 | 1.62 | 1.22 | Glyma.16G175800 | Glycosyl hydrolases family 32 protein |
| Root | −1.96 | −1.66 | −1.22 | 1.60 | 1.36 | Glyma.05G095600 | Amino acid kinase family protein |
| Root | −1.71 | −1.82 | −1.10 | 1.55 | 1.65 | Glyma.16G173000 | chitinase A |
| Root | 1.56 | 1.70 | 1.03 | 1.52 | 1.66 | Glyma.16G199800 | |
| Root | 1.58 | 1.36 | 1.04 | 1.51 | 1.30 | Glyma.16G165400 | Tetratricopeptide repeat (TPR)-like superfamily protein |
| Root | −1.86 | −1.81 | 1.07 | −1.74 | −1.69 | Glyma.16G159200 | Disease resistance protein (TIR-NBS-LRR class) family |

TABLE 7F
Transcripts that are up- or down- regulated at least 1.5 fold higher or lower in the beneficial
Penicillium strain Strain A or Strain B as compared to the control Penicillium strain Strain F.

| | | | | Strain | Strain | | |
|---|---|---|---|---|---|---|---|
| | | | | B/ | A/ | | |
| | Strain | Strain | Strain | Strain | Strain | | |
| Tissue | B | A | F | F | F | Transcript Name | Transcript Description |
| Leaf | 2.44 | 2.56 | 1.14 | 2.14 | 2.24 | Glyma.16G169400.1 | receptor like protein 19 |
| Leaf | −1.81 | −2.12 | −1.02 | 1.76 | 2.07 | Glyma.16G168400.1 | VIRE2-interacting protein 1 |
| Leaf | 1.82 | 2.27 | 1.04 | 1.76 | 2.19 | Glyma.03G028300.1 | S-adenosyl-L-methionine-dependent methyltransferases |
| | | | | | | | superfamily protein |
| Leaf | 1.81 | 2.02 | 1.08 | 1.67 | 1.87 | Glyma.16G170800.1 | disease resistance family protein/LRR family protein |
| Leaf | 1.70 | 1.91 | 1.03 | 1.65 | 1.85 | Glyma.16G169900.1 | disease resistance family protein/LRR family protein |
| Leaf | 1.87 | 2.09 | 1.15 | 1.63 | 1.82 | Glyma.16G171800.1 | receptor like protein 15 |
| Leaf | −1.67 | −1.82 | −1.04 | 1.61 | 1.75 | Glyma.U044100.2 | Arabidopsis thaliana protein of unknown function (DUF821) |
| Leaf | 1.58 | 1.80 | 1.04 | 1.52 | 1.73 | Glyma.16G174600.1 | disease resistance family protein/LRR family protein |
| Leaf | −1.64 | −2.30 | −1.09 | 1.51 | 2.12 | Glyma.16G195900.1 | Tetratricopeptide repeat (TPR)-like superfamily protein |
| Leaf | 1.68 | 2.37 | 1.14 | 1.48 | 2.08 | Glyma.11G104300.1 | Phototropic-responsive NPH3 family protein |
| Leaf | 1.46 | 1.74 | 1.01 | 1.44 | 1.72 | Glyma.16G197900.5 | F-box family protein |
| Leaf | 1.49 | 1.72 | 1.04 | 1.44 | 1.66 | Glyma.16G156100.1 | leucine-rich repeat transmembrane protein kinase family protein |
| Leaf | 1.47 | 1.65 | 1.03 | 1.43 | 1.60 | Glyma.11G086600.2 | |
| Leaf | 1.46 | 1.68 | 1.03 | 1.43 | 1.64 | Glyma.U006500.1 | disease resistance family protein/LRR family protein |
| Leaf | 1.69 | 2.58 | 1.20 | 1.40 | 2.14 | Glyma.01G006500.8 | nuclear RNA polymerase C2 |
| Leaf | 1.71 | 1.88 | 1.23 | 1.39 | 1.53 | Glyma.16G170700.1 | disease resistance family protein/LRR family protein |
| Leaf | −1.53 | −1.86 | −1.11 | 1.38 | 1.68 | Glyma.20G107700.3 | GCIP-interacting family protein |
| Leaf | 1.43 | 1.66 | 1.04 | 1.37 | 1.60 | Glyma.16G171200.1 | disease resistance family protein/LRR family protein |
| Leaf | 1.44 | 1.82 | 1.05 | 1.37 | 1.73 | Glyma.16G191600.1 | Arabidopsis thaliana protein of unknown function (DUF821) |
| Leaf | −1.48 | −1.68 | −1.08 | 1.37 | 1.55 | Glyma.16G173000.1 | chitinase A |
| Leaf | 1.60 | 2.01 | 1.17 | 1.36 | 1.71 | Glyma.03G046400.2 | prenylated RAB acceptor 1.B4 |
| Leaf | −1.36 | −1.54 | −1.00 | 1.36 | 1.54 | Glyma.16G188100.1 | disease resistance family protein/LRR family protein |
| Leaf | 1.40 | 1.54 | 1.03 | 1.36 | 1.50 | Glyma.16G106200.1 | disease resistance family protein/LRR family protein |
| Leaf | 1.49 | 1.73 | 1.13 | 1.32 | 1.53 | Glyma.16G172500.2 | multidrug resistance-associated protein 14 |
| Leaf | 1.32 | 1.74 | 1.01 | 1.30 | 1.71 | Glyma.16G169700.1 | receptor like protein 27 |
| Leaf | 1.37 | 1.62 | 1.07 | 1.28 | 1.51 | Glyma.17G249800.2 | |
| Leaf | 1.61 | 2.01 | 1.26 | 1.27 | 1.59 | Glyma.16G172600.1 | multidrug resistance-associated protein 14 |
| Leaf | −1.56 | −1.90 | −1.24 | 1.26 | 1.53 | Glyma.05G184500.1 | WRKY DNA-binding protein 51 |
| Leaf | −1.29 | −2.38 | −1.03 | 1.26 | 2.32 | Glyma.07G238000.2 | WRKY DNA-binding protein 23 |
| Leaf | 1.40 | 2.01 | 1.11 | 1.26 | 1.81 | Glyma.16G169600.1 | receptor like protein 6 |
| Leaf | 1.25 | 1.54 | 1.00 | 1.25 | 1.54 | Glyma.20G112800.2 | kow domain-containing transcription factor 1 |
| Leaf | 1.31 | 1.65 | 1.06 | 1.24 | 1.57 | Glyma.09G272300.7 | Leucine-rich repeat protein kinase family protein |
| Leaf | 1.39 | 1.86 | 1.12 | 1.24 | 1.65 | Glyma.13G223000.1 | GDSL-like Lipase/Acylhydrolase superfamily protein |
| Leaf | −1.29 | −1.63 | −1.05 | 1.22 | 1.54 | Glyma.13G232600.2 | nudix hydrolase homolog 15 |
| Leaf | −1.36 | −2.15 | −1.12 | 1.21 | 1.92 | Glyma.02G258000.1 | Plant protein of unknown function (DUF828) |
| Leaf | −1.59 | −2.00 | −1.32 | 1.21 | 1.52 | Glyma.09G044000.2 | Heat shock protein DnaJ, N-terminal with domain of unknown |
| | | | | | | | function (DUF1977) |
| Leaf | −1.25 | −1.57 | −1.04 | 1.20 | 1.50 | Glyma.08G019800.2 | |
| Leaf | 1.32 | 1.68 | 1.11 | 1.19 | 1.52 | Glyma.06G313600.4 | zinc induced facilitator-like 1 |
| Leaf | −1.39 | −2.00 | −1.19 | 1.17 | 1.69 | Glyma.03G264600.3 | RNA-binding protein |
| Leaf | −1.21 | −1.55 | −1.03 | 1.17 | 1.51 | Glyma.06G305900.1 | Polynucleotidyl transferase, ribonuclease H fold protein with |
| | | | | | | | HRDC domain |
| Leaf | 1.27 | 1.70 | 1.08 | 1.17 | 1.57 | Glyma.19G240400.1 | copper/zinc superoxide dismutase 1 |
| Leaf | −1.36 | −1.76 | −1.16 | 1.17 | 1.52 | Glyma.06G196200.2 | CCT motif -containing response regulator protein |
| Leaf | 1.22 | 1.83 | 1.06 | 1.15 | 1.73 | Glyma.16G138400.1 | RAD-like 6 |
| Leaf | −1.19 | −1.68 | −1.04 | 1.15 | 1.62 | Glyma.10G166000.3 | threonine aldolase 1 |
| Leaf | −1.34 | −1.89 | −1.20 | 1.12 | 1.58 | Glyma.03G166800.1 | SET-domain containing protein lysine methyltransferase family |
| | | | | | | | protein |
| Leaf | 1.18 | 1.60 | 1.06 | 1.12 | 1.52 | Glyma.02G183500.2 | Protein kinase superfamily protein |
| Leaf | 1.14 | 1.59 | 1.03 | 1.11 | 1.54 | Glyma.15G074000.3 | glucan synthase-like 1 |
| Leaf | −1.14 | −1.57 | −1.04 | 1.10 | 1.51 | Glyma.17G137200.1 | Nucleotide-sugar transporter family protein |
| Leaf | −1.23 | −1.70 | −1.12 | 1.10 | 1.52 | Glyma.16G132600.4 | folate transporter 1 |
| Leaf | 1.22 | 1.76 | 1.11 | 1.10 | 1.59 | Glyma.02G288600.1 | Leucine-rich repeat transmembrane protein kinase |
| Leaf | −1.11 | 1.68 | −1.03 | 1.08 | −1.64 | Glyma.09G054100.4 | Ankyrin repeat family protein |
| Leaf | −1.17 | −3.08 | −1.09 | 1.08 | 2.83 | Glyma.05G207400.3 | Protein kinase protein with tetratricopeptide repeat domain |
| Leaf | 1.15 | 2.10 | 1.07 | 1.08 | 1.97 | Glyma.05G091800.4 | Plant protein of unknown function (DUF869) |
| Leaf | 1.09 | −1.55 | 1.01 | 1.08 | −1.53 | Glyma.13G213400.2 | squamosa promoter binding protein-like 7 |
| Leaf | 1.09 | 1.67 | 1.02 | 1.08 | 1.65 | Glyma.03G176600.5 | WRKY family transcription factor family protein |
| Leaf | 1.15 | 4.98 | 1.07 | 1.08 | 4.66 | Glyma.15G167100.7 | |
| Leaf | −1.16 | −1.69 | −1.08 | 1.07 | 1.56 | Glyma.07G189000.1 | Octicosapeptide/Phox/Bem1p family protein |
| Leaf | −1.10 | −1.55 | −1.03 | 1.07 | 1.51 | Glyma.14G153000.2 | SWITCH/sucrose nonfermenting 3C |
| Leaf | −1.17 | −1.92 | −1.10 | 1.07 | 1.75 | Glyma.15G124400.3 | SNARE associated Golgi protein family |
| Leaf | 1.22 | 1.73 | 1.14 | 1.07 | 1.51 | Glyma.09G055700.1 | Protein kinase superfamily protein |
| Leaf | −1.11 | −1.61 | −1.05 | 1.06 | 1.53 | Glyma.14G100200.1 | |
| Leaf | 1.05 | −1.69 | 1.01 | 1.05 | −1.68 | Glyma.U007400.1 | |
| Leaf | −1.08 | −1.66 | −1.04 | 1.04 | 1.60 | Glyma.03G202400.4 | BTB/POZ domain-containing protein |
| Leaf | −1.10 | −1.62 | −1.06 | 1.04 | 1.53 | Glyma.01G126600.1 | Disease resistance-responsive (dirigent-like protein) family |
| | | | | | | | protein |
| Leaf | 1.04 | −1.52 | 1.00 | 1.04 | −1.52 | Glyma.08G154000.1 | hAT dimerisation domain-containing protein |
| Leaf | 1.09 | 1.62 | 1.05 | 1.04 | 1.55 | Glyma.04G076500.2 | Cellulose-synthase-like C5 |
| Leaf | −1.16 | −1.81 | −1.12 | 1.04 | 1.62 | Glyma.15G251100.2 | ubiquitin-specific protease 25 |
| Leaf | −1.16 | −2.77 | −1.12 | 1.04 | 2.48 | Glyma.12G211300.3 | Protein of unknown function (DUF688) |
| Leaf | −1.17 | −2.14 | −1.13 | 1.04 | 1.90 | Glyma.08G073700.2 | Polymerase/histidinol phosphatase-like |
| Leaf | 1.04 | 1.52 | 1.01 | 1.03 | 1.51 | Glyma.15G092400.5 | multidrug resistance-associated protein 2 |
| Leaf | −1.07 | −5.38 | −1.04 | 1.03 | 5.16 | Glyma.17G170900.7 | PLC-like phosphodiesterases superfamily protein |
| Leaf | 1.10 | 1.64 | 1.08 | 1.03 | 1.53 | Glyma.11G248400.6 | FAD/NAD(P)-binding oxidoreductase family protein |
| Leaf | 1.14 | 1.86 | 1.11 | 1.03 | 1.67 | Glyma.20G173200.3 | FORMS APLOID AND BINUCLEATE CELLS 1A |
| Leaf | −1.07 | −2.83 | −1.05 | 1.02 | 2.70 | Glyma.01G099800.2 | delta 1-pyrroline-5-carboxylate synthase 2 |
| Leaf | −1.05 | −1.54 | −1.02 | 1.02 | 1.51 | Glyma.12G095300.5 | BRI1 suppressor 1 (BSU1)-like 2 |
| Leaf | −1.06 | −1.59 | −1.03 | 1.02 | 1.54 | Glyma.05G084500.1 | |
| Leaf | 1.05 | −1.58 | 1.03 | 1.02 | −1.54 | Glyma.14G063700.1 | HSP20-like chaperones superfamily protein |
| Leaf | −1.06 | −1.74 | −1.05 | 1.01 | 1.66 | Glyma.05G081200.1 | |
| Leaf | 1.11 | 1.78 | 1.11 | 1.01 | 1.61 | Glyma.14G195200.3 | Protein phosphatase 2C family protein |
| Leaf | −1.12 | −3.13 | −1.11 | 1.01 | 2.81 | Glyma.04G165100.1 | Plant protein of unknown function (DUF863) |
| Leaf | −1.07 | −1.77 | −1.06 | 1.01 | 1.67 | Glyma.16G093800.17 | Helicase/SANT-associated, DNA binding protein |
| Leaf | 1.04 | 3.66 | 1.04 | 1.01 | 3.54 | Glyma.15G111700.1 | Ribosomal protein L4/L1 family |
| Leaf | −1.19 | −1.78 | −1.18 | 1.00 | 1.51 | Glyma.20G116300.2 | Regulator of Vps4 activity in the MVB pathway protein |
| Leaf | 1.03 | 1.71 | 1.03 | 1.00 | 1.66 | Glyma.20G062200.4 | homolog of yeast ADA2 2A |
| Leaf | −1.08 | −1.72 | −1.08 | 1.00 | 1.60 | Glyma.12G128700.2 | Mitogen activated protein kinase kinase kinase-related |
| Leaf | 1.04 | −1.81 | 1.04 | 1.00 | −1.75 | Glyma.13G012200.1 | |
| Leaf | 1.02 | 1.62 | 1.02 | 1.00 | 1.58 | Glyma.20G122300.1 | 2-oxoglutarate (2OG) and Fe(II)-dependent oxygenase |
| | | | | | | | superfamily protein |
| Leaf | −1.14 | −1.90 | −1.14 | 1.00 | 1.66 | Glyma.12G084000.2 | Lecithin:cholesterol acyltransferase family protein |
| Leaf | −1.16 | −1.88 | −1.16 | 1.00 | 1.62 | Glyma.04G256800.3 | P-loop containing nucleoside triphosphate hydrolases |
| | | | | | | | superfamily protein |
| Leaf | −1.21 | −2.57 | −1.21 | 1.00 | 2.12 | Glyma.17G098900.2 | basic helix-loop-helix (bHLH) DNA-binding superfamily protein |
| Leaf | 1.04 | 3.51 | 1.04 | 1.00 | 3.38 | Glyma.12G225400.2 | embryo defective 2737 |
| Leaf | 1.03 | 1.76 | 1.04 | 1.00 | 1.70 | Glyma.U011100.4 | Protein of unknown function (DUF1997) |
| Leaf | 1.01 | −1.77 | 1.01 | 1.00 | −1.75 | Glyma.13G024200.1 | |
| Leaf | 1.02 | −1.75 | 1.03 | 1.00 | −1.71 | Glyma.13G020800.1 | |
| Leaf | 1.01 | −1.77 | 1.01 | 1.00 | −1.75 | Glyma.13G018100.1 | |
| Leaf | 1.23 | 1.87 | 1.23 | 0.99 | 1.52 | Glyma.05G067400.3 | Zinc-binding ribosomal protein family protein |
| Leaf | 1.02 | −1.78 | 1.02 | 0.99 | −1.74 | Glyma.U005800.1 | |
| Leaf | 1.03 | 1.56 | 1.04 | 0.99 | 1.50 | Glyma.17G258000.3 | |
| Leaf | 1.03 | −1.78 | 1.04 | 0.99 | −1.72 | Glyma.13G021800.1 | |
| Leaf | −1.03 | −1.73 | −1.04 | 0.99 | 1.67 | Glyma.08G041700.4 | CRS1/YhbY (CRM) domain-containing protein |
| Leaf | −1.12 | −1.72 | −1.13 | 0.99 | 1.52 | Glyma.17G203800.2 | RING/U-box superfamily protein |
| Leaf | 1.02 | −1.74 | 1.03 | 0.99 | −1.69 | Glyma.13G011200.1 | |
| Leaf | 1.02 | 1.78 | 1.03 | 0.99 | 1.73 | Glyma.13G317500.1 | homolog of histone chaperone HIRA |
| Leaf | 1.13 | 1.78 | 1.15 | 0.99 | 1.55 | Glyma.02G262200.1 | Glycosyl hydrolase family 85 |
| Leaf | 1.05 | 1.67 | 1.06 | 0.98 | 1.57 | Glyma.20G133100.3 | Phototropic-responsive NPH3 family protein |
| Leaf | 1.05 | 2.57 | 1.07 | 0.98 | 2.41 | Glyma.05G131800.3 | K+ uptake permease 11 |
| Leaf | −1.05 | −1.63 | −1.07 | 0.98 | 1.52 | Glyma.09G133900.1 | |
| Leaf | 1.04 | 2.48 | 1.07 | 0.98 | 2.33 | Glyma.03G195300.3 | |
| Leaf | 1.04 | 2.40 | 1.07 | 0.98 | 2.25 | Glyma.15G178400.5 | aldehyde dehydrogenase 7B4 |
| Leaf | −1.12 | −2.27 | −1.14 | 0.98 | 1.98 | Glyma.01G052500.4 | O-fucosyltransferase family protein |
| Leaf | 1.04 | 2.22 | 1.07 | 0.98 | 2.08 | Glyma.11G211100.1 | Leucine-rich repeat protein kinase family protein |
| Leaf | −1.10 | −5.14 | −1.13 | 0.98 | 4.56 | Glyma.20G156500.2 | Homeobox-leucine zipper family protein/lipid-binding START |
| | | | | | | | domain-containing protein |
| Leaf | 1.03 | 1.79 | 1.06 | 0.98 | 1.69 | Glyma.01G221700.3 | Nuclear transport factor 2 (NTF2) family protein |
| Leaf | −1.01 | −1.67 | −1.04 | 0.97 | 1.60 | Glyma.01G163400.1 | |
| Leaf | 1.04 | 1.75 | 1.07 | 0.97 | 1.64 | Glyma.06G213300.3 | Stabilizer of iron transporter SufD/Polynucleotidyl transferase |
| Leaf | 1.10 | 1.74 | 1.14 | 0.97 | 1.53 | Glyma.13G053600.1 | Malectin/receptor-like protein kinase family protein |
| Leaf | −1.05 | −3.72 | −1.09 | 0.97 | 3.42 | Glyma.17G126200.1 | Transducin/WD40 repeat-like superfamily protein |
| Leaf | −1.08 | −2.28 | −1.11 | 0.97 | 2.05 | Glyma.01G215800.3 | Pleckstrin homology (PH) domain superfamily protein |
| Leaf | 1.13 | 1.84 | 1.17 | 0.96 | 1.57 | Glyma.10G034100.3 | IND1(iron-sulfur protein required for NADH dehydrogenase)-like |
| Leaf | −1.00 | −1.62 | −1.04 | 0.96 | 1.55 | Glyma.09G164000.1 | |
| Leaf | 1.11 | 1.77 | 1.16 | 0.96 | 1.52 | Glyma.02G101300.1 | Cytochrome b561/ferric reductase transmembrane protein |
| | | | | | | | family |
| Leaf | 1.02 | 1.62 | 1.07 | 0.96 | 1.52 | Glyma.18G001900.3 | |
| Leaf | −1.02 | −1.68 | −1.07 | 0.96 | 1.57 | Glyma.08G269900.1 | |
| Leaf | 1.07 | 1.70 | 1.12 | 0.95 | 1.51 | Glyma.02G228200.2 | Protein phosphatase 2C family protein |
| Leaf | 1.01 | −1.81 | 1.08 | 0.94 | −1.68 | Glyma.13G015400.1 | |
| Leaf | −1.32 | −3.08 | −1.41 | 0.93 | 2.18 | Glyma.01G013600.1 | basic region/leucine zipper transcription factor 16 |
| Leaf | −1.05 | 1.83 | −1.12 | 0.93 | −1.63 | Glyma.19G027200.2 | U-box domain-containing protein |
| Leaf | 1.00 | −1.68 | 1.08 | 0.93 | −1.56 | Glyma.13G015600.1 | |
| Leaf | −1.06 | −1.80 | −1.15 | 0.93 | 1.57 | Glyma.05G174800.3 | Protein of unknown function (DUF707) |
| Leaf | 1.05 | 4.33 | 1.19 | 0.88 | 3.63 | Glyma.02G035800.1 | SBP (S-ribonuclease binding protein) family protein |
| Leaf | 1.05 | 6.03 | 1.20 | 0.87 | 5.01 | Glyma.07G144800.2 | Transducin/WD40 repeat-like superfamily protein |
| Leaf | 1.02 | 1.80 | 1.18 | 0.86 | 1.53 | Glyma.03G015800.1 | BON association protein 2 |
| Leaf | 1.01 | 2.17 | 1.20 | 0.85 | 1.82 | Glyma.20G017100.2 | sulfate transporter 91 |
| Leaf | 1.06 | 2.17 | 1.27 | 0.84 | 1.72 | Glyma.04G212600.1 | |
| Leaf | −1.11 | −2.30 | −1.43 | 0.78 | 1.61 | Glyma.19G250900.7 | |
| Leaf | 1.03 | −1.86 | −1.15 | −0.90 | 1.63 | Glyma.08G220800.2 | ARIA-interacting double AP2 domain protein |
| Leaf | 1.00 | −1.64 | −1.09 | −0.92 | 1.51 | Glyma.08G211300.3 | O-fucosyltransferase family protein |
| Leaf | −1.01 | 1.64 | 1.09 | −0.92 | 1.50 | Glyma.02G218100.3 | indole-3-acetic acid inducible 9 |
| Leaf | −1.03 | 1.69 | 1.09 | −0.95 | 1.56 | Glyma.11G080500.2 | RING/U-box superfamily protein |
| Leaf | −1.02 | −1.75 | 1.05 | −0.97 | −1.66 | Glyma.13G024100.1 | |
| Leaf | −1.00 | −1.75 | 1.04 | −0.97 | −1.69 | Glyma.13G013600.1 | |
| Leaf | 1.00 | −1.72 | −1.02 | −0.98 | 1.67 | Glyma.13G023200.1 | |
| Leaf | −1.07 | −1.72 | 1.08 | −0.99 | −1.60 | Glyma.06G289600.5 | Peptidase family M48 family protein |
| Leaf | −1.01 | −1.66 | 1.03 | −0.99 | −1.62 | Glyma.18G097000.5 | peptidemethionine sulfoxide reductase 3 |
| Leaf | 1.02 | 1.55 | −1.03 | −0.99 | −1.51 | Glyma.08G326200.1 | multidrug resistance-associated protein 3 |
| Leaf | −1.03 | −1.59 | 1.04 | −0.99 | −1.53 | Glyma.13G021100.1 | |
| Leaf | 1.07 | −1.68 | −1.07 | −1.00 | 1.57 | Glyma.13G022700.1 | |
| Leaf | −1.02 | −1.52 | 1.01 | −1.01 | −1.51 | Glyma.07G079100.1 | |
| Leaf | 1.02 | −1.57 | −1.01 | −1.01 | 1.56 | Glyma.13G015000.1 | |
| Leaf | 1.08 | 2.76 | −1.07 | −1.02 | −2.59 | Glyma.12G040400.13 | ARM repeat superfamily protein |
| Leaf | −1.05 | 1.69 | 1.03 | −1.02 | 1.63 | Glyma.01G177700.6 | aminopeptidase P1 |
| Leaf | 1.05 | 1.67 | −1.02 | −1.03 | −1.64 | Glyma.05G175000.2 | beta-1,4-N-acetylglucosaminyltransferase family protein |
| Leaf | 1.08 | 1.66 | −1.05 | −1.03 | −1.59 | Glyma.13G029700.2 | GDA1/CD39 nucleoside phosphatase family protein |
| Leaf | −1.04 | −1.80 | 1.00 | −1.04 | −1.79 | Glyma.08G060600.2 | Pentatricopeptide repeat (PPR) superfamily protein |
| Leaf | −1.07 | −1.56 | 1.03 | −1.04 | −1.51 | Glyma.U007200.1 | |
| Leaf | 1.09 | 1.65 | −1.05 | −1.04 | −1.58 | Glyma.01G149300.1 | S-adenosyl-L-methionine-dependent methyltransferases |
| | | | | | | | superfamily protein |
| Leaf | −1.17 | −1.67 | 1.11 | −1.05 | −1.51 | Glyma.01G080700.6 | NAD(P)-binding Rossmann-fold superfamily protein |
| Leaf | −1.10 | 1.61 | 1.04 | −1.06 | 1.55 | Glyma.08G114800.3 | Peptidyl-tRNA hydrolase family protein |
| Leaf | 1.09 | 1.56 | −1.03 | −1.06 | −1.50 | Glyma.02G026200.1 | NB-ARC domain-containing disease resistance protein |
| Leaf | −1.13 | 1.61 | 1.07 | −1.06 | 1.51 | Glyma.07G069400.2 | transcription regulatory protein SNF2, putative |
| Leaf | 1.07 | 1.99 | −1.00 | −1.07 | −1.98 | Glyma.11G144800.5 | Protein kinase superfamily protein |
| Leaf | −1.11 | −1.57 | 1.04 | −1.07 | −1.52 | Glyma.18G038600.1 | pfkB-like carbohydrate kinase family protein |
| Leaf | 1.09 | 1.57 | −1.02 | −1.07 | −1.54 | Glyma.19G165500.4 | Transcription factor jumonji (jmj) family protein/zinc finger |
| | | | | | | | (C5HC2 type) family protein |
| Leaf | −1.13 | −2.01 | 1.04 | −1.08 | −1.93 | Glyma.10G102200.3 | |
| Leaf | 1.15 | −1.67 | −1.06 | −1.09 | 1.59 | Glyma.13G014300.1 | |
| Leaf | −1.19 | 1.63 | 1.08 | −1.10 | 1.50 | Glyma.12G209700.7 | |
| Leaf | 1.23 | 1.65 | −1.10 | −1.12 | −1.51 | Glyma.13G321100.1 | terpene synthase 03 |
| Leaf | −1.13 | −1.68 | 1.01 | −1.12 | −1.67 | Glyma.16G186700.1 | disease resistance family protein/LRR family protein |
| Leaf | 1.13 | 1.66 | −1.00 | −1.13 | −1.66 | Glyma.11G077300.2 | calcium-dependent protein kinase 28 |
| Leaf | 1.25 | 1.76 | −1.09 | −1.15 | −1.62 | Glyma.01G192400.5 | |
| Leaf | −1.16 | −1.54 | 1.00 | −1.16 | −1.54 | Glyma.10G221400.4 | carboxypeptidase D, putative |
| Leaf | 1.18 | 1.55 | −1.01 | −1.18 | −1.54 | Glyma.12G124100.1 | SAUR-like auxin-responsive protein family |
| Leaf | 1.26 | 1.60 | −1.03 | −1.22 | −1.55 | Glyma.16G174100.1 | disease resistance family protein/LRR family protein |
| Leaf | −1.31 | −1.72 | 1.03 | −1.26 | −1.66 | Glyma.19G191900.2 | ENTH/VHS family protein |
| Leaf | −1.35 | −1.55 | 1.03 | −1.31 | −1.51 | Glyma.02G184200.1 | UDP-Glycosyltransferase superfamily protein |
| Leaf | 1.41 | 1.73 | −1.02 | −1.38 | −1.70 | Glyma.16G174800.1 | disease resistance family protein/LRR family protein |
| Leaf | 1.53 | 1.53 | −1.01 | −1.51 | −1.51 | Glyma.16G171600.1 | receptor like protein 46 |
| Leaf | 1.55 | 1.72 | −1.03 | −1.51 | −1.66 | Glyma.16G158400.2 | Laccase/Diphenol oxidase family protein |
| Leaf | 1.52 | 1.60 | −1.00 | −1.52 | −1.59 | Glyma.16G197500.1 | 2-oxoglutarate (2OG) and Fe(II)-dependent oxygenase |
| | | | | | | | superfamily protein |
| Leaf | 1.66 | 1.84 | −1.07 | −1.55 | −1.72 | Glyma.14G223100.4 | Ubiquitin-like superfamily protein |
| Leaf | −1.63 | −2.08 | 1.03 | −1.58 | −2.02 | Glyma.16G174300.1 | disease resistance family protein/LRR family protein |
| Leaf | −1.60 | −1.85 | 1.01 | −1.58 | −1.83 | Glyma.16G183100.1 | Bifunctional inhibitor/lipid-transfer protein/seed storage 2S |
| | | | | | | | albumin superfamily protein |
| Leaf | −1.65 | −1.98 | 1.03 | −1.59 | −1.92 | Glyma.16G175000.1 | disease resistance family protein/LRR family protein |
| Leaf | 1.62 | 1.58 | −1.00 | −1.61 | −1.57 | Glyma.01G084500.1 | Plasma-membrane choline transporter family protein |
| Leaf | 1.68 | 1.95 | −1.01 | −1.66 | −1.93 | Glyma.16G187100.1 | disease resistance family protein/LRR family protein |
| Leaf | 1.55 | 1.27 | −1.03 | −1.50 | −1.23 | Glyma.15G083100.4 | peroxin 7 |
| Leaf | 1.64 | 1.26 | −1.09 | −1.50 | −1.16 | Glyma.16G126100.1 | disease resistance family protein/LRR family protein |
| Leaf | 1.53 | 1.53 | −1.01 | −1.51 | −1.51 | Glyma.16G171600.1 | receptor like protein 46 |
| Leaf | 1.55 | 1.72 | −1.03 | −1.51 | −1.66 | Glyma.16G158400.2 | Laccase/Diphenol oxidase family protein |
| Leaf | 1.52 | 1.60 | −1.00 | −1.52 | −1.59 | Glyma.16G197500.1 | 2-oxoglutarate (2OG) and Fe(II)-dependent oxygenase |
| | | | | | | | superfamily protein |
| Leaf | −1.63 | −1.54 | 1.07 | −1.52 | −1.44 | Glyma.16G182400.1 | Plant regulator RWP-RK family protein |
| Leaf | 1.66 | 1.84 | −1.07 | −1.55 | −1.72 | Glyma.14G223100.4 | Ubiquitin-like superfamily protein |
| Leaf | 1.64 | 1.36 | −1.05 | −1.56 | −1.30 | Glyma.U029000.3 | pre-mRNA-processing protein 40A |
| Leaf | 1.62 | 1.45 | −1.03 | −1.56 | −1.40 | Glyma.08G222100.1 | Ribosomal protein L30/L7 family protein |
| Leaf | −1.68 | −1.54 | 1.07 | −1.56 | −1.44 | Glyma.16G159300.1 | Disease resistance protein (TIR-NBS-LRR class) family |
| Leaf | 1.63 | 1.02 | −1.03 | −1.58 | −0.99 | Glyma.1OG175300.5 | |
| Leaf | −1.63 | −2.08 | 1.03 | −1.58 | −2.02 | Glyma.16G174300.1 | disease resistance family protein/LRR family protein |
| Leaf | −1.60 | −1.85 | 1.01 | −1.58 | −1.83 | Glyma.16G183100.1 | Bifunctional inhibitor/lipid-transfer protein/seed storage 2S |
| | | | | | | | albumin superfamily protein |
| Leaf | −1.68 | −1.34 | 1.06 | −1.59 | −1.27 | Glyma.01G137500.1 | SAUR-like auxin-responsive protein family |
| Leaf | −1.65 | −1.98 | 1.03 | −1.59 | −1.92 | Glyma.16G175000.1 | disease resistance family protein/LRR family protein |
| Leaf | 1.62 | 1.58 | −1.00 | −1.61 | −1.57 | Glyma.01G084500.1 | Plasma-membrane choline transporter family protein |
| Leaf | 1.68 | 1.95 | −1.01 | −1.66 | −1.93 | Glyma.16G187100.1 | disease resistance family protein/LRR family protein |
| Leaf | −1.70 | −1.09 | 1.02 | −1.67 | −1.07 | Glyma.06G009800.1 | myo-inositol monophosphatase like 2 |
| Leaf | −1.81 | 1.29 | 1.04 | −1.75 | 1.25 | Glyma.13G216400.3 | Galactose oxidase/kelch repeat superfamily protein |
| Leaf | −1.99 | −1.10 | 1.06 | −1.87 | −1.03 | Glyma.13G359600.1 | Pentatricopeptide repeat (PPR-like) superfamily protein |
| Leaf | −1.99 | 1.11 | 1.00 | −1.99 | 1.11 | Glyma.09G213200.1 | |
| Leaf | −2.31 | 1.48 | 1.11 | −2.08 | 1.33 | Glyma.19G013200.2 | senescence-associated gene 101 |
| Leaf | −2.51 | −1.09 | 1.01 | −2.48 | −1.08 | Glyma.02G162300.8 | protein kinase family protein/protein phosphatase 2C (PP2C) |
| | | | | | | | family protein |
| Root | 4.07 | 4.76 | 1.06 | 3.83 | 4.49 | Glyma.08G196700.6 | |
| Root | 4.46 | 3.72 | 1.23 | 3.62 | 3.02 | Glyma.08G047900.3 | Protein of unknown function (DUF1712) |
| Root | −4.00 | −5.20 | −1.24 | 3.21 | 4.17 | Glyma.14G098000.3 | G protein alpha subunit 1 |
| Root | 2.95 | 4.37 | 1.07 | 2.76 | 4.08 | Glyma.05G018500.5 | galacturonosyltransferase 11 |
| Root | 3.16 | 4.59 | 1.15 | 2.76 | 4.00 | Glyma.13G152800.2 | |
| Root | 2.10 | 1.93 | 1.03 | 2.04 | 1.87 | Glyma.03G226200.1 | |
| Root | 2.14 | 4.13 | 1.06 | 2.02 | 3.89 | Glyma.16G105400.1 | tetratricopeptide repeat (TPR)-containing protein |
| Root | 2.39 | 2.63 | 1.20 | 1.99 | 2.19 | Glyma.01G197900.1 | basic helix-loop-helix (bHLH) DNA-binding superfamily protein |
| Root | 2.01 | 1.59 | 1.04 | 1.93 | 1.53 | Glyma.04G254800.2 | TGA1A-related gene 3 |
| Root | −1.93 | −2.01 | −1.03 | 1.88 | 1.96 | Glyma.16G168400.1 | VIRE2-interacting protein 1 |
| Root | 2.20 | 2.51 | 1.23 | 1.79 | 2.05 | Glyma.16G173600.4 | SLAC1 homologue 3 |
| Root | 2.13 | 3.05 | 1.22 | 1.75 | 2.51 | Glyma.08G005500.2 | |
| Root | −2.02 | −1.82 | −1.18 | 1.71 | 1.54 | Glyma.01G152500.5 | |
| Root | −1.74 | −1.96 | −1.03 | 1.69 | 1.91 | Glyma.16G182400.1 | Plant regulator RWP-RK family protein |
| Root | −1.66 | −1.97 | −1.01 | 1.65 | 1.96 | Glyma.16G195900.1 | Tetratricopeptide repeat (TPR)-like superfamily protein |
| Root | 1.95 | 3.99 | 1.19 | 1.64 | 3.36 | Glyma.09G249300.3 | Eukaryotic aspartyl protease family protein |
| Root | −1.93 | −3.83 | −1.20 | 1.62 | 3.20 | Glyma.13G337100.2 | TRF-like 2 |
| Root | −1.70 | −1.75 | −1.09 | 1.56 | 1.60 | Glyma.16G173000.1 | chitinase A |
| Root | −1.58 | −1.89 | −1.03 | 1.54 | 1.84 | Glyma.16G187200.1 | disease resistance family protein/LRR family protein |
| Root | −1.61 | −1.81 | −1.05 | 1.54 | 1.73 | Glyma.09G233200.3 | nucleotide-sensitive chloride conductance regulator (ICln) family |
| | | | | | | | protein |
| Root | −1.75 | −2.03 | −1.14 | 1.53 | 1.78 | Glyma.16G195700.1 | Tetratricopeptide repeat (TPR)-like superfamily protein |
| Root | 1.54 | 1.66 | 1.01 | 1.53 | 1.65 | Glyma.16G199800.1 | |
| Root | 1.52 | 1.55 | 1.00 | 1.51 | 1.54 | Glyma.09G146100.1 | SEUSS-like 1 |
| Root | −1.65 | −1.83 | −1.11 | 1.49 | 1.65 | Glyma.04G100600.2 | RNA-binding (RRM/RBD/RNP motifs) family protein |
| Root | 1.69 | 1.74 | 1.16 | 1.46 | 1.50 | Glyma.07G018200.2 | photosystem II 11 kDa protein-related |
| Root | 1.48 | 1.61 | 1.03 | 1.43 | 1.56 | Glyma.16G189900.1 | CAP160 protein |
| Root | 1.47 | 1.61 | 1.04 | 1.41 | 1.55 | Glyma.08G364200.1 | HEAT repeat-containing protein |
| Root | −1.83 | −2.15 | −1.31 | 1.40 | 1.64 | Glyma.04G199400.9 | |
| Root | −1.40 | −1.54 | −1.01 | 1.39 | 1.53 | Glyma.11G222900.2 | BTB/POZ/MATH-domains containing protein |
| Root | −1.48 | −1.93 | −1.07 | 1.39 | 1.81 | Glyma.10G159500.2 | ARM repeat superfamily protein |
| Root | −1.38 | −1.81 | −1.00 | 1.38 | 1.81 | Glyma.16G155600.3 | decapping 2 |
| Root | 1.62 | 1.83 | 1.20 | 1.35 | 1.52 | Glyma.20G192100.1 | OPC-8:0 CoA ligase1 |
| Root | 1.39 | 1.66 | 1.04 | 1.34 | 1.60 | Glyma.16G169700.1 | receptor like protein 27 |
| Root | 1.34 | 1.97 | 1.01 | 1.33 | 1.95 | Glyma.14G059200.2 | phospholipase C 2 |
| Root | 1.35 | 1.60 | 1.01 | 1.33 | 1.57 | Glyma.16G174800.1 | disease resistance family protein/LRR family protein |
| Root | −1.41 | −1.63 | −1.07 | 1.32 | 1.52 | Glyma.18G072300.1 | Chaperone DnaJ-domain superfamily protein |
| Root | 1.40 | 1.68 | 1.07 | 1.31 | 1.57 | Glyma.18G043200.1 | TPX2 (targeting protein for Xklp2) protein family |
| Root | 1.33 | 1.53 | 1.02 | 1.31 | 1.50 | Glyma.16G129300.1 | disease resistance family protein/LRR family protein |
| Root | −1.40 | −2.20 | −1.08 | 1.29 | 2.04 | Glyma.16G155700.1 | glutathione-disulfide reductase |
| Root | −1.44 | −1.74 | −1.12 | 1.29 | 1.55 | Glyma.02G174100.3 | SNF2 domain-containing protein/helicase domain-containing |
| | | | | | | | protein |
| Root | 1.30 | 2.23 | 1.01 | 1.29 | 2.21 | Glyma.09G230700.2 | RNA polymerase III RPC4 |
| Root | 1.39 | 1.82 | 1.09 | 1.27 | 1.67 | Glyma.08G120300.1 | receptor-like kinase in flowers 1 |
| Root | 1.31 | 1.71 | 1.03 | 1.27 | 1.65 | Glyma.17G070100.1 | Eukaryotic aspartyl protease family protein |
| Root | 1.45 | 1.79 | 1.14 | 1.27 | 1.56 | Glyma.13G129700.2 | Protein of unknown function (DUF3741) |
| Root | −1.38 | −2.00 | −1.09 | 1.27 | 1.83 | Glyma.18G212500.1 | AZA-guanine resistant1 |
| Root | −1.46 | −1.83 | −1.16 | 1.26 | 1.58 | Glyma.19G213100.1 | Integrase-type DNA-binding superfamily protein |
| Root | −1.26 | −1.75 | −1.01 | 1.25 | 1.73 | Glyma.06G031000.1 | |
| Root | 1.33 | 1.93 | 1.06 | 1.25 | 1.82 | Glyma.06G201000.1 | exocyst subunit exo70 family protein B1 |
| Root | 1.44 | 2.45 | 1.15 | 1.25 | 2.12 | Glyma.13G156600.1 | Protein of unknown function (DUF1336) |
| Root | 1.85 | 2.33 | 1.50 | 1.24 | 1.56 | Glyma.14G218100.2 | Putative lysine decarboxylase family protein |
| Root | −1.24 | −2.05 | −1.01 | 1.23 | 2.03 | Glyma.17G129100.2 | |
| Root | 1.39 | 1.79 | 1.13 | 1.23 | 1.58 | Glyma.20G110000.1 | TRICHOME BIREFRINGENCE-LIKE 36 |
| Root | −1.39 | −1.75 | −1.13 | 1.23 | 1.55 | Glyma.10G283800.2 | Major facilitator superfamily protein |
| Root | 1.24 | 1.56 | 1.01 | 1.23 | 1.55 | Glyma.13G240800.6 | RING/U-box superfamily protein |
| Root | 1.28 | 1.73 | 1.04 | 1.22 | 1.66 | Glyma.12G006700.1 | P-loop containing nucleoside triphosphate hydrolases |
| | | | | | | | superfamily protein |
| Root | 1.27 | 1.76 | 1.04 | 1.22 | 1.70 | Glyma.15G046400.8 | Phosphatidylinositol-4-phosphate 5-kinase family protein |
| Root | 1.26 | 1.62 | 1.03 | 1.22 | 1.56 | Glyma.03G008500.16 | SKP1-like 21 |
| Root | 1.26 | 1.86 | 1.03 | 1.22 | 1.81 | Glyma.19G226400.1 | shaggy-related kinase 11 |
| Root | −1.55 | −2.09 | −1.28 | 1.22 | 1.64 | Glyma.07G074400.3 | NAD(P)-binding Rossmann-fold superfamily protein |
| Root | −1.29 | −1.70 | −1.06 | 1.21 | 1.60 | Glyma.16G186100.1 | disease resistance family protein/LRR family protein |
| Root | 1.33 | 1.73 | 1.12 | 1.19 | 1.54 | Glyma.01G203000.1 | |
| Root | 1.33 | 1.71 | 1.12 | 1.19 | 1.53 | Glyma.11G211100.1 | Leucine-rich repeat protein kinase family protein |
| Root | −1.37 | −2.03 | −1.15 | 1.19 | 1.76 | Glyma.10G039200.2 | no pollen germination related 2 |
| Root | 1.21 | 1.68 | 1.02 | 1.18 | 1.65 | Glyma.13G289300.2 | Ras-related small GTP-binding family protein |
| Root | −1.18 | −1.89 | −1.00 | 1.18 | 1.89 | Glyma.16G183400.1 | disease resistance family protein/LRR family protein |
| Root | 1.18 | 1.52 | 1.01 | 1.17 | 1.51 | Glyma.13G132700.1 | DNAse I-like superfamily protein |
| Root | −1.26 | −1.70 | −1.09 | 1.16 | 1.56 | Glyma.19G012300.2 | BAH domain; TFIIS helical bundle-like domain |
| Root | −1.22 | −1.58 | −1.06 | 1.16 | 1.50 | Glyma.16G195800.1 | cytochrome P450, family 71, subfamily A, polypeptide 22 |
| Root | −1.17 | −2.08 | −1.01 | 1.16 | 2.06 | Glyma.04G245400.2 | histone methyltransferases(H3-K4 specific); histone |
| | | | | | | | methyltransferases(H3-K36 specific) |
| Root | −1.32 | −2.26 | −1.14 | 1.15 | 1.98 | Glyma.09G215000.2 | HAC13 protein (HAC13) |
| Root | 1.21 | 3.51 | 1.06 | 1.15 | 3.33 | Glyma.08G288500.1 | K+ uptake permease 6 |
| Root | −1.18 | −2.01 | −1.02 | 1.15 | 1.96 | Glyma.13G333500.1 | Ras-related small GTP-binding family protein |
| Root | 1.29 | 1.75 | 1.13 | 1.15 | 1.55 | Glyma.12G211000.4 | Mitogen activated protein kinase kinase kinase-related |
| Root | −1.31 | −1.73 | −1.14 | 1.15 | 1.52 | Glyma.11G104200.5 | homolog of X-ray repair cross complementing 2 (XRCC2) |
| Root | 1.21 | 2.35 | 1.05 | 1.15 | 2.24 | Glyma.16G002600.1 | Octicosapeptide/Phox/Bem1p family protein |
| Root | −1.17 | −2.36 | −1.02 | 1.14 | 2.31 | Glyma.05G114100.1 | Protein kinase superfamily protein |
| Root | −1.25 | −1.80 | −1.09 | 1.14 | 1.65 | Glyma.17G061200.6 | villin 4 |
| Root | 1.27 | 1.70 | 1.12 | 1.14 | 1.51 | Glyma.20G145700.3 | D-aminoacid aminotransferase-like PLP-dependent enzymes |
| | | | | | | | superfamily protein |
| Root | −1.26 | −2.83 | −1.11 | 1.14 | 2.54 | Glyma.09G135200.5 | NAD(P)-binding Rossmann-fold superfamily protein |
| Root | −1.20 | −1.66 | −1.05 | 1.14 | 1.57 | Glyma.15G207700.3 | Protein of unknown function (DUF1637) |
| Root | 1.21 | 2.33 | 1.06 | 1.13 | 2.19 | Glyma.03G021100.6 | |
| Root | −1.42 | −2.16 | −1.25 | 1.13 | 1.72 | Glyma.13G210700.3 | alpha/beta-Hydrolases superfamily protein |
| Root | −1.31 | −1.79 | −1.16 | 1.13 | 1.55 | Glyma.02G308800.1 | 5\′-AMP-activated protein kinase beta-2 subunit protein |
| Root | 1.21 | 1.68 | 1.08 | 1.12 | 1.56 | Glyma.06G029000.4 | Tetrapyrrole (Corrin/Porphyrin) Methylases |
| Root | 1.35 | 1.83 | 1.21 | 1.12 | 1.51 | Glyma.05G188000.1 | |
| Root | 1.18 | 1.80 | 1.05 | 1.12 | 1.71 | Glyma.01G132000.1 | with no lysine (K) kinase 5 |
| Root | −1.16 | −1.62 | −1.04 | 1.12 | 1.56 | Glyma.14G060900.3 | Ribosomal protein L10 family protein |
| Root | 1.16 | 1.66 | 1.04 | 1.12 | 1.60 | Glyma.18G040600.1 | O-Glycosyl hydrolases family 17 protein |
| Root | −1.18 | −1.97 | −1.06 | 1.11 | 1.85 | Glyma.15G099100.2 | RING/U-box superfamily protein |
| Root | 1.17 | 1.68 | 1.05 | 1.11 | 1.59 | Glyma.03G253500.3 | non-photochemical quenching 1 |
| Root | −1.12 | −1.73 | −1.00 | 1.11 | 1.72 | Glyma.14G050300.2 | |
| Root | −1.22 | −1.67 | −1.09 | 1.11 | 1.52 | Glyma.08G117300.3 | RING/FYVE/PHD zinc finger superfamily protein |
| Root | 1.19 | 1.81 | 1.08 | 1.11 | 1.68 | Glyma.13G328900.1 | ROP interactive partner 5 |
| Root | 1.11 | 1.53 | 1.00 | 1.11 | 1.52 | Glyma.06G049200.1 | Integrase-type DNA-binding superfamily protein |
| Root | 1.14 | 1.77 | 1.03 | 1.11 | 1.72 | Glyma.01G196500.1 | |
| Root | −1.22 | −1.84 | −1.11 | 1.11 | 1.66 | Glyma.20G247400.2 | Major facilitator superfamily protein |
| Root | 1.26 | 1.81 | 1.14 | 1.11 | 1.59 | Glyma.04G123000.1 | |
| Root | 2.37 | 3.37 | 2.15 | 1.10 | 1.57 | Glyma.11G154300.2 | NagB/RpiA/CoA transferase-like superfamily protein |
| Root | −1.11 | −1.54 | −1.00 | 1.10 | 1.53 | Glyma.11G058500.1 | |
| Root | −1.36 | −1.94 | −1.24 | 1.10 | 1.57 | Glyma.12G189500.3 | BRI1 suppressor 1 (BSU1)-like 2 |
| Root | 1.15 | 1.82 | 1.05 | 1.10 | 1.73 | Glyma.06G086700.3 | |
| Root | −1.19 | −1.67 | −1.09 | 1.09 | 1.53 | Glyma.05G215100.2 | myosin heavy chain-related |
| Root | −1.22 | −2.17 | −1.13 | 1.08 | 1.92 | Glyma.19G012300.5 | BAH domain; TFIIS helical bundle-like domain |
| Root | −1.11 | 1.57 | −1.03 | 1.08 | −1.53 | Glyma.10G182000.7 | |
| Root | 1.09 | 1.55 | 1.02 | 1.08 | 1.52 | Glyma.04G192200.5 | retinoblastoma-related 1 |
| Root | 1.17 | 1.66 | 1.09 | 1.07 | 1.52 | Glyma.U034500.5 | pseudo-response regulator 3 |
| Root | −1.17 | −2.20 | −1.10 | 1.07 | 2.01 | Glyma.19G012300.6 | BAH domain; TFIIS helical bundle-like domain |
| Root | 1.16 | 1.68 | 1.09 | 1.07 | 1.55 | Glyma.06G124000.2 | Glycolipid transfer protein (GLTP) family protein |
| Root | −1.21 | −14.60 | −1.14 | 1.06 | 12.85 | Glyma.02G282700.2 | helicase in vascular tissue and tapetum |
| Root | 1.12 | 1.83 | 1.06 | 1.06 | 1.73 | Glyma.09G103300.3 | nucleic acid binding; methyltransferases |
| Root | −1.25 | −1.82 | −1.18 | 1.06 | 1.54 | Glyma.20G149400.2 | Ribosomal protein S3Ae |
| Root | −1.22 | −3.76 | −1.15 | 1.06 | 3.27 | Glyma.11G232900.1 | P-loop containing nucleoside triphosphate hydrolases |
| | | | | | | | superfamily protein |
| Root | −1.32 | −2.12 | −1.25 | 1.06 | 1.70 | Glyma.10G024500.3 | Tudor/PWWP/MBT domain-containing protein |
| Root | 1.07 | 1.61 | 1.02 | 1.06 | 1.58 | Glyma.10G145100.1 | ADP-ribosylation factor A1F |
| Root | −1.19 | −1.78 | −1.13 | 1.05 | 1.58 | Glyma.04G009800.1 | myo-inositol monophosphatase like 2 |
| Root | −1.07 | −1.59 | −1.02 | 1.05 | 1.56 | Glyma.11G067300.2 | U1 small nuclear ribonucleoprotein-70K |
| Root | 1.13 | 1.63 | 1.08 | 1.05 | 1.51 | Glyma.01G053600.2 | Protein of unknown function (DUF1336) |
| Root | 1.10 | 1.61 | 1.05 | 1.04 | 1.52 | Glyma.02G301400.1 | Plant protein of unknown function (DUF827) |
| Root | 1.06 | 1.63 | 1.03 | 1.04 | 1.59 | Glyma.19G129400.1 | Arv1-like protein |
| Root | −1.07 | −1.61 | −1.03 | 1.04 | 1.57 | Glyma.03G176300.3 | Glutathione S-transferase family protein |
| Root | −1.18 | −1.95 | −1.14 | 1.03 | 1.71 | Glyma.03G019300.3 | AGAMOUS-like 20 |
| Root | −1.07 | −1.59 | −1.04 | 1.03 | 1.53 | Glyma.18G016200.1 | DNAJ heat shock family protein |
| Root | 1.09 | 1.64 | 1.07 | 1.02 | 1.54 | Glyma.08G055000.3 | non-intrinsic ABC protein 3 |
| Root | 1.20 | 2.24 | 1.18 | 1.02 | 1.90 | Glyma.18G156100.1 | NAD(P)-binding Rossmann-fold superfamily protein |
| Root | 1.20 | 2.24 | 1.18 | 1.02 | 1.90 | Glyma.18G156100.3 | NAD(P)-binding Rossmann-fold superfamily protein |
| Root | −1.04 | −1.98 | −1.02 | 1.02 | 1.93 | Glyma.04G072500.1 | |
| Root | −1.12 | −1.79 | −1.10 | 1.02 | 1.62 | Glyma.20G157600.10 | homolog of DNA mismatch repair protein MSH3 |
| Root | 1.17 | 1.77 | 1.15 | 1.01 | 1.54 | Glyma.18G101700.2 | |
| Root | 1.01 | −1.59 | 1.00 | 1.01 | −1.59 | Glyma.20G238800.1 | RING/U-box protein |
| Root | −1.04 | −2.02 | −1.03 | 1.01 | 1.96 | Glyma.05G216800.1 | Pleckstrin homology (PH) domain-containing protein |
| Root | 1.22 | 2.46 | 1.20 | 1.01 | 2.05 | Glyma.19G233000.2 | Protein of unknown function (DUF1624) |
| Root | −1.17 | −2.55 | −1.15 | 1.01 | 2.21 | Glyma.02G228600.2 | |
| Root | 1.07 | 1.67 | 1.05 | 1.01 | 1.59 | Glyma.02G122700.5 | 18S pre-ribosomal assembly protein gar2-related |
| Root | 1.39 | 2.42 | 1.38 | 1.01 | 1.76 | Glyma.17G245000.2 | Protein of unknown function, DUF547 |
| Root | −1.08 | −1.66 | −1.07 | 1.01 | 1.55 | Glyma.09G275900.7 | Protein kinase superfamily protein |
| Root | 1.20 | 2.24 | 1.19 | 1.01 | 1.89 | Glyma.09G254200.2 | jasmonic acid carboxyl methyltransferase |
| Root | 1.05 | 1.58 | 1.04 | 1.01 | 1.52 | Glyma.19G255100.2 | alpha/beta-Hydrolases superfamily protein |
| Root | −1.12 | −2.26 | −1.11 | 1.01 | 2.04 | Glyma.04G155000.2 | Family of unknown function (DUF566) |
| Root | −1.08 | −1.64 | −1.08 | 1.00 | 1.52 | Glyma.20G161400.7 | UB-like protease 1A |
| Root | −1.06 | −1.63 | −1.06 | 1.00 | 1.54 | Glyma.19G047800.1 | abscisic acid (aba)-deficient 4 |
| Root | −1.02 | 1.68 | −1.02 | 1.00 | −1.65 | Glyma.06G317700.4 | U-box domain-containing protein kinase family protein |
| Root | 1.20 | 1.92 | 1.20 | 1.00 | 1.60 | Glyma.13G130300.1 | Ankyrin repeat family protein |
| Root | −1.04 | −1.62 | −1.05 | 1.00 | 1.55 | Glyma.03G172700.5 | pyrophosphorylase 4 |
| Root | 1.05 | 3.13 | 1.05 | 1.00 | 2.97 | Glyma.11G112000.4 | squalene synthase 1 |
| Root | 1.06 | 3.03 | 1.06 | 1.00 | 2.84 | Glyma.10G035200.6 | alpha/beta-Hydrolases superfamily protein |
| Root | 1.06 | 2.26 | 1.07 | 0.99 | 2.12 | Glyma.07G102800.3 | Vacuolar protein sorting-associated protein 26 |
| Root | 1.05 | 2.89 | 1.05 | 0.99 | 2.74 | Glyma.10G083600.6 | |
| Root | 1.05 | 2.09 | 1.06 | 0.99 | 1.98 | Glyma.17G112800.1 | |
| Root | 1.06 | 3.23 | 1.07 | 0.99 | 3.02 | Glyma.20G220200.1 | phloem protein 2-B10 |
| Root | 1.06 | 1.83 | 1.07 | 0.99 | 1.72 | Glyma.17G051300.2 | phospholipid:diacylglycerol acyltransferase |
| Root | −1.02 | −1.69 | −1.03 | 0.99 | 1.64 | Glyma.05G209400.2 | ubiquitin-conjugating enzyme 5 |
| Root | −1.06 | −1.97 | −1.07 | 0.99 | 1.84 | Glyma.06G192200.1 | ribosomal protein L29 family protein |
| Root | 1.05 | 5.92 | 1.07 | 0.98 | 5.52 | Glyma.16G155800.1 | histidinol phosphate aminotransferase 1 |
| Root | 1.20 | 3.40 | 1.22 | 0.98 | 2.79 | Glyma.06G107200.2 | electron carriers; protein disulfide oxidoreductases |
| Root | 2.26 | 3.93 | 2.31 | 0.98 | 1.70 | Glyma.08G117300.2 | RING/FYVE/PHD zinc finger superfamily protein |
| Root | 1.03 | 2.13 | 1.06 | 0.97 | 2.00 | Glyma.19G250900.4 | |
| Root | −1.00 | −1.81 | −1.03 | 0.97 | 1.75 | Glyma.16G219900.9 | B-block binding subunit of TFIIIC |
| Root | −1.02 | −1.93 | −1.05 | 0.97 | 1.83 | Glyma.09G229600.1 | P-loop containing nucleoside triphosphate hydrolases |
| | | | | | | | superfamily protein |
| Root | −1.15 | −2.15 | −1.19 | 0.96 | 1.81 | Glyma.04G185600.2 | |
| Root | −1.15 | −1.82 | −1.19 | 0.96 | 1.53 | Glyma.18G234900.3 | ARID/BRIGHT DNA-binding domain; ELM2 domain protein |
| Root | −1.11 | −3.07 | −1.16 | 0.96 | 2.64 | Glyma.04G185500.2 | |
| Root | 1.04 | 1.72 | 1.09 | 0.95 | 1.57 | Glyma.04G228900.1 | |
| Root | −1.06 | −9.31 | −1.11 | 0.95 | 8.36 | Glyma.06G098900.2 | phosphoenolpyruvate carboxylase-related kinase 2 |
| Root | 1.01 | 1.90 | 1.06 | 0.95 | 1.79 | Glyma.05G201200.1 | dual specificity protein phosphatase family protein |
| Root | 1.07 | 1.79 | 1.13 | 0.95 | 1.58 | Glyma.15G035700.2 | Chaperone DnaJ-domain superfamily protein |
| Root | 1.05 | 1.85 | 1.11 | 0.95 | 1.66 | Glyma.12G074600.2 | hercules receptor kinase 1 |
| Root | −1.06 | −2.10 | −1.12 | 0.94 | 1.87 | Glyma.20G101400.1 | aluminum-activated, malate transporter 12 |
| Root | −1.01 | −1.63 | −1.08 | 0.94 | 1.51 | Glyma.05G009000.1 | early nodulin-related |
| Root | −1.12 | −2.88 | −1.20 | 0.93 | 2.41 | Glyma.07G002800.2 | Rad23 UV excision repair protein family |
| Root | 1.24 | 2.05 | 1.34 | 0.93 | 1.53 | Glyma.09G072000.1 | Integrase-type DNA-binding superfamily protein |
| Root | −1.01 | −1.70 | −1.10 | 0.92 | 1.54 | Glyma.05G104400.5 | 2-oxoglutarate (2OG) and Fe(II)-dependent oxygenase |
| | | | | | | | superfamily protein |
| Root | −1.03 | −2.07 | −1.14 | 0.91 | 1.82 | Glyma.17G178800.3 | sucrose nonfermenting 1(SNF1)-related protein kinase 2.3 |
| Root | 1.01 | 1.70 | 1.12 | 0.90 | 1.52 | Glyma.03G101700.2 | Fatty acid hydroxylase superfamily |
| Root | −1.09 | −1.87 | −1.21 | 0.90 | 1.55 | Glyma.02G033300.1 | Adaptor protein complex AP-1, gamma subunit |
| Root | 1.07 | 3.44 | 1.20 | 0.89 | 2.87 | Glyma.16G040900.3 | adenosine-5\′-phosphosulfate (APS) kinase 3 |
| Root | 1.07 | 5.61 | 1.21 | 0.89 | 4.62 | Glyma.15G234500.1 | |
| Root | −1.03 | −6.78 | −1.16 | 0.88 | 5.84 | Glyma.14G108700.2 | Major facilitator superfamily protein |
| Root | 1.06 | 2.48 | 1.20 | 0.88 | 2.06 | Glyma.19G009300.5 | Pre-rRNA-processing protein TSR2, conserved region |
| Root | 1.06 | 2.27 | 1.21 | 0.88 | 1.88 | Glyma.05G248100.3 | alpha/beta-Hydrolases superfamily protein |
| Root | 1.06 | 2.04 | 1.22 | 0.87 | 1.68 | Glyma.09G209200.2 | Protein of unknown function (DUF760) |
| Root | 1.09 | 12.94 | 1.25 | 0.87 | 10.31 | Glyma.15G053000.6 | dentin sialophosphoprotein-related |
| Root | 1.15 | 2.17 | 1.33 | 0.86 | 1.63 | Glyma.15G180000.1 | Integrase-type DNA-binding superfamily protein |
| Root | 1.07 | 2.74 | 1.24 | 0.86 | 2.21 | Glyma.02G172900.2 | Protein phosphatase 2C family protein |
| Root | −1.19 | −6.95 | −1.47 | 0.81 | 4.73 | Glyma.13G332300.3 | sequence-specific DNA binding; sequence-specific DNA binding |
| | | | | | | | transcription factors |
| Root | −1.06 | −2.45 | −1.63 | 0.65 | 1.50 | Glyma.02G198100.10 | |
| Root | 1.00 | −1.95 | −1.30 | −0.77 | 1.50 | Glyma.05G116900.3 | tobamovirus multiplication 1 |
| Root | 1.04 | −1.88 | −1.25 | −0.83 | 1.50 | Glyma.13G042600.4 | GTP cyclohydrolase II |
| Root | 1.02 | −1.74 | −1.11 | −0.92 | 1.57 | Glyma.13G199600.1 | Erv1/Alr family protein |
| Root | 1.01 | −1.65 | −1.09 | −0.92 | 1.51 | Glyma.01G048500.3 | galactosyltransferase1 |
| Root | −1.01 | 1.96 | 1.09 | −0.93 | 1.80 | Glyma.16G207200.1 | histone-lysine N-methyltransferase ASHH3 |
| Root | 1.02 | −3.22 | −1.08 | −0.94 | 2.98 | Glyma.04G105000.2 | tRNA modification GTPase, putative |
| Root | −1.04 | 1.74 | 1.08 | −0.96 | 1.61 | Glyma.11G179600.3 | NIMA (never in mitosis, gene A)-related 6 |
| Root | 1.02 | −1.62 | −1.06 | −0.96 | 1.53 | Glyma.07G232500.1 | CRT (chloroquine-resistance transporter)-like transporter 3 |
| Root | −1.03 | −1.85 | 1.07 | −0.97 | −1.73 | Glyma.19G243200.2 | Plant protein of unknown function (DUF868) |
| Root | 1.01 | 2.16 | −1.04 | −0.97 | −2.07 | Glyma.10G209600.2 | Zinc finger (C2H2 type) family protein/transcription factor |
| | | | | | | | jumonji (jmj) family protein |
| Root | 1.07 | −1.68 | −1.10 | −0.97 | 1.53 | Glyma.18G230900.3 | Ribosomal protein S5 domain 2-like superfamily protein |
| Root | −1.05 | 1.65 | 1.07 | −0.98 | 1.54 | Glyma.16G178400.1 | Heavy metal transport/detoxification superfamily protein |
| Root | −1.05 | −1.66 | 1.06 | −0.98 | −1.56 | Glyma.05G104400.3 | 2-oxoglutarate (2OG) and Fe(II)-dependent oxygenase |
| | | | | | | | superfamily protein |
| Root | −1.02 | 1.71 | 1.02 | −0.99 | 1.67 | Glyma.06G135900.1 | |
| Root | 1.00 | −1.67 | −1.00 | −1.00 | 1.67 | Glyma.02G095900.6 | Phosphatidylinositol N-acetyglucosaminlytransferase subunit P- |
| | | | | | | | related |
| Root | −1.05 | 2.14 | 1.04 | −1.01 | 2.06 | Glyma.17G242500.2 | plant glycogenin-like starch initiation protein 3 |
| Root | 1.05 | −1.70 | −1.01 | −1.04 | 1.69 | Glyma.17G242500.1 | plant glycogenin-like starch initiation protein 3 |
| Root | 1.07 | 1.56 | −1.02 | −1.04 | −1.53 | Glyma.01G064600.1 | ACT domain repeat 4 |
| Root | −1.08 | −1.78 | 1.03 | −1.06 | −1.73 | Glyma.19G247600.1 | Homeodomain-like superfamily protein |
| Root | 1.11 | 1.69 | −1.04 | −1.07 | −1.62 | Glyma.06G215200.1 | Cytochrome P450 superfamily protein |
| Root | 1.08 | −1.56 | −1.01 | −1.07 | 1.55 | Glyma.13G161900.1 | purple acid phosphatase 26 |
| Root | −1.15 | −8.39 | 1.07 | −1.07 | −7.86 | Glyma.13G031600.3 | amino acid permease 2 |
| Root | −1.15 | 4.09 | 1.06 | −1.08 | 3.85 | Glyma.13G124800.1 | AGC (cAMP-dependent, cGMP-dependent and protein kinase C) |
| | | | | | | | kinase family protein |
| Root | −1.08 | −1.54 | 1.00 | −1.08 | −1.54 | Glyma.01G089500.7 | |
| Root | 1.14 | 1.84 | −1.04 | −1.09 | −1.76 | Glyma.17G066700.3 | Protein kinase superfamily protein |
| Root | −1.12 | −1.70 | 1.01 | −1.10 | −1.68 | Glyma.05G232400.8 | Fibronectin type III domain-containing protein |
| Root | −1.13 | −1.64 | 1.02 | −1.11 | −1.61 | Glyma.01G045800.1 | peroxisomal adenine nucleotide carrier 1 |
| Root | 1.12 | 1.57 | −1.01 | −1.11 | −1.57 | Glyma.10G038700.1 | Ypt/Rab-GAP domain of gyp1p superfamily protein |
| Root | −1.13 | −1.52 | 1.00 | −1.13 | −1.52 | Glyma.15G069200.6 | Protein of unknown function (DUF707) |
| Root | −1.15 | −1.53 | 1.00 | −1.14 | −1.52 | Glyma.14G162700.1 | hAT transposon superfamily |
| Root | 1.17 | 1.66 | −1.01 | −1.16 | −1.65 | Glyma.08G198900.1 | Leucine-rich repeat protein kinase family protein |
| Root | −1.22 | −1.63 | 1.02 | −1.19 | −1.59 | Glyma.19G071700.1 | Ribosomal protein S24e family protein |
| Root | 1.23 | 1.89 | −1.01 | −1.21 | −1.87 | Glyma.06G122800.2 | serine carboxypeptidase-like 25 |
| Root | −1.23 | −1.84 | 1.01 | −1.22 | −1.82 | Glyma.10G052700.2 | vacuolar membrane ATPase 10 |
| Root | −1.26 | −1.74 | 1.03 | −1.22 | −1.69 | Glyma.09G215800.2 | SH3 domain-containing protein |
| Root | −1.27 | −1.56 | 1.03 | −1.24 | −1.52 | Glyma.03G236900.3 | homolog of yeast sucrose nonfermenting 4 |
| Root | −1.31 | −1.80 | 1.06 | −1.24 | −1.71 | Glyma.16G175500.1 | UDP-glucosyl transferase 88A1 |
| Root | −1.33 | −1.58 | 1.02 | −1.30 | −1.54 | Glyma.16G190100.1 | CAP160 protein |
| Root | 1.37 | 1.59 | −1.06 | −1.30 | −1.50 | Glyma.14G116000.7 | Protein kinase superfamily protein |
| Root | −1.38 | −1.60 | 1.03 | −1.35 | −1.56 | Glyma.13G217600.3 | RNA-binding protein |
| Root | 1.41 | 1.59 | −1.01 | −1.40 | −1.58 | Glyma.02G083700.2 | |
| Root | 1.43 | 1.76 | −1.01 | −1.41 | −1.73 | Glyma.02G248400.1 | XH/XS domain-containing protein |
| Root | −1.60 | −1.98 | 1.05 | −1.53 | −1.89 | Glyma.16G169200.1 | receptor like protein 6 |
| Root | 1.56 | 1.67 | −1.01 | −1.55 | −1.66 | Glyma.16G174900.1 | P-loop containing nucleoside triphosphate hydrolases |
| | | | | | | | superfamily protein |
| Root | −1.75 | −1.96 | 1.07 | −1.63 | −1.82 | Glyma.16G159300.1 | Disease resistance protein (TIR-NBS-LRR class) family |
| Root | 1.67 | 1.54 | −1.00 | −1.66 | −1.53 | Glyma.18G030100.1 | Mitochondrial substrate carrier family protein |
| Root | −1.72 | −1.55 | 1.03 | −1.68 | −1.51 | Glyma.16G159200.1 | Disease resistance protein (TIR-NBS-LRR class) family |

TABLE 8
Plant Proteomics
Fold change increase or decrease of plant proteins secreted under normal watering and water-
limited conditions, in root and leaf tissues of plants grown from seeds treated with Strain B,
compared to plants grown from seeds treated with the formulation control

| | Normal | | Water- | |
|---|---|---|---|---|
| | Watering | | Limited | |
| | Conditions | | Conditions | |
| Protein Name | Root | Leaf | Root | Leaf |
| alcohol dehydrogenase 1 | 0.73 | | | 1.39 |
| chloroplast stem-loop binding protein of 41 kDa a, chloroplastic-like | 0.87 | 1.17 | | 1.45 |
| cucumisin-like isoform X2 | 0.89 | 1.23 | | 1.39 |
| cytochrome b6-f complex iron-sulfur subunit, chloroplastic-like | 1.16 | | | 1.31 |
| sedoheptulose-1,7-bisphosphatase, chloroplastic-like isoform 1 | | 1.15 | | 1.26 |
| ATP synthase CF1 alpha subunit | | 1.16 | | 1.30 |
| methionine synthase | | 1.16 | | 1.61 |
| LOW QUALITY PROTEIN: 50S ribosomal protein L11, chloroplastic-like | | 1.18 | | 1.41 |
| ruBisCO-associated protein | | 1.24 | | 2.06 |
| peroxidase 50-like | | | 0.79 | 1.45 |
| aconitate hydratase 2, mitochondrial-like | | | 0.87 | 1.33 |
| lipoxygenase | | | 1.14 | 1.32 |
| cytochrome f | | | | 1.27 |
| aspartate aminotransferase glyoxysomal isozyme AAT1 precursor | | | | 1.34 |
| vicianin hydrolase-like | | | | 1.54 |
| 3-ketoacyl-CoA thiolase 2, peroxisomal-like isoform X1 | | 0.90 | 1.33 | 0.71 |
| protein CURVATURE THYLAKOID 1A, chloroplastic-like | | 0.79 | 1.31 | 0.60 |
| alcohol dehydrogenase class-3 | 0.75 | | 1.53 | |
| glutamine synthetase precursor isoform X1 | 0.80 | | 1.27 | |
| UTP--glucose-1-phosphate uridylyltransferase-like | 0.85 | 0.88 | 1.27 | |
| enolase-phosphatase E1-like | 0.86 | | 1.52 | |
| luminal-binding protein | 0.86 | | 1.33 | 1.24 |
| ketol-acid reductoisomerase, chloroplastic-like | 0.87 | 0.90 | 1.39 | |
| beta-amylase precursor | 1.17 | 0.88 | 1.28 | |
| peptidyl-prolyl cis-trans isomerase, chloroplastic isoform 1 | | | 1.27 | |
| aldehyde dehydrogenase family 3 member I1, chloroplastic-like | | | 1.30 | |
| 50S ribosomal protein L6, chloroplastic-like isoform 1 | | | 1.30 | |
| lipoxygenase-9 | | | 1.39 | 1.16 |
| 26S protease regulatory subunit 10B homolog A-like | | | 1.40 | |
| heat shock protein 90-1 | | | 1.46 | |
| DNA-damage-repair/toleration protein DRT100-like precursor | | | 1.51 | 1.14 |
| glutamate--glyoxylate aminotransferase 2-like isoform 2 | 0.72 | | 1.39 | 1.26 |
| fructose-bisphosphate aldolase 1, chloroplastic-like | 0.88 | 1.27 | 1.22 | 1.20 |
| stromal 70 kDa heat shock-related protein, chloroplastic-like | 0.88 | 1.30 | 1.19 | |
| thioredoxin M4, chloroplastic-like | 1.11 | 1.29 | | |
| thylakoid lumenal 16.5 kDa protein, chloroplastic-like isoform X1 | 1.17 | 1.51 | | |
| 1-deoxy-D-xylulose 5-phosphate reductoisomerase, chloroplastic-like | 1.21 | 1.32 | | |
| histone H4-like | 1.23 | 1.35 | 0.76 | |
| thylakoid lumenal 29 kDa protein, chloroplastic-like | | 1.32 | 0.82 | |
| probable 60S ribosomal protein L14-like | | 1.32 | | |
| dirigent protein 1-like | | 1.33 | | |
| photosystem II 44 kDa protein | | 1.36 | | |
| lipoxygenase L-5 | | 1.36 | 1.12 | |
| CDGSH iron-sulfur domain-containing protein NEET-like | | 1.47 | | |
| photosystem II protein H | | 1.62 | | |
| thioredoxin-like protein CDSP32, chloroplastic-like | | 1.34 | 0.87 | 1.62 |
| asparagine synthetase, root [glutamine-hydrolyzing]-like | 1.30 | | 0.88 | 0.76 |
| THO complex subunit 4-like isoform X1 | 1.31 | 0.87 | 0.71 | 0.87 |
| peptidyl-prolyl cis-trans isomerase CYP20-2, chloroplastic-like isoform 1 | 1.26 | | | |
| DNA repair and recombination protein RAD26-like | 1.28 | | | 1.13 |
| mitochondrial dicarboxylate/tricarboxylate transporter DTC-like | 1.29 | | 0.90 | 1.10 |
| proteasome subunit alpha type-5-like | 1.33 | | | |
| bifunctional aspartate aminotransferase and glutamate/aspartate- | 1.34 | 0.87 | 0.83 | |
| prephenate aminotransferase-like | | | | |
| cysteine proteinase RD21a-like | 1.43 | | 0.82 | |
| isocitrate dehydrogenase [NADP] | 1.36 | | 1.22 | 1.41 |
| putative glucose-6-phosphate 1-epimerase-like | 0.89 | | | 0.67 |
| putative lactoylglutathione lyase-like isoform X2 | | 0.90 | | 0.73 |
| delta-1-pyrroline-5-carboxylate synthase-like | | | 0.88 | 0.74 |
| probable histone H2B.3 | | | | 0.72 |
| ornithine carbamoyltransferase, chloroplastic-like | | | | 0.73 |
| chaperonin CPN60-like 2, mitochondrial-like | | | | 0.73 |
| ATP synthase CF1 epsilon subunit | | | | 0.75 |
| photosystem I P700 apoprotein A2 | 1.24 | | 0.62 | 1.15 |
| calcium-transporting ATPase 4, plasma membrane-type-like | | | 0.65 | |
| abscisic stress ripening-like protein | | | 0.65 | |
| 3′-hydroxy-N-methyl-(S)-coclaurine4 ′-O-methyltransferase-like | | | 0.69 | |
| sulfite reductase [ferredoxin], chloroplastic | | | 0.70 | |
| 60S ribosomal protein L4-like isoform 1 | | | 0.72 | |
| isocitrate dehydrogenase [NAD] catalytic subunit 5, mitochondrial-like | | | 0.74 | |
| transketolase, chloroplastic | | | 0.76 | |
| putative dihydroxy-acid dehydratase, mitochondrial-like | | 0.89 | 0.64 | 0.78 |
| inositol-3-phosphate synthase | | 0.70 | | 1.20 |
| protease Do-like 2, chloroplastic-like | | 0.77 | | 0.83 |
| T-complex protein 1 subunit gamma-like | | 0.77 | 0.83 | |
| cell division protein FtsZ homolog 1, chloroplastic-like | | 0.79 | 1.19 | 0.82 |
| magnesium-chelatase subunit ChII, chloroplastic | 0.62 | 0.85 | | |
| endoplasmin homolog isoform X2 | 0.63 | | | |
| chloroplast stem-loop binding protein of 41 kDa b, chloroplastic-like | 0.67 | | | |
| isoform X2 | | | | |
| leucine aminopeptidase 3, chloroplastic-like | 0.71 | | | |
| peroxisomal (S)-2-hydroxy-acid oxidase GLO1-like isoform X1 | 0.72 | | 1.18 | |
| patellin-3-like isoform X3 | 0.73 | | | |
| NADP-dependent malic enzyme-like | 0.77 | | 1.23 | |

### Table 9: Plant Hormone Analysis Results

Plant hormone analysis of plants grown from seeds treated with Strain B, as compared to plants grown from seeds treated with the formulation control, under normal watering and water-limited conditions. The values indicate Strain B/control fold change. Mass spectra of 8 plant hormones were obtained: jasmonic acid (JA), jasmonic acid-isoleucine (JA-Ile), salicylic acid (SA), abscisic acid (ABA), 12-oxo-phytodienoic acid (OPDA), 10-oxo-11 phytoenoic acid (OPEA), traumatic acid (TA) and cinnaminic acid (CA).

### Table 10: Metabolomics Results

TABLE 10A
Metabolic analysis of plants grown from seeds treated with Strain
B under normal (well-watered) conditions. “+” and “−”
denote a relative increase or decrease, respectively, when compared
to control plants grown in similar conditions (formulation control).

| Metabolic process | Metabolite | root | stem | leaf |
|---|---|---|---|---|
| Alkaloid metabolism | tryptophan | − | + | + |
| | phenylalanine | − | | + |
| | tyrosine | − | + | |
| | tryptamine | | − | − |
| | benzoic acid | | | |
| | pipecolic acid | | | + |
| | nicotinic acid | − | | |
| Phenylpropanoid | phenylalanine | − | | + |
| metabolism | shikimic acid | | | |
| | tyrosine | − | + | |
| | quinic acid | − | | |
| | sinapic acid | − | | + |
| | ferulic acid | − | | |
| | caffeic acid | − | + | |
| Flavonoid/isoflavonoid | quinic acid | − | | |
| biosynthesis | shikimic acid | | | |
| | hesperetin | | | |
| | daidzein | − | − | + |
| Lipid metabolism/fatty | ethanolamine | − | | |
| alcohols | ethanolaminephosphate | − | − | + |
| | sphingosine | − | | + |
| | glycerol | − | | |
| | hexadecanoic acid | − | − | |
| | octadecadienoic acid | | − | |
| | octadecanoic acid | − | − | + |
| | dodecanol | − | − | − |
| | campesterol | − | | + |
| Nitrogen | alanine | − | | |
| metabolism/amino | β-alanine | − | | |
| acids | allantoin | − | + | |
| | asparagine | − | − | − |
| | aspartic acid | | + | |
| | glutamic acid | − | + | |
| | glutamine | − | + | − |
| | histidine | − | | |
| | isoleucine | − | + | + |
| | leucine | − | | |
| | methionine | − | | |
| | phenylalanine | − | | + |
| | proline | − | + | + |
| | serine | − | | |
| | threonine | − | | |
| | tryptophan | − | + | + |
| | tyrosine | − | + | |
| | valine | − | − | |
| Carbohydrates | D-glucopyranose | − | + | − |
| | galactose | − | + | + |
| | lyxose | − | | |
| | sucrose | + | + | |
| | threose | − | | + |
| | trehalose | | + | + |
| | xylose | − | | |
| Other | salicylic acid | − | | |
| | pyrogallol | − | + | − |
| | hydroxyquinol | | | |
| | vanillic acid | | − | |
| | gallic acid | − | + | |
| | beta tocopherol | − | | + |
| | galacturonic acid | − | − | + |
| | lumichrome | | − | − |

TABLE 10B
Metabolic analysis of plants grown from seeds treated with Strain
B under water-limited conditions. “+” and “−”
denote a relative increase or decrease, respectively, when compared
to control plants grown in similar conditions (formulation control).

| Metabolic process | Metabolite | root | stem | leaf |
|---|---|---|---|---|
| Alkaloid metabolism | tryptophan | | − | |
| | phenylalanine | | | |
| | tyrosine | | − | |
| | tryptamine | | | |
| | benzoic acid | | + | |
| | pipecolic acid | | | |
| | nicotinic acid | + | | |
| Phenylpropanoid | phenylalanine | | | |
| metabolism | shikimic acid | | | |
| | tyrosine | | − | |
| | quinic acid | | | |
| | sinapic acid | | | + |
| | ferulic acid | + | + | |
| | caffeic acid | + | | + |
| Flavonoid/isoflavonoid | quinic acid | | | |
| biosynthesis | shikimic acid | | | |
| | hesperetin | − | + | − |
| | daidzein | | | |
| Lipid metabolism/fatty | ethanolamine | | | |
| alcohols | ethanolaminephosphate | | | |
| | sphingosine | | | − |
| | glycerol | | | |
| | hexadecanoic acid | | | − |
| | octadecadienoic acid | | + | − |
| | octadecanoic acid | − | − | − |
| | dodecanol | | + | |
| | campesterol | | | |
| Nitrogen | alanine | | + | |
| metabolism/amino | β-alanine | | | + |
| acids | allantoin | | − | |
| | asparagine | | − | + |
| | aspartic acid | | | |
| | glutamic acid | | | |
| | glutamine | | | + |
| | histidine | | − | + |
| | isoleucine | | + | |
| | leucine | | + | + |
| | methionine | | | |
| | phenylalanine | | | |
| | proline | − | − | − |
| | serine | | − | + |
| | threonine | | | |
| | tryptophan | | − | |
| | tyrosine | | − | |
| | valine | | + | + |
| Carbohydrates | D-glucopyranose | + | | − |
| | galactose | | − | |
| | lyxose | | − | |
| | sucrose | | | |
| | threose | | | |
| | trehalose | | | |
| | xylose | | | |
| Other | salicylic acid | | | |
| | pyrogallol | − | + | |
| | hydroxyquinol | | | |
| | vanillic acid | | | |
| | gallic acid | | | |
| | beta tocopherol | | | + |
| | galacturonic acid | + | | + |
| | lumichrome | | + | |

### Table 11: Community Sequencing

TABLE 11A
The average abundance of bacterial genera, as a proportion of the microbial
community, in leaf tissue of water stressed soybean plants grown from seeds treated
with the control Penicillium endophyte Strain F, the beneficial Penicillium endophyte
Strain B, and formulation control are shown. The average abundance of organisms in the
Escherica-Shigella genera were reduced from approximately 22.8% of the bacterial
community of untreated soybean leaves to approximately 16.8% of the bacterial
community in Strain B treated soybean leaves and 15.1% of the bacterial community in
Strain F treated leaves. Treatment with Strain B reduced the abundance of bacteria
in the Escherica-Shigella genera on soybean leaves by 26.6% relative to untreated
controls; treatment with Strain F reduced the abundance of bacteria in the
Escherica-Shigella genera on soybean leaves by 34% relative to untreated controls.

| | | | | | Formulation |
|---|---|---|---|---|---|
| Genus | Strain B | Genus | Strain F | Genus | Control |
| Escherichia- | 0.1676 | Escherichia- | 0.1506 | Escherichia- | 0.2283 |
| Shigella | | Shigella | | Shigella | |
| Bradyrhizobium | 0.0915 | Bradyrhizobium | 0.0636 | Bradyrhizobium | 0.1341 |
| Cellvibrio | 0.0425 | Cellvibrio | 0.0393 | Cellvibrio | 0.0323 |
| Flavobacterium | 0.0239 | Flavobacterium | 0.0330 | Flavobacterium | 0.0273 |
| Piscinibacter | 0.0196 | Burkholderia | 0.0317 | Piscinibacter | 0.0246 |
| Rhizobium | 0.0177 | Rhizobium | 0.0298 | Rhizobium | 0.0213 |
| Methylotenera | 0.0161 | Piscinibacter | 0.0254 | Methylotenera | 0.0188 |
| Massilia | 0.0148 | Massilia | 0.0207 | Massilia | 0.0172 |
| Devosia | 0.0141 | Methylotenera | 0.0166 | Hydrogenophaga | 0.0155 |
| Hydrogenophaga | 0.0120 | Hydrogenophaga | 0.0161 | Pseudomonas | 0.0134 |
| Pseudomonas | 0.0119 | Devosia | 0.0132 | Devosia | 0.0102 |
| Bacillus | 0.0101 | Pseudomonas | 0.0121 | Ohtaekwangia | 0.0077 |
| Ohtaekwangia | 0.0085 | Shinella | 0.0121 | Asticcacaulis | 0.0073 |
| Streptomyces | 0.0079 | Ohtaekwangia | 0.0113 | Bacillus | 0.0062 |
| Methylibium | 0.0073 | Asticcacaulis | 0.0101 | Shinella | 0.0061 |
| Asticcacaulis | 0.0065 | Leptothrix | 0.0093 | Enterococcus | 0.0061 |
| Arthrobacter | 0.0063 | Streptomyces | 0.0078 | Methylibium | 0.0060 |
| Shinella | 0.0063 | Methylibium | 0.0075 | Acidovorax | 0.0054 |
| Acidovorax | 0.0046 | Bacillus | 0.0066 | Niastella | 0.0053 |
| Niastella | 0.0040 | Arthrobacter | 0.0054 | Arthrobacter | 0.0048 |

TABLE 11B
The average abundance of fungal genera, as a proportion of the microbial
community, in root tissue of water stressed soybean plants grown from seeds treated
with the control Penicillium endophyte Strain F, the beneficial Penicillium endophyte
Strain B, and formulation control are shown. The average abundance of fungi in the
Rhizophagus genera were increased from approximately 9.7% of the fungal community of
untreated soybean roots and 9.8% of the fungal community in Strain F treated soybean
roots to approximately 30.5% of the fungal community of Strain B treated soybean roots.
Treatment with Strain B resulted in a 214.4% increase in the abundance of fungi in the
Rhizophagus genera in soybean roots relative to untreated controls.

| | | | | | Formulation |
|---|---|---|---|---|---|
| Genus | Strain B | Genus | Strain F | Genus | Control |
| Rhizophagus | 0.3050 | Claroideoglomus | 0.1993 | Claroideoglomus | 0.2105 |
| Claroideoglomus | 0.1591 | Olpidium | 0.1390 | Olpidium | 0.2040 |
| Funneliformis | 0.0789 | Rhizophagus | 0.0980 | Funneliformis | 0.0977 |
| Olpidium | 0.0504 | Funneliformis | 0.0774 | Rhizophagus | 0.0970 |
| Podospora | 0.0495 | Podospora | 0.0384 | Glomus | 0.0693 |
| Haematonectria | 0.0424 | Haematonectria | 0.0335 | Podospora | 0.0421 |
| Glomus | 0.0337 | Glomus | 0.0295 | Haematonectria | 0.0326 |
| Ilyonectria | 0.0194 | Fusarium | 0.0270 | Ilyonectria | 0.0147 |
| Codinaeopsis | 0.0150 | unidentified | 0.0169 | Fusarium | 0.0145 |
| Fusarium | 0.0113 | Coprinellus | 0.0165 | Chaetomium | 0.0068 |
| Chaetomium | 0.0066 | Conocybe | 0.0145 | unidentified | 0.0062 |
| unidentified | 0.0055 | Chaetomium | 0.0058 | Thielaviopsis | 0.0036 |
| Corollospora | 0.0044 | Corollospora | 0.0057 | Coprinellus | 0.0033 |
| Conocybe | 0.0042 | Zopfiella | 0.0055 | Zopfiella | 0.0024 |
| Zopfiella | 0.0042 | Cladosporium | 0.0034 | Corollospora | 0.0019 |
| unidentified | 0.0032 | Hydnomerulius | 0.0032 | Clonostachys | 0.0019 |
| Lophiostoma | 0.0031 | Myrothecium | 0.0029 | Paraglomus | 0.0016 |
| Thielaviopsis | 0.0029 | unidentified | 0.0024 | Myrothecium | 0.0016 |
| Ambispora | 0.0024 | Clonostachys | 0.0021 | Aureobasidium | 0.0015 |
| Pseudeurotium | 0.0024 | Paraphaeosphaeria | 0.0021 | Sarocladium | 0.0009 |

TABLE 11C
The average abundance of bacterial families, as a proportion of the microbial
community, in leaf tissue of water stressed soybean plants grown from seeds treated
with the control Penicillium endophyte Strain F, the beneficial Penicillium endophyte
Strain B, and formulation control are shown.

| | | | | | Formulation |
|---|---|---|---|---|---|
| Family | Strain B | Family | Strain F | Family | Control |
| Enterobacteriaceae | 0.1687 | Enterobacteriaceae | 0.1552 | Enterobacteriaceae | 0.2297 |
| Bradyrhizobiaceae | 0.0954 | Comamonadaceae | 0.0885 | Bradyrhizobiaceae | 0.1382 |
| Comamonadaceae | 0.0682 | Bradyrhizobiaceae | 0.0649 | Comamonadaceae | 0.0799 |
| Pseudomonadaceae | 0.0544 | Pseudomonadaceae | 0.0514 | Pseudomonadaceae | 0.0457 |
| Flavobacteriaceae | 0.0278 | Rhizobiaceae | 0.0422 | Flavobacteriaceae | 0.0311 |
| Rhizobiaceae | 0.0245 | Flavobacteriaceae | 0.0356 | Rhizobiaceae | 0.0274 |
| Methylophilaceae | 0.0214 | Burkholderiaceae | 0.0355 | Methylophilaceae | 0.0222 |
| Cytophagaceae | 0.0212 | Cytophagaceae | 0.0292 | Cytophagaceae | 0.0212 |
| Oxalobacteraceae | 0.0180 | Methylophilaceae | 0.0247 | Oxalobacteraceae | 0.0184 |
| Hyphomicrobiaceae | 0.0151 | Oxalobacteraceae | 0.0231 | Anaerolineaceae | 0.0118 |
| Anaerolineaceae | 0.0132 | Hyphomicrobiaceae | 0.0149 | Chitinophagaceae | 0.0117 |
| Caulobacteraceae | 0.0115 | Caulobacteraceae | 0.0143 | Hyphomicrobiaceae | 0.0110 |
| Bacillaceae | 0.0108 | Chitinophagaceae | 0.0108 | Caulobacteraceae | 0.0090 |
| Chitinophagaceae | 0.0101 | Anaerolineaceae | 0.0081 | Sphingomonadaceae | 0.0081 |
| Planctomycetaceae | 0.0093 | Xanthomonadaceae | 0.0080 | Bacillaceae | 0.0077 |
| Streptomycetaceae | 0.0079 | Streptomycetaceae | 0.0078 | SHA-31 | 0.0063 |
| SHA-31 | 0.0068 | Planctomycetaceae | 0.0077 | Enterococcaceae | 0.0061 |
| Xanthomonadaceae | 0.0064 | Bacillaceae | 0.0066 | Rhodospirillaceae | 0.0053 |
| Micrococcaceae | 0.0063 | Sphingomonadaceae | 0.0060 | Xanthomonadaceae | 0.0051 |
| Rhodospirillaceae | 0.0052 | Saprospiraceae | 0.0058 | Micrococcaceae | 0.0048 |

TABLE 11D
The average abundance of bacterial families, as a proportion of the microbial
community, in root tissue of water stressed soybean plants grown from seeds
treated with th control Penicillium endophyte Strain F, the beneficial
Penicillium endophyte Strain B, and formulation control are shown.

| | | | | | Formulation |
|---|---|---|---|---|---|
| Family | Strain B | Family | Strain F | Family | Control |
| Bradyrhizobiaceae | 0.1337 | Comamonadaceae | 0.1061 | Bradyrhizobiaceae | 0.2016 |
| Pseudomonadaceae | 0.1085 | Pseudomonadaceae | 0.0999 | Comamonadaceae | 0.0858 |
| Comamonadaceae | 0.1014 | Bradyrhizobiaceae | 0.0861 | Pseudomonadaceae | 0.0748 |
| Flavobacteriaceae | 0.0554 | Flavobacteriaceae | 0.0586 | Flavobacteriaceae | 0.0590 |
| Cytophagaceae | 0.0431 | Cytophagaceae | 0.0455 | Cytophagaceae | 0.0455 |
| Methylophilaceae | 0.0376 | Methylophilaceae | 0.0455 | Methylophilaceae | 0.0381 |
| Rhizobiaceae | 0.0292 | Oxalobacteraceae | 0.0357 | Rhizobiaceae | 0.0271 |
| Caulobacteraceae | 0.0149 | Rhizobiaceae | 0.0320 | Chitinophagaceae | 0.0205 |
| Oxalobacteraceae | 0.0147 | Caulobacteraceae | 0.0197 | Caulobacteraceae | 0.0177 |
| Hyphomicrobiaceae | 0.0140 | Chitinophagaceae | 0.0185 | Oxalobacteraceae | 0.0165 |
| Chitinophagaceae | 0.0135 | Hyphomicrobiaceae | 0.0133 | Hyphomicrobiaceae | 0.0120 |
| Streptomycetaceae | 0.0134 | Streptomycetaceae | 0.0112 | Streptomycetaceae | 0.0097 |
| Micrococcaceae | 0.0058 | Micrococcaceae | 0.0063 | Chromatiaceae | 0.0069 |
| Anaerolineaceae | 0.0055 | Rhodospirillaceae | 0.0060 | Anaerolineaceae | 0.0057 |
| Opitutaceae | 0.0051 | Sphingomonadaceae | 0.0049 | Micrococcaceae | 0.0055 |
| Rhodospirillaceae | 0.0045 | Anaerolineaceae | 0.0049 | Burkholderiaceae | 0.0051 |
| Bacillaceae | 0.0044 | Burkholderiaceae | 0.0044 | Rhodospirillaceae | 0.0046 |
| Alteromonadaceae | 0.0044 | Acidobacteria | 0.0042 | Xanthomonadaceae | 0.0042 |
| | | Subgroup 4, | | | |
| | | unidentified family | | | |
| Xanthomonadaceae | 0.0040 | Opitutaceae | 0.0041 | Acidobacteria | 0.0035 |
| | | | | Subgroup 4, | |
| | | | | unidentified family | |
| Chromatiaceae | 0.0039 | Nitrosomonadaceae | 0.0033 | Sphingomonadaceae | 0.0034 |

TABLE HE
The following OTUs were found in all biological replicates of samples of plants grown from seeds treated
with Strain B, but not found in plants grown from seeds treated with Strain F or formulation control.

| | Tis- | Do- | | | | | |
|---|---|---|---|---|---|---|---|
| QTU | sue | main | Phylum | Class | Order | Family | Genus |
| B1.0\|REF97_V4\|126313 | leaf | Bacteria | Chloroflexi | Anaerolineae | Anaerolineales | Anaerolineaceae | |
| B1.0\|REF97_V4\|24209 | leaf | Bacteria | Proteobacteria | Alphaproteobacteria | Sphingomonadales | Erythro- | Erythrobacter |
| | | | | | | bacteraceae | |
| B1.0\|REF97_V4\|244563 | leaf | Bacteria | Proteobacteria | Gammaproteobacteria | Order_Incer- | Family_Incer- | Marinicella |
| | | | | | tae_Sedis | tae_Sedis | |
| B1.0\|REF97_V4\|30291 | leaf | Bacteria | Proteobacteria | Gammaproteobacteria | Xanthomonadales | | |
| B1.0\|REF97_V4\|33060 | leaf | Bacteria | Actinobacteria | Acidimicrobiia | Acidimicrobiales | lamiaceae | lamia |
| B1.0\|REF97_V4\|42838 | leaf | Bacteria | Chloroflexi | KD4-96 | | | |
| B1.0\|REF97_V4\|44325 | leaf | Bacteria | Proteobacteria | Alphaproteobacteria | Caulobacterales | Hyphomonadaceae | Hirschia |
| B1.0\|REF97_V4\|58280 | leaf | Bacteria | Proteobacteria | Alphaproteobacteria | Caulobacterales | Hyphomonadaceae | Hirschia |
| B1.0\|REF99_V4\|112628 | leaf | Bacteria | Proteobacteria | Gammaproteobacteria | Xanthomonadales | Xanthomonadaceae | Thermomonas |
| B1.0\|REF99_V4\|125928 | leaf | Bacteria | Proteobacteria | Betaproteobacteria | TRA3-20 | | |
| B1.0\|REF99_V4\|127439 | leaf | Bacteria | Gemma- | Gemm-1 | | | |
| | | | timonadetes | | | | |
| B1.0\|REF99_V4\|13061 | leaf | Bacteria | Verrucomicrobia | Opitutae | Opitutales | Opitutaceae | Opitutus |
| B1.0\|REF99_V4\|147488 | leaf | Bacteria | Chloroflexi | Anaerolineae | Anaerolineales | Anaerolineaceae | |
| B1.0\|REF99_V4\|16720 | leaf | Bacteria | Chloroflexi | Anaerolineae | Anaerolineales | Anaerolineaceae | |
| B1.0\|REF99_V4\|217009 | leaf | Bacteria | Acidobacteria | Acidobacteria | Subgroup_6 | | |
| B1.0\|REF99_V4\|217669 | leaf | Bacteria | Planctomycetes | Planctomycetacia | Planctomycetales | Planctomycetaceae | |
| B1.0\|REF99_V4\|2218 | leaf | Bacteria | Actinobacteria | Actinobacteria | Propionibacteriales | Nocardioidaceae | Nocardioides |
| B1.0\|REF99_V4\|231107 | leaf | Bacteria | Proteobacteria | Gammaproteobacteria | Oceanospirillales | Oceanospirillaceae | Pseudospirillum |
| B1.0\|REF99_V4\|26043 | leaf | Bacteria | Actinobacteria | Actinobacteria | Micrococcales | Microbacteriaceae | Schumannella |
| B1.0\|REF99_V4\|30882 | leaf | Bacteria | Proteobacteria | Gammaproteobacteria | Xanthomonadales | Solimonadaceae | Fontimonas |
| B1.0\|REF99_V4\|3454 | leaf | Bacteria | Proteobacteria | Alphaproteobacteria | Rhizobiales | Xanthobacteraceae | |
| B1.0\|REF99_V4\|4087 | leaf | Bacteria | Proteobacteria | Alphaproteobacteria | Sphingomonadales | Sphingomonadaceae | Novosphingobium |
| B1.0\|REF99_V4\|4443 | leaf | Bacteria | Proteobacteria | Gammaproteobacteria | Xanthomonadales | | |
| B1.0\|REF99_V4\|7734 | leaf | Bacteria | Actinobacteria | Actinobacteria | Micrococcales | Microbacteriaceae | Lysinimonas |
| B1.0\|REF99_V4\|78003 | leaf | Bacteria | Proteobacteria | Alphaproteobacteria | Rhizobiales | Xanthobacteraceae | Pseudolabrys |
| B1.0\|REF99_V4\|7892 | leaf | Bacteria | Proteobacteria | Alphaproteobacteria | Sphingomonadales | Sphingomonadaceae | Sphingomonas |
| B1.0\|SYM97_V4\|694 | leaf | Bacteria | Planctomycetes | OM190 | agg27 | | |
| B1.0\|REF97_V4\|10576 | root | Bacteria | Bacteroidetes | Flavobacteriia | Flavobacteriales | Flavobacteriaceae | |
| B1.0\|REF97_V4\|137844 | root | Bacteria | Proteobacteria | Gammaproteobacteria | Xanthomonadales | Xanthomonadaceae | |
| B1.0\|REF97_V4\|145009 | root | Bacteria | Proteobacteria | Deltaproteobacteria | Myxococcales | | |
| B1.0\|REF97_V4\|214952 | root | Bacteria | Verrucomicrobia | Opitutae | Opitutales | Opitutaceae | Opitutus |
| B1.0\|REF97_V4\|66257 | root | Bacteria | Acidobacteria | Acidobacteria | Subgroup_17 | | |
| B1.0\|REF97_V4\|74770 | root | Bacteria | Firmicutes | Erysipelotrichia | Erysipelotrichales | Erysipelotrichaceae | Asteroleplasma |
| B1.0\|REF97_V4\|76872 | root | Bacteria | Proteobacteria | Gammaproteobacteria | Xanthomonadales | Xanthomonadaceae | Stenotrophomonas |
| B1.0\|REF99_V4\|10645 | root | Bacteria | Proteobacteria | Gammaproteobacteria | Pseudomonadales | Pseudomonadaceae | Pseudomonas |
| B1.0\|REF99_V4\|109230 | root | Bacteria | Proteobacteria | Alphaproteobacteria | Rhodospirillales | Rhodospirillaceae | Defluviicoccus |
| B1.0\|REF99_V4\|122980 | root | Bacteria | Acidobacteria | Acidobacteria | Subgroup_3 | PAUC26f | |
| B1.0\|REF99_V4\|139185 | root | Bacteria | Proteobacteria | Deltaproteobacteria | Myxococcales | | |
| B1.0\|REF99_V4\|142 | root | Bacteria | Proteobacteria | Gammaproteobacteria | Pseudomonadales | Pseudomonadaceae | Pseudomonas |
| B1.0\|REF99_V4\|148539 | root | Bacteria | Bacteroidetes | Flavobacteriia | Flavobacteriales | Cryomorphaceae | Fluviicola |
| B1.0\|REF99_V4\|151 | root | Bacteria | Firmicutes | Bacilli | Bacillales | Bacillaceae | Bacillus |
| B1.0\|REF99_V4\|191 | root | Bacteria | Proteobacteria | Alphaproteobacteria | Rhizobiales | Bradyrhizobiaceae | Bradyrhizobium |
| B1.0\|REF99_V4\|1984 | root | Bacteria | Actinobacteria | Actinobacteria | Micromonosporales | Micro- | Actinoplanes |
| | | | | | | monosporaceae | |
| B1.0\|REF99_V4\|199235 | root | Bacteria | Proteobacteria | Betaproteobacteria | | | |
| B1.0\|REF99_V4\|211 | root | Bacteria | Actinobacteria | Actinobacteria | Streptomycetales | Streptomycetaceae | Streptomyces |
| B1.0\|REF99_V4\|28603 | root | Bacteria | Actinobacteria | Actinobacteria | Streptomycetales | Streptomycetaceae | Streptomyces |
| B1.0\|REF99_V4\|33692 | root | Bacteria | Proteobacteria | Betaproteobacteria | Burkholderiales | Comamonadaceae | |
| B1.0\|REF99_V4\|49912 | root | Bacteria | Proteobacteria | Gammaproteobacteria | Alteromonadales | Alteromonadaceae | Marinobacter |
| B1.0\|REF99_V4\|64 | root | Bacteria | Proteobacteria | Gammaproteobacteria | Pseudomonadales | Pseudomonadaceae | Pseudomonas |
| B1.0\|REF99_V4\|643 | root | Bacteria | Actinobacteria | Actinobacteria | Micrococcales | Micrococcaceae | Arthrobacter |
| B1.0\|REF99_V4\|74770 | root | Bacteria | Firmicutes | Erysipelotrichia | Erysipelotrichales | Erysipelotrichaceae | Asteroleplasma |
| B1.0\|REF99_V4\|88218 | root | Bacteria | Proteobacteria | Gammaproteobacteria | Alteromonadales | Alteromonadaceae | Glaciecola |
| B1.0\|SYM97_V4\|1685 | root | Bacteria | Proteobacteria | Gammaproteobacteria | | | |
| F1.0\|SYM97_ITS2\|1122 | root | Fungi | Ascomycota | Eurotiomycetes | Eurotiales | Frichocomaceae | Penicillium |
| F1.0\|SYM97_ITS2\|1601 | root | Fungi | Glomeromycota | Glomeromycetes | Glomerales | Glomeraceae | Rhizophagus |
| F1.0\|SYM97_ITS2\|1660 | root | Fungi | Glomeromycota | Glomeromycetes | Paraglomerales | Paraglomeraceae | Paraglomus |
| F1.0\|SYM97_ITS2\|662 | root | Fungi | Ascomycota | Dothideomycetes | Incertae sedis | Pseudeurotiaceae | Pseudeurotium |
| F1.0\|UDYN_ITS2\|460 | root | Fungi | Ascomycota | Dothideomycetes | Pleosporales | | |

TABLE 11F
The following OTUs were found to have significant differences in abundance between plants grown from
seeds treated with Strain B versus plants grown from seeds treated with the formulation control.

| | | | | For- | | | | | | |
|---|---|---|---|---|---|---|---|---|---|---|
| | tis- | Strain | Strain | mula- | King- | | | | | |
| OTU | sue | B | F | tion | dom | Phylum | Class | Order | Family | Genus |
| F1.0 | roots | 9423 | 84 | 0 | Fungi | Ascomycota | Dothideo- | Incertae sedis | Pseudeurotiaceae | Pseudeurotium |
| SYM97_ITS2 | | | | | | | mycetes | | | |
| 662 | | | | | | | | | | |
| B1.0 | leaf | 5166 | 872 | 0 | Bac- | Chloroflexi | Anaer- | | | |
| SYM97_V4 | | | | | teria | | olineae | | | |
| 2013 | | | | | | | | | | |
| B1.0 | leaf | 2997 | 0 | 0 | Bac- | Firmicutes | Clostridia | Clostridiales | Ruminococcaceae | |
| REF99_V4 | | | | | teria | | | | | |
| 120275 | | | | | | | | | | |
| B1.0 | leaf | 2904 | 1281 | 0 | Bac- | Proteo- | Alpha- | Sphin- | Sphin- | Sphingomonas |
| REF99_V4 | | | | | teria | bacteria | proteo- | gomonadales | gomonadaceae | |
| 7892 | | | | | | | bacteria | | | |
| B1.0 | leaf | 1968 | 0 | 0 | Bac- | Chloroflexi | KD4-96 | | | |
| REF97_V4 | | | | | teria | | | | | |
| 42838 | | | | | | | | | | |
| B1.0 | leaf | 2490 | 0 | 0 | Bac- | Proteo- | Alpha- | Sphin- | Erythro- | Altereryth- |
| REF99_V4 | | | | | teria | bacteria | proteo- | gomonadales | bacteraceae | robacter |
| 180402 | | | | | | | bacteria | | | |
| B1.0 | leaf | 3152 | 1722 | 0 | Bac- | Chloroflexi | Anaer- | SBR1031 | A4b | |
| REF99_V4 | | | | | teria | | olineae | | | |
| 144927 | | | | | | | | | | |
| B1.0 | leaf | 2645 | 131 | 0 | Bac- | Proteo- | Beta- | TRA3-20 | | |
| REF99_V4 | | | | | teria | bacteria | proteo- | | | |
| 125928 | | | | | | | bacteria | | | |
| B1.0 | leaf | 2416 | 204 | 0 | Bac- | Actino- | Acidimi- | Acidimi- | lamiaceae | lamia |
| REF97_V4 | | | | | teria | bacteria | crobiia | crobiales | | |
| 69488 | | | | | | | | | | |
| B1.0 | leaf | 2630 | 1300 | 0 | Bac- | Firmicutes | Bacilli | Bacillales | Paenibacillaceae | |
| REF99_V4 | | | | | teria | | | | | |
| 26508 | | | | | | | | | | |
| B1.0 | leaf | 6336 | 219 | 158 | Bac- | Chloroflexi | Anaer- | Anaerolineales | Anaerolineaceae | |
| REF99_V4 | | | | | teria | | olineae | | | |
| 16720 | | | | | | | | | | |
| F1.0 | root | 23400 | 5700 | 168 | Fungi | Ascomycota | Sordario- | Sordariales | Lasiosphaeriaceae | Podospora |
| U97_ITS2 | | | | | | | mycetes | | | |
| 425 | | | | | | | | | | |
| F1.0 | root | 59809 | 1805 | 1576 | Fungi | Ascomycota | Sordario- | Chae- | Chae- | Codinaeopsis |
| SYM97_ITS2 | | | | | | | mycetes | tosphaeriales | tosphaeriaceae | |
| 1288 | | | | | | | | | | |
| F1.0 | root | 36851 | 4247 | 1175 | Fungi | Glom- | Glomero- | Glomerales | Glomeraceae | Rhizophagus |
| SYM97_ITS2 | | | | | | eromycota | mycetes | | | |
| 1548 | | | | | | | | | | |
| F1.0 | root | 464106 | 50464 | 31780 | Fungi | Glom- | Glomero- | Glomerales | Glomeraceae | Rhizophagus |
| SYM97_ITS2 | | | | | | eromycota | mycetes | | | |
| 1518 | | | | | | | | | | |
| B1.0 | root | 1754 | 426 | 247 | Bac- | Proteo- | Gamma- | Alteromonadales | Alteromonadaceae | Glaciecola |
| REF99_V4 | | | | | teria | bacteria | proteo- | | | |
| 88218 | | | | | | | bacteria | | | |
| B1.0 | root | 5866 | 17608 | 20295 | Bac- | Proteo- | Beta- | Burkholderiales | Burkholderiaceae | Candida- |
| SYM97_V4 | | | | | teria | bacteria | proteo- | | | tus_Glomeribacter |
| 231 | | | | | | | bacteria | | | |
| B1.0 | root | 548 | 417 | 2711 | Bac- | Proteo- | Gamma- | Entero- | Entero- | Klebsiella |
| REF99_V4 | | | | | teria | bacteria | proteo- | bacteriales | bacteriaceae | |
| 1 | | | | | | | bacteria | | | |
| B1.0 | leaf | 18 | 3199 | 2229 | Bac- | Chloroflexi | Anaer- | | | |
| SYM97_V4 | | | | | teria | | olineae | | | |
| 455 | | | | | | | | | | |
| B1.0 | leaf | 54 | 15017 | 8129 | Bac- | Proteo- | Alpha- | Rhizobiales | Rhizobiaceae | Rhizobium |
| REF99_V4 | | | | | teria | bacteria | proteo- | | | |
| 142567 | | | | | | | bacteria | | | |
| B1.0 | leaf | 0 | 1976 | 1805 | Bac- | Proteo- | Beta- | Burkholderiales | Comamonadaceae | Comamonas |
| REF99_V4 | | | | | teria | bacteria | proteo- | | | |
| 660 | | | | | | | bacteria | | | |
| B1.0 | leaf | 0 | 708 | 2454 | Bac- | Proteo- | Gamma- | | | |
| SYM97_V4 | | | | | teria | bacteria | proteo- | | | |
| 2277 | | | | | | | bacteria | | | |
| B1.0 | leaf | 0 | 0 | 3990 | Bac- | Firmicutes | | | | |
| SYM97_V4 | | | | | teria | | | | | |
| 1678 | | | | | | | | | | |
| B1.0 | leaf | 0 | 0 | 4108 | Bac- | Chloroflexi | Anaer- | H39 | | |
| REF97_V4 | | | | | teria | | olineae | | | |
| 241577 | | | | | | | | | | |

TABLE 11G
The average abundance of bacterial genera, as a proportion of the microbial
community, in root tissue of water stressed soybean plants grown from seeds treated
with the control Penicillium endophyte Strain F, the beneficial Penicillium
endophyte Strain B, and formulation control are shown.

| | | | | | Formulation |
|---|---|---|---|---|---|
| Genus | Strain B | Genus | Strain F | Genus | Control |
| Bradyrhizobium | 0.1310 | Bradyrhizobium | 0.0832 | Bradyrhizobium | 0.1989 |
| Cellvibrio | 0.0872 | Cellvibrio | 0.0787 | Cellvibrio | 0.0620 |
| Flavobacterium | 0.0479 | Flavobacterium | 0.0533 | Flavobacterium | 0.0548 |
| Piscinibacter | 0.0284 | Methylotenera | 0.0356 | Methylotenera | 0.0294 |
| Methylotenera | 0.0283 | Massilia | 0.0336 | Rhizobium | 0.0219 |
| Rhizobium | 0.0217 | Piscinibacter | 0.0264 | Piscinibacter | 0.0199 |
| Pseudomonas | 0.0212 | Rhizobium | 0.0243 | Hydrogenophaga | 0.0180 |
| Hydrogenophaga | 0.0208 | Hydrogenophaga | 0.0231 | Asticcacaulis | 0.0177 |
| Ohtaekwangia | 0.0197 | Pseudomonas | 0.0212 | Ohtaekwangia | 0.0164 |
| Devosia | 0.0140 | Ohtaekwangia | 0.0180 | Massilia | 0.0136 |
| Asticcacaulis | 0.0139 | Asticcacaulis | 0.0174 | Pseudomonas | 0.0128 |
| Massilia | 0.0137 | Devosia | 0.0133 | Dyadobacter | 0.0124 |
| Streptomyces | 0.0134 | Streptomyces | 0.0112 | Devosia | 0.0120 |
| Methylibium | 0.0080 | Dyadobacter | 0.0103 | Streptomyces | 0.0097 |
| Acidovorax | 0.0077 | Acidovorax | 0.0095 | Niastella | 0.0089 |
| Shinella | 0.0075 | Niastella | 0.0083 | Rheinheimera | 0.0069 |
| Dyadobacter | 0.0062 | Shinella | 0.0077 | Acidovorax | 0.0065 |
| Niastella | 0.0059 | Leptothrix | 0.0067 | Methylibium | 0.0056 |
| Arthrobacter | 0.0058 | Methylibium | 0.0064 | Arthrobacter | 0.0055 |
| Pseudorhodoferax | 0.0051 | Arthrobacter | 0.0063 | Shinella | 0.0052 |

TABLE 11H
The average abundance of fungal families, as a proportion of the microbial community, in root tissue
of water stressed soybean plants grown from seeds treated with the control Penicillium endophyte
Strain F, the beneficial Penicillium endophyte Strain B, and formulation control are shown.

| | | | | | Formulation |
|---|---|---|---|---|---|
| Family | Strain B | Family | Strain F | Family | Control |
| Glomeraceae | 0.4383 | Glomeraceae | 0.2573 | Glomeraceae | 0.2825 |
| Claroideoglomeraceae | 0.1611 | Nectriaceae | 0.2098 | Claroideoglomeraceae | 0.2156 |
| Nectriaceae | 0.1351 | Claroideoglomeraceae | 0.2038 | Olpidiaceae | 0.2040 |
| Lasiosphaeriaceae | 0.0666 | Olpidiaceae | 0.1390 | Nectriaceae | 0.1374 |
| Olpidiaceae | 0.0504 | Lasiosphaeriaceae | 0.0573 | Lasiosphaeriaceae | 0.0658 |
| Hypocreales, | 0.0199 | Psathyrellaceae | 0.0165 | Hypocreales, | 0.0171 |
| unidentified family | | | | unidentified family | |
| Chaetosphaeriaceae | 0.0150 | Bolbitiaceae | 0.0145 | Chaetomiaceae | 0.0068 |
| Chaetomiaceae | 0.0078 | Chaetomiaceae | 0.0058 | Ceratocystidaceae | 0.0036 |
| Halosphaeriaceae | 0.0044 | Halosphaeriaceae | 0.0057 | Auriculariales, | 0.0035 |
| | | | | unidentified family | |
| Bolbitiaceae | 0.0042 | Hypocreales, | 0.0038 | Psathyrellaceae | 0.0033 |
| | | unidentified family | | | |
| Pleosporales, | 0.0032 | Davidiellaceae | 0.0034 | Halosphaeriaceae | 0.0019 |
| unidentified family | | | | | |
| Lophiostomataceae | 0.0031 | Paxillaceae | 0.0032 | Bionectriaceae | 0.0019 |
| Ceratocystidaceae | 0.0029 | Helotiales, | 0.0026 | Paraglomeraceae | 0.0016 |
| | | unidentified family | | | |
| Ambisporaceae | 0.0024 | Pleosporales, | 0.0024 | Dothioraceae | 0.0015 |
| | | unidentified family | | | |
| Pseudeurotiaceae | 0.0024 | Bionectriaceae | 0.0021 | Helotiales, | 0.0010 |
| | | | | unidentified family | |
| Davidiellaceae | 0.0023 | Montagnulaceae | 0.0021 | Agaricaceae | 0.0007 |
| Bionectriaceae | 0.0023 | Eremotheciaceae | 0.0018 | Paraglomerales, | 0.0007 |
| | | | | unidentified family | |
| Glomerales, | 0.0016 | Hypocreaceae | 0.0016 | Clavicipitaceae | 0.0007 |
| unidentified family | | | | | |
| Paraglomeraceae | 0.0015 | Agaricaceae | 0.0012 | Archaeosporaceae | 0.0006 |
| Trichocomaceae | 0.0013 | Auriculariales, | 0.0012 | Eremotheciaceae | 0.0005 |
| | | unidentified family | | | |

### Table 12: Field Trial Results

TABLE 12A
Soybean field trial results. No negative impact on soybean
plants grown from seeds treated with Strain B was observed
during well-watered field trial conditions.

| | Average across 1 location | | |
|---|---|---|---|
| | Harvest Yield | Weight | |
| Treatment | bu/ac | lb/2row | Moisture % |
| Variety 1_Formulation | 63.34 | 17.44 | 12.94 |
| Variety 1_Strain B | 61.93 | 17.08 | 13.09 |
| Variety 2_Formulation | 61.98 | 17.12 | 13.24 |
| Variety 2_Strain B | 62.33 | 17.18 | 13.05 |

TABLE 12B
Maize field trial results. No negative impact on maize
plants grown from seeds treated with Strain B was observed
during well-watered field trial conditions.

Average across 2 locations

| | Combine Moisture | Combine |
|---|---|---|
| Treatment | (%) | Test Wt |
| Variety1_Formulation | 26.20 | 54.87 |
| Variety1_Strain B | 26.10 | 54.60 |
| Variety2_Formulation | 29.08 | 51.94 |
| Variety2_Strain B | 29.04 | 52.58 |

### REFERENCES

Abarenkov, K., Henrik Nilsson, R., Larsson, K.-H., Alexander, I. J., Eberhardt, U., Erland, S., . . . Koljalg, U. (2010, April). The UNITE database for molecular identification of fungi—recent updates and future perspectives. The New Phytologist. England.
Agudelo-Romero, P., Erban, A., Sousa, L., Pais, M. S., Kopka, J., and Fortes, A. M. (2013). Search for Transcriptional and Metabolic Markers of Grape Pre-Ripening and Ripening and Insights into Specific Aroma Development in Three Portuguese Cultivars. PLoS ONE 8, e60422.
Albers, E. (2009). Metabolic characteristics and importance of the universal methionine salvage pathway recycling methionine from 5′-methylthioadenosine. IUBMB Life 61, 1132-1142.
Albrigo, L. Gene; Aritua, Valente; Wang, Nian; & Achor, Diann. (2014). Phloem disruption from HLB infection in canopy and root framework. Journal of Citrus Pathology, 1(1).
Allen, J. F. (2004). Cytochrome b6f: structure for signalling and vectorial metabolism. Trends Plant Sci. 9, 130-137.
Amann et al. (2001) Current Opinion in Biotechnology 12:231-236
Ambrose, K. V. and Belanger, F. C., 2012. SOLiD-SAGE of endophyte-infected red fescue reveals numerous effects on host transcriptome and an abundance of highly expressed fungal secreted proteins. PLoS One, 7(12), p.e53214.
Ana Victoria Garcia, Mohamed Al-Yousif, Heribert Hirt. 2012. Role of AGC kinases in plant growth and stress responses. Cellular and Molecular Life Sciences. 69(19):3259-67.
Anderson, J., Chow, W., and Goodchild, D. (1988). Thylakoid Membrane Organisation in Sun/Shade Acclimation. Funct. Plant Biol. 15, 11-26.
Andersson, M. X., Hamberg, M., Kourtchenko, O., Brunnstrom, A., McPhail, K. L., Gerwick, W. H., Göbel, C., Feussner, I., and Ellerstrom, M. (2006). Oxylipin profiling of the hypersensitive response in Arabidopsis thaliana. Formation of a novel oxo-phytodienoic acid-containing galactolipid, arabidopside E. J. Biol. Chem. 281, 31528-31537.
Angelini, R., Bragaloni, M., Federico, R., Infantino, A., and Porta-Pugua, A. (1993). Involvement of Polyamines, Diamine Oxidase and Peroxidase in Resistance of Chickpea to Ascochyta rabiei. J. Plant Physiol. 142, 704-709.
Antunes, F., Aguilar, M., Pineda, M., and Sodek, L. (2008). Nitrogen stress and the expression of asparagine synthetase in roots and nodules of soybean (Glycine max). Physiol. Plant. 133, 736-743.
Appleby, C. A. (1984). Leghemoglobin and Rhizobium respiration. Annu. Rev. Plant Physiol. 35, 443-478.
Arnaud, N., Ravet, K., Borlotti, A., Touraine, B., Boucherez, J., Fizames, C., Briat, J.-F., Cellier, F., and Gaymard, F. (2007). The iron-responsive element (IRE)/iron-regulatory protein 1 (IRP1)-cytosolic aconitase iron-regulatory switch does not operate in plants. Biochem. J. 405, 523.
Aviezer-Hagai, K., Skovorodnikova, J., Galigniana, M., Farchi-Pisanty, O., Maayan, E., Bocovza, S., Efrat, Y., Koskull-Döring, P. von, Ohad, N., and Breiman, A. (2006). Arabidopsis immunophilins ROF1 (AtFKBP62) and ROF2 (AtFKBP65) exhibit tissue specificity, are heat-stress induced, and bind HSP90. Plant Mol. Biol. 63, 237-255.
Azuma, Y., Yamagishi, M., Ueshima, R., and Ishihama, A. (1991). Cloning and sequence determination of the Schizosaccharomyces pombe rpb 1 gene encoding the largest subunit of RNA polymerase II. Nucleic Acids Res. 19, 461-468.
Bais, H. P., and Ravishankar, G. A. (2002). Role of polyamines in the ontogeny of plants and their biotechnological applications. Plant Cell Tissue Organ Cult. 69, 1-34.
Baker, J., dennSteele, C. V., and Iii, L. D. (1988). Sequence and characterization of 6 Lea proteins and their genes from cotton. Plant Mol. Biol. 11, 277-291.
Balmer, Y., Vensel, W. H., Cai, N., Manieri, W., Schürmann, P., Hurkman, W. J., and Buchanan, B. B. (2006). A complete ferredoxin/thioredoxin system regulates fundamental processes in amyloplasts. Proc. Natl. Acad. Sci. U.S.A 103, 2988-2993.
Bargmann, B. O. R., and Munnik, T. (2006). The role of phospholipase D in plant stress responses. Curr. Opin. Plant Biol. 9, 515-522.
Barka et al. 2006, Appl. Environ. Microbiol. 72:7246-7252
Bartsch, M., Kimura, M., and Subramanian, A.-R. (1982). Purification, primary structure, and homology relationships of a chloroplast ribosomal protein. Proc. Natl. Acad. Sci. 79, 6871-6875
Batista-Santos, P., Lidon, F. C., Fortunato, A., Leitao, A. E., Lopes, E., Partelli, F., Ribeiro, A. I., and Ramalho, J. C. (2011). The impact of cold on photosynthesis in genotypes of Coffea spp.—Photosystem sensitivity, photoprotective mechanisms and gene expression. J. Plant Physiol. 168, 792-806.
Beckers, G. J. M., and Spoel, S. H. (2006). Fine-Tuning Plant Defence Signalling: Salicylate versus Jasmonate. Plant Biol. 8, 1-10.
Begum, A. A., Leibovitch, S., Migner, P., and Zhang, F. 2001. Specific flavonoids induced nod gene expression and pre-activated nod genes of Rhizobium leguminosarum increased pea (Pisum sativum L.) and lentil (Lens culinaris L.) nodulation in controlled growth chamber environments. J. Exp. Bot. 52, (360) 1537-1543.
Ben-Shem, A., Loubresse, N. G. de, Melnikov, S., Jenner, L., Yusupova, G., and Yusupov, M.
(2011). The Structure of the Eukaryotic Ribosome at 3.0 Å Resolution. Science 334, 1524-1529
Ben-Shem, A., Loubresse, N. G. de, Melnikov, S., Jenner, L., Yusupova, G., and Yusupov, M. (2011). The Structure of the Eukaryotic Ribosome at 3.0 Å Resolution. Science 334, 1524-1529.
Berger, S., Bell, E., Sadka, A., and Mullet, J. E. (1995). Arabidopsis thaliana Atvsp is homologous to soybean VspA and VspB, genes encoding vegetative storage protein acid phosphatases, and is regulated similarly by methyl jasmonate, wounding, sugars, light and phosphate. Plant Mol. Biol. 27, 933-942.
Bissoli, G., Niñoles, R., Fresquet, S., Palombieri, S., Bueso, E., Rubio, L., Garcia-Sánchez, M. J., Fernández, J. A., Mulet, J. M., and Serrano, R. (2012). Peptidyl-prolyl cis-trans isomerase ROF2 modulates intracellular pH homeostasis in Arabidopsis. Plant J. 70, 704-716.
Blatch, G. L. and Lässie, M. (1999). The tetratricopeptide repeat: a structural motif mediating protein-protein interactions. Bioessays. 21: 932-939.
Bordiec, S., Paquis, S., Lacroix, H., Dhondt, S., Ait Barka, E., Kauffmann, S., Jeandet, P., Mazeyrat-Gourbeyre, F., Clement, C., Baillieul, F., et al. (2011). Comparative analysis of defence responses induced by the endophytic plant growth-promoting rhizobacterium Burkholderia phytofirmans strain PsJN and the non-host bacterium Pseudomonas syringae pv. pisi in grapevine cell suspensions. J. Exp. Bot. 62, 595-603.
Boursiac, Y., Lee, S. M., Romanowsky, S., Blank, R., Sladek, C., Chung, W. S., and Harper, J. F. (2010). Disruption of the Vacuolar Calcium-ATPases in Arabidopsis Results in the Activation of a Salicylic Acid-Dependent Programmed Cell Death Pathway. Plant Physiol. 154, 1158-1171.
Bradley, D. J., Kjellbom, P., and Lamb, C. J. (1992). Elicitor- and wound-induced oxidative cross-linking of a proline-rich plant cell wall protein: A novel, rapid defense response. Cell 70, 21-30.
Brakhage, A. A., 2013. Regulation of fungal secondary metabolism. Nature Reviews Microbiology, 11(1), pp. 21-32.
Briat, J.-F., Ravet, K., Arnaud, N., Duc, C., Boucherez, J., Touraine, B., Cellier, F., and
Gaymard, F. (2010). New insights into ferritin synthesis and function highlight a link between iron homeostasis and oxidative stress in plants. Ann. Bot. 105, 811-822.
Brisson, L. F., Tenhaken, R., and Lamb, C. (1994). Function of Oxidative Cross-Linking of Cell Wall Structural Proteins in Plant Disease Resistance. Plant Cell 6, 1703-1712.
Brocker, C., Vasiliou, M., Carpenter, S., Carpenter, C., Zhang, Y., Wang, X., Kotchoni, S. O., Wood, A. J., Kirch, H.-H., Kopečný, D., et al. (2013). Aldehyde dehydrogenase (ALDH) superfamily in plants: gene nomenclature and comparative genomics. Planta 237, 189-210.
Bruce, R. J., and West, C. A. (1989). Elicitation of Lignin Biosynthesis and Isoperoxidase Activity by Pectic Fragments in Suspension Cultures of Castor Bean. Plant Physiol. 91, 889-897.
Buchanan, B. (1980). Role of Light in the Regulation of Chloroplast Enzymes. Annu. Rev. Plant Physiol. Plant Mol. Biol. 31, 341-374.
Burd et al. 2000, Can. J. Microbiol. 46:237-245
Bustin, M., and Reeves, R. (1996). High-Mobility-Group Chromosomal Proteins: Architectural Components That Facilitate Chromatin Function. In Progress in Nucleic Acid Research and Molecular Biology, W. E. C. and K. Moldave, ed. (Academic Press), pp. 35-100b.
Büttner, M. (2010). The Arabidopsis sugar transporter (AtSTP) family: an update. Plant Biol. 12, 35-41.
Caffall, K. H., and Mohnen, D. (2009). The structure, function, and biosynthesis of plant cell wall pectic polysaccharides. Carbohydr. Res. 344, 1879-1900.
Cao J. 2012. The Pectin Lyases in Arabidopsis thaliana: Evolution, Selection and Expression Profiles. PLoS One, 7(10): e46944.
Capaldi, R. A. (1990). Structure and Function of Cytochrome c Oxidase. Annu. Rev. Biochem. 59, 569-596.
Caplan, J., Padmanabhan, M., and Dinesh-Kumar, S. P. (2008). Plant NB-LRR Immune Receptors: From Recognition to Transcriptional Reprogramming. Cell Host Microbe 3, 126-135
Carpita, N. C., and Gibeaut, D. M. (1993). Structural models of primary cell walls in flowering plants: consistency of molecular structure with the physical properties of the walls during growth. Plant J. 3, 1-30.
Carvalho, A. de O., and Gomes, V. M. (2007). Role of plant lipid transfer proteins in plant cell physiology—A concise review. Peptides 28, 1144-1153.
Carvalho, G. A. B. de, Batista, J. S. S., Marcelino-Guimardes, F. C., Nascimento, L. C. do, and Hungria, M. (2013). Transcriptional analysis of genes involved in nodulation in soybean roots inoculated with Bradyrhizobium japonicum strain CPAC 15. BMC Genomics 14, 153.
Cawthorn, T. R., Moreno, J. C., Dharsee, M., Tran-Thanh, D., Ackloo, S., Zhu, P. H., Sardana, G., Chen, J., Kupchak, P., Jacks, L. M., et al. (2012). Proteomic Analyses Reveal High Expression of Decorin and Endoplasmin (HSP90B1) Are Associated with Breast Cancer Metastasis and Decreased Survival. PLoS ONE 7, e30992.
Ceccaroli, P., Buffalini, M., Saltarelli, R., Barbieri, E., Polidori, E., Ottonello, S., Kohler, A., Tisserant, E., Martin, F. and Stocchi, V., 2011. Genomic profiling of carbohydrate metabolism in the ectomycorrhizal fungus Tuber melanosporum. New Phytologist, 189(3), pp. 751-764.
Cha et al. (1998), Mol Plant-Microbe Interact 11:1119-1129
Cha, J.-Y., Su'udi, M., Kim, W.-Y., Kim, D. R., Kwak, Y.-S., and Son, D. (2012). Functional characterization of orchardgrass cytosolic Hsp70 (DgHsp70) and the negative regulation by Ca2+/AtCaM2 binding. Plant Physiol. Biochem. 58, 29-36.
Chen, B., Zhong, D., and Monteiro, A. (2006). Comparative genomics and evolution of the HSP90 family of genes across all kingdoms of organisms. BMC Genomics 7, 156.
Chen, H., McCaig, B. C., Melotto, M., He, S. Y., and Howe, G. A. (2004). Regulation of Plant Arginase by Wounding, Jasmonate, and the Phytotoxin Coronatine. J. Biol. Chem. 279, 45998-46007.
Chen, L., Ding, C., Zhao, X., Xu, J., Mohammad, A. A., Wang, S., and Ding, Y. (2014). Differential regulation of proteins in rice (Oryza sativa L.) under iron deficiency. Plant Cell Rep. 34, 83-96.
Cheng Wang, HaiFeng Chen, QingNan Hao, AiHua Sha, ZhiHui Shan, LiMiao Chen, Rong Zhou, HaiJian Zhi, XinAn Zhou. 2012. Transcript Profile of the Response of Two Soybean Genotypes to Potassium Deficiency. PLOS One. Published: Jul. 5, 2012. Accessed: May 27, 2016. <http://dx.doi.org/10.1371/journal.pone.0039856>
Cheong, Y. H., Chang, H., Gupta, R., Wang, X., Zhu, T., Luan, S. 2002. Transcriptional profiling reveals novel interactions between wounding, pathogen, abiotic stress, and hormonal responses in Arabidopsis. Plant Physiol. 129, 661-677.
Chern, M., Bai, W., Chen, X., Canlas, P. E., and Ronald, P. C. (2013). Reduced expression of glycolate oxidase leads to enhanced disease resistance in rice. PeerJ 1, e28.
Chernin et al. (1998) J Bacteriol 180:4435-4441
Chihaoui, S.-A., Mhadhbi, H., and Mhamdi, R. (2012). The antibiosis of nodule-endophytic agrobacteria and its potential effect on nodule functioning of Phaseolus vulgaris. Arch. Microbiol. 194, 1013-1021.
Chipman, D., Barak, Z., and Schloss, J. V. (1998). Biosynthesis of 2-aceto-2-hydroxy acids: acetolactate synthases and acetohydroxyacid synthases. Biochim. Biophys. Acta 1385, 401-419.
Choi et al. 2001, Planta 213:45-50
Chrisostomos Prodromou, Giuliano Siligardi, Ronan O'Brien, Derek N. Woolfson, Lynne Regan, Barry Panaretou, John E. Ladbury, Peter W. Piper, Laurence H. Pearl. 1999. Regulation of Hsp90 ATPase activity by tetratricopeptide repeat (TPR)-domain co-chaperones. The EMBO Journal. 18(3):754-62.
Christ, B., Süssenbacher, I., Moser, S., Bichsel, N., Egert, A., Müller, T., Kräutler, B., and Hörtensteiner, S. (2013). Cytochrome P450 CYP89A9 Is Involved in the Formation of Major Chlorophyll Catabolites during Leaf Senescence in Arabidopsis. Plant Cell 25, 1868-1880.
Christensen, S., Kaplan, F., Huffaker, A., Sims, J., Doehlemann, G., Teal, P., and Schmelz, E. (2015). The maize death acids, 10-oxo-11-phytoenoic acid and derivatives, demonstrate specificity in jasmonate-related signaling and defense. In 57th Annual Maize Genetics Conference, (Pheasant Run, St. Charles, Ill.).
Chung, H.-J., and Ferl, R. J. (1999). Arabidopsis Alcohol Dehydrogenase Expression in Both Shoots and Roots Is Conditioned by Root Growth Environment. Plant Physiol. 121, 429-436.
Churin, Y., Schilling, S., and Börner, T. (1999). A gene family encoding glutathione peroxidase homologues in Hordeum vulgare (barley)1. FEBS Lett. 459, 33-38.
Colletti, K. S., Tattersall, E. A., Pyke, K. A., Froelich, J. E., Stokes, K. D., and Osteryoung, K. W. (2000). A homologue of the bacterial cell division site-determining factor MinD mediates placement of the chloroplast division apparatus. Curr. Biol. 10, 507-516.
Compant, S., Reiter, B., Sessitsch, A., Nowak, J., Clément, C., and Ait Barka, E. (2005). Endophytic colonization of Vitis vinifera L. by plant growth-promoting bacterium Burkholderia sp. strain PsJN. Appl. Environ. Microbiol. 71, 1685-1693.
Cook, C. M., Daudi, A., Millar, D. J., Bindschedler, L. V., Khan, S., Bolwell, G. P., and Devoto, A. (2012). Transcriptional changes related to secondary wall formation in xylem of transgenic lines of tobacco altered for lignin or xylan content which show improved saccharification. Phytochemistry 74, 79-89.
Córdoba-Cañero, D., Dubois, E., Ariza, R. R., Doutriaux, M.-P., and Roldán-Arjona, T. (2010). Arabidopsis Uracil DNA Glycosylase (UNG) Is Required for Base Excision Repair of Uracil and Increases Plant Sensitivity to 5-Fluorouracil. J. Biol. Chem. 285, 7475-7483.
Cosgrove, D. J. (2000). Loosening of plant cell walls by expansins. Nature 407, 321-326.
Cox (1994) Methods Enzymol 235: 315-329
Crowell, D. N., John, M. E., Russell, D., and Amasino, R. M. (1992). Characterization of a stress-induced, developmentally regulated gene family from soybean. Plant Mol. Biol. 18, 459-466.
Cruz, J. A., Harfe, B., Radkowski, C. A., Dann, M. S., and McCarty, R. E. (1995). Molecular dissection of the epsilon subunit of the chloroplast ATP synthase of spinach. Plant Physiol. 109, 1379-1388.
Curi, G. C., Welchen, E., Chan, R. L., and Gonzalez, D. H. (2003). Nuclear and mitochondrial genes encoding cytochrome c oxidase subunits respond differently to the same metabolic factors. Plant Physiol. Biochem. 41, 689-693.
D. Xu, X. Duan, B. Wang, B. Hong, THD. Ho and R. Wu. 1996. Expression of a Late Embryogenesis Abundant Protein Gene, HVA1, from Barley Confers Tolerance to Water Deficit and Salt Stress in Transgenic Rice. Plant Physiology. 110(1):249-57.
Daniels et al., (2006). PNAS 103: 14965-14970
Dao, T. T. H., Linthorst, H. J. M., and Verpoorte, R. (2011). Chalcone synthase and its functions in plant resistance. Phytochem. Rev. 10, 397-412.
Dao, T. V., Nomura, M., Hamaguchi, R., Kato, K., Itakura, M., Minamisawa, K., Sinsuwongwat, S., Le, H. T.-P., Kaneko, T., Tabata, S., et al. (2008). NAD-Malic Enzyme Affects Nitrogen Fixing Activity of Bradyrhizobium japonicum USDA 110 Bacteroids in Soybean Nodules. Microbes Environ. 23, 215-220.
Davin, L. B., and Lewis, N. G. (2005). Dirigent phenoxy radical coupling: advances and challenges. Curr. Opin. Biotechnol. 16, 398-406.
De Luca, V. and St Pierre, B. 2000. The cell and developmental biology of alkaloid biosynthesis. Trends in Plant Science. 5, (4) 168-173.
De Maio, A. (1999). Heat shock proteins: facts, thoughts, and dreams. Shock Augusta Ga 11, 1-12.
Dennis and Webster 1971, Trans Brit Mycol Soc 57:25-39
Deshpande, N., Wilkins, M. R., Packer, N. and Nevalainen, H., 2008. Protein glycosylation pathways in filamentous fungi. Glycobiology, 18(8), pp. 626-637.
Desikan, R., Griffiths, R., Hancock, J., and Neill, S. (2002). A new role for an old enzyme: Nitrate reductase-mediated nitric oxide generation is required for abscisic acid-induced stomatal closure in Arabidopsis thaliana. Proc. Natl. Acad. Sci. 99, 16314-16318.
Devaiah, S. P., Roth, M. R., Baughman, E., Li, M., Tamura, P., Jeannotte, R., Welti, R., and Wang, X. (2006). Quantitative profiling of polar glycerolipid species from organs of wild-type Arabidopsis and a phospholipase Dalphal knockout mutant. Phytochemistry 67, 1907-1924.
Dhanapal, A. P., Ray, J. D., Singh, S. K., Hoyos-Villegas, V., Smith, J. R., Purcell, L. C., . . . & Fritschi, F. B. (2015). Genome-wide association analysis of diverse soybean genotypes reveals novel markers for nitrogen traits. The Plant Genome 8(3): 1-15
Di Pietro, A., González-Roncero, I., and Ruiz-Roldán, M. C. 2009. From tools of survival to weapons of destruction: The role of cell-wall degrading enzymes in plant infection. Plant Relationships. H. B. Deising, ed. Springer-Verlag, Berlin, pp. 275-306.
Ding, X., Cao, Y., Huang, L., Zhao, J., Xu, C., Li, X., and Wang, S. (2008). Activation of the Indole-3-Acetic Acid-Amido Synthetase GH3-8 Suppresses Expansin Expression and Promotes Salicylate- and Jasmonate-Independent Basal Immunity in Rice. Plant Cell 20, 228-240.
Dixon, R. A., and Paiva, N. L. (1995). Stress-Induced Phenylpropanoid Metabolism. Plant Cell 7, 1085-1097.
Donà, M., Macovei, A., Faè, M., Carbonera, D., and Balestrazzi, A. (2013). Plant hormone signaling and modulation of DNA repair under stressful conditions. Plant Cell Rep. 32, 1043-1052.
Dong, X. (2004). NPR1, all things considered. Curr. Opin. Plant Biol. 7, 547-552.
Kozich, J. J., Westcott, S. L., Baxter, N. T., Highlander, S. K., & Schloss, P. D. (2013). Development of a dual-index sequencing strategy and curation pipeline for analyzing amplicon sequence data on the miseq illumina sequencing platform. Applied and Environmental Microbiology, 79(17), 5112-5120.
Dupont, P. Y., Eaton, C. J., Wargent, J. J., Fechtner, S., Solomon, P., Schmid, J., Day, R. C.,
Scott, B. and Cox, M. P., 2015. Fungal endophyte infection of ryegrass reprograms host metabolism and alters development. New Phytologist, 208(4), pp. 1227-1240.
Eberhard et al., (1981). Biochemistry 20 (9): 2444-2449
el-Basyouni, S. Z., Chen, D., Ibrahim, R. K., Neish, A. C., and Towers, G. H. N. (1964). The biosynthesis of hydroxybenzoic acids in higher plants. Phytochemistry 3, 485-492.
Elliott, C., MüLler, J., Miklis, M., Bhat, R. A., Schulze-Lefert, P., and Panstruga, R. (2005). Conserved extracellular cysteine residues and cytoplasmic loop-loop interplay are required for functionality of the heptahelical MLO protein. Biochem. J. 385, 243.
Eriksson, S., Stransfeld, L., Adamski, N. M., Breuninger, H., and Lenhard, M. (2010). KLUH/CYP78A5-Dependent Growth Signaling Coordinates Floral Organ Growth in Arabidopsis. Curr. Biol. 20, 527-532.
Fang, Y., and Xiong, L. (2014). General mechanisms of drought response and their application in drought resistance improvement in plants. Cell. Mol. Life Sci. 72, 673-689.
Ferro, M., Brugière, S., Salvi, D., Seigneurin-Berny, D., Court, M., Moyet, L., Ramus, C., Miras, S., Mellal, M., Le Gall, S., et al. (2010). AT_CHLORO, a Comprehensive Chloroplast Proteome Database with Subplastidial Localization and Curated Information on Envelope Proteins. Mol. Cell. Proteomics MCP 9, 1063-1084.
Fiehn et al., (2000) Nature Biotechnol., 18, 1157-1161
Fink, A. L. (1999). Chaperone-Mediated Protein Folding. Physiol. Rev. 79, 425-449.
Flint, D. H., and Emptage, M. H. (1988). Dihydroxy acid dehydratase from spinach contains a [2Fe-2S] cluster. J. Biol. Chem. 263, 3558-3564.
Foreman, P. K., Brown, D., Dankmeyer, L., Dean, R., Diener, S., Dunn-Coleman, N. S., Goedegebuur, F., Houfek, T. D., England, G. J., Kelley, A. S. and Meerman, H. J., 2003. Transcriptional regulation of biomass-degrading enzymes in the filamentous fungus Trichoderma reesei. Journal of Biological Chemistry, 278(34), pp. 31988-31997.
Fortin, M. G., Zelechowska, M., and Verma, D. P. S. (1985). Specific targeting of membrane nodulins to the bacteroid-enclosing compartment in soybean nodules. EMBO J. 4, 3041-3046.
Franssen, H. J., Nap, J.-P., Gloudemans, T., Stiekema, W., Dam, H. V., Govers, F., Louwerse, J., Kammen, A. V., and Bisseling, T. (1987). Characterization of cDNA for nodulin-75 of soybean: A gene product involved in early stages of root nodule development. Proc. Natl. Acad. Sci. 84, 4495-4499.
Frenzel, T., and Zenk, M. H. (1990). S-adenosyl-1-methionine: 3′-hydroxy-N-methyl-(S)-coclaurine-4′-O-methyl transferase, a regio- and stereoselective enzyme of the (S)-reticuline pathway. Phytochemistry 29, 3505-3511.
Friml, J. (2003). Auxin transport—shaping the plant. Curr. Opin. Plant Biol. 6, 7-12.
Fujioka, S. and Yokota, T. 2003. Biosynthesis and metabolism of brassinosteroids. Annu. Rev. Plant Biol. 54, 137-164.
Ge, X., Chen, J., Li, N., Lin, Y., Sun, C., and Cao, K. (2003). Resistance function of rice lipid transfer protein LTP110. J. Biochem. Mol. Biol. 36, 603-607.
Germain, V., Rylott, E. L., Larson, T. R., Sherson, S. M., Bechtold, N., Carde, J.-P., Bryce, J. H., Graham, I. A., and Smith, S. M. (2001). Requirement for 3-ketoacyl-CoA thiolase-2 in peroxisome development, fatty acid β-oxidation and breakdown of triacylglycerol in lipid bodies of Arabidopsis seedlings. Plant J. 28, 1-12.
Ghosh, H. P., and Preiss, J. (1966). Adenosine diphosphate glucose pyrophosphorylase. A regulatory enzyme in the biosynthesis of starch in spinach leaf chloroplasts. J. Biol. Chem. 241, 4491-4504.
Giaquinta, R. T. (1983). Phloem Loading of Sucrose. Annu. Rev. Plant Physiol. 34, 347-387.
Gibson, K. E., Kobayashi, H., and Walker, G. 2008. Molecular determinants of a symbiotic chronic infection. Annu. Rev. Genet. 42, 413-41.
Gilbert, H. F., Lennox, B. J., Mossman, C. D., and Carle, W. C. (1981). The relation of acyl transfer to the overall reaction of thiolase I from porcine heart. J. Biol. Chem. 256, 7371-7377.
Gilmour et al. (2000) Plant Physiol. 124: 1854-1865
Ginzberg, I., Stein, H., Kapulnik, Y., Szabados, L., Strizhov, N., Schell, J., Koncz, C., and Zilberstein, A. (1998). Isolation and characterization of two different cDNAs of A 1-pyrroline-5-carboxylate synthase in alfalfa, transcriptionally induced upon salt stress. Plant Mol. Biol. 38, 755-764.
Göbel, C., and Feussner, I. (2009). Methods for the analysis of oxylipins in plants. Phytochemistry 70, 1485-1503.
Goldgur, Y., Rom, S., Ghirlando, R., Shkolnik, D., Shadrin, N., Konrad, Z., and Bar-Zvi, D. (2007). Desiccation and Zinc Binding Induce Transition of Tomato Abscisic Acid Stress Ripening 1, a Water Stress- and Salt Stress-Regulated Plant-Specific Protein, from Unfolded to Folded State. Plant Physiol. 143, 617-628.
Gottwald, S., Samans, B., Lück, S., & Friedt, W. (2012). Jasmonate and ethylene dependent defence gene expression and suppression of fungal virulence factors: two essential mechanisms of Fusarium head blight resistance in wheat?. BMC genomics 13, 1.
Grabau, E. A. (1986). Nucleotide sequence of the cytochrome oxidase subunit I gene from soybean mitochondria. Plant Mol. Biol. 7, 377-384.
Grimaud, F., Renaut, J., Dumont, E., Sergeant, K., Lucau-Danila, A., Blervacq, A.-S., Sellier, H., Bahrman, N., Lejeune-Hénaut, I., Delbreil, B., et al. (2013). Exploring chloroplastic changes related to chilling and freezing tolerance during cold acclimation of pea (Pisum sativum L.). J. Proteomics 80, 145-159.
Guo, C., Ge, X., and Ma, H. (2013). The rice OsDIL gene plays a role in drought tolerance at vegetative and reproductive stages. Plant Mol. Biol. 82, 239-253.
Haake et al. (2002) Plant Physiol. 130: 639-648
Hai Du, Yong-Bin Wang, Yi Xie, Zhe Liang, San-Jie Jiang, Shuang-Shuang Zhang, Yu-Bi Huang, and Yi-Xiong Tang. 2013. Genome-Wide Identification and Evolutionary and Expression Analyses of MYB-Related Genes in Land Plants. DNA Research. doi: 10.1093/dnares/dst021
Hajeri, Shubash; El-Mohtar, Choaa; Dawson, William O.; & Gowda, Siddarame. (2014). Citrus tristeza virus-based RNA-interference (RNAi) vector and its potential in combating citrus Huanglongbing (HLB). Journal of Citrus Pathology, 1(1).
Haldrup, A., Simpson, D. J., and Scheller, H. V. (2000). Down-regulation of the PSI-F subunit of photosystem I (PSI) in Arabidopsis thaliana. The PSI-F subunit is essential for photoautotrophic growth and contributes to antenna function. J. Biol. Chem. 275, 31211-31218.
Halic, M., Becker, T., Pool, M. R., Spahn, C. M. T., Grassucci, R. A., Frank, J., and Beckmann, R. (2004). Structure of the signal recognition particle interacting with the elongation-arrested ribosome. Nature 427, 808-814.
Hallman, J., et al., (1997) Canadian Journal of Microbiology. 43(10): 895-914
Handa, S., Bressan, R. A., Handa, A. K., and Carpita, N. C. 1983. Solutes contributing to osmotic adjustment in cultured plant cells adapted to water stress.
Hanfrey, C., Sommer, S., Mayer, M. J., Burtin, D., and Michael, A. J. (2001). Arabidopsis polyamine biosynthesis: absence of ornithine decarboxylase and the mechanism of arginine decarboxylase activity. Plant J. 27, 551-560.
Hata, S., Kouchi, H., Suzuka, I., and Ishii, T. (1991). Isolation and characterization of cDNA clones for plant cyclins. EMBO J. 10, 2681-2688.
Hays, J. B., and Pang, Q. (1994). UV—B-Inducible and Constitutive Genes that Mediate Repair and Toleration Of UV-Damaged DNA in the Plant Arabidopsis Thaliana. In Stratospheric Ozone Depletion/UV-B Radiation in the Biosphere, R. H. Biggs, and M. E. B. Joyner, eds. (Springer Berlin Heidelberg), pp. 107-122.
He, Y. (2009). Control of the Transition to Flowering by Chromatin Modifications. Mol. Plant 2, 554-564.
Heitzeberg, F., Chen, I.-P., Hartung, F., Orel, N., Angelis, K. J., and Puchta, H. (2004). The Rad17 homologue of Arabidopsis is involved in the regulation of DNA damage repair and homologous recombination. Plant J. 38, 954-968.
Hertzberg, M., Aspeborg, H., Schrader, J., Andersson, A., Erlandsson, R., Blomqvist, K., Bhalerao, R., Uhlen, M., Teeri, T. T., Lundeberg, J., et al. (2001). A transcriptional roadmap to wood formation. Proc. Natl. Acad. Sci. 98, 14732-14737.
Hesse, H., Kreft, O., Maimann, S., Zeh, M., and Hoefgen, R. (2004). Current understanding of the regulation of methionine biosynthesis in plants. J. Exp. Bot. 55, 1799-1808.
Hirose, T., Ohdan, T., Nakamura, Y., and Terao, T. (2006). Expression profiling of genes related to starch synthesis in rice leaf sheaths during the heading period. Physiol. Plant. 128, 425-435.
Ho, C.-L., and Saito, K. (2001). Molecular biology of the plastidic phosphorylated serine biosynthetic pathway in Arabidopsis thaliana. Amino Acids 20, 243-259.
Hong, Z., Lakkineni, K., Zhang, Z., and Verma, D. P. S. (2000). Removal of Feedback Inhibition of Δ 1-Pyrroline-5-Carboxylate Synthetase Results in Increased Proline Accumulation and Protection of Plants from Osmotic Stress. Plant Physiol. 122, 1129-1136.
Hu C A, Delauney A J, Verma D P. 1992. A bifunctional enzyme (delta 1-pyrroline-5-carboxylate synthetase) catalyzes the first two steps in proline biosynthesis in plants. PNAS. 89(19):9354-58.
Hui-Wen Wang, Jin-Song Zhang, Jun-Yi Gai, Shou-Yi Chen. 2006. Cloning and comparative analysis of the gene encoding diacylglycerol acyltransferase from wild type and cultivated soybean. Theoretical and Applied Genetics. 112(6):1086-97.
Huttová, J., Mistrík, I., OIle-Simonovicova, M., and Tamás, L. (2006). Cadmium induced changes in cell wall peroxidase isozyme pattern in barley root tips. Plant Soil Environ. 52, 250.
Ishikawa, K., Nakatani, H., Katsuya, Y., and Fukazawa, C. (2007). Kinetic and Structural Analysis of Enzyme Sliding on a Substrate: Multiple Attack in β-Amylase‡. Biochemistry (Mosc.) 46, 792-798.
Ishikawa, T., Akita, S., and Li, Q. (1993). Relationship Between Content of Nonstructural Carbohydrates Before Panicle Initiation Stage and Grain-Yield in Rice (oryza-Sativa L). Jpn. J. Crop Sci. 62, 130-131.
Ito, T., Shiraishi, H., Okada, K., and Shimura, Y. (1994). Two amidophosphoribosyltransferase genes of Arabidopsis thaliana expressed in different organs. Plant Mol. Biol. 26, 529-533.
Jami, S. K., Clark, G. B., Turlapati, S. A., Handley, C., Roux, S. J., and Kirti, P. B. (2008). Ectopic expression of an annexin from Brassica juncea confers tolerance to abiotic and biotic stress treatments in transgenic tobacco. Plant Physiol. Biochem. 46, 1019-1030.
Jeng, W.-Y., Wang, N.-C., Lin, M.-H., Lin, C.-T., Liaw, Y.-C., Chang, W.-J., Liu, C.-I., Liang, P.-H., and Wang, A. H.-J. (2011). Structural and functional analysis of three β-glucosidases from bacterium Clostridium cellulovorans, fungus Trichoderma reesei and termite Neotermes koshunensis. J. Struct. Biol. 173, 46-56.
Jessica S. Kelsey, Nathan M. Fastman and Daphne D. Blumberg. 2012. Evidence of an Evolutionarily Conserved LMBR1 Domain-Containing Protein That Associates with Endocytic Cups and Plays a Role in Cell Migration in Dictyostelium discoideum. Eukaryotic Cell. 11(4):401-16.
Jimeno, S., Rondón, A. G., Luna, R., and Aguilera, A. (2002). The yeast THO complex and mRNA export factors link RNA metabolism with transcription and genome instability. EMBO J. 21, 3526-3535.
Johnston-Monje and Raizada (2011)
Juan et al. 1998 Appl Environ Microbiol 64:4600-4602 Jung Ro Lee, Kara A. Boltz, Sang Yeol Lee. 2014. Molecular chaperone function of Arabidopsis thaliana phloem protein 2-A1, encodes a protein similar to phloem lectin. Biochemical and Biophysical Research Communications. 443(1):18-21.
Kalifa, Y., Gilad, A., Konrad, Z., Zaccai, M., Scolnik, P. A., and Bar-Zvi, D. (2004). The water- and salt-stress-regulated Asr1 (abscisic acid stress ripening) gene encodes a zinc-dependent DNA-binding protein. Biochem. J. 381, 373.
Kathrin Schrick, Diana Nguyen, Wojciech M Karlowski and Klaus FX Mayer. 2004. START lipid/sterol-binding domains are amplified in plants and are predominantly associated with homeodomain transcription factors. Genome Biology. 5:R41.
Kato, Y., Konishi, M., Shigyo, M., Yoneyama, T., and Yanagisawa, S. (2010). Characterization of plant eukaryotic translation initiation factor 6 (eIF6) genes: The essential role in embryogenesis and their differential expression in Arabidopsis and rice. Biochem. Biophys. Res. Commun. 397, 673-678.
Kawashima, N., and Wildman, S. G. (1972). Studies on fraction I protein. IV. Mode of inheritance of primary structure in relation to whether chloroplast or nuclear DNA contains the code for a chloroplast protein. Biochim. Biophys. Acta 262, 42-49.
Kazama, T., Ichihashi, Y., Murata, S., and Tsukaya, H. (2010). The Mechanism of Cell Cycle Arrest Front Progression Explained by a KLUH/CYP78A5-dependent Mobile Growth Factor in Developing Leaves of Arabidopsis thaliana. Plant Cell Physiol. 51, 1046-1054.
Kereszt, A., Mergaert, P., and Kondorosi, E. (2011). Bacteroid Development in Legume Nodules: Evolution of Mutual Benefit or of Sacrificial Victims? Mol. Plant. Microbe Interact. 24, 1300-1309.
Khan, W., Balakrishnan, P., and Smith, D. L. 2008. Nod factor [Nod Bj V (C18:1, MeFuc)] and lumichrome enhance photosynthesis and growth of corn and soybean.
Khazaei, H., O'Sullivan, D. M., Sillanpää, M. J., and Stoddard, F. L. (2014). Use of synteny to identify candidate genes underlying QTL controlling stomatal traits in faba bean (Vicia faba L.). Theor. Appl. Genet. 127, 2371-2385.
Khripach, V., Zhabinskii, V., and De Groot, A. 2000. Twenty years of brassinosteroids:
steroidal plant hormones warrant better crops for the XXI century. Annals of Botany. 86, 441-447.
Kidd et al. 2007, Chemosphere 66:1458-1467
Kim, J. H., Delauney, A. J., and Verma, D. P. (1995). Control of de novo purine biosynthesis genes in ureide-producing legumes: induction of glutamine phosphoribosylpyrophosphate amidotransferase gene and characterization of its cDNA from soybean and Vigna. Plant J. Cell Mol. Biol. 7, 77-86.
Kim, S., Choi, H. I., Ryu, H. J., Park, J. H., Kim, M. D., & Kim, S. Y. (2004). ARIA, an Arabidopsis arm repeat protein interacting with a transcriptional regulator of abscisic acid-responsive gene expression, is a novel abscisic acid signaling component. Plant Physiology 136, 3639-3648.
Kim, Y.-C., Kim, S.-Y., Paek, K.-H., Choi, D., and Park, J. M. (2006). Suppression of CaCYP1, a novel cytochrome P450 gene, compromises the basal pathogen defense response of pepper plants. Biochem. Biophys. Res. Commun. 345, 638-645.
Klxmbt, H. D. (1962). Conversion in plants of benzoic acid to salicylic acid and its [beta]d-glucoside. Nature 196, 491.
Kohzuma, K., Cruz, J. A., Akashi, K., Hoshiyasu, S., Munekage, Y. N., Yokota, A., and Kramer, D. M. (2009). The long-term responses of the photosynthetic proton circuit to drought. Plant Cell Environ. 32, 209-219.
Koizumi, N. (1996). Isolation and Responses to Stress of a Gene That Encodes a Luminal Binding Protein in Arabidopsis thaliana. Plant Cell Physiol. 37, 862-865.
Kolattukudy, P. E. (1980). Biopolyester Membranes of Plants: Cutin and Suberin. Science 208, 990-1000.
Kollmar, R., and Farnham, P. J. (1993). Site-Specific Initiation of Transcription by RNA Polymerase II. Exp. Biol. Med. 203, 127-139.
Kollöffel, C., and Stroband, H. W. J. (1973). Ornithine carbamyltransferase activity from the cotyledons of developing and germinating seeds of Vicia faba. Phytochemistry 12, 2635-2638.
Kong, W., Chen, N., Liu, T., Zhu, J., Wang, J., He, X. and Jin, Y., 2015. Large-scale transcriptome analysis of cucumber and Botrytis cinerea during infection. PloS one, 10(11), p. e0142221.
Konishi, H., Yamane, H., Maeshima, M., and Komatsu, S. (2005). Characterization of fructose-bisphosphate aldolase regulated by gibberellin in roots of rice seedling. Plant Mol. Biol. 56, 839-848.
Konishi, T., Maruta, Y., Shinohara, K., and Watanabe, A. (1993). Transit Peptides of Thylakoid Luminal Proteins: the Sites of Stromal Processing are Conserved among Higher Plants. Plant Cell Physiol. 34, 1081-1087.
Kreuz, K., Dehesh, K., and Apel, K. (1986). The light-dependent accumulation of the P700 chlorophyll a protein of the photosystem I reaction center in barley. Eur. J. Biochem. 159, 459-467.
Kuc, J. (1987). Translocated Signals for Plant Immunization. Ann. N. Y. Acad. Sci. 494, 221-223.
Kukavica, B. M., Veljovice-Jovanovicć, S. D., Menckhoff, L., and Liithje, S. (2012). Cell wall-bound cationic and anionic class III isoperoxidases of pea root: biochemical characterization and function in root growth. J. Exp. Bot.
Kumar et al. 1995, Environ. Sci. Technol. 29:1232-1238
Kuras, R., Büschlen, S., and Wollman, F.-A. (1995). Maturation of Pre-apocytochrome fin Vivo: a site-directed mutagenesis study in Chlamydomonas reinhardtii. J. Biol. Chem. 270, 27797-27803.
Kusuda, H., Koga, W., Kusano, M., Oikawa, A., Saito, K., Hirai, M. Y., and Yoshida, K. T.
(2015). Ectopic expression of myo-inositol 3-phosphate synthase induces a wide range of metabolic changes and confers salt tolerance in rice. Plant Sci. 232, 49-56.
Kyoichi Isono, Hiroshi Yamamoto, Kimiyuki Satoh and Hirokazu Kobayashi. 1999. An Arabidopsis cDNA encoding a DNA-binding protein that is highly similar to the DEAH family of RNA/DNA helicase genes. Nucleic Acids Research. 27(18):3728-35.
Lange, B. M., and Croteau, R. (1999). Isoprenoid Biosynthesis via a Mevalonate-Independent Pathway in Plants: Cloning and Heterologous Expression of 1-Deoxy-d-xylulose-5-phosphate Reductoisomerase from Peppermint. Arch. Biochem. Biophys. 365, 170-174.
Lara, M., Cullimore, J. V., Lea, P. J., Miflin, B. J., Johnston, A. W. B., and Lamb, J. W. (1983). Appearance of a novel form of plant glutamine synthetase during nodule development in Phaseolus vulgaris L. Planta 157, 254-258.
Laux, T., Seurinck, J., and Goldberg, R. B. (1991). A soybean embryo cDNA encodes a DNA binding protein with histone and HMG-protein-like domains. Nucleic Acids Res. 19, 4768.
Lee, H. I., León, J., and Raskin, I. (1995). Biosynthesis and metabolism of salicylic acid. Proc. Natl. Acad. Sci. 92, 4076-4079.
Lee, S.-B., Jung, S.-J., Go, Y.-S., Kim, H.-U., Kim, J.-K., Cho, H.-J., Park, O. K., and Suh, M.-C. (2009). Two Arabidopsis 3-ketoacyl CoA synthase genes, KCS20 and KCS2/DAISY, are functionally redundant in cuticular wax and root suberin biosynthesis, but differentially controlled by osmotic stress. Plant J. 60, 462-475.
Legocki, R. P., and Verma, D. P. S. (1980). Identification of “nodule-specific” host proteins (nodulins) involved in the development of Rhizobium-Legume symbiosis. Cell 20, 153-163.
Levey, T., Westhoff, P., and Meierhoff, K. (2014). Expression of a nuclear-encoded psbH gene complements the plastidic RNA processing defect in the PSII mutant hcf107 in Arabidopsis thaliana. Plant J. 80, 292-304.
Li et al., (2004) Mycologia 96: 526-536
Li, C., Ng, C. K.-Y., and Fan, L.-M. (2015). MYB transcription factors, active players in abiotic stress signaling. Environ. Exp. Bot. 114, 80-91.
Li, H. M., Sullivan, R., Moy, M., Kobayashi, D. Y. and Belanger, F. C., 2004. Expression of a novel chitinase by the fungal endophyte in Poa ampla. Mycologia, 96(3), pp. 526-536.
Liepman, A. H., and Olsen, L. J. (2003). Alanine Aminotransferase Homologs Catalyze the Glutamate: Glyoxylate Aminotransferase Reaction in Peroxisomes of Arabidopsis. Plant Physiol. 131, 215-227.
Lin, H., Yang, Y., Quan, R., Mendoza, I., Wu, Y., Du, W., Zhao, S., Schumaker, K. S., Pardo, J. M., and Guo, Y. (2009). Phosphorylation of SOS3-LIKE CALCIUM BINDING PROTEIN8 by SOS2 Protein Kinase Stabilizes Their Protein Complex and Regulates Salt Tolerance in Arabidopsis. Plant Cell 21, 1607-1619.
Lindquist, E., and Aronsson, H. (2014). Proteins affecting thylakoid morphology—the key to understanding vesicle transport in chloroplasts? Plant Signal. Behay. 9, e977205.
Lindstrom, J. T., Sun, S. and Belanger, F. C., 1993. A novel fungal protease expressed in endophytic infection of Poa species. Plant physiology, 102(2), pp. 645-650.
Liu, Z., Hossain, G. S., Islas-Osuna, M. A., Mitchell, D. L., and Mount, D. W. (2000). Repair of UV damage in plants by nucleotide excision repair: Arabidopsis UVH1 DNA repair gene is a homolog of Saccharomyces cerevisiae Radl. Plant J. 21, 519-528.
Lo Presti, L., Lanver, D., Schweizer, G., Tanaka, S., Liang, L., Tollot, M., Zuccaro, A., Reissmann, S. and Kahmann, R., 2015. Fungal effectors and plant susceptibility. Annual review of plant biology, 66, pp. 513-545.
Madsen, N. B (1986). The Enzymes (Academic Press, Inc., New York.).
Mahato, S., De, D., Dutta, D., Kundu, M., Bhattacharya, S., Schiavone, M. T., and Bhattacharya, S. K. (2004). Potential use of sugar binding proteins in reactors for regeneration of CO2 fixation acceptor D-Ribulose-1,5-bisphosphate. Microb. Cell Factories 3, 7.
Marc Libault, Gary Stacey. 2010. Evolution of FW2.2-like (FWL) and PLAC8 genes in eukaryotes. Plant Signaling & Behavior. 5(10):1226-28.
Matamoros, M. A., Dalton, D. A., Ramos, J., Clemente, M. R., Rubio, M. C., and Becana, M. (2003). Biochemistry and Molecular Biology of Antioxidants in the Rhizobia-Legume Symbiosis. Plant Physiol. 133, 499-509.
Mateos et al. 1992, Appl Environ Microbiol 58:1816-1822
Matiru, V. N. and Dakora, F. D. 2005. Xylem transport and shoot accumulation of lumichrome, a newly recongnized rhizobial signal, alters root respiration, stomatal conductance, leaf transpiration and photosynthetic rates in legumes and cereals. New Phytologist. 165, 847-855.
Mattiacci, L., Dicke, M., and Posthumus, M. A. (1995). beta-Glucosidase: an elicitor of herbivore-induced plant odor that attracts host-searching parasitic wasps. Proc. Natl. Acad. Sci. U.S.A 92, 2036-2040.
Mayer K, Schüller C, Wambutt R, Murphy G, Volckaert G, McCombie W R. 1999.
Sequence and analysis of chromosome 4 of the plant Arabidopsis thaliana. Nature. 402:769-77.
McAinsh, M. R. (1990). Abscisic acid-induced elevation of guard cell cytosolic Ca2+ precedes stomatal closure. Nature 343, 186-188.
McCarty, R. E. (1992). A plant biochemist's view of H+ ATPases and ATP synthases. J. Exp. Biol. 172, 431-441.
McClung, C. R., Hsu, M., Painter, J. E., Gagne, J. M., Karlsberg, S. D., and Salome, P. A. (2000). Integrated Temporal Regulation of the Photorespiratory Pathway. Circadian Regulation of Two Arabidopsis Genes Encoding Serine Hydroxymethyltransferase. Plant Physiol. 123, 381-392.
McClure, P. R. and Israel, D. W. 1979. Transport of nitrogen in the xylem of soybean plants. Plant Physiol. 64, 411-416.
McCormack, E., Tsai, Y.-C., and Braam, J. (2005). Handling calcium signaling: Arabidopsis CaMs and CMLs. Trends Plant Sci. 10, 383-389.
McDonald, D., Price, M. N., Goodrich, J., Nawrocki, E. P., DeSantis, T. Z., Probst, A., . . . Hugenholtz, P. (2012). An improved Greengenes taxonomy with explicit ranks for ecological and evolutionary analyses of bacteria and archaea. The ISME Journal, 6(3), 610-8.
Mechthild Tegeder. 2012. Transporters for amino acids in plant cells: some functions and many unknowns. Current Opinion in Plant Biology. 15(3):315-21.
Medina and Baresi 2007, J Microbiol Methods 69:391-393
Mei Guo, Mary A. Rupe, Jo Ann Dieter, Jijun Zou, Daniel Spielbauer, Keith E. Duncan,
Richard J. Howard, Zhenglin Hou and Carl R. Simmons. 2010. Cell Number Regulator1 Affects Plant and Organ Size in Maize: Implications for Crop Yield Enhancement and Heterosis. The Plant Cell. 22(4):1057-73.
Meng Zhang, Jilian Fan, David C. Taylor and John B. Ohlrogge. 2009. DGAT1 and PDAT1 Acyltransferases Have Overlapping Functions in Arabidopsis Triacylglycerol Biosynthesis and Are Essential for Normal Pollen and Seed Development. The Plant Cell. 21(12):3885-3901
Menotta, M., Amicucci, A., Basili, G., Polidori, E., Stocchi, V. and Rivero, F., 2008. Molecular and functional characterization of a Rho GDP dissociation inhibitor in the filamentous fungus Tuber borchii. BMC microbiology, 8(1), p. 57.
Michal Malewicz, Thomas Perlmann. 2014. Function of transcription factors at DNA lesions in DNA repair. Experimental Cell Research. 329(1):94-100.
Michel, H., Behr, J., Harrenga, A., and Kannt, A. (1998). CYTOCHROME C OXIDASE: Structure and Spectroscopy. Annu. Rev. Biophys. Biomol. Struct. 27, 329-356.
Ming-Der Shih, Folkert A. Hoekstra, Yue-Ie C. Hsing. 2008. Late Embryogenesis Abundant Proteins. Advances in Botanical Research. 48:211-55.
Miyagawa, Y., Tamoi, M., and Shigeoka, S. (2001). Overexpression of a cyanobacterial fructose-1,6-/sedoheptulose-1,7-bisphosphatase in tobacco enhances photosynthesis and growth. Nat. Biotechnol. 19, 965-969.
Monde, R. A., Schuster, G., and Stern, D. B. (2000). Processing and degradation of chloroplast mRNA. Biochimie 82, 573-582.
Moreno, J. I., Martin, R., and Castresana, C. (2005). Arabidopsis SHMT1, a serine hydroxymethyltransferase that functions in the photorespiratory pathway influences resistance to biotic and abiotic stress. Plant J. 41, 451-463.
Mori, M., Murata, K., Kubota, H., Yamamoto, A., Matsushiro, A., and Monta, T. (1992). Cloning of a cDNA encoding the Tcp-1 (t complex polypeptide 1) homologue of Arabidopsis thaliana. Gene 122, 381-382.
Mortvedt 1996, Fertilizer Res. 43:55-61
Moura, J. C. M. S., Bonine, C. A. V., Viana, J. O. F., Dornelas, M. C., and Mazzafera, P. 2010. Abiotic and biotic stresses and changes in the lignin content and composition in plants. J. Int. Plant Biol. 53, (4) 360-376.
Müller A. H., and Hansson, M. (2009). The Barley Magnesium Chelatase 150-kD Subunit Is Not an Abscisic Acid Receptor. Plant Physiol. 150, 157-166.
Mustafiz, A., Singh, A. K., Pareek, A., Sopory, S. K., and Singla-Pareek, S. L. (2011). Genome-wide analysis of rice and Arabidopsis identifies two glyoxalase genes that are highly expressed in abiotic stresses. Funct. Integr. Genomics 11, 293-305.
Navarova, H., Bernsdorff, F., Doring, A., and Zeier, J. 2012. Pipecolic acid, an endogenous mediator of defense amplification and priming, is a critical regulator of inducible plant immunity. The Plant Cell. 24, 5123-5141.
Neha Vaid, Prashant Kumar Pandey, Narendra Tutej a. 2012. Genome-wide analysis of lectin receptor-like kinase family from Arabidopsis and rice. Plant Molec Biol. 80(4):365-88.
Nelson, D., and Werck-Reichhart, D. (2011). A P450-centric view of plant evolution. Plant J. 66, 194-211.
Newton, K. J. (1988). Plant Mitochondrial Genomes: Organization, Expression and Variation. Annu. Rev. Plant Physiol. Plant Mol. Biol. 39, 503-532.
Nishizawa, A. N., and Buchanan, B. B. (1981). Enzyme regulation in C4 photosynthesis. Purification and properties of thioredoxin-linked fructose bisphosphatase and sedoheptulose bisphosphatase from corn leaves. J. Biol. Chem. 256, 6119-6126.
Nordberg, J., and Arnér, E. S. J. (2001). Reactive oxygen species, antioxidants, and the mammalian thioredoxin systeml. Free Radic. Biol. Med. 31, 1287-1312.
Ooka, H., Satoh, K., Doi, K., Nagata, T., Otomo, Y., Murakami, K., Matsubara, K., Osato, N., Kawai, J., Carninci, P., et al. (2003). Comprehensive analysis of NAC family genes in Oryza sativa and Arabidopsis thaliana. DNA Res. Int. J. Rapid Publ. Rep. Genes Genomes 10, 239-247.
Oppenheimer, D. G., Haas, N., Silflow, C. D., and Snustad, D. P. (1988). The β-tubulin gene family of Arabidopsis thaliana: preferential accumulation of the β1 transcript in roots. Gene 63, 87-102.
Osman, S. F. and Fett, W. F. 1982. Isoflavone glucoside stress metabolites of soybean leaves. Phytochem. 22, 1921-1923.
Pang, Q., Hays, J. B., Rajagopal, I., and Schaefer, T. S. (1993). Selection of Arabidopsis cDNAs that partially correct phenotypes of Escherichia coli DNA-damage-sensitive mutants and analysis of two plant cDNAs that appear to express UV-specific dark repari activities. Plant Mol. Biol. 22, 411-426.
Pearson, Methods Enzymol. 183:63-98 (1990).
Peiro, A., Izquierdo-Garcia, A. C., Sanchez-Navarro, J. A., Pallas, V., Mulet, J. M., and Aparicio, F. (2014). Patellins 3 and 6, two members of the Plant Patellin family, interact with the movement protein of Alfalfa mosaic virus and interfere with viral movement. Mol. Plant Pathol. 15, 881-891.
Peng Wang, Zhaowei Li, Jingshuang Wei, Zenglin Zhao, Daye Sun, and Sujuan Cui. 2012. A Na+/Ca2+ Exchanger-like Protein (AtNCL) Involved in Salt Stress in Arabidopsis. J Biol Chem. 287(53): 44062-44070.
Pérez-Miranda S., Journal of Microbiological Methods, Volume 70, Issue 1, Pages 127-131 (2007)
Peyret, P., Perez, P., and Alric, M. (1995). Structure, Genomic Organization, and Expression of the Arabidopsis thaliana Aconitase Gene PLANT ACONITASE SHOW SIGNIFICANT HOMOLOGY WITH MAMMALIAN IRON-RESPONSIVE ELEMENT-BINDING PROTEIN. J. Biol. Chem. 270, 8131-8137.
Phalip et al., (1994) J Basic Microbiol 34: 277-280
Phillips, D. A., Joseph, C. M., Yang, G., Martinez-Romero, E., Sanborn, J. R., and Volpin, H. 1999. Identification of lumichrome as a Sinorhizobium enhancer of alfalfa root respiration and shoot growth. PNAS. 96, (22) 12275-12280.
Pii, Y., Molesini, B., Masiero, S., and Pandolfini, T. (2012). The non-specific lipid transfer protein N5 of Medicago truncatula is implicated in epidermal stages of rhizobium-host interaction. BMC Plant Biol. 12, 233.
Polacco, J. C., and Holland, M. A. (1993). Roles of Urease in Plant Cells. In International Review of Cytology, K. W. J. and J. Jarvik, ed. (Academic Press), pp. 65-103.
Pollard, M., Beisson, F., Li, Y., and Ohlrogge, J. B. (2008). Building lipid barriers: biosynthesis of cutin and suberin. Trends Plant Sci. 13, 236-246.
Ponting, C. P. (1997). Evidence for PDZ domains in bacteria, yeast, and plants. Protein Sci. 6, 464-468.
Prabhu, V., Chatson, K. B., Lui, H., Abrams, G. D., and King, J. (1998). Effects of Sulfanilamide and Methotrexate on 13C Fluxes through the Glycine Decarboxylase/Serine Hydroxymethyltransferase Enzyme System in Arabidopsis. Plant Physiol. 116, 137-144.
Priya, R., and Siva, R. (2015). Analysis of phylogenetic and functional diverge in plant nine-cis epoxycarotenoid dioxygenase gene family. J. Plant Res. 1-16.
Qian, W., Yu, C., Qin, H., Liu, X., Zhang, A., Johansen, I. E., and Wang, D. (2007). Molecular and functional analysis of phosphomannomutase (PMM) from higher plants and genetic evidence for the involvement of PMM in ascorbic acid biosynthesis in Arabidopsis and Nicotiana benthamiana: Functional analysis of plant phosphomannomutase. Plant J. 49, 399-413.
Quadt-Hallmann et al., (1997) Can. J. Microbiol., 43: 577-582
Quast, C., Pruesse, E., Yilmaz, P., Gerken, J., Schweer, T., Glo, F. O., & Yarza, P. (2013). The SILVA ribosomal RNA gene database project: improved data processing and web-based tools. Nucleic Acids Research, 41(November 2012), 590-596.
Quiroga, M., de Forchetti, S. M., Taleisnik, E., and Tigier, H. A. (2001). Tomato root peroxidase isoenzymes: kinetic studies of the coniferyl alcohol peroxidase activity, immunological properties and role in response to salt stress. J. Plant Physiol. 158, 1007-1013.
Raimund Tenhaken, Tobias Doerks, and Peer Bork. 2005. DCD—a novel plant specific domain in proteins involved in development and programmed cell death. BMC Bioinformatics. 6:169.
Rajkumar et al. 2009, Chemosphere 77:153-160
Ramos, J., Matamoros, M. A., Naya, L., James, E. K., Rouhier, N., Sato, S., Tabata, S., and
Becana, M. (2009). The glutathione peroxidase gene family of Lotus japonicus: characterization of genomic clones, expression analyses and immunolocalization in legumes. New Phytol. 181, 103-114.
Rashid, N., Morikawa, M., and Imanaka, T. (1997). Gene cloning and characterization of recombinant ribose phosphate pyrophosphokinase from a hyperthermophilic archaeon. J. Ferment. Bioeng. 83, 412-418.
Reddy, P. V., Lam, C. K. and Belanger, F. C., 1996. Mutualistic fungal endophytes express a proteinase that is homologous to proteases suspected to be important in fungal pathogenicity. Plant Physiology, 111(4), pp. 1209-1218.
Regalado, A., Pierri, C. L., Bitetto, M., Laera, V. L., Pimentel, C., Francisco, R., Passarinho, J., Chaves, M. M., and Agrimi, G. (2012). Characterization of mitochondrial dicarboxylate/tricarboxylate transporters from grape berries. Planta 237, 693-703.
Reinbothe, H. and Mothes, K. 1962. Urea, ureides and guanidine in plants. Annu. Rev. Plant Physiol. 13, 129-149.
Reinhold-Hurek, B., and Hurek, T. (2011). Living inside plants: bacterial endophytes. Curr. Opin. Plant Biol. 14, 435-443.
Rexroth, S., Poetsch, A., Rogner, M., Hamann, A., Werner, A., Osiewacz, H. D., Schafer, E. R., Seelert, H. and Dencher, N. A., 2012. Reactive oxygen species target specific tryptophan site in the mitochondrial ATP synthase. Biochimica et Biophysica Acta (BBA)-Bioenergetics, 1817(2), pp. 381-387.
Ribichich, K. F., Tioni, M. F., Chan, R. L., and Gonzalez, D. H. (2001). Cell-Type-Specific Expression of Plant Cytochrome cmRNA in Developing Flowers and Roots. Plant Physiol. 125, 1603-1610.
Roberto Schmidt, Harald Stransky, Wolfgang Koch. 2007. The amino acid permease AAP8 is important for early seed development in Arabidopsis thaliana. Planta. 226(4):805-13.
Robinson, D. L., Kahn, M. L., and Vance, C. P. (1994). Cellular localization of nodule-enhanced aspartate aminotransferase in Medicago sativa L. Planta 192, 202-210.
Rocha, A. G., Mehlmer, N., Stael, S., Mair, A., Parvin, N., Chigri, F., Teige, M., and Vothknecht, U. C. (2014). Phosphorylation of Arabidopsis transketolase at Ser 428 provides a potential paradigm for the metabolic control of chloroplast carbon metabolism. Biochem. J. 458, 313-322.
Rodrigues, S. M., Andrade, M. O., Gomes, A. P. S., DaMatta, F. M., Baracat-Pereira, M. C., and Fontes, E. P. B. (2006). Arabidopsis and tobacco plants ectopically expressing the soybean antiquitin-like ALDH7 gene display enhanced tolerance to drought, salinity, and oxidative stress. J. Exp. Bot. 57, 1909-1918.
Roessner et al., (2001) Plant Cell, 13, 11-29
Rondón, A. G., Jimeno, S., Garcia-Rubio, M., and Aguilera, A. (2003). Molecular Evidence That the Eukaryotic THO/TREX Complex Is Required for Efficient Transcription Elongation. J. Biol. Chem. 278, 39037-39043.
Rongvaux, A., Andris, F., Van Gool, F., and Leo, 0. (2003). Reconstructing eukaryotic NAD metabolism. BioEssays News Rev. Mol. Cell. Dev. Biol. 25, 683-690.
Roppolo, D., Boeckmann, B., Pfister, A., Boutet, E., Rubio, M. C., Denervaud-Tendon, V., Vermeer, J. E. M., Gheyselinck, J., Xenarios, I., and Geldner, N. (2014). Functional and Evolutionary Analysis of the CASPARIAN STRIP MEMBRANE DOMAIN PROTEIN Family. Plant Physiol. 165, 1709-1722.
Ruiter, H. de, and Kollöffel, C. (1982). Activity of Enzymes of Arginine Metabolism in the Cotyledons of Developing and Germinating Pea Seeds. Plant Physiol. 70, 313-315.
S E Jacobsen, K A Binkowski, and N E Olszewski. 1996. SPINDLY, a tetratricopeptide repeat protein involved in gibberellin signal transduction in Arabidopsis. PNAS. 93(17):9292-9296.
Sainz, G., Carrell, C. J., Ponamarev, M. V., Soriano, G. M., Cramer, W. A., and Smith, J. L. (2000). Interruption of the Internal Water Chain of Cytochrome f Impairs Photosynthetic Function†,‡. Biochemistry (Mosc.) 39, 9164-9173.
Samuel, M. A., Salt, J. N., Shiu, S. H., & Goring, D. R. (2006). Multifunctional arm repeat domains in plants. International review of cytology 253, 1-26.
Sauer, N., and Tanner, W. (1993). Molecular-Biology of Sugar Transporters in Plants. Bot. Acta 106, 277-286.
Sauveplane, V., Kandel, S., Kastner, P.-E., Ehlting, J., Compagnon, V., Werck-Reichhart, D., and Pinot, F. (2009). Arabidopsis thaliana CYP77A4 is the first cytochrome P450 able to catalyze the epoxidation of free fatty acids in plants. FEBS J. 276, 719-735.
Schaller, A. (2004). A cut above the rest: the regulatory function of plant proteases—Springer. Planta 220, 183-197.
Scheible, W.-R., Krapp, A., and Stitt, M. (2000). Reciprocal diurnal changes of phosphoenolpyruvate carboxylase expression and cytosolic pyruvate kinase, citrate synthase and NADP-isocitrate dehydrogenase expression regulate organic acid metabolism during nitrate assimilation in tobacco leaves. Plant Cell Environ. 23, 1155-1167.
Schomburg, P. D. D., and Stephan, D. D. (1995). (S)-2-Hydroxy-acid oxidase. In Enzyme Handbook 10, P. D. D. Schomburg, and D. D. Stephan, eds. (Springer Berlin Heidelberg), pp. 413-422.
Schomburg, P. D. D., and Stephan, D. D. (1997). Ribose-phosphate pyrophosphokinase. In Enzyme Handbook, P. D. D. Schomburg, and D. D. Stephan, eds. (Springer Berlin Heidelberg), pp. 421-427.
Schopfer, C. R., and Ebel, J. (1998). Identification of elicitor-induced cytochrome P450s of soybean (Glycine max L.) using differential display of mRNA. Mol. Gen. Genet. MGG 258, 315-322.
Schöttler, M. A., Fliigel, C., Thiele, W., and Bock, R. (2007a). Knock-out of the Plastid-encoded PetL Subunit Results in Reduced Stability and Accelerated Leaf Age-dependent Loss of the Cytochrome b6f Complex. J. Biol. Chem. 282, 976-985.
Schöttler, M. A., Flügel, C., Thiele, W., Stegemann, S., and Bock, R. (2007). The plastome-encoded PsaJ subunit is required for efficient Photosystem I excitation, but not for plastocyanin oxidation in tobacco. Biochem. J. 403, 251.
Schuhmann, H., and Adamska, I. (2012). Deg proteases and their role in protein quality control and processing in different subcellular compartments of the plant cell. Physiol. Plant. 145, 224-234.
Schultz, C. J., Hsu, M., Miesak, B., and Coruzzi, G. M. (1998). Arabidopsis Mutants Define an in Vivo Role for Isoenzymes of Aspartate Aminotransferase in Plant Nitrogen Assimilation. Genetics 149, 491-499.
Schultze, M., and Kondorosi, A. (1998). Regulation of Symbiotic Root Nodule Development. Annu. Rev. Genet. 32, 33-57.
Scott, D. B., Farnden, K. J. F., and Robertson, J. G. (1976). Ammonia assimilation in lupin nodules. Nature 263, 703-705.
Scranton, M. A., Fowler, J. H., Girke, T., and Walling, L. L. (2013). Microarray Analysis of Tomato's Early and Late Wound Response Reveals New Regulatory Targets for Leucine Aminopeptidase A. PLoS ONE 8, e77889.
Seki, M., Narusaka, M., Ishida, J., Nanjo, T., Fujita, M., Oono, Y., Kamiya, A., Nakajima, M., Enju, A., Sakurai, T., et al. (2002). Monitoring the expression profiles of 7000 Arabidopsis genes under drought, cold and high-salinity stresses using a full-length cDNA microarray. Plant J. 31, 279-292.
Sekine, K., Fujiwara, M., Nakayama, M., Takao, T., Hase, T., and Sato, N. (2007). DNA binding and partial nucleoid localization of the chloroplast stromal enzyme ferredoxin:sulfite reductase. FEBS J. 274, 2054-2069.
Sengupta-Gopalan, C., and Pitas, J. W. (1986). Expression of nodule-specific glutamine synthetase genes during nodule development in soybeans. Plant Mol. Biol. 7, 189-199.
Serraj, R., Sinclair, T. R., and Purcell, L. C. 1999. Symbiotic N2 fixation response to drought. J. Exp. Bot. 50, 143-155.
Shah, J. (2005). Lipids, Lipases, and Lipid-Modifying Enzymes in Plant Disease Resistance. Annu. Rev. Phytopathol. 43, 229-260.
Shah, J., and Klessig, D. (1999). Chapter 23 Salicylic acid: signal perception and transduction. In New Comprehensive Biochemistry, M. A. H. and K. R. L. P. J. J. Hooykaas, ed. (Elsevier), pp. 513-541.
Shen, Y.-Y., Wang, X.-F., Wu, F.-Q., Du, S.-Y., Cao, Z., Shang, Y., Wang, X.-L., Peng, C.-C., Yu, X.-C., Zhu, S.-Y., et al. (2006). The Mg-chelatase H subunit is an abscisic acid receptor. Nature 443, 823-826.
Shibuya, M., Hoshino, M., Katsube, Y., Hayashi, H., Kushiro, T., and Ebizuka, Y. (2006). Identification of β-amyrin and sophoradiol 24-hydroxylase by expressed sequence tag mining and functional expression assay. FEBS J. 273, 948-959.
Shinozaki, K., and Yamaguchi-Shinozaki, K. (2000). Molecular responses to dehydration and low temperature: differences and cross-talk between two stress signaling pathways. Curr. Opin. Plant Biol. 3, 217-223.
Siebertz, B., Logemann, J., Willmitzer, L., and Schell, J. (1989). cis-analysis of the wound-inducible promoter wunl in transgenic tobacco plants and histochemical localization of its expression. Plant Cell 1, 961-968.
Simm S, Papasotiriou DG, Ibrahim M, Leisegang M S, Muller B, Schorge T, Karas M, Minis O, Sommer M S, and Schleiff E. 2013. Defining the core proteome of the chloroplast envelope membranes. Frontiers in Plant Science. 4:176-193.
Sinclair, T. R. and Serraj, R. 1995. Legume nitrogen fixation and drought. Nature. 378. 344.
Smalle, J., Kurepa, J., Yang, P., Babiychuk, E., Kushnir, S., Durski, A., and Vierstra, R. D. (2002). Cytokinin Growth Responses in Arabidopsis Involve the 26S Proteasome Subunit RPN12. Plant Cell 14, 17-32.
Smart, C. J., Monéger, F., and Leaver, C. J. (1994). Cell-specific regulation of gene expression in mitochondria during anther development in sunflower. Plant Cell 6, 811-825.
Smibert and Krieg 1994, In: Gerhardt P, Murray R, Wood W, Krieg N (Eds) Methods for General and Molecular Bacteriology, ASM Press, Washington, D.C., pp 615-640
Soares et al. (1999). Revista de Microbiolgia 30(4): 299-303
Soriani, F. M., Kress, M. R., de Gouvea, P. F., Malavazi, I., Savoldi, M., Gallmetzer, A., Strauss, J., Goldman, M. H. S. and Goldman, G. H., 2009. Functional characterization of the Aspergillus nidulans methionine sulfoxide reductases (msrA and msrB). Fungal Genetics and Biology, 46(5), pp. 410-417.
Spiekermann et al. 1999, Arch Microbiol 171:73-80
Staswick, P. (1997). The occurrence and gene expression of vegetative storage proteins and a Rubisco Complex Protein in several perennial soybean species. J. Exp. Bot. 48, 2031-2036.
Staswick, P. E., Crafts-Brandner, S. J., and Salvucci, M. E. (1994). cDNA Sequence for the Ribulose 1,5 Bisphosphate Carboxylase/Oxygenase Complex Protein (A Protein that Accumulates in Soybean Leaves in Response to Fruit Removal). Plant Physiol. 105, 1445-1446.
Strepp, R., Scholz, S., Kruse, S., Speth, V., and Reski, R. (1998). Plant nuclear gene knockout reveals a role in plastid division for the homolog of the bacterial cell division protein FtsZ, an ancestral tubulin. Proc. Natl. Acad. Sci. 95, 4368-4373.
Strong, F. E., and Kruitwagen, E. (1967). Traumatic Acid: an Accelerator of Abscission in Cotton Explants. Nature 215, 1380-1381.
Su, L.-W., Chang, S. H., Li, M.-Y., Huang, H.-Y., Jane, W.-N., and Yang, J.-Y. (2013). Purification and biochemical characterization of Arabidopsis At-NEET, an ancient iron-sulfur protein, reveals a conserved cleavage motif for subcellular localization. Plant Sci. 213, 46-54.
Suárez, M. B., Sanz, L., Chamorro, M. I., Rey, M., González, F. J., Llobell, A. and Monte, E., 2005. Proteomic analysis of secreted proteins from Trichoderma harzianum: identification of a fungal cell wall-induced aspartic protease. Fungal Genetics and Biology, 42(11), pp. 924-934.
Sun, X.-L., Yu, Q.-Y., Tang, L.-L., Ji, W., Bai, X., Cai, H., Liu, X.-F., Ding, X.-D., and Zhu, Y.-M. (2013). GsSRK, a G-type lectin S-receptor-like serine/threonine protein kinase, is a positive regulator of plant tolerance to salt stress. J. Plant Physiol. 170, 505-515.
Sun, Y., Yi, X., Peng, M., Zeng, H., Wang, D., Li, B., Tong, Z., Chang, L., Jin, X., and Wang, X. (2014). Proteomics of Fusarium oxysporum Race 1 and Race 4 Reveals Enzymes Involved in Carbohydrate Metabolism and Ion Transport That Might Play Important Roles in Banana Fusarium Wilt. PLoS ONE 9, e113818.
Suorsa, M., Regel, R. E., Paakkarinen, V., Battchikova, N., Herrmann, R. G., and Aro, E.-M. (2004). Protein assembly of photosystem II and accumulation of subcomplexes in the absence of low molecular mass subunits PsbL and PsbJ. Eur. J. Biochem. 271, 96-107.
Sushil Tripathi, Karen R. Christie, Rama Balakrishnan, Rachael Huntley, David P. Hill, Liv Thommesen, Judith A. Blake, Martin Kuiper and Astrid Lxgreid. 2013. Gene Ontology annotation of sequence-specific DNA binding transcription factors: setting the stage for a large-scale curation effort. Database. 2013:bat062.
Suto et al., (2002) J Biosci Bioeng. 93:88-90
Suzuki, H., Takahashi, S., Watanabe, R., Fukushima, Y., Fujita, N., Noguchi, A., Yokoyama, R., Nishitani, K., Nishino, T., and Nakayama, T. (2006). An isoflavone conjugate-hydrolyzing beta-glucosidase from the roots of soybean (Glycine max) seedlings: purification, gene cloning, phylogenetics, and cellular localization. J. Biol. Chem. 281, 30251-30259.
Swiatek, M., Kuras, R., Sokolenko, A., Higgs, D., Olive, J., Cinque, G., Müller, B., Eichacker, L. A., Stern, D. B., Bassi, R., et al. (2001). The Chloroplast Gene ycf9 Encodes a Photosystem II (PSII) Core Subunit, PsbZ, That Participates in PSII Supramolecular Architecture. Plant Cell 13, 1347-1368.
T. Clint Nesbitt, Steven D. Tanksley. fw2.2 Directly Affects the Size of Developing Tomato Fruit, with Secondary Effects on Fruit Number and Photosynthate Distribution. Plant Physiol. 127(2):575-83.
Takahashi, A., Casais, C., Ichimura, K., and Shirasu, K. (2003). HSP90 interacts with RAR1 and SGT1 and is essential for RPS2-mediated disease resistance in Arabidopsis. Proc. Natl. Acad. Sci. 100, 11777-11782.
Takahashi, T., Gasch, A., Nishizawa, N., and Chua, N. H. (1995). The DIMINUTO gene of Arabidopsis is involved in regulating cell elongation. Genes Dev. 9, 97-107.
Takeuchi, Y., Yoshikawa, M., Takeba, G., Tanaka, K., Shibata, D., and Horino, 0. (1990). Molecular Cloning and Ethylene Induction of mRNA Encoding a Phytoalexin Elicitor-Releasing Factor, beta-1,3-Endoglucanase, in Soybean. Plant Physiol. 93, 673-682.
Tamir, S., Paddock, M. L., Darash-Yahana-Baram, M., Holt, S. H., Sohn, Y. S., Agranat, L., Michaeli, D., Stofleth, J. T., Lipper, C. H., Morcos, F., et al. (2015). Structure-function analysis of NEET proteins uncovers their role as key regulators of iron and ROS homeostasis in health and disease. Biochim. Biophys. Acta BBA—Mol. Cell Res. 1853, 1294-1315.
Terol, J., Soler, G., Talon, M., and Cercos, M. (2010). The aconitate hydratase family from Citrus. BMC Plant Biol. 10, 222.
Thomashow (2001) Plant Physiol. 125: 89-93
Torabi, S., Umate, P., Manayski, N., Plochinger, M., Kleinknecht, L., Bogireddi, H., Herrmann, R. G., Wanner, G., Schroder, W. P., and Meurer, J. (2014). PsbN Is Required for Assembly of the Photosystem II Reaction Center in Nicotiana tabacum. Plant Cell 26, 1183-1199.
UniProt Consortium (2015a). Glucan endo-1,3-beta-glucosidase GI—Hordeum vulgare (Barley).
UniProt Consortium (2015b). Protein P21—Glycine max (Soybean).
Unseld, M., Marienfeld, J. R., Brandt, P., and Brennicke, A. (1997). The mitochondrial genome of Arabidopsis thaliana contains 57 genes in 366,924 nucleotides. Nat. Genet. 15, 57-61
Urao, T., Yamaguchi-Shinozaki, K., Urao, S., and Shinozaki, K. (1993). An Arabidopsis myb homolog is induced by dehydration stress and its gene product binds to the conserved MYB recognition sequence. Plant Cell 5, 1529-1539.
Usadel & Fernie, (2013). Front Plant Sci. 4:48
Valente, M. A. S., Faria, J. A. Q. A., Soares-Ramos, J. R. L., Reis, P. A. B., Pinheiro, G. L., Piovesan, N. D., Morais, A. T., Menezes, C. C., Cano, M. A. O., Fietto, L. G., et al. (2009). The ER luminal binding protein (BiP) mediates an increase in drought tolerance in soybean and delays drought-induced leaf senescence in soybean and tobacco. J. Exp. Bot. 60, 533-546.
Van Gool, A. J., Verhage, R., Swagemakers, S. M., van de Putte, P., Brouwer, J., Troelstra, C., Bootsma, D., and Hoeijmakers, J. H. (1994). RAD26, the functional S. cerevisiae homolog of the Cockayne syndrome B gene ERCC6. EMBO J. 13, 5361-5369.
Van Hoof, P., and Gaspar, T. (1976). Peroxidase and isoperoxidase changes in relation to root initiation of Asparagus cultured in vitro. Sci. Hortic. 4, 27-31.
Vick, B., and Zimmerman, D. (1987). Oxidative systems for modification of fatty acids: the lipoxygenase pathway. In Lipids: Structure and Function. The Biochemistry of Plants, (New York), pp. 53-90.
Vining et al., (2013) BMC Plant Biol. 13:92
Waditee-Sirisattha, R., Hattori, A., Shibato, J., Rakwal, R., Sirisattha, S., Takabe, T., and Tsujimoto, M. (2011). Role of the Arabidopsis leucine aminopeptidase 2. Plant Signal. Behav. 6, 1581-1583.
Walter, K. L., Strachan, S. D., Ferry, N. M., Albert, H. H., Castle, L. A., and Sebastian, S. A. (2014). Molecular and phenotypic characterization of Als1 and Als2 mutations conferring tolerance to acetolactate synthase herbicides in soybean. Pest Manag. Sci. 70, 1831-1839.
Wan, T.-F., Shao, G.-H., Shan, X.-C., Zeng, N.-Y., and Lam, H.-M. (2006). Correlation between AS1 Gene Expression and Seed Protein Contents in Different Soybean (Glycine Max [L.] Merr.) Cultivars. Plant Biol. 8, 271-276.
Wang, L., Ma, H., Song, L., Shu, Y., and Gu, W. (2012). Comparative proteomics analysis reveals the mechanism of pre-harvest seed deterioration of soybean under high temperature and humidity stress. J. Proteomics 75, 2109-2127.
Wasternack, C. and Kombrink, E. 2009. Jasmonates: structural requirements for lipid-derived signals active in plant stress responses and development.
Weisshaar, B., and Jenkins, G. I. (1998). Phenylpropanoid biosynthesis and its regulation. Curr. Opin. Plant Biol. 1, 251-257.
Whetten, R. W., and Sederoff, R. R. (1992). Phenylalanine Ammonia-Lyase from Loblolly Pine Purification of the Enzyme and Isolation of Complementary DNA Clones. Plant Physiol. 98, 380-386.
Wong, S.-H., Lonhienne, T. G. A., Winzor, D. J., Schenk, G., and Guddat, L. W. (2012). Bacterial and Plant Ketol-Acid Reductoisomerases Have Different Mechanisms of Induced Fit during the Catalytic Cycle. J. Mol. Biol. 424, 168-179.
Wurster, B., and Hess, B. (1972). Glucose-6-phosphate-1-epimerase from baker's yeast. A new enzyme. FEBS Lett. 23, 341-344.
Yamagata, H., Masuzawa, T., Nagaoka, Y., Ohnishi, T., and Iwasaki, T. (1994). Cucumisin, a serine protease from melon fruits, shares structural homology with subtilisin and is generated from a large precursor. J. Biol. Chem. 269, 32725-32731.
Yan Y, Borrego E, and Kolomiets MV (2013). Jasmonate Biosynthesis, Perception and Function in Plant Development and Stress Responses. In Lipid Metabolism, (Intech), pp. 393-442.
Yan, Y., Borrego, E., and V., M. (2013). Jasmonate Biosynthesis, Perception and Function in Plant Development and Stress Responses. In Lipid Metabolism, R. Valenzuela Baez, ed. (InTech),
Yanagita, T. and Foster, J. W. 1956. A bacterial riboflavin hydrolase. J. Biol. Chem. 221, 593-607.
Yang, J., Schuster, G., and Stern, D. B. (1996). CSP41, a sequence-specific chloroplast mRNA binding protein, is an endoribonuclease. Plant Cell 8, 1409-1420.
Yang, X.-D., Li, W.-J., and Liu, J.-Y. (2005). Isolation and characterization of a novel PHGPx gene in Raphanus sativus. Biochim. Biophys. Acta BBA—Gene Struct. Expr. 1728, 199-205.
Yen, S.-K., Chung, M.-C., Chen, P.-C., and Yen, H. E. (2001). Environmental and Developmental Regulation of the Wound-Induced Cell Wall Protein WI12 in the Halophyte Ice Plant. Plant Physiol. 127, 517-528.
Yu, L., Niu, J.-S., Chen, P.-D., Ma, Z.-Q., and Liu, D.-J. (2005). Cloning, Physical Mapping and Expression Analysis of a Wheat Mlo-like Gene. J. Integr. Plant Biol. 47, 214-222.
Zakataeva, N. P., Romanenkov, D. V., Skripnikova, V. S., Vitushkina, M. V., Livshits, V. A., Kivero, A. D., and Novikova, A. E. (2011). Wild-type and feedback-resistant phosphoribosyl pyrophosphate synthetases from Bacillus amyloliquefaciens: purification, characterization, and application to increase purine nucleoside production. Appl. Microbiol. Biotechnol. 93, 2023-2033.
Zhang, D., Liang, W., Yin, C., Zong, J., Gu, F., and Zhang, D. (2010). OsC6, Encoding a Lipid Transfer Protein, Is Required for Postmeiotic Anther Development In Rice. Plant Physiol. 154, 149-162.
Zhang, J., Song, Q., Cregan, P. B., Nelson, R. L., Wang, X., Wu, J., and Jiang, G.-L. (2015). Genome-wide association study for flowering time, maturity dates and plant height in early maturing soybean (Glycine max) germplasm. BMC Genomics 16, 217.
Zhang, M., Wang, C., Lin, Q., Liu, A., Wang, T., Feng, X., Liu, J., Han, H., Ma, Y., Bonea, D., Zhao, R. and Hua, X. (2015), A tetratricopeptide repeat domain-containing protein SSR1 located in mitochondria is involved in root development and auxin polar transport in Arabidopsis. Plant J, 83: 582-599.
Zhang, Y., and Shih, D. S. (2007). Isolation of an osmotin-like protein gene from strawberry and analysis of the response of this gene to abiotic stresses. J. Plant Physiol. 164, 68-77.
Zhang, Y., Zhang, Y., Qiu, D., Zeng, H., Guo, L. and Yang, X., 2015. BcGs1, a glycoprotein from Botrytis cinerea, elicits defence response and improves disease resistance in host plants. Biochemical and biophysical research communications, 457(4), pp. 627-634.
Zhao, Z., Liu, H., Wang, C. and Xu, J. R., 2014. Erratum to: Comparative analysis of fungal genomes reveals different plant cell wall degrading capacity in fungi. BMC genomics, 15(1), p. 1.
Zhihong Hul, Fan Xu1, Liping Guan, Pingping Qian, Yaqiong Liu, Huifang Zhang, Yan Huang and Suiwen Hou. 2014. The tetratricopeptide repeat-containing protein slow green1 is required for chloroplast development in Arabidopsis. J Exp Bot. 65 (4): 1111-1123.
Zweib, C., Van Neus, R. W., Rosenblad, M. A., Brown, J. D., and Samuelsson, T. (2005). A nomenclature for all signal recognition particle RNAs. RNA 11, 7-13.

We claim:

1. A synthetic combination comprising a reproductive element from a plant, wherein said reproductive element is treated with a formulation comprising a purified Penicillium endophyte population, wherein said Penicillium endophyte is heterologous to the plant reproductive element, and comprises at least one composition selected from the group consisting of:
a. at least 500 nucleotides at least 95% identical to a nucleic acid sequence selected from the group consisting of: SEQ ID NO:1, SEQ ID NO: 2, SEQ ID NO: 4, SEQ ID NO:5;
b. at least 100 nucleotides at least 95% identical to SEQ ID NO: 3;
c. a strain selected from the group consisting of: ______ Deposit ID ______, ______ Deposit ID ______, ______ Deposit ID ______, ______ Deposit ID ______, or IDAC Deposit ID 081111-01; or
d. a Penicillium species selected from the group consisting of: SMCD2206, chrysogenum, olsonii, griseofulvum, or janthinellum;
wherein the endophyte is present in the synthetic combination in an amount capable of modulating at least one of: a trait of agronomic importance, the expression of a gene, the level of a transcript, the expression of a protein, the level of a hormone, the level of a metabolite, the population of endogenous microbes in plants grown from said plant reproductive element, as compared to an isoline plant grown from a plant reproductive element not contacted with said fungal endophyte.

2. The synthetic combination of claim 1, wherein said plant is selected from the group consisting of: soybean, and maize.

3. The synthetic combination of claim 1, wherein the formulation comprises a purified population of the Penicillium endophyte at a concentration of at least about 10̂2 CFU/ml in a liquid formulation or about 10̂2 CFU/gm in a non-liquid formulation.

4. The synthetic combination of claim 1, wherein said Penicillium endophyte is capable of auxin production, nitrogen fixation, production of an antimicrobial compound, mineral phosphate solubilization, siderophore production, cellulase production, chitinase production, xylanase production, or acetoin production.

5. The synthetic combination of claim 1, wherein said trait of agronomic importance is selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, or improved plant visual appearance.

6. The synthetic combination of claim 1, wherein said Penicillium endophyte is capable of localizing in a plant element of a plant grown from said seed, said plant element selected from the group consisting of: whole plant, seedling, meristematic tissue, ground tissue, vascular tissue, dermal tissue, seed, leaf, root, shoot, stem, flower, fruit, stolon, bulb, tuber, corm, keikis, or bud.

7. The synthetic combination of claim 1, wherein said plant reproductive element is a seed.

8. The synthetic combination of claim 1, wherein said plant reproductive element is placed into a substrate that promotes plant growth.

9. The synthetic combination of claim 8, wherein said substrate that promotes plant growth is soil.

10. The synthetic combination of claim 9, wherein a plurality of said plant reproductive elements are placed in the soil in rows, with substantially equal spacing between each seed within each row.

11. The synthetic combination of any of claims 1-10, wherein said formulation further comprises one or more of the following: stabilizer, preservative, carrier, surfactant, anticomplex agent, or any combination thereof.

12. The synthetic combination of any of claims 1-10, wherein said formulation further comprises one or more of the following: fungicide, nematicide, bactericide, insecticide, or herbicide.

13. The synthetic combination of any of claims 1-10, wherein said formulation further comprises at least one additional endophyte.

14. The synthetic combination of any of claims 1-10, wherein said plant reproductive element is a transgenic seed.

15. A plurality of synthetic combinations of claim 1, wherein said compositions are confined within an object selected from the group consisting of: bottle, jar, ampule, package, vessel, bag, box, bin, envelope, carton, container, silo, shipping container, truck bed, or case.

16. The synthetic combination of claim 1, wherein the Penicillium endophyte associated with the plant reproductive element is present in an amount capable of providing a benefit to said plant reproductive element or to a plant derived from said plant reproductive element.

17. The synthetic combination of claim 1, wherein the endophyte is present in at least two compartments of the plant reproductive element, selected from the group consisting of: embryo, seed coat, endosperm, cotyledon, hypocotyl, or radicle.

18. A plurality of synthetic combinations of claim 1, wherein the synthetic combinations are shelf-stable.

19. A plant grown from the synthetic combination of claim 1 under water-limited conditions, wherein said plant comprises at least one feature selected from the group consisting of:
a. at least 500 nucleotides at least 95% identical to a nucleic acid sequence selected from the group consisting of: SEQ ID NO:1, SEQ ID NO: 2, SEQ ID NO: 4, SEQ ID NO:5;
b. at least 100 nucleotides at least 95% identical to SEQ ID NO: 3;
c. an endophyte comprising a strain selected from the group consisting of: ______ Deposit ID ______, ______ Deposit ID ______, ______ Deposit ID ______, ______ Deposit ID ______, or IDAC Deposit ID 081111-01;
d. an endophyte comprising a Penicillium species selected from the group consisting of: SMCD2206, chrysogenum, olsonii, griseofulvum, or janthinellum;
e. at least one upregulated gene in root tissue, selected from the upregulated genes listed in Tables 7A, 7B, 7C, 7D, and 7E;
f. at least one upregulated gene in leaf tissue, selected from the upregulated genes listed in Tables 7A, 7B, 7C, 7D, and 7E;
g. at least one upregulated gene in stem tissue, selected from the upregulated genes listed in Tables 7A, 7B, 7C, 7D, and 7E;
h. at least one downregulated gene in root tissue, selected from the downregulated genes listed in Tables 7A, 7B, 7C, 7D, and 7E;
i. at least one downregulated gene in leaf tissue, selected from the downregulated genes listed in Tables 7A, 7B, 7C, 7D, and 7E;
j. at least one downregulated gene in stem tissue, selected from the downregulated genes listed in Tables 7A, 7B, 7C, 7D, and 7E;
k. at least one upregulated transcript in root tissue, selected from the upregulated transcripts listed in Table 7F;
l. at least one upregulated transcript in leaf tissue, selected from the upregulated transcripts listed in Table 7F;
m. at least one upregulated transcript in stem tissue, selected from the upregulated transcripts listed in Table 7F;
n. at least one downregulated transcript in root tissue, selected from the downregulated transcripts listed in Table 7F;
o. at least one downregulated transcript in leaf tissue, selected from the downregulated transcripts listed in Table 7F;
p. at least one downregulated transcript in stem tissue, selected from the downregulated transcripts listed in Table 7F;
q. increase in hormone level in root tissue, selected from the group consisting of: jasmonic acid, JA0ILE, 12-oxo-phytodienoic acid, 10-oxo-11-phytoenoic acid, traumatic acid;
r. decrease in hormone level in root tissue, selected from the group consisting of: abscisic acid, salicylic acid, CA;
s. increase in hormone level in stem tissue, selected from the group consisting of: salicylic acid, cinnamic acid, jasmonic acid isoleucine, 12-oxo-phytodienoic acid, 10-oxo-11-phytoenoic acid;
t. decrease in hormone level in stem tissue, selected from the group consisting of: abscisic acid, jasmonic acid, traumatic acid;
u. increase in hormone level in leaf tissue, selected from the group consisting of: salicylic acid, cinnamic acid, 12-oxo-phytodienoic acid, traumatic acid;
v. decrease in hormone level in leaf tissue, selected from the group consisting of: abscisic acid, jasmonic acid, jasmonic acid isoleucine, 10-oxo-11-phytoenoic acid;
w. increase in metabolite level in root tissue, selected from an increased metabolite in root tissue listed in Table 10;
x. decrease in metabolite level in root tissue, selected from a decreased metabolite in root tissue listed in Table 10;
y. increase in metabolite level in stem tissue, selected from an increased metabolite in stem tissue listed in Table 10;
z. decrease in metabolite level in stem tissue, selected from a decreased metabolite in stem tissue listed in Table 10;
aa. increase in metabolite level in leaf tissue, selected from an increased metabolite in leaf tissue listed in Table 10;
bb. decrease in metabolite level in leaf tissue, selected from a decreased metabolite in leaf tissue listed in Table 10;
cc. at least a 5% difference in prevalence of a taxonomic genus in leaf tissue, selected from a genus described in Table 11A;
dd. at least a 5% difference in prevalence of a taxonomic family in leaf tissue, selected from a family described in Table 11C;
ee. at least a 5% difference in prevalence of a taxonomic genus in root tissue, selected from a genus described in Table 11B or Table 11G;
ff. at least a 5% difference in prevalence of a taxonomic family in root tissue, selected from a family described in Table 11D or 11H;
gg. increase in abundance of microorganisms of the family Glomeraceae;
hh. increase in abundance of microorganisms of the genus Rhizophagus;
ii. increase in abundance of microorganisms of the genus Glomus;
jj. decrease in abundance of microorganisms of the family Enterobacteriaceae;
kk. decrease in abundance of microorganisms of the genus Escherhia-Shigella;
ll. presence of at least one OTU described in Table 11E;
mm. modulation of level of presence of at least one OTU selected from Table 11F.

20. A plant grown from the synthetic combination of claim 1, said plant exhibiting a trait of agronomic interest, selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, or improved plant visual appearance.

21. The plant of claim 20, wherein said plant is selected from the group consisting of: soybean, or maize.

22. The plant or progeny of the plant of claim 20, wherein said plant or progeny of the plant comprises in at least one of its plant elements said Penicillium endophyte.

23. A method for preparing a plant reproductive element synthetic combination, comprising contacting the surface of a plant reproductive element of a plant with a formulation comprising a purified microbial population that comprises a Penicillium endophyte that is heterologous to the plant reproductive element, and comprises at least one composition selected from the group consisting of:
a. at least 500 nucleotides at least 95% identical to a nucleic acid sequence selected from the group consisting of: SEQ ID NO:1, SEQ ID NO: 2, SEQ ID NO: 4, SEQ ID NO:5;
b. at least 100 nucleotides at least 95% identical to SEQ ID NO: 3;
c. a Deposit selected from the group consisting of: ______ Deposit ID ______, ______ Deposit ID ______, ______ Deposit ID ______, ______ Deposit ID ______, or IDAC Deposit ID 081111-01; or
d. a Penicillium species selected from the group consisting of: SMCD2206, chrysogenum, olsonii, griseofulvum, or janthinellum;
wherein the endophyte is present in the formulation in an amount capable of modulating at least one of: trait of agronomic importance, transcription of a gene, level of a transcript, the expression of a protein, level of a hormone, level of a metabolite, and population of endogenous microbes; in plants grown from said plant reproductive elements, as compared to isoline plants grown from plant reproductive elements not contacted with said formulation.

24. A method of modulating a trait of agronomic importance in a plant derived from a plant reproductive element, comprising treating said plant reproductive element with a formulation comprising a Penicillium endophyte that comprises at least one feature selected from the group consisting of:
a. at least 500 nucleotides at least 95% identical to a nucleic acid sequence selected from the group consisting of: SEQ ID NO:1, SEQ ID NO: 2, SEQ ID NO: 4, SEQ ID NO:5;
b. at least 100 nucleotides at least 95% identical to SEQ ID NO: 3;
c. ability to modulate the production of auxin;
d. ability to modulate the production of acetoin;
e. ability to modulate the production of a siderophore;
f. utilization of a primary carbon source selected from the group consisting of: L-Arabinose, L-Proline, D-Xylose, L-Glutamic acid, D-Ribose, L-Asparagine, Sucrose, Tween 80, Adonitol, L-Alanine, L-Alanyl-Glycine, L-Galactonic-acid-γ-lactone, β-Methyl-D-glucoside, m-Inositol, D-Galactose, D-Trehalose, D-Glucuronic acid, D-Gluconic acid, D-Mannitol, D-L-Malic acid, a-D-Glucose, Maltose, D-Melibiose, Maltotriose, Pyruvic acid, D-Galacturonic acid, D-Mannose, L-Threonine, Inosine, L-Lyxose, D-Alanine, L-Lactic acid, D-Galactonic acid-γ-lactone, Uridine, α-Hydroxy Glutaric acid-γ-lactone, D-L-α-Glycerol phosphate;
g. secretes at least one protein listed in Table 4C with at least a 2× higher rate, as compared to the strain represented by SEQ ID NO:6;
h. secretes at least one protein selected listed in Table 4D with at least a 0.8× lower rate, as compared to the strain represented by SEQ ID NO: 6;
i. secretes at least one protein selected from Table 4A; or
j. does not secrete a protein selected from the proteins listed in Table 4B.

25. A method of modulating a trait of agronomic importance in a plant derived from a plant reproductive element under water-limited conditions, comprising associating said plant reproductive element with a formulation comprising a Penicillium endophyte that is heterologous to the plant reproductive element, as compared to an isoline plant grown from a reproductive element not treated with said Penicillium endophyte; wherein said Penicillium endophyte comprises a composition selected from the group consisting of:
a. at least 500 nucleotides at least 95% identical to a nucleic acid sequence selected from the group consisting of: SEQ ID NO:1, SEQ ID NO: 2, SEQ ID NO: 4, SEQ ID NO:5;
b. at least 100 nucleotides at least 95% identical to SEQ ID NO: 3;
c. a Deposit selected from the group consisting of: ______ Deposit ID ______, ______ Deposit ID ______, ______ Deposit ID ______, ______ Deposit ID ______, or IDAC Deposit ID 081111-01; or
d. a Penicillium species selected from the group consisting of: SMCD2206, chrysogenum, olsonii, griseofulvum, or janthinellum;
and wherein said trait of agronomic importance is selected from the group consisting of:
e. upregulation of at least one gene in root tissue, selected from the upregulated genes listed in Tables 7A, 7B, 7C, 7D, and 7E;
f. upregulation of at least one gene in leaf tissue, selected from the upregulated genes listed in Tables 7A, 7B, 7C, 7D, and 7E;
g. upregulation of at least one gene in stem tissue, selected from the upregulated genes listed in Tables 7A, 7B, 7C, 7D, and 7E;
h. downregulation of at least one gene in root tissue, selected from the downregulated genes listed in Tables 7A, 7B, 7C, 7D, and 7E;
i. downregulation of at least one gene in leaf tissue, selected from the downregulated genes listed in Tables 7A, 7B, 7C, 7D, and 7E;
j. downregulation of at least one gene in stem tissue, selected from the downregulated genes listed in Tables 7A, 7B, 7C, 7D, and 7E;
k. upregulation of at least one transcript in root tissue, selected from the upregulated transcripts listed in Table 7F;
l. upregulation of at least one transcript in leaf tissue, selected from the upregulated transcripts listed in Table 7F;
m. upregulation of at least one transcript in stem tissue, selected from the upregulated transcripts listed in Table 7F;
n. downregulation of at least one transcript in root tissue, selected from the downregulated transcripts listed in Table 7F;
o. downregulation of at least one transcript in leaf tissue, selected from the downregulated transcripts listed in Table 7F;
p. downregulation of at least one transcript in stem tissue, selected from the downregulated transcripts listed in Table 7F;
q. increase in hormone level in root tissue, selected from the group consisting of: jasmonic acid, JAOILE, 12-oxo-phytodienoic acid, 10-oxo-11-phytoenoic acid, traumatic acid;
r. decrease in hormone level in root tissue, selected from the group consisting of: abscisic acid, salicylic acid, CA;
s. increase in hormone level in stem tissue, selected from the group consisting of: salicylic acid, cinnamic acid, jasmonic acid isoleucine, 12-oxo-phytodienoic acid, 10-oxo-11-phytoenoic acid;
t. decrease in hormone level in stem tissue, selected from the group consisting of: abscisic acid, jasmonic acid, traumatic acid;
u. increase in hormone level in leaf tissue, selected from the group consisting of: salicylic acid, cinnamic acid, 12-oxo-phytodienoic acid, traumatic acid;
v. decrease in hormone level in leaf tissue, selected from the group consisting of: abscisic acid, jasmonic acid, jasmonic acid isoleucine, 10-oxo-11-phytoenoic acid;
w. increase in metabolite level in root tissue, selected from an increased metabolite in root tissue listed in Table 10;
x. decrease in metabolite level in root tissue, selected from a decreased metabolite in root tissue listed in Table 10;
y. increase in metabolite level in stem tissue, selected from an increased metabolite in stem tissue listed in Table 10;
z. decrease in metabolite level in stem tissue, selected from a decreased metabolite in stem tissue listed in Table 10;
aa. increase in metabolite level in leaf tissue, selected from an increased metabolite in leaf tissue listed in Table 10;
bb. decrease in metabolite level in leaf tissue, selected from a decreased metabolite in leaf tissue listed in Table 10;
cc. at least a 5% difference in prevalence of a taxonomic genus in leaf tissue, selected from a genus described in Table 11A;
dd. at least a 5% difference in prevalence of a taxonomic family in leaf tissue, selected from a family described in Table 11C;
ee. at least a 5% difference in prevalence of a taxonomic genus in root tissue, selected from a genus described in Table 11B or Table 11G;
ff. at least a 5% difference in prevalence of a taxonomic family in root tissue, selected from a family described in Table 11D or 11H;
gg. increase in abundance of microorganisms of the family Glomeraceae;
hh. increase in abundance of microorganisms of the genus Rhizophagus;
ii. increase in abundance of microorganisms of the genus Glomus;
jj. decrease in abundance of microorganisms of the family Enterobacteriaceae;
kk. decrease in abundance of microorganisms of the genus Escherhia-Shigella;
ll. presence of at least one OTU described in Table 11E;
mm. modulation of level of presence of at least one OTU selected from Table 11F.

26. The method of any of claims 23-24, wherein said plant is selected from the group consisting of: soybean, or maize.

27. The method of any of claims 23-24, wherein the formulation comprises a purified population of the Penicillium endophyte at a concentration of at least about 10̂2 CFU/ml in a liquid formulation or about 10̂2 CFU/gm in a non-liquid formulation.

28. The method of any of claims 23-24, wherein said Penicillium endophyte is capable of auxin production, nitrogen fixation, production of an antimicrobial compound, mineral phosphate solubilization, siderophore production, cellulase production, chitinase production, xylanase production, or acetoin production.

29. The method of any of claims 23-24, wherein said trait of agronomic importance is selected from the group consisting of: disease resistance, drought tolerance, heat tolerance, cold tolerance, salinity tolerance, metal tolerance, herbicide tolerance, chemical tolerance, improved water use efficiency, improved nitrogen utilization, improved nitrogen fixation, pest resistance, herbivore resistance, pathogen resistance, increase in yield, increase in yield under water-limited conditions, health enhancement, vigor improvement, growth improvement, photosynthetic capability improvement, nutrition enhancement, altered protein content, altered oil content, increase in biomass, increase in shoot length, increase in root length, improved root architecture, increase in seed weight, altered seed carbohydrate composition, altered seed oil composition, increase in radical length, number of pods, delayed senescence, stay-green, altered seed protein composition, increase in dry weight of mature plant reproductive elements, increase in fresh weight of mature plant reproductive elements, increase in number of mature plant reproductive elements per plant, increase in chlorophyll content, increase in number of pods per plant, increase in length of pods per plant, reduced number of wilted leaves per plant, reduced number of severely wilted leaves per plant, increase in number of non-wilted leaves per plant, or improved plant visual appearance.

30. The method of any of claims 23-24, wherein said Penicillium endophyte is capable of localizing in a plant element of said plant, said plant element selected from the group consisting of: whole plant, seedling, meristematic tissue, ground tissue, vascular tissue, dermal tissue, seed, leaf, root, shoot, stem, flower, fruit, stolon, bulb, tuber, corm, keikis, or bud.

31. The method of any of claims 23-24, wherein said plant reproductive element is a seed.

32. The method of claim 31, wherein said seed is a transgenic seed.

33. The method of any of claims 23-24, wherein said plant reproductive element is placed into a substrate that promotes plant growth.

34. The method of claim 33, wherein said substrate that promotes plant growth is soil.

35. The method of claim 34, wherein a plurality of said plant reproductive elements are placed in the soil in rows, with substantially equal spacing between each within each row.

36. The method of any of claims 23-24, wherein said formulation further comprises one or more of the following: stabilizer, preservative, carrier, surfactant, anticomplex agent, or any combination thereof.

37. The method of any of claims 23-24, wherein said formulation further comprises one or more of the following: fungicide, nematicide, bactericide, insecticide, or herbicide.

38. The method of any of claims 23-24, wherein said formulation further comprises at least one additional endophyte.

39. The method of claim 23, wherein said plant reproductive element is a seed, and wherein the Penicillium endophyte is present in at least two compartments of the seed, selected from the group consisting of: embryo, seed coat, endosperm, cotyledon, hypocotyl, or radicle.

40. The method of claim 23, wherein the Penicillium endophyte is present in the plant reproductive element in an amount capable of providing a benefit to a plant derived from the plant reproductive element, as compared to a plant derived from a plant reproductive element not treated with said Penicillium endophyte.
